SEQUENCE LISTING (396 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Homo sapiens
  MOD_RES 30: note = AMIDATION
HAEGTFTSDVSSYLEGQAAKEFIAWLVKGR

SEQ ID NO: 3 - protein, 31 aa, synthetic construct
  REGION 1..31: note = Synthetic construct; Formula I
  VARIANT 1: note = Xaa at position 1 is selected from the group consisting of Y, H, D-Tyr, F, desH, and desY, or Xaa at position 1 and Xaa at position 2 combine to form desH-psi[NHCO]-Aib
  MOD_RES 1: note = The N-terminus of Xaa at position 1 is modified with R1, wherein the modification is selected from the group consisting of Ac and absent.
  VARIANT 2: note = Xaa at position 2 is selected from the group consisting of Aib, alpha-MeP, A, P, and D-Ala, or Xaa at position 1 combines with Xaa at position 2 to form desH-psi[NHCO]-Aib
  VARIANT 3: note = Xaa at position 3 is selected from the group of E, N, Aad, and cTA
  VARIANT 6: note = Xaa at position 6 is selected from the group consisting of F, alpha-MeF, and alpha-MeF(2F)
  VARIANT 10: note = Xaa at position 10 is selected from the group consisting of A, L, H, 3Pal, 4Pal, V, Y, E, alpha-MeF, alpha-MeF(2F), I, alpha-MeY, Q, D-His, D-Tyr, cTA, and K
  VARIANT 10: note = when Xaa at position 10 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  REGION 10..31: note = MISC_FEATURE - When Xaa is K(2-[2-(2-amino-ethoxy)- ethoxy]-acetyl)2-(gamma-Glu)-CO-(CH2)q-CO 2H at positions 10, 12, 13, 14, 16, 17, 19, 20, 21, 23, 24, 26, 27, 28, 29, 30 or 31, q is 14, 15, 16, 17, 18, 19 or 20.
  VARIANT 11: note = Xaa at position 11 is selected from the group consisting of S, alpha-MeS, or D-Ser
  VARIANT 12: note = Xaa at position 12 is selected from the group consisting of I, S, D-Ile, and K
  MOD_RES 12: note = when Xaa at position 12 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 13: note = Xaa at position 13 is selected from the group consisting of Nle, Aib, L, alpha-MeL, and K
  MOD_RES 13: note = when Xaa at position 13 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 14: note = Xaa at position 14 is selected from the group consisting of L and K, wherein K is conjugated to a C16-C22 fatty acid wherein said fatty acid is optionally conjugated to said K via a linker
  VARIANT 16: note = Xaa at position 16 is selected from the group consisting of E, Orn, Dab, Dap, S, T, H, Aib, alpha-MeK, R, and K
  MOD_RES 16: note = when Xaa at position 16 is K, then K is optionally chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 17: note = Xaa at position 17 is selected from the group consisting of K, Q, I, and an amino acid conjugated to a C16-C22 fatty acid wherein said fatty acid is optionally conjugated to said amino acid via a linker
  VARIANT 19: note = Xaa at position 19 is selected from the group consisting of Q, A, and K
  MOD_RES 19: note = when Xaa at position 19 is K, then K is chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 20: note = Xaa at position 20 is selected from the group consisting of Aib, Q, H, R, K, and alpha-MeK
  MOD_RES 20: note = when Xaa at position 20 is K, then K is optionally chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 21: note = Xaa at position 21 is selected from the group consisting of H, Aad, D, Aib, T, A, E, I, and K
  MOD_RES 21: note = when Xaa at position 21 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 22: note = Xaa at position 22 is selected from the group consisting of F and alpha-MeF
  VARIANT 23: note = Xaa at position 23 is selected from the group consisting of I, L, A, G, F, H, E, V, and K
  MOD_RES 23: note = when Xaa at position 23 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 24: note = Xaa at position 24 is selected from the group consisting of S, Aad, D-Glu, E, Aib, H, V, A, Q, D, P, and K
  MOD_RES 24: note = when Xaa at position 24 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 25: note = Xaa at position 25 is selected from the group consisting of Y or alpha-MeY
  VARIANT 26: note = Xaa at position 26 is selected from the group consisting of L, alpha-MeL, and K
  MOD_RES 26: note = when Xaa at position 26 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 27: note = Xaa at position 27 is selected from the group consisting of L, I, and K
  MOD_RES 27: note = when Xaa at position 27 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 28: note = Xaa at position 28 is selected from the group consisting of E, A, S, D-Glu, and K
  MOD_RES 28: note = when Xaa at position 28 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 29: note = Xaa at position 29 is selected from the group consisting of Aib, G, A, and K
  MOD_RES 29: note = when Xaa at position 29 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 30: note = Xaa at position 30 is selected from the group consisting of C, G, G-R2, and K, wherein R2 is a modification of the C-terminal group, wherein the modification is NH2 or absent, wherein if X30 is G-R2, then X31 is absent.
  MOD_RES 30: note = when Xaa at position 30 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 31: note = Xaa at position 31 is absent or is selected from the group consisting of SEQ ID NO:4, SEQ ID NO:5, SEQ ID:6, SEQ ID NO:7, SEQ ID NO:8, and SEQ ID NO:9
  SITE 31: note = MISC_FEATURE - wherein no more than one of X10, X12, X13, X14, X16, X17, X19, X20, X21, X23, X24, X26, X27, X28, X29, X30, X31, X32, X33, X34, X35, X36, X37, X38, X39, and X40 may be a substituent that contains a fatty acid
  SITE 31: note = MISC_FEATURE - wherein no more than one of X30, X34, X39, and X40 may be C
  SITE 31: note = MISC_FEATURE - wherein if one of X30, X34, X39, and X40 is C, then none of X10, X12, X13, X14, X16, X17, X19, X20, X21, X23, X24, X26, X27, X28, X29, X30, X31, X32, X33, X34, X35, X36, X37, X38, X39, and X40 is a substituent that contains a fatty acid
  MOD_RES 31: note = Xaa at position 31 is modified with R2, wherein the modification is NH2 to form a C-terminal amide or absent
XXXGTXTSDXXXXXDXXAXXXXXXXXXXXXX

SEQ ID NO: 4-9 - skipped (6 entries)

SEQ ID NO: 10 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = The Serine at position 39 is amidated
YXEGTXTSDYSIXLDEKAQXEFIXYLIEGGPSSGAPPPS

SEQ ID NO: 11 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = The Serine at position 39 is amidated
YXEGTXTSDYSIXLDXKAQXEFIXYLIEGGPSSGAPPPS

SEQ ID NO: 12 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)18-CO 2H
  SITE 20: note = MISC_FEATURE - Xaa at position 20 is Aib
  SITE 24: note = MISC_FEATURE - Xaa at position 24 is D-Glu
  SITE 39: note = MISC_FEATURE - The Serine at position 39 is amidated

SEQ ID NO: 13 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 10: note = MISC_FEATURE - Xaa at position 10 is 4Pal
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-MeY
  MOD_RES 39: note = The Serine at position 39 is amidated
YXEGTXTSDXSIXLDXKAQXEFIXXLIEGGPSSGAPPPS

SEQ ID NO: 14 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-MeY
  MOD_RES 39: note = The Serine at position 39 is amidated
YXEGTXTSDVSIXLDXKAQXEFIXXLIEGGPSSGAPPPS

SEQ ID NO: 15 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXAFIEYLLEGGPSSGAPPPS

SEQ ID NO: 16 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDSKAQXAFIEYLLEGGPSSGAPPPS

SEQ ID NO: 17 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXAFIEYLIEGGPSSGAPPPS

SEQ ID NO: 18 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDSKAQXAFIEYLIEGGPSSGAPPPS

SEQ ID NO: 19 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 24: note = Lys at position 24 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 29: note = MISC_FEATURE - Xaa at position 29 is Aib
  MOD_RES 39: note = Ser at position 39 is amidated
YXEGTFTSDYSILLDSIAQXAFIKYLLAXGPSSGAPPPS

SEQ ID NO: 20 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 29: note = MISC_FEATURE - Xaa at position 29 is Aib
  MOD_RES 39: note = Ser at position 39 is amidated
YXEGTFTSDYSILLDSIAQXAFIEYLLKXGPSSGAPPPS

SEQ ID NO: 21 - protein, 39 aa, synthetic construct
  REGION 1..39: note = Synthetic Construct
  SITE 2: note = MISC_FEATURE - Xaa at position 2 is Aib
  MOD_RES 20: note = Lys at position 20 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQKAFIEYLIEGGPSSGAPPPS

SEQ ID NO: 22 - protein, 39 aa, synthetic construct
  REGION 1..39: note = Synthetic Construct
  SITE 2: note = MISC_FEATURE - Xaa at position 2 is Aib
  SITE 16: note = MISC_FEATURE - Xaa at position 16 is Aib
  MOD_RES 20: note = Lys at position 20 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDXIAQKAFIEYLIEGGPSSGAPPPS

SEQ ID NO: 23 - protein, 39 aa, synthetic construct
  REGION 1..39: note = Synthetic Construct
  SITE 2: note = MISC_FEATURE - Xaa at position 2 is Aib
  MOD_RES 20: note = Lys at position 20 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 29: note = MISC_FEATURE - Xaa at position 29 is Aib
  MOD_RES 39: note = Ser at position 39 is amidated
YXEGTFTSDYSILLDKIAQKEFIQYLLEXGPSSGAPPPS

SEQ ID NO: 24 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 29: note = MISC_FEATURE - Xaa at position 29 is Aib
  MOD_RES 39: note = Ser at position 39 is amidated
HXEGTFTSDYSILLDKKAQXAFIEYLLEXGPSSGAPPPS

SEQ ID NO: 25 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 29: note = MISC_FEATURE - Xaa at position 29 is Aib
  MOD_RES 39: note = Ser at position 39 is amidated
HXEGTFTSDYSILLDKKAQXAFIEYLIEXGPSSGAPPPS

SEQ ID NO: 26 - protein, 34 aa, synthetic construct
  REGION 1..34: note = Synthetic Construct
  SITE 2: note = MISC_FEATURE - Xaa at position 2 is Aib
  MOD_RES 20: note = Lys at position 20 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 29: note = MISC_FEATURE - Xaa at position 29 is Aib
  MOD_RES 34: note = Ser at position 34 is amidated
HXEGTFTSDYSILLDKIAQKAFIEYLLEXGPSSG

SEQ ID NO: 27 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-L
  MOD_RES 17: note = Lys at position 17 is chemically modified by conjugation of the epsilon-amino group of the Lys side chain with Dab-(2-[2-(2-Amino-ethoxy)-ethoxy]-acetyl)-Dab- (2-[2-(2-Amino-eth oxy)-ethoxy]-acetyl)-CO-(CH2)18-CO2H
  SITE 20: note = MISC_FEATURE - Xaa at position 20 is alpha-methyl-Lys
  SITE 29: note = MISC_FEATURE - Xaa at position 29 is Aib
  MOD_RES 39: note = Ser at position 39 is amidated
HXEGTFTSDYSIXLDKKAQXAFIQYLLAXGPSSGAPKPS

SEQ ID NO: 28 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with Dab-(2-[2-(2-Amino-ethoxy)-ethoxy]-acetyl)-Dab- (2-[2-(2-Amino-eth oxy)-ethoxy]-acetyl)-CO-(CH2)18-CO2H
  SITE 20: note = MISC_FEATURE - Xaa at position 20 is alpha-methyl-Lys
  SITE 29: note = MISC_FEATURE - Xaa at position 29 is Aib
  MOD_RES 39: note = Ser at position 39 is amidated
HXEGTFTSDYSIXLDKKAQXAFIQYLLAXGPSSGAPPPS

SEQ ID NO: 29 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDKKAQXAFIEYLIEGGPSSGAPPPS

SEQ ID NO: 30 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is alpha-methyl-Lys
  MOD_RES 39: note = Ser at position 39 is amidated
YXEGTFTSDYSIXLDEKAQXAFIEYLLEGGPSSGAPPPS

SEQ ID NO: 31 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is alpha-methyl-Lys
  SITE 29: note = MISC_FEATURE - Xaa at position 29 is Aib
  MOD_RES 39: note = Ser at position 39 is amidated
YXEGTFTSDYSIXLDEKAQXAFIEYLLEXGPSSGAPPPS

SEQ ID NO: 32 - protein, 39 aa, synthetic construct
  REGION 1..39: note = Synthetic Construct
  SITE 2: note = MISC_FEATURE - Xaa at position 2 is Aib
  MOD_RES 10: note = Lys at position 10 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDKSILLDKIAQXAFIEYLIEGGPSSGAPPPS

SEQ ID NO: 33 - protein, 39 aa, synthetic construct
  REGION 1..39: note = Synthetic Construct
  SITE 2: note = MISC_FEATURE - Xaa at position 2 is Aib
  MOD_RES 11: note = Lys at position 11 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYKILLDKIAQXAFIEYLIEGGPSSGAPPPS

SEQ ID NO: 34 - protein, 39 aa, synthetic construct
  REGION 1..39: note = Synthetic Construct
  SITE 2: note = MISC_FEATURE - Xaa at position 2 is Aib
  MOD_RES 12: note = Lys at position 12 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSKLLDKIAQXAFIEYLIEGGPSSGAPPPS

SEQ ID NO: 35 - protein, 39 aa, synthetic construct
  REGION 1..39: note = Synthetic Construct
  SITE 2: note = MISC_FEATURE - Xaa at position 2 is Aib
  MOD_RES 13: note = Lys at position 13 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIKLDKIAQXAFIEYLIEGGPSSGAPPPS

SEQ ID NO: 36 - protein, 39 aa, synthetic construct
  REGION 1..39: note = Synthetic Construct
  SITE 2: note = MISC_FEATURE - Xaa at position 2 is Aib
  MOD_RES 14: note = Lys at position 14 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILKDKIAQXAFIEYLIEGGPSSGAPPPS

SEQ ID NO: 37 - protein, 39 aa, synthetic construct
  REGION 1..39: note = Synthetic Construct
  SITE 2: note = MISC_FEATURE - Xaa at position 2 is Aib
  MOD_RES 15: note = Lys at position 15 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLKKIAQXAFIEYLIEGGPSSGAPPPS

SEQ ID NO: 38 - protein, 39 aa, synthetic construct
  REGION 1..39: note = Synthetic Construct
  SITE 2: note = MISC_FEATURE - Xaa at position 2 is Aib
  MOD_RES 16: note = Lys at position 16 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKIAQXAFIEYLIEGGPSSGAPPPS

SEQ ID NO: 39 - protein, 39 aa, synthetic construct
  REGION 1..39: note = Synthetic Construct
  SITE 2: note = MISC_FEATURE - Xaa at position 2 is Aib
  MOD_RES 18: note = Lys at position 18 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKIKQXAFIEYLIEGGPSSGAPPPS

SEQ ID NO: 40 - protein, 39 aa, synthetic construct
  REGION 1..39: note = Synthetic Construct
  SITE 2: note = MISC_FEATURE - Xaa at position 2 is Aib
  MOD_RES 19: note = Lys at position 19 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKIAKXAFIEYLIEGGPSSGAPPPS

SEQ ID NO: 41 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 21: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXKFIEYLIEGGPSSGAPPPS

SEQ ID NO: 42 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 23: note = Lys at position 23 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFKEYLIEGGPSSGAPPPS

SEQ ID NO: 43 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 24: note = Lys at position 24 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIKYLIEGGPSSGAPPPS

SEQ ID NO: 44 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 26: note = Lys at position 26 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYKIEGGPSSGAPPPS

SEQ ID NO: 45 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 27: note = Lys at position 27 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLKEGGPSSGAPPPS

SEQ ID NO: 46 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIKGGPSSGAPPPS

SEQ ID NO: 47 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 29: note = Lys at position 29 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEKGPSSGAPPPS

SEQ ID NO: 48 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 30: note = Lys at position 30 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGKPSSGAPPPS

SEQ ID NO: 49 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 31: note = Lys at position 31 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGKSSGAPPPS

SEQ ID NO: 50 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 32: note = Lys at position 32 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPKSGAPPPS

SEQ ID NO: 51 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 33: note = Lys at position 33 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSKGAPPPS

SEQ ID NO: 52 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 34: note = Lys at position 34 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSKAPPPS

SEQ ID NO: 53 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 35: note = Lys at position 35 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGKPPPS

SEQ ID NO: 54 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 36: note = Lys at position 36 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGAKPPS

SEQ ID NO: 55 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 37: note = Lys at position 37 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGAPKPS

SEQ ID NO: 56 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 38: note = Lys at position 38 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGAPPKS

SEQ ID NO: 57 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 39: note = Lys at position 39 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Lys at position 39 is amidated
YXEGTFTSDYSILLDKIAQXAFIEYLIEGGPSSGAPPPK

SEQ ID NO: 58 - protein, 40 aa, synthetic construct
  REGION 1..40: note = Synthetic Construct
  SITE 2: note = MISC_FEATURE - Xaa at position 2 is Aib
  SITE 20: note = MISC_FEATURE - Xaa at position 20 is Aib
  MOD_RES 40: note = Lys at position 40 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 40: note = Lys at position 40 is amidated
YXEGTFTSDYSILLDKIAQXAFIEYLIEGGPSSGAPPPSK

SEQ ID NO: 59 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTFTSDYSILLDKKAQXAFIEYLIEGGPSSG

SEQ ID NO: 60 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSILLDKKAQXAFIEYLIEGG

SEQ ID NO: 61 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDSKAQXAFIEYLIEGGPSSGAPPPS

SEQ ID NO: 62 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
HXEGTFTSDYSIXLDKKAQXAFIEYLIEGGPSSGAPPPS

SEQ ID NO: 63 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
HXEGTFTSDYSIXLDKKAQHAFIEYLIEGGPSSGAPPPS

SEQ ID NO: 64 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
HXEGTFTSDYSIXLDEKAQHAFIEYLIEGGPSSGAPPPS

SEQ ID NO: 65 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSIXLDEKAQHAFIEYLIEGGPSSGAPPPS

SEQ ID NO: 66 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSILLDKKAQXAFIEYLIEGGPSSGAPPPS

SEQ ID NO: 67 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSSLLDKKAQXAFIEYLIEGGPSSGAPPPS

SEQ ID NO: 68 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDEKAQXAFIEYLIEGGPSSGAPPPS

SEQ ID NO: 69 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDXKAQXAFIEYLIEGGPSSGAPPPS

SEQ ID NO: 70 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDSKAQXAFIEYLLEGGPSSGAPPPS

SEQ ID NO: 71 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDKKAQXAFIEYLIEGG

SEQ ID NO: 72 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDEKAQXAFIEYLIEGG

SEQ ID NO: 73 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDSKAQXAFIEYLIEGG

SEQ ID NO: 74 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDTKAQXAFIEYLIEGG

SEQ ID NO: 75 - protein, 39 aa, synthetic construct; identical to SEQ ID NO: 30
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 76 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 22: note = MISC_FEATURE - Xaa at position 22 is alpha-methyl-Phe
  MOD_RES 39: note = Ser at position 39 is amidated
YXEGTFTSDYSILLDKKAQXAXIEYLIEGGPSSGAPPPS

SEQ ID NO: 77 - protein, 39 aa, synthetic construct
  REGION 1..39: note = Synthetic Construct
  SITE 2: note = MISC_FEATURE - Xaa at position 2 is Aib
  SITE 11: note = MISC_FEATURE - Xaa at position 11 is alpha-methyl-Ser
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYXILLDKKAQXAFIEYLIEGGPSSGAPPPS

SEQ ID NO: 78 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXAFIEYLIEGGPSSGAPPPS

SEQ ID NO: 79 - protein, 30 aa, synthetic construct
  REGION 1..30: note = Synthetic Construct
  SITE 2: note = MISC_FEATURE - Xaa at position 2 is Aib
  MOD_RES 10: note = Lys at position 10 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDKSILLDKIAQXAFIEYLIEGG

SEQ ID NO: 80 - protein, 30 aa, synthetic construct
  REGION 1..30: note = Synthetic Construct
  SITE 2: note = MISC_FEATURE - Xaa at position 2 is Aib
  MOD_RES 14: note = Lys at position 14 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSILKDKIAQXAFIEYLIEGG

SEQ ID NO: 81 - protein, 30 aa, synthetic construct
  REGION 1..30: note = Synthetic Construct
  SITE 2: note = MISC_FEATURE - Xaa at position 2 is Aib
  SITE 20: note = MISC_FEATURE - Xaa at position 20 is Aib
  MOD_RES 21: note = Lys at position 21 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTFTSDYSILLDKIAQXKFIEYLIEGG

SEQ ID NO: 82 - protein, 30 aa, synthetic construct
  REGION 1..30: note = Synthetic Construct
  SITE 2: note = MISC_FEATURE - Xaa at position 2 is Aib
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTFTSDYSILLDKIAQXAFIEYLIKGG

SEQ ID NO: 83 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 22: note = MISC_FEATURE - Xaa at position 22 is alpha-methyl-Phe
  MOD_RES 39: note = Ser at position 39 is amidated
YXEGTFTSDYSIXLDKKAQXAXIEYLIEGGPSSGAPPPS

SEQ ID NO: 84 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 26: note = MISC_FEATURE - Xaa at position 26 is alpha-methyl-Leu
  MOD_RES 39: note = Ser at position 39 is amidated
YXEGTFTSDYSIXLDKKAQXAFIEYXIEGGPSSGAPPPS

SEQ ID NO: 85 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 22: note = MISC_FEATURE - Xaa at position 22 is alpha-methyl-Phe
  MOD_RES 39: note = Ser at position 39 is amidated
YXEGTXTSDYSIXLDKKAQXAXIEYLIEGGPSSGAPPPS

SEQ ID NO: 86 - protein, 38 aa, synthetic construct
  REGION 1..38: note = Synthetic Construct
  SITE 2: note = MISC_FEATURE - Xaa at position 2 is Aib
  SITE 19: note = MISC_FEATURE - Xaa at position 19 is Aib
  MOD_RES 37: note = Lys at position 37 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 38: note = Ser at position 38 is amidated
YXEGTFTSDYSILLKIAQXAFIEYLIEGGPSSGAPPKS

SEQ ID NO: 87 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSILLDKKAQXAFIEYLIEGGPSSGAPPPS

SEQ ID NO: 88 - protein, 39 aa, synthetic construct
  REGION 1..39: note = Synthetic Construct
  SITE 1: note = MISC_FEATURE - Xaa at position 1 is D-Tyr
  MOD_RES 1: note = The N-terminal amino is acetylated
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XAEGTFTSDYSILLDKKAQXAFIEYLIEGGPSSGAPPPS

SEQ ID NO: 89 - protein, 39 aa, synthetic construct; identical to SEQ ID NO: 17
  REGION 1..39: note = Synthetic Construct
  SITE 2: note = MISC_FEATURE - Xaa at position 2 is D-Ala
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 90 - protein, 39 aa, synthetic construct; identical to SEQ ID NO: 77
  REGION 1..39: note = Synthetic Construct
  SITE 2: note = MISC_FEATURE - Xaa at position 2 is Aib
  SITE 11: note = MISC_FEATURE - Xaa at position 11 is D-Ser
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 91 - protein, 39 aa, synthetic construct
  REGION 1..39: note = Synthetic Construct
  SITE 2: note = MISC_FEATURE - Xaa at position 2 is Aib
  SITE 12: note = MISC_FEATURE - Xaa at position 12 is D-Ile
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSXLLDKKAQXAFIEYLIEGGPSSGAPPPS

SEQ ID NO: 92 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 28: note = MISC_FEATURE - Xaa at position 28 is D-Glu
  MOD_RES 39: note = Ser at position 39 is amidated
YXEGTFTSDYSILLDKKAQXAFIEYLIXGGPSSGAPPPS

SEQ ID NO: 93 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTFTSDYSILLDKKAQXAFIXYLIEGGPSSGAPPPS

SEQ ID NO: 94 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDASILLDKKAQXAFIEYLIEGGPSSGAPPPS

SEQ ID NO: 95 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXAFIEYLIEAGPSSGAPPPS

SEQ ID NO: 96 - protein, 39 aa, synthetic construct; identical to SEQ ID NO: 17
  REGION 1..39: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 97 - protein, 39 aa, synthetic construct
  REGION 1..39: note = Synthetic Construct
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YPEGTFTSDYSILLDKKAQXAFIEYLIEGGPSSGAPPPS

SEQ ID NO: 98 - protein, 39 aa, synthetic construct
  REGION 1..39: note = Synthetic Construct
  SITE 2: note = MISC_FEATURE - Xaa at position 2 is Aib
  SITE 3: note = MISC_FEATURE - Xaa at position 3 is Aad
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXXGTFTSDYSILLDKKAQXAFIEYLIEGGPSSGAPPPS

SEQ ID NO: 99 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXNGTFTSDYSILLDKKAQXAFIEYLIEGGPSSGAPPPS

SEQ ID NO: 100 - protein, 39 aa, synthetic construct; identical to SEQ ID NO: 98
  REGION 1..39: note = Synthetic Construct
  SITE 2: note = MISC_FEATURE - Xaa at position 2 is Aib
  SITE 3: note = MISC_FEATURE - Xaa at position 3 is gamma-Glu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 101 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 10: note = Lys at position 10 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDKSILLDKIAQXAFIEYLIEGG

SEQ ID NO: 102 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 14: note = Lys at position 14 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 30: note = MISC_FEATURE - Gly at position 30 is amidated
YXEGTXTSDYSILKDKIAQXAFIEYLIEGG

SEQ ID NO: 103 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 20: note = MISC_FEATURE - Xaa at position 20 is Aib
  MOD_RES 21: note = Lys at position 21 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 30: note = MISC_FEATURE - Gly at position 30 is amidated
YXEGTXTSDYSILLDKIAQXKFIEYLIEGG

SEQ ID NO: 104 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTXTSDYSILLDKIAQXAFIEYLIKGG

SEQ ID NO: 105 - protein, 30 aa, synthetic construct
  REGION 1..30: note = Synthetic Construct
  SITE 2: note = MISC_FEATURE - Xaa at position 2 is Aib
  MOD_RES 10: note = Lys at position 10 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 13: note = MISC_FEATURE - Xaa at position 13 is alpha-methyl-Leu
  SITE 20: note = MISC_FEATURE - Xaa at position 20 is Aib
  MOD_RES 30: note = Gly at position 30 is amidated
YXEGTFTSDKSIXLDKIAQXAFIEYLIEGG

SEQ ID NO: 106 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTFTSDYSIXLDKIAQXAFIEYLIKGG

SEQ ID NO: 107 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 13 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSILLDKKAQXAFIEYLIEGG

SEQ ID NO: 108 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSIXLDKKAQXAFIEYLIEGG

SEQ ID NO: 109 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSIXLDEKAQXAFIEYLIEGG

SEQ ID NO: 110 - protein, 30 aa, synthetic construct; identical to SEQ ID NO: 109
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated

SEQ ID NO: 111 - protein, 30 aa, synthetic construct; identical to SEQ ID NO: 109
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated

SEQ ID NO: 112 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSIXLDEKAQXEFIEYLIEGG

SEQ ID NO: 113 - protein, 30 aa, synthetic construct; identical to SEQ ID NO: 112
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated

SEQ ID NO: 114 - protein, 39 aa, synthetic construct; identical to SEQ ID NO: 87
  REGION 1..39: note = Synthetic Construct
  REGION 1..2: note = MISC_FEATURE - Xaa at position 1 combines with Xaa at position 2 to form desHis-psi[NHCO]-Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 115 - protein, 39 aa, synthetic construct; identical to SEQ ID NO: 87
  REGION 1..39: note = Synthetic Construct
  REGION 1..2: note = MISC_FEATURE - Xaa at position 1 combines with Xaa at position 2 to form desHis-Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 116 - protein, 39 aa, synthetic construct; identical to SEQ ID NO: 87
  REGION 1..39: note = Synthetic Construct
  SITE 1: note = MISC_FEATURE - Xaa at position 1 is Des-Tyr
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 117 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-AOC-(gam ma-Glu)-CO-(CH2)18 -CO2H
  SITE 20: note = MISC_FEATURE - Xaa at position 20 is Aib
  MOD_RES 39: note = Ser at position 39 is amidated

SEQ ID NO: 118 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with AOC-(2-[2-(2-amino-ethoxy)-ethoxy]-acetyl)-(gam ma-Glu)-CO-(CH2)18 -CO2H
  SITE 20: note = MISC_FEATURE - Xaa at position 20 is Aib
  MOD_RES 39: note = Ser at position 39 is amidated

SEQ ID NO: 119 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-(Trx)-CO-(CH2) 18-CO2H
  SITE 20: note = MISC_FEATURE - Xaa at position 20 is Aib
  MOD_RES 39: note = Ser at position 39 is amidated

SEQ ID NO: 120 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(Trx)-(g amma-Glu)-CO-(CH2) 18-CO2H
  SITE 20: note = MISC_FEATURE - Xaa at position 20 is Aib
  MOD_RES 39: note = Ser at position 39 is amidated

SEQ ID NO: 121 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(epsilon -Lys)-(gamma-Glu)- CO-(CH2)18-CO2H
  SITE 20: note = MISC_FEATURE - Xaa at position 20 is Aib
  MOD_RES 39: note = Ser at position 39 is amidated

SEQ ID NO: 122 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  MOD_RES 39: note = Ser at position 39 is amidated

SEQ ID NO: 123 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(epsilon -Lys)-(epsilon-Lys )-CO-(CH2)18-CO2H
  SITE 20: note = MISC_FEATURE - Xaa at position 2 is Aib
  MOD_RES 39: note = Ser at position 39 is amidated

SEQ ID NO: 124 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXAFIEYLIEGGPSSGAPPPS

SEQ ID NO: 125 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIEYLIEGGPSSGAPPPS

SEQ ID NO: 126 - protein, 39 aa, synthetic construct; identical to SEQ ID NO: 124
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 127 - protein, 39 aa, synthetic construct; identical to SEQ ID NO: 125
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 128 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
FXEGTXTSDYSIXLDEKAQXAFIEYLIEGGPSSGAPPPS

SEQ ID NO: 129 - protein, 39 aa, synthetic construct
  REGION 1..39: note = Synthetic Construct
  SITE 2: note = MISC_FEATURE - Xaa at position 2 is Aib
  SITE 3: note = MISC_FEATURE - Xaa at position 3 is cTA
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXXGTXTSDYSIXLDEKAQXAFIEYLIEGGPSSGAPPPS

SEQ ID NO: 130 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTXTSDYSIXLDEKAQQAFIEYLIEGGPSSGAPPPS

SEQ ID NO: 131 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 29: note = MISC_FEATURE - Xaa at position 29 is Aib
  MOD_RES 39: note = Ser at position 39 is amidated
YXEGTXTSDYSIXLDEKAQQAFIEYLIEXGPSSGAPPPS

SEQ ID NO: 132 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTXTSDYSIXLDKKAQXEFIEYLIEGGPSSG

SEQ ID NO: 133 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTXTSDYSIXLDEKAQXEFIEYLIEGGPSSG

SEQ ID NO: 134 - protein, 39 aa, synthetic construct; identical to SEQ ID NO: 78
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)2-CO-(CH2)18-C O2H
  SITE 20: note = MISC_FEATURE - Xaa at position 20 is Aib
  MOD_RES 39: note = Ser at position 39 is amidated

SEQ ID NO: 135 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXAFIEYLIEAGPSSGAPPPS

SEQ ID NO: 136 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDISILLDKKAQXAFIEYLIEGGPSSGAPPPS

SEQ ID NO: 137 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDHSILLDKKAQXAFIEYLIEGGPSSGAPPPS

SEQ ID NO: 138 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDLSILLDKKAQXAFIEYLIEGGPSSGAPPPS

SEQ ID NO: 139 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDESILLDKKAQXAFIEYLIEGGPSSGAPPPS

SEQ ID NO: 140 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 10: note = MISC_FEATURE - Xaa at position 10 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDXSILLDKKAQXAFIEYLIEGGPSSGAPPPS

SEQ ID NO: 141 - protein, 39 aa, synthetic construct; identical to SEQ ID NO: 140
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 10: note = MISC_FEATURE - Xaa at position 10 is 3Pal
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 142 - protein, 39 aa, synthetic construct
  REGION 1..39: note = Synthetic Construct
  SITE 1: note = MISC_FEATURE - Xaa at position 1 is DesTyr
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTXTSDYSIXLDEKAQXAFIEYLIEGGPSSGAPPPS

SEQ ID NO: 143 - protein, 39 aa, synthetic construct
  REGION 1..39: note = Synthetic Construct
  SITE 1: note = MISC_FEATURE - Xaa at position 1 is DesTyr
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTXTSDYSIXLDEKAQXEFIEYLIEGGPSSGAPPPS

SEQ ID NO: 144 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
HXNGTFTSDYSILLDKKAQXAFIEYLIEGGPSSGAPPPS

SEQ ID NO: 145 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDASILLDKKAQXAFIEYLIEAGPSSGAPPPS

SEQ ID NO: 146 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 21: note = MISC_FEATURE - Xaa at position 21 is Aad
  MOD_RES 39: note = Ser at position 39 is amidated
YXEGTXTSDYSIXLDKKAQXXFIEYLIEGGPSSGAPPPS

SEQ ID NO: 147 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXSFIEYLIEGGPSSGAPPPS

SEQ ID NO: 148 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXTFIEYLIEGGPSSGAPPPS

SEQ ID NO: 149 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXDFIEYLIEGGPSSGAPPPS

SEQ ID NO: 150 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXIFIEYLIEGGPSSGAPPPS

SEQ ID NO: 151 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 2 is Aib
  MOD_RES 39: note = Ser at position 39 is amidated
YXEGTXTSDYSIXLDKKAQXHFIEYLIEGGPSSGAPPPS

SEQ ID NO: 152 - protein, 39 aa, synthetic construct; identical to SEQ ID NO: 146
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 21: note = MISC_FEATURE - Xaa at position 21 is Aib
  MOD_RES 39: note = Ser at position 39 is amidated

SEQ ID NO: 153 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 21: note = MISC_FEATURE - Xaa at position 21 is Aib
  MOD_RES 39: note = Ser at position 39 is amidated
YXEGTXTSDYSIXLDKKAQHXFIEYLIEGGPSSGAPPPS

SEQ ID NO: 154 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDASIXLDKKAQXAFIEYLIEGGPSSGAPPPS

SEQ ID NO: 155 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIQYLIEGGPSSGAPPPS

SEQ ID NO: 156 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aad
  MOD_RES 39: note = Ser at position 39 is amidated

SEQ ID NO: 157 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIAYLIEGGPSSGAPPPS

SEQ ID NO: 158 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIVYLIEGGPSSGAPPPS

SEQ ID NO: 159 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFISYLIEGGPSSGAPPPS

SEQ ID NO: 160 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIPYLIEGGPSSGAPPPS

SEQ ID NO: 161 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aib
  MOD_RES 39: note = Ser at position 39 is amidated

SEQ ID NO: 162 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIHYLIEGGPSSGAPPPS

SEQ ID NO: 163 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDHSIXLDEKAQXEFIEYLIEGGPSSGAPPPS

SEQ ID NO: 164 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is cTA
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDXSIXLDEKAQXEFIEYLIEGGPSSGAPPPS

SEQ ID NO: 165 - protein, 39 aa, synthetic construct; identical to SEQ ID NO: 164
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 2Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 166 - protein, 39 aa, synthetic construct; identical to SEQ ID NO: 164
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 167 - protein, 39 aa, synthetic construct; identical to SEQ ID NO: 164
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 168 - protein, 39 aa, synthetic construct; identical to SEQ ID NO: 164
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 169 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDEKAQXTFIXYLIEGGPSSGAPPPS

SEQ ID NO: 170 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)2-CO-(CH2)18-C O2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDHSIXLDEKAQXTFIXYLIEGGPSSG

SEQ ID NO: 171 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)2-CO-(CH2)18-C O2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDHSIXLDEKAQXTFIXHLIEGGPSSG

SEQ ID NO: 172 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
HXEGTXTSDYSIXLDEKAQXEFIEYLIEGGPSSGAPPPS

SEQ ID NO: 173 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  SITE 28: note = MISC_FEATURE - Xaa at position 28 is D-Glu
  MOD_RES 39: note = Ser at position 39 is amidated
YXEGTXTSDHSIXLDEKAQXTFIXYLIXGGPSSGAPPPS

SEQ ID NO: 174 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Phe
  SITE 28: note = MISC_FEATURE - Xaa at position 28 is D-Glu
  MOD_RES 39: note = Ser at position 39 is amidated
YXEGTXTSDHSIXLDEKAQXTFIXXLIXGGPSSGAPPPS

SEQ ID NO: 175 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 30: note = Gly at position 30 is amidated
YXEGTXTSDHSIXLDEKAQXTFIXYLIEGG

SEQ ID NO: 176 - protein, 34 aa, synthetic construct; identical to SEQ ID NO: 170
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated

SEQ ID NO: 177 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDEKAQXTFIXYLIEGGPSSGAPPPS

SEQ ID NO: 178 - protein, 39 aa, synthetic construct; identical to SEQ ID NO: 177
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 179 - protein, 39 aa, synthetic construct; identical to SEQ ID NO: 177
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)14-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 180 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDXKAQXTFIXYLIEGGPSSGAPPPS

SEQ ID NO: 181 - protein, 39 aa, synthetic construct; identical to SEQ ID NO: 180
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is a Aib
  SITE 24: note = MISC_FEATURE - Xaa at position 24 is D-Glu
  MOD_RES 39: note = Ser at position 39 is amidated

SEQ ID NO: 182 - protein, 39 aa, synthetic construct; identical to SEQ ID NO: 180
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 183 - protein, 39 aa, synthetic construct; identical to SEQ ID NO: 177
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 184 - protein, 39 aa, synthetic construct; identical to SEQ ID NO: 177
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 185 - protein, 39 aa, synthetic construct; identical to SEQ ID NO: 177
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)16- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 186 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDEKAQXHFIXYLIEGGPSSGAPPPS

SEQ ID NO: 187 - protein, 39 aa, synthetic construct; identical to SEQ ID NO: 186
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)14-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 188 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDHSIXLDEKAQXHFIXYLIEGGPSSG

SEQ ID NO: 189 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 30: note = Gly at position 30 is amidated
YXEGTXTSDHSIXLDEKAQXHFIXYLIEGG

SEQ ID NO: 190 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDEKAQXEFIXYLIEGGPSSGAPPPS

SEQ ID NO: 191 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 192 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDLSIXLDEKAQXEFIXYLIEGGPSSGAPPPS

SEQ ID NO: 193 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is D-Tyr
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 194 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is D-His
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 195 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is alpha-methyl-Tyr
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 196 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDQSIXLDEKAQXEFIXYLIEGGPSSGAPPPS

SEQ ID NO: 197 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aib
  MOD_RES 39: note = Ser at position 39 is amidated

SEQ ID NO: 198 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aib
  MOD_RES 39: note = Ser at position 39 is amidated

SEQ ID NO: 199 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
HXEGTXTSDXSIXLDEKAQXEFIXYLIEGGPSSGAPPPS

SEQ ID NO: 200 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDVSIXLDEKAQXEFIXYLIEGGPSSGAPPPS

SEQ ID NO: 201 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDASIXLDEKAQXEFIXYLIEGGPSSGAPPPS

SEQ ID NO: 202 - protein, 39 aa, synthetic construct
  REGION 1..39: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDEKAQQAFIEYLIEGGPSSGAPPPS

SEQ ID NO: 203 - protein, 39 aa, synthetic construct
  REGION 1..39: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDEKAQHAFIEYLIEGGPSSGAPPPS

SEQ ID NO: 204 - protein, 39 aa, synthetic construct
  REGION 1..39: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDRKAQQAFIEYLIEGGPSSGAPPPS

SEQ ID NO: 205 - protein, 39 aa, synthetic construct
  REGION 1..39: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDRKAQHAFIEYLIEGGPSSGAPPPS

SEQ ID NO: 206 - protein, 34 aa, synthetic construct
  REGION 1..34: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 34: note = Gly at position 34 is amidated
YXEGTFTSDYSILLDEKAQQAFIEYLIEGGPSSG

SEQ ID NO: 207 - protein, 34 aa, synthetic construct
  REGION 1..34: note = Synthetic Construct
  SITE 1: note = MISC_FEATURE - Xaa at position 1 is D-Tyr
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 34: note = Gly at position 34 is amidated
XXEGTFTSDYSILLDEKAQQAFIEYLIEGGPSSG

SEQ ID NO: 208 - protein, 39 aa, synthetic construct
  REGION 1..39: note = Synthetic Construct
  SITE 1: note = MISC_FEATURE - Xaa at position 1 is DesTyr
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
XXEGTFTSDYSILLDEKAQQAFIEYLIEGGPSSGAPPPS

SEQ ID NO: 209 - protein, 39 aa, synthetic construct
  REGION 1..39: note = Synthetic Construct
  SITE 1: note = MISC_FEATURE - Xaa at position 1 is DesTyr
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
XAEGTFTSDYSILLDEKAQQAFIEYLIEGGPSSGAPPPS

SEQ ID NO: 210 - protein, 39 aa, synthetic construct; identical to SEQ ID NO: 208
  REGION 1..39: note = Synthetic Construct
  SITE 1: note = MISC_FEATURE - Xaa at position 1 is DesHis
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated

SEQ ID NO: 211 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)-(gamma-G lu)-(2-[2-(2-Amino -ethoxy)-ethoxy]-acetyl)-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDKKAQXEFIXYLIEGGPSSGAPPPS

SEQ ID NO: 212 - protein, 39 aa, synthetic construct; identical to SEQ ID NO: 211
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 213 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 214 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)16-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 215 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 216 - protein, 39 aa, synthetic construct; identical to SEQ ID NO: 190
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 217 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)14-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 218 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 219 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 220 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)-(gamma-G lu)-(2-[2-(2-Amino -ethoxy)-ethoxy]-acetyl)-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDKKAQXEFIXYLIEGGPSSGAPPPS

SEQ ID NO: 221 - protein, 39 aa, synthetic construct; identical to SEQ ID NO: 220
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 222 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 223 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)16-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 224 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 225 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 226 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 227 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 228 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDYSIXLDEKAQXEFIXYLIEGGPSSG

SEQ ID NO: 229 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 30: note = Gly at position 30 is amidated
YXEGTXTSDYSIXLDEKAQXEFIXYLIEGG

SEQ ID NO: 230 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 231 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 alpha-methyl-Tyr
  MOD_RES 39: note = Ser at position 39 is amidated
YXEGTXTSDYSIXLDEKAQXEFIXXLIEGGPSSGAPPPS

SEQ ID NO: 232 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
YXEGTXTSDYSIXLDEKAQXEFIEXLIEGGPSSGAPPPS

SEQ ID NO: 233 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 234 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 235 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSILLDEKAQXEFIXYLIEGGPSSGAPPPS

SEQ ID NO: 236 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 237 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDYSIXLDKKAQXEFIXYLIEGGPSSG

SEQ ID NO: 238 - protein, 38 aa, synthetic construct
  REGION 1..38: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 12: note = MISC_FEATURE - Xaa at position 12 is alpha-methyl-Leu
  MOD_RES 16: note = Lys at position 16 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 19: note = MISC_FEATURE - Xaa at position 19 is Aib
  SITE 23: note = MISC_FEATURE - Xaa at position 23 is D-Glu
  MOD_RES 38: note = Ser at position 39 is amidated
YXEGTXTSDYSXLDEKAQXEFIXYLIEGGPSSGAPPPS

SEQ ID NO: 239 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDHKAQXEFIXYLIEGGPSSGAPPPS

SEQ ID NO: 240 - protein, 39 aa, synthetic construct; identical to SEQ ID NO: 220
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 241 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDXKAQXEFIXYLIEGGPSSGAPPPS

SEQ ID NO: 242 - protein, 39 aa, synthetic construct; identical to SEQ ID NO: 241
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 243 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSILLDXKAQXEFIXYLIEGGPSSGAPPPS

SEQ ID NO: 244 - protein, 39 aa, synthetic construct; identical to SEQ ID NO: 241
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 245 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDYSIXLDXKAQXEFIXXLIEGGPSSGAPPPS

SEQ ID NO: 246 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 alpha-methyl-Tyr
  MOD_RES 39: note = Ser at position 39 is amidated
YXEGTXTSDASIXLDXKAQXEFIXXLIEGGPSSGAPPPS

SEQ ID NO: 247 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXEFIXXLIEGGPSSGAPPPS

SEQ ID NO: 248 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDXSIXLDXKAQXTFIXXLIEGGPSSGAPPPS

SEQ ID NO: 249 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDXSIXLDXKAQXAFIXXLIEGGPSSGAPPPS

SEQ ID NO: 250 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDVSIXLDXKAQXTFIXXLIEGGPSSGAPPPS

SEQ ID NO: 251 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDVSIXLDXKAQXAFIXXLIEGGPSSGAPPPS

SEQ ID NO: 252 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXTFIXXLIEGGPSSGAPPPS

SEQ ID NO: 253 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXAFIXXLIEGGPSSGAPPPS

SEQ ID NO: 254 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDASIXLDXKAQXTFIXXLIEGGPSSGAPPPS

SEQ ID NO: 255 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDASIXLDXKAQXAFIXXLIEGGPSSGAPPPS

SEQ ID NO: 256 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 257 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSILLDXKAQXEFIXYLIEGGPSSGAPPPS

SEQ ID NO: 258 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Nle
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 259 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 260 - protein, 39 aa, synthetic construct; identical to SEQ ID NO: 249
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated

SEQ ID NO: 261 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDXSIXLDXKAQXAFIXXLIAGGPSSGAPPPS

SEQ ID NO: 262 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQAAFIEYLIEGGPSSGAPPPS

SEQ ID NO: 263 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQQAFIEYLIEGGPSSGAPPPS

SEQ ID NO: 264 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQHAFIEYLIEGGPSSGAPPPS

SEQ ID NO: 265 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQKAFIEYLIEGGPSSGAPPPS

SEQ ID NO: 266 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQRAFIEYLIEGGPSSGAPPPS

SEQ ID NO: 267 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDEKAQKAFIEYLIEGGPSSGAPPPS

SEQ ID NO: 268 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDEKAQXAFIEYLIEGGPSSGAPPPS

SEQ ID NO: 269 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 39: note = MISC_FEATURE - Ser at position 39 is amidated
YXEGTFTSDHSILLDKKAQXAFIEYLIEGGPSSGAPPPS

SEQ ID NO: 270 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQEAFIEYLIEGGPSSGAPPPS

SEQ ID NO: 271 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQTAFIEYLIEGGPSSGAPPPS

SEQ ID NO: 272 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKKAQSAFIEYLIEGGPSSGAPPPS

SEQ ID NO: 273 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 34: note = Gly at position 34 is amidated
YXEGTXTSDXSIXLDXKAQXEFIXXLIEGGPSSG

SEQ ID NO: 274 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 13 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 30: note = Gly at position 30 is amidated
YXEGTXTSDXSIXLDXKAQXEFIXXLIEGG

SEQ ID NO: 275 - protein, 34 aa, synthetic construct; identical to SEQ ID NO: 59
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 276 - protein, 30 aa, synthetic construct; identical to SEQ ID NO: 60
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated

SEQ ID NO: 277 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDEKAQXAFIEYLIEGGPSSG

SEQ ID NO: 278 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 30: note = MISC_FEATURE - Gly at position 30 is amidated
YXEGTFTSDYSILLDEKAQXAFIEYLIEGG

SEQ ID NO: 279 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXEFIEYLIEGGPSSG

SEQ ID NO: 280 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated
YXEGTFTSDYSILLDKKAQXEFIEYLIEGG

SEQ ID NO: 281 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXAFIEYLIAGGPSSG

SEQ ID NO: 282 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated
YXEGTFTSDYSILLDKKAQXAFIEYLIAGG

SEQ ID NO: 283 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 34: note = Gly at position 34 is amidated
YXEGTFTSDYSILLDKKAQPAFIEYLIEGGPSSG

SEQ ID NO: 284 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 30: note = Gly at position 30 is amidated
YXEGTFTSDYSILLDKKAQPAFIEYLIEGG

SEQ ID NO: 285 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 286 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDEQAQXEFIXYLIEGGPSSGAPPPS

SEQ ID NO: 287 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 20: note = MISC_FEATURE - Xaa at position 20 is Aib
  SITE 24: note = MISC_FEATURE - Xaa at position 24 is D-Glu
  MOD_RES 40: note = Cys at position 40 is amidated
YXEGTXTSDYSIXLDEKAQXEFIXYLIEGGPSSGAPPPSC

SEQ ID NO: 288 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 20: note = MISC_FEATURE - Xaa at position 20 is Aib
  SITE 24: note = MISC_FEATURE - Xaa at position 24 is D-Glu
  MOD_RES 40: note = Cys at position 40 is amidated
YXEGTXTSDYSIXLDEQAQXEFIXYLIEGGPSSGAPPPSC

SEQ ID NO: 289 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 290 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDXQAQXEFIXYLIEGGPSSGAPPPS

SEQ ID NO: 291 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  MOD_RES 40: note = Cys at position 40 is amidated
YXEGTXTSDYSIXLDXKAQXEFIXYLIEGGPSSGAPPPSC

SEQ ID NO: 292 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  MOD_RES 40: note = Cys at position 40 is amidated
YXEGTXTSDYSIXLDXQAQXEFIXYLIEGGPSSGAPPPSC

SEQ ID NO: 293 - protein, 39 aa, synthetic construct; identical to SEQ ID NO: 13
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated

SEQ ID NO: 294 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  SITE 39: note = mod - Ser at position 39 is amidated
YXEGTXTSDXSIXLDXQAQXEFIXXLIEGGPSSGAPPPS

SEQ ID NO: 295 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 40: note = Cys at position 40 is amidated
YXEGTXTSDXSIXLDXKAQXEFIXXLIEGGPSSGAPPPSC

SEQ ID NO: 296 - protein, 40 aa, synthetic construct
  REGION 1..40: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 40: note = Cys at position 40 is amidated
YXEGTXTSDXSIXLDXQAQXEFIXXLIEGGPSSGAPPPSC

SEQ ID NO: 297 - protein, 4 aa, synthetic construct
  REGION 1..4: note = Synthetic Construct
  MOD_RES 4: note = The C-terminal Gly is modified with R2 to provide PSSG-R2, wherein the modification is NH2 or absent.
PSSG

SEQ ID NO: 298 - protein, 9 aa, synthetic construct
  REGION 1..9: note = Synthetic Construct
  MOD_RES 9: note = The C-terminal Ser is modified with R2 to provide PSSGAPPPS-R2, wherein the modification is NH2 or absent.
PSSGAPPPS

SEQ ID NO: 299 - protein, 4 aa, synthetic construct
  REGION 1..4: note = Synthetic Construct
PSSG

SEQ ID NO: 300 - protein, 4 aa, synthetic construct
  REGION 1..4: note = Synthetic Construct
  MOD_RES 4: note = Gly at position 4 is amidated
PSSG

SEQ ID NO: 301 - protein, 9 aa, synthetic construct
  REGION 1..9: note = Synthetic Construct
PSSGAPPPS

SEQ ID NO: 302 - protein, 9 aa, synthetic construct
  REGION 1..9: note = Synthetic Construct
  MOD_RES 9: note = Ser at position 9 is amidated
PSSGAPPPS

SEQ ID NO: 303 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXAFIEYLIAGGPSSGAPPPS

SEQ ID NO: 304 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDKKAQXEFIEYLIAGGPSSGAPPPS

SEQ ID NO: 305 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDKKAQXEFIEYLIAGGPSSGAPPPS

SEQ ID NO: 306 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDXKAQXAFIXYLIAGGPSSGAPPPS

SEQ ID NO: 307 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDKKAQXEFIEXLIAGGPSSGAPPPS

SEQ ID NO: 308 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDXKAQXEFIEXLIAGGPSSGAPPPS

SEQ ID NO: 309 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 30: note = Gly at position 30 is amidated
YXEGTFTSDYSILLDKKAQQAFIEYLIEGG

SEQ ID NO: 310 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 30: note = Gly at position 30 is amidated
YXEGTFTSDYSILLDKKAQHAFIEYLIEGG

SEQ ID NO: 311 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 34: note = Gly at position 34 is amidated
YXEGTFTSDYSILLDKKAQKAFIEYLIEGGPSSG

SEQ ID NO: 312 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated
YXEGTFTSDYSILLDKKAQXEFIEYLIAGG

SEQ ID NO: 313 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXAFVEYLIEGGPSSG

SEQ ID NO: 314 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXAFLEYLIEGGPSSG

SEQ ID NO: 315 - protein, 34 aa, synthetic construct
  REGION 1..34: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSIXLDKKAQXAFIEYLIEGGPSSG

SEQ ID NO: 316 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTXTSDYSILLDKKAQXAFIEYLIEGGPSSG

SEQ ID NO: 317 - protein, 34 aa, synthetic construct; identical to SEQ ID NO: 316
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 318 - protein, 34 aa, synthetic construct
  REGION 1..34: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSIXLDEKAQXAFIEYLIEGGPSSG

SEQ ID NO: 319 - protein, 34 aa, synthetic construct
  REGION 1..34: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSIXLDKKAQXEFIEYLIEGGPSSG

SEQ ID NO: 320 - protein, 34 aa, synthetic construct
  REGION 1..34: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSIXLDKKAQXAFIEYLIAGGPSSG

SEQ ID NO: 321 - protein, 34 aa, synthetic construct
  REGION 1..34: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSIXLDKKAQXEFIEYLIAGGPSSG

SEQ ID NO: 322 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTXTSDYSILLDEKAQXAFIEYLIEGGPSSG

SEQ ID NO: 323 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTXTSDYSILLDKKAQXEFIEYLIEGGPSSG

SEQ ID NO: 324 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTXTSDYSILLDKKAQXAFIEYLIAGGPSSG

SEQ ID NO: 325 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTXTSDYSILLDKKAQXEFIEYLIAGGPSSG

SEQ ID NO: 326 - protein, 34 aa, synthetic construct; identical to SEQ ID NO: 322
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 327 - protein, 34 aa, synthetic construct; identical to SEQ ID NO: 323
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 328 - protein, 34 aa, synthetic construct; identical to SEQ ID NO: 324
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 329 - protein, 34 aa, synthetic construct; identical to SEQ ID NO: 325
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated

SEQ ID NO: 330 - protein, 39 aa, synthetic construct; identical to SEQ ID NO: 17
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 39: note = Ser at position 39 is amidated

SEQ ID NO: 331 - protein, 39 aa, synthetic construct; identical to SEQ ID NO: 303
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 39: note = Ser at position 39 is amidated

SEQ ID NO: 332 - protein, 34 aa, synthetic construct; identical to SEQ ID NO: 281
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated

SEQ ID NO: 333 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTFTSDYSILLDKKAQXEFIEYLIAGGPSSG

SEQ ID NO: 334 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXEFIEYLIAGGPSSGAPPPS

SEQ ID NO: 335 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXDFIEYLIEGGPSSG

SEQ ID NO: 336 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXTFIEYLIEGGPSSG

SEQ ID NO: 337 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 34: note = Gly at position 34 is amidated
YXEGTFTSDYSILLDKKAQXHFIEYLIEGGPSSG

SEQ ID NO: 338 - protein, 39 aa, synthetic construct; identical to SEQ ID NO: 68
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 39: note = Ser at position 39 is amidated

SEQ ID NO: 339 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDKKAQXAFIEYLIAGGPSSGAPPPS

SEQ ID NO: 340 - protein, 39 aa, synthetic construct; identical to SEQ ID NO: 66
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 341 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
FXEGTFTSDYSILLDKKAQXAFIEYLIEGGPSSGAPPPS

SEQ ID NO: 342 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
FXEGTFTSDYSIXLDKKAQXAFIEYLIEGGPSSGAPPPS

SEQ ID NO: 343 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 39: note = Ser at position 39 is amidated
FXEGTFTSDYSIXLDKKAQXAFIEYLIEAGPSSGAPPPS

SEQ ID NO: 344 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTXTSDYSILLDKKAQXAFIEYLIEGGPSSGAPPPS

SEQ ID NO: 345 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDEKAQXAFIEYLIAGGPSSGAPPPS

SEQ ID NO: 346 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDEKAQXEFIEYLIAGGPSSGAPPPS

SEQ ID NO: 347 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 2 is Aib
  MOD_RES 39: note = Ser at position 39 is amidated
YXEGTFTSDYSIXLDEKAQXAFIEYLIAGGPSSGAPPPS

SEQ ID NO: 348 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDKKAQXEFIEYLIEGGPSSGAPPPS

SEQ ID NO: 349 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDKKAQXAFIEYLIEGGPSSGAPPPS

SEQ ID NO: 350 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTXTSDYSIXLDKKAQXAFIEYLIEGGPSSGAPPPS

SEQ ID NO: 351 - protein, 39 aa, synthetic construct; identical to SEQ ID NO: 350
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 352 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
FXEGTFTSDYSIXLDKKAQXAFIEYLIAGGPSSGAPPPS

SEQ ID NO: 353 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
FXEGTFTSDYSIXLDKKAQXEFIEYLIAGGPSSGAPPPS

SEQ ID NO: 354 - protein, 39 aa, synthetic construct; identical to SEQ ID NO: 304
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-CO-(CH2)18-CO2 H
  SITE 20: note = MISC_FEATURE - Xaa at position 20 is Aib
  MOD_RES 39: note = Ser at position 39 is amidated

SEQ ID NO: 355 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDKKAQXAFIEYLIAGGPSSGAPPPS

SEQ ID NO: 356 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDEKAQXAFIEYLIAGGPSSGAPPPS

SEQ ID NO: 357 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDEKAQXEFIEYLIAGGPSSGAPPPS

SEQ ID NO: 358 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTFTSDYSIXLDEKAQXAFIXYLIAGGPSSGAPPPS

SEQ ID NO: 359 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDEKAQXDFIEYLIAGGPSSGAPPPS

SEQ ID NO: 360 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTXTSDYSIXLDKKAQXEFIEYLIAGGPSSGAPPPS

SEQ ID NO: 361 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXAFIEYLIAGGPSSGAPPPS

SEQ ID NO: 362 - protein, 39 aa, synthetic construct; identical to SEQ ID NO: 361
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 363 - protein, 39 aa, synthetic construct; identical to SEQ ID NO: 361
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 364 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXEFIEYLIAGGPSSGAPPPS

SEQ ID NO: 365 - protein, 39 aa, synthetic construct; identical to SEQ ID NO: 364
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 366 - protein, 39 aa, synthetic construct; identical to SEQ ID NO: 364
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 367 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDKKAQXAFIXYLIAGGPSSGAPPPS

SEQ ID NO: 368 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDKKAQXEFIXYLIEGGPSSGAPPPS

SEQ ID NO: 369 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDXKAQXEFIXYLIAGGPSSGAPPPS

SEQ ID NO: 370 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDXKAQXEFIXYLIEGGPSSGAPPPS

SEQ ID NO: 371 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDKKAQXAFIXYLIEGGPSSGAPPPS

SEQ ID NO: 372 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDXKAQXAFIXYLIEGGPSSGAPPPS

SEQ ID NO: 373 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXAFVEYLIAGGPSSGAPPPS

SEQ ID NO: 374 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXEFVEYLIAGGPSSGAPPPS

SEQ ID NO: 375 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXAFVEYLIEGGPSSGAPPPS

SEQ ID NO: 376 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDKKAQXAFVXYLIAGGPSSGAPPPS

SEQ ID NO: 377 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
XXEGTFTSDYSIXLDXKAQXAFVXYLIAGGPSSGAPPPS

SEQ ID NO: 378 - protein, 39 aa, synthetic construct; identical to SEQ ID NO: 306
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 379 - protein, 39 aa, synthetic construct; identical to SEQ ID NO: 377
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 380 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDKKAQXAFIEXLIAGGPSSGAPPPS

SEQ ID NO: 381 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDXKAQXAFIEXLIAGGPSSGAPPPS

SEQ ID NO: 382 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
YXEGTFTSDYSIXLDKKAQXEFIEXLIAGGPSSGAPPPS

SEQ ID NO: 383 - protein, 39 aa, synthetic construct; identical to SEQ ID NO: 364
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 39: note = Ser at position 39 is amidated

SEQ ID NO: 384 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
XXEGTFTSDYSIXLDXKAQXDFIEYLIAGGPSSGAPPPS

SEQ ID NO: 385 - protein, 34 aa, synthetic construct
  REGION 1..34: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
XXEGTFTSDYSIXLDXKAQXEFIEYLIAGGPSSG

SEQ ID NO: 386 - protein, 39 aa, synthetic construct; identical to SEQ ID NO: 364
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-CO-(CH2)18-CO2 H
  SITE 20: note = MISC_FEATURE - Xaa at position 20 is Aib
  MOD_RES 39: note = Ser at position 39 is amidated

SEQ ID NO: 387 - protein, 39 aa, synthetic construct; identical to SEQ ID NO: 307
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-CO-(CH2)18-CO2 H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 388 - protein, 39 aa, synthetic construct; identical to SEQ ID NO: 380
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-CO-(CH2)18-CO2 H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 389 - protein, 39 aa, synthetic construct; identical to SEQ ID NO: 307
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 390 - protein, 39 aa, synthetic construct; identical to SEQ ID NO: 308
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 391 - protein, 39 aa, synthetic construct; identical to SEQ ID NO: 380
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 392 - protein, 39 aa, synthetic construct; identical to SEQ ID NO: 381
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated

SEQ ID NO: 393 - protein, 39 aa, synthetic construct; identical to SEQ ID NO: 305
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-CO-(CH2)18-CO2 H
  SITE 20: note = MISC_FEATURE - Xaa at position 20 is Aib
  MOD_RES 39: note = Ser at position 39 is amidated

SEQ ID NO: 394 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDKKAQXEFIEXLIEGGPSSGAPPPS

SEQ ID NO: 395 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDXKAQXEFIEXLIEGGPSSGAPPPS

SEQ ID NO: 396 - protein, 39 aa, synthetic construct
  REGION 1..39: note = Synthetic Construct
  SITE 1: note = MISC_FEATURE - Xaa at position 1 is D-Tyr
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
XXEGTFTSDYSIXLDEKAQXAFIEXLIAGGPSSGAPPPS

(19) United States
(12) Patent Application Publication
(10) Pub. No.: US 2025/0051415 A1
(43) Pub. Date: Feb. 13, 2025

(54) GIP/GLP1 CO-AGONIST COMPOUNDS

(71) Applicant: Eli Lilly and Company, Indianapolis, IN (US)

(72) Inventors: Jorge ALSINA-FERNANDEZ, Indianapolis, IN (US); Robert Andrew BROWN, Carmel, IN (US); Mohamed E.H. ELSAYED, Fishers, IN (US); Hongchang QU, Carmel, IN (US); Thi Thanh Huyen TRAN, Bargersville, IN (US); Aktham ABURUB, Carmel, IN (US); Phenil Jayantilal PATEL, Carmel, IN (US)

(21) Appl. No.: 18/919,541

(22) Filed: Oct. 18, 2024

Related U.S. Application Data
(62) Division of application No. 17/366,998, filed on Jul. 2, 2021.
Application No. 17/366,998 is a division of application No. 16/518,468, filed on Jul. 22, 2019, now Pat. No. 11,084,861.
(60) Provisional application No. 62/740,596, filed on Oct. 3, 2018.
(60) Provisional application No. 62/730,563, filed on Sep. 13, 2018.
(60) Provisional application No. 62/702,072, filed on Jul. 23, 2018.

(51) Int. Cl. C07K 14/605; A61K 9/00; A61K 9/20; A61K 9/48; A61K 38/56; A61P 3/04

(52) U.S. Cl. CPC C07K 14/605; A61K 9/2013; A61K 9/4858; A61K 38/56; A61P 3/04; A61K 9/0019; A61K 9/0053

(57) ABSTRACT

The present invention relates to compounds having activity at both the human glucose-dependent insulinotropic polypeptide (GIP) and glucagon-like peptide-1 (GLP-1) receptors. The present invention also relates to compounds having an extended duration of action at each of these receptors. Furthermore, the present invention relates to compounds that may be administered orally. Compounds may be useful in the treatment of type 2 diabetes mellitus (“T2DM”). Also, the compounds may be useful in the treatment of obesity.

The present application is being filed along with a Sequence Listing in ST.26 XML format. The Sequence Listing is provided as a file titled “X21852B.xml” created Oct. 17, 2024, and is 1,426 kilobytes in size. The Sequence Listing information in the ST.26 XML format is incorporated herein by reference in its entirety. No new matter is added herewith.

The present invention relates to compounds having activity at both the human glucose-dependent insulinotropic polypeptide (GIP) and glucagon-like peptide-1 (GLP-1) receptors. The present invention also relates to compounds having an extended duration of action at each of these receptors. Furthermore, the present invention relates to compounds that may be administered orally. Compounds may be useful in the treatment of type 2 diabetes mellitus (“T2DM”). Also, the compounds may be useful in the treatment of obesity.

Over the past several decades, the prevalence of diabetes has continued to rise. T2DM is the most common form of diabetes accounting for approximately 90% of all diabetes. T2DM is characterized by high blood glucose levels associated mainly with insulin resistance. The current standard of care for T2DM includes diet and exercise, treatment with oral medications, and injectable glucose lowering drugs, including incretin-based therapies, such as GLP-1 receptor agonists. A variety of GLP-1 receptor agonists are currently available for treatment of T2DM, although currently marketed GLP-1 receptor agonists are generally dose-limited by gastrointestinal side effects such as nausea and vomiting. Subcutaneous injection is the typical route of administration for the available GLP-1 receptor agonists. When treatment with oral medications and incretin-based therapies are insufficient, insulin treatment is considered. Despite the advances in treatment available today, many patients with T2DM are unable to reach their glycemic control goals. Uncontrolled diabetes leads to several conditions associated with increased morbidity and mortality of patients. There is a need for a treatment to enable more patients with T2DM to reach their glycemic treatment goal.

Obesity is a complex medical disorder resulting in excessive accumulation of adipose tissue mass. Today obesity is a global public health concern that is associated with undesired health outcomes and morbidities. Desired treatments for patients with obesity strive to reduce excess body weight, improve obesity-related co-morbidities, and maintain long-term weight reduction. Available treatments for obesity are particularly unsatisfactory for patients with severe obesity. There is a need for alternative treatment options to induce therapeutic weight loss in patients in need of such treatment.

WO2016/111971 describes peptides stated to have GLP-1 and GIP activity. WO2013/164483 also discloses compounds stated to have GLP-1 and GIP activity.

There is a need for T2DM treatments capable of providing effective glucose control for a larger portion of the patients in need of such treatment. There is a further need for T2D treatments capable of providing effective glucose control and with a favorable side effect profile. There is a need for alternate treatment options to provide therapeutic weight loss in a patient in need of such treatment. There is a need for an alternate treatment option for a patient in need of treatment for severe obesity.

There is a desire for compounds having agonist activity at the GIP and GLP-1 receptors that are suitable for oral administration. Compounds with extended duration of action at each of the GIP and GLP-1 receptors are desirable to allow for less frequent dosing of the compound.

Accordingly, the present invention provides a compound of Formula I:

(SEQ ID NO: 3)
R₁X₁X₂X₃GTX₆TSDX₁₀X₁₁X₁₂X₁₃X₁₄DX₁₆X₁₇AX₁₉X₂₀X₂₁X₂₂
X₂₃X₂₄X₂₅X₂₆X₂₇X₂₈X₂₉X₃₀X₃₁

wherein:
R₁ is a modification of the N-terminal amino group wherein the modification is selected from the group consisting of Ac and absent;
X₁ is selected from the group consisting of Y, H, D-Tyr, F, desH, and desY,
X₂ is selected from the group consisting of Aib, αMeP, A, P, and D-Ala;
or X₁ and X₂ combine to form desH-ψ[NHCO]-Aib;
X₃ is selected from the group consisting of E, N, Aad, and cTA;
X₆ is selected from the group consisting of F, αMeF, and αMeF(2F);
X₁₀ is selected from the group consisting of A, L, H, 3Pal, 4Pal, V, Y, E, αMeF, αMeF(2F), I, αMeY, Q, D-His, D-Tyr, cTA, and K(2-[2-(2-amino-ethoxy)-ethoxy]-acetyl)₂-(γ-Glu)-CO—(CH₂)qCO₂H;
X₁₁ is selected from the group consisting of S, αMeS, and D-Ser;
X₁₂ is selected from the group consisting of I, S, D-I1e, and K(2-[2-(2-amino-ethoxy)-ethoxy]-acetyl)₂-(γ-Glu)-CO—(CH₂)qCO₂H;
X₁₃ is selected from the group consisting of Nle, Aib, L, αMeL, and K(2-[2-(2-amino-ethoxy)-ethoxy]-acetyl)₂-(γ-Glu)-CO—(CH₂)qCO₂H;
X₁₄ is selected from the group consisting of L and K, wherein K is conjugated to a C₁₆-C₂₂ fatty acid wherein said fatty acid is optionally conjugated to said K via a linker;
X₁₆ is selected from the group consisting of K, E, Orn, Dab, Dap, S, T, H, Aib, αMeK, R, and K(2-[2-(2-amino-ethoxy)-ethoxy]-acetyl)₂-(γ-Glu)-CO—(CH₂)qCO₂H;
X₁₇ is selected from the group consisting of K, Q, I, and an amino acid conjugated to a C₁₆-C₂₂ fatty acid wherein said fatty acid is optionally conjugated to said amino acid via a linker;
X₁₉ is selected from the group consisting of Q, A, and K(2-[2-(2-amino-ethoxy)-ethoxy]-acetyl)₂-(γ-Glu)-CO—(CH₂)qCO₂H;
X₂₀ is selected from the group consisting of Aib, Q, H, R, K, αMeK, and K(2-[2-(2-amino-ethoxy)-ethoxy]-acetyl)₂-(γ-Glu)-CO—(CH₂)qCO₂H;
X₂₁ is selected from the group consisting of H, Aad, D, Aib, T, A, E, I, and K(2-[2-(2-amino-ethoxy)-ethoxy]-acetyl)₂-(γ-Glu)-CO—(CH₂)qCO₂H;
X₂₂ is selected from the group consisting of F and αMeF;
X₂₃ is selected from the group consisting of I, L, A, G, F, H, E, V, and K(2-[2-(2-amino-ethoxy)-ethoxy]-acetyl)₂-(γ-Glu)-CO—(CH₂)qCO₂H;
X₂₄ is selected from the group consisting of S, Aad, D-Glu, E, Aib, H, V, A, Q, D, P, and K(2-[2-(2-amino-ethoxy)-ethoxy]-acetyl)₂-(γ-Glu)-CO—(CH₂)qCO₂H;
X₂₅ is selected from the group consisting of Y and αMeY;
X₂₆ is selected from the group consisting of L, αMeL, and K(2-[2-(2-amino-ethoxy)-ethoxy]-acetyl)₂-(γ-Glu)-CO—(CH₂)qCO₂H;
X₂₇ is selected from the group consisting of L, I, and K(2-[2-(2-amino-ethoxy)-ethoxy]-acetyl)₂-(γ-Glu)-CO—(CH₂)qCO₂H;
X₂₈ is selected from the group consisting of E, A, S, D-Glu, and K(2-[2-(2-amino-ethoxy)-ethoxy]-acetyl)₂-(γ-Glu)-CO—(CH₂)qCO₂H;
X₂₉ is selected from the group consisting of Aib, G, A, and K(2-[2-(2-amino-ethoxy)-ethoxy]-acetyl)₂-(γ-Glu)-CO—(CH₂)qCO₂H;
X₃₀ is selected from the group consisting of C, G, G-R₂ and K(2-[2-(2-amino-ethoxy)-ethoxy]-acetyl)₂-(γ-Glu)-CO—(CH₂)_{q}—CO₂H;
X₃₁ is absent or is selected from the group consisting of PX₃₂X₃₃X₃₄-R₂ (SEQ ID NO:4), PX₃₂X₃₃X₃₄X₃₅X₃₆X₃₇X₃₈X₃₉-R₂ (SEQ ID NO:5), PX₃₂X₃₃X₃₄X₃₅X₃₆X₃₇X₃₈X₃₉X₄₀-R₂ (SEQ ID NO:6), K[(2-[2-(2-amino-ethoxy)-ethoxy]-acetyl)₂-(γ-Glu)-CO—(CH₂)_{q}—CO₂H]X₃₂X₃₃X₃₄-R₂ (SEQ ID NO:7), K[(2-[2-(2-amino-ethoxy)-ethoxy]-acetyl)₂-(γ-Glu)-CO—(CH₂)_{q}—CO₂H]X₃₂X₃₃X₃₄X₃₅X₃₆X₃₇X₃₈X₃₉-R₂ (SEQ ID NO:8), and K[(2-[2-(2-amino-ethoxy)-ethoxy]-acetyl)₂-(γ-Glu)-CO—(CH₂)_{q}—CO₂H]X₃₂X₃₃X₃₄X₃₅X₃₆X₃₇X₃₈X₃₉X₄₀-R₂ (SEQ ID NO:9);
   wherein:
   X₃₂ is S or K[(2-[2-(2-amino-ethoxy)-ethoxy]-acetyl)₂-(γ-Glu)-CO—(CH₂)_{q}—CO₂H];
   X₃₃ is S or K[(2-[2-(2-amino-ethoxy)-ethoxy]-acetyl)₂-(γ-Glu)-CO—(CH₂)_{q}—CO₂H];
   X₃₄ is selected from the group consisting of G, C, and K[(2-[2-(2-amino-ethoxy)-ethoxy]-acetyl)₂-(γ-Glu)-CO—(CH₂)_{q}—CO₂H];
   X₃₅ is A or K[(2-[2-(2-amino-ethoxy)-ethoxy]-acetyl)₂-(γ-Glu)-CO—(CH₂)_{q}—CO₂H];
   X₃₆ is P or K[(2-[2-(2-amino-ethoxy)-ethoxy]-acetyl)₂-(γ-Glu)-CO—(CH₂)_{q}—CO₂H];
   X₃₇ is P or K[(2-[2-(2-amino-ethoxy)-ethoxy]-acetyl)₂-(γ-Glu)-CO—(CH₂)_{q}—CO₂H];
   X₃₈ is P or K[(2-[2-(2-amino-ethoxy)-ethoxy]-acetyl)₂-(γ-Glu)-CO—(CH₂)_{q}—CO₂H];
   X₃₉ is selected from the group consisting of C, S, and K[(2-[2-(2-amino-ethoxy)-ethoxy]-acetyl)₂-(γ-Glu)-CO—(CH₂)_{q}—CO₂H];
   X₄₀ is selected from the group consisting of C and K[(2-[2-(2-amino-ethoxy)-ethoxy]-acetyl)₂-(γ-Glu)-CO—(CH₂)_{q}—CO₂H];
q is selected from the group consisting of 14, 15, 16, 17, 18, 19, and 20; and
R₂ is a modification of the C-terminal group, wherein the modification is NH₂ or absent;
or a pharmaceutically acceptable salt thereof;
wherein if X₃₀ is G-R₂, then X₃₁ is absent;
wherein no more than one of X₁₀, X₁₂, X₁₃, X₁4, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ may be a substituent that contains a fatty acid; and
wherein no more than one of X₃₀, X₃₄, X₃₉, and X₄₀ may be C; and
wherein if one of X₃₀, X₃₄, X₃₉, and X₄₀ is C, then none of X₁₀, X₁₂, X₁₃, X₁4, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ is a substituent that contains a fatty acid.

In an embodiment is a compound of Formula I, or a pharmaceutically acceptable salt thereof, wherein q is 16. In an embodiment is a compound of Formula I, or a pharmaceutically acceptable salt thereof, wherein X₃₁ is selected from the group consisting of SEQ ID NO:5 and SEQ ID NO:8. In an embodiment is a compound of Formula I, or a pharmaceutically acceptable salt thereof, wherein the X₁₇ amino acid that is conjugated to a fatty acid is a natural amino acid. In an embodiment is a compound of Formula I, or a pharmaceutically acceptable salt thereof, wherein X₁₇ is selected from the group consisting of K, Q and I.

In an embodiment is a compound of Formula I, or a pharmaceutically acceptable salt thereof, wherein K is conjugated to a C₁₆-C₂₂ fatty acid wherein said fatty acid is optionally conjugated to said K via a linker.

In an embodiment is a compound of Formula I, or a pharmaceutically acceptable salt thereof, wherein X₁₄ or X₁₇ is selected from the group consisting of K(2-[2-(2-Amino-ethoxy)-ethoxy]-acetyl)₂-(γ-Glu)-CO—(CH₂)₁₆—CO₂H, K(2-[2-(2-Amino-ethoxy)-ethoxy]-acetyl)₂-(γ-Glu)-CO—(CH₂)₁₈—CO₂H, K(2-[2-(2-Amino-ethoxy)-ethoxy]-acetyl)₂-(γ-Glu)-CO—(CH₂)₁₄—CO₂H, K(2-[2-(2-Amino-ethoxy)-ethoxy]-acetyl)₂-CO—(CH₂)₁₈—CO₂H, K(2-[2-(2-Amino-ethoxy)-ethoxy]-acetyl)-(γ-Glu)-(Trx)-CO—(CH₂)₁₈—CO₂H, K(2-[2-(2-Amino-ethoxy)-ethoxy]-acetyl)-(Trx)-(γ-Glu)-CO—(CH₂)₁₈—CO₂H, K(2-[2-(2-Amino-ethoxy)-ethoxy]-acetyl)-(εK)-(γ-Glu)-CO—(CH₂)₁₈—CO₂H, K(2-[2-(2-Amino-ethoxy)-ethoxy]-acetyl)-(εK)-(εK)—CO—(CH₂)₁₈—CO₂H, K(2-[2-(2-Amino-ethoxy)-ethoxy]-acetyl)₂-(γ-Glu)₂-CO—(CH₂)₁₈—CO₂H, K(2-[2-(2-Amino-ethoxy)-ethoxy]-acetyl)₂-CO—(CH₂)₁₈—CO₂H, K(2-[2-(2-Amino-ethoxy)-ethoxy]-acetyl)₂-(εK)—CO—(CH₂)₁₆—CO₂H, K(2-[2-(2-Amino-ethoxy)-ethoxy]-acetyl)₂-(εK)—CO—(CH₂)₁₄—CO₂H, and KDab-(2-[2-(2-Amino-ethoxy)-ethoxy]-acetyl)-Dab-(2-[2-(2-Amino-ethoxy)-ethoxy]-acetyl)-CO—(CH₂)₁₈—CO₂H.

In an embodiment is a compound of Formula I, or a pharmaceutically acceptable salt thereof, wherein X₁₄ or X₁₇ is selected from the group consisting of K(2-[2-(2-Amino-ethoxy)-ethoxy]-acetyl)₂-(γ-Glu)-CO—(CH₂)₁₆—CO₂H, K(2-[2-(2-Amino-ethoxy)-ethoxy]-acetyl)₂-(γ-Glu)-CO—(CH₂)₁₈—CO₂H, K(2-[2-(2-Amino-ethoxy)-ethoxy]-acetyl)₂-(γ-Glu)-CO—(CH₂)₁₄—CO₂H, and K(2-[2-(2-Amino-ethoxy)-ethoxy]-acetyl)₂-CO—(CH₂)₁₈—CO₂H.

In an embodiment is a compound of Formula I, or a pharmaceutically acceptable salt thereof, wherein X₁₄ or X₁₇ is selected from the group consisting of K(2-[2-(2-Amino-ethoxy)-ethoxy]-acetyl)₂-(γ-Glu)-CO—(CH₂)₁₆—CO₂H, K(2-[2-(2-Amino-ethoxy)-ethoxy]-acetyl)₂-(γ-Glu)-CO—(CH₂)₁₈—CO₂H, and K(2-[2-(2-Amino-ethoxy)-ethoxy]-acetyl)₂-(γ-Glu)-CO—(CH₂)₁₄—CO₂H. In an embodiment is a compound of Formula I, or a pharmaceutically acceptable salt thereof, wherein X₁₄ or X₁₇ is selected from the group consisting of K(2-[2-(2-Amino-ethoxy)-ethoxy]-acetyl)₂-(γ-Glu)-CO—(CH₂)₁₈—CO₂H and K(2-[2-(2-Amino-ethoxy)-ethoxy]-acetyl)₂-(γ-Glu)-CO—(CH₂)₁₆—CO₂H. In an embodiment is a compound of Formula I, or pharmaceutically acceptable salt thereof, wherein X₁₄ or X₁₇ is K(2-[2-(2-amino-ethoxy)-ethoxy]-acetyl)ₐ-(γ-Glu)_{b}-CO—(CH₂)_{q}—CO₂H, wherein a is 2, b is 1, and q is selected from the group consisting of 18 and 20. In an embodiment is a compound of Formula I, or pharmaceutically acceptable salt thereof, wherein X₁₄ or X₁₇ is K(2-[2-(2-amino-ethoxy)-ethoxy]-acetyl)ₐ-(γ-Glu)_{b}-CO—(CH₂)_{q}—CO₂H, wherein a is 2, b is 1 and q is 18. In an embodiment is a compound of Formula I, or pharmaceutically acceptable salt thereof, wherein X₁₄ or X₁₇ is K(2-[2-(2-amino-ethoxy)-ethoxy]-acetyl)ₐ-(γ-Glu)_{b}-CO—(CH₂)_{q}—CO₂H, wherein, a is 2, b is 1, and q is 20.

In an embodiment is a Formula I compound, or pharmaceutically acceptable salt thereof, wherein X₁ and X₂ do not combine to form desH-ψ/[NHCO]-Aib (hereafter a “Formula II” compound).

In an embodiment is a compound of Formula I, or pharmaceutically acceptable salt thereof, wherein:
X₁₇ is an amino acid conjugated to a C₁₆-C₂₂ fatty acid wherein said fatty acid is optionally conjugated to said amino acid via a linker; and
X₃₀ is selected from the group consisting of G-R₂ and G;
wherein if X₃₀ is G, then X₃₁ is selected from the group consisting of PX₃₂X₃₃X₃₄—R₂ (SEQ ID NO:4), wherein X₃₂ is S, X₃₃ is S and X₃₄ is G (SEQ ID NO:297), and PX₃₂X₃₃X₃₄X₃₅X₃₆X₃₇X₃₈X₃₉-R₂ (SEQ ID NO:5), wherein X₃₂ is S, X₃₃ is S, X₃₄ is G,
X₃₅ is A, X₃₆ is P, X₃₇ is P, X₃₈ is P and X₃₉ is S (SEQ ID NO:298) (hereafter a “Formula III” compound).

In an embodiment is a compound of Formula III, or a pharmaceutically acceptable salt thereof, wherein the X₁₇ amino acid is conjugated to the fatty acid via a linker (hereafter a “Formula IIIa” compound).

In an embodiment is a compound of Formula III and IIIa, or a pharmaceutically acceptable salt thereof, wherein:
X₁₀ is selected from the group consisting of A, L, H, 3Pal, 4Pal, V, Y, E, αMeF, αMeF(2F), I, αMeY, Q, D-His, D-Tyr, and cTA;
X₁₂ is selected from the group consisting of I, S, and D-I1e;
X₁₃ is selected from the group consisting of Nle, Aib, L, and αMeL;
X₁₄ is selected from the group consisting of L and K;
X₁₆ is selected from the group consisting of K, E, Orn, Dab, Dap, S, T, H, Aib, αMeK, and R;
X₁₉ is selected from the group consisting of Q, and A;
X₂₀ is selected from the group consisting of Aib, Q, H, R, K, and αMeK;
X₂₁ is selected from the group consisting of H, Aad, D, Aib, T, A, E, and I;
X₂₃ is selected from the group consisting of I, L, A, G, F, H, E, and V;
X₂₄ is selected from the group consisting of S, Aad, D-Glu, E, Aib, H, V, A, Q, D, and P;
X₂₆ is selected from the group consisting of L, and αMeL;
X₂₇ is selected from the group consisting of L, and I;
X₂₈ is selected from the group consisting of E, A, S, and D-Glu;
X₂₉ is selected from the group consisting of Aib, G, and A;
X₃₀ is selected from the group consisting of G and G-R₂;
wherein if X₃₀ is G; then X₃₁ is selected from the group consisting of PX₃₂X₃₃X₃₄-R₂ (SEQ ID NO:4), wherein X₃₂ is S, X₃₃ is S and X₃₄ is G (SEQ ID NO:297) and PX₃₂X₃₃X₃₄X₃₅X₃₆X₃₇X₃₈X₃₉-R₂ (SEQ ID NO:5), wherein X₃₂ is S, X₃₃ is S, X₃₄ is G, X₃₅ is A, X₃₆ is P, X₃₇ is P, X₃₈ is P and X₃₉ is S (SEQ ID NO:298) (hereafter a “Formula IIIb” compound).

In an embodiment, is a compound of Formula III, IIIa and IIIb, or a pharmaceutically acceptable salt thereof, wherein the linker comprises from 1 to 2 amino acids, and in a further embodiment of these particular Formula III, IIIa and IIIb compounds are those wherein the linker amino acids are independently selected from the group consisting of Glu and v-Glu. In another embodiment is a compound of Formula III, IIIa and IIIb, or a pharmaceutically acceptable salt thereof, wherein the linker comprises from one or two (2-[2-(2-amino-ethoxy)-ethoxy]-acetyl) moieties and in a further embodiment of these particular formula III, IIIa and IIIb compounds are those where the linker is (2-[2-(2-amino-ethoxy)-ethoxy]-acetyl)ₐ-(γ-Glu)_{b}, wherein a is selected from the group consisting of 1 or 2; and b is selected from the group consisting of 1 or 2.

In an embodiment is a compound of Formula III, or a pharmaceutically acceptable salt thereof, wherein X₁₇ is an amino acid conjugated to a C₁₆-C₂₂ fatty acid, wherein the amino acid is K and wherein said fatty acid is optionally conjugated to said amino acid via a linker.

In an embodiment is a compound of Formula III, or pharmaceutically acceptable salt thereof, wherein:
R₁ is absent;
X₁ and X₂ do not combine to form desH-ψ[NHCO]-Aib;
X₁₇ is K conjugated to a C₁₆-C₂₂ fatty acid wherein said fatty acid is optionally conjugated to said amino acid via a linker.

In an embodiment is a compound of Formula III, or pharmaceutically acceptable salt thereof, wherein:
X₁ is Y;
X₂ is Aib;
X₃ is E;
X₁₀ is selected from the group consisting of A, L, H, 3Pal, 4Pal, V, and Y;
X₁₁ is S;
X₁₂ is I;
X₁₄ is L;
X₁₆ is selected from the group consisting of K, E, Orn, Dab, and Dap;
X₁₇ is K conjugated to a C₁₆-C₂₂ fatty acid wherein said fatty acid is optionally conjugated to said amino acid via a linker;
X₉ is Q;
X₂₀ is Aib;
X₂₁ is selected from the group consisting of H, Aad, D, Aib, T, A, and E;
X₂₂ is F;
X₂₃ is I;
X₂₄ is selected from the group consisting of S, Aad, D-Glu, and E;
X₂₆ is L; and
X₂₈ is selected from the group consisting of E and A.

In an embodiment is a compound of Formula III, or pharmaceutically acceptable salt thereof, wherein:
X₁ is Y;
X₂ is Aib;
X₃ is E;
X₆ is αMeF(2F);
X₁₀ is selected from the group consisting of Y, 4-Pal, and V;
X₁ is S;
X₁₂ is I;
X₁₃ is selected from the group consisting of L, Aib, and αMeL;
X₁₄ is L;
X₁₆ is selected from the group consisting of E, K, and Orn;
X₁₇ is K conjugated to a C₁₆-C₂₂ fatty acid wherein said fatty acid is optionally conjugated to said amino acid via a linker;
X₁₉ is Q;
X₂₀ is Aib
X₂₁ is selected from the group consisting of E, A, and T;
X₂₂ is F;
X₂₃ is I;
X₂₄ is D-Glu;
X₂₅ is selected from the group consisting of Y and αMeY;
X₂₆ is L;
X₂₇ is I;
X₂₈ is E;
X₂₉ is G;
X₃₀ is G; and
X₃₁ is PX₃₂X₃₃X₃₄X₃₅X₃₆X₃₇X₃₈X₃₉-R₂ (SEQ ID NO:5), wherein X₃₂ is S, X₃₃ is S,
X₃₄ is G, X₃₅ is A, X₃₆ is P, X₃₇ is P, X₃₈ is P, X₃₉ is S (SEQ ID NO:298).

In an embodiment is a compound of Formula III, IIIa and IIIb, or a pharmaceutically acceptable salt thereof, wherein R₂ is absent.

In an embodiment is a compound of Formula III, IIIa and IIIb, or a pharmaceutically acceptable salt thereof, wherein R₂ is NH₂.

In an embodiment is a compound of Formula III, IIIa and IIIb, or a pharmaceutically acceptable salt thereof, wherein X₁₃ is αMeL.

In an embodiment is a compound of Formula III, IIIa and IIIb, or a pharmaceutically acceptable salt thereof, wherein X₂₅ is Y and Xu is αMeL.

In an embodiment is a compound of Formula III, IIIa and IIIb, or a pharmaceutically acceptable salt thereof, wherein X₁₇ is K conjugated to a fatty acid via a linker to the epsilon-amino group of the K side-chain wherein said fatty acid and linker have the following formula:

(2-[2-(2-amino-ethoxy)-ethoxy]-acetyl)ₐ-(γ-Glu)_{b}-CO—(CH₂)_{q}—CO₂H, wherein a is 1 or 2; b is 1 or 2; and q is selected from the group consisting of 14 to 20.

In an embodiment is a compound of Formula III, IIIa and IIIb, or a pharmaceutically acceptable salt thereof, wherein X₁₆ is Orn, X₁₃ is αMeL, and X₂₅ is Y. In an embodiment is a compound of Formula III, IIIa and IIIb, or a pharmaceutically acceptable salt thereof, wherein X₁₆ is E, X₁₃ is αMeL, and X₂₅ is Y. In an embodiment, is a compound of Formula III, IIIa and IIIb, or a pharmaceutically acceptable salt thereof, wherein X₁₆ is E, X₁₃ is αMeL, X₁₀ is Y, and X₂₅ is MeY. In an embodiment is a compound of Formula III, IIIa and IIIb, or a pharmaceutically acceptable salt thereof, wherein X₁₆ is Orn, X₁₃ is αMeL, X₁₀ is 4Pal, and X₂₅ is Y. In an embodiment is a compound of Formula III, IIIa and IIIb, or a pharmaceutically acceptable salt thereof, wherein X₁₆ is Orn, X₁₃ is αMeL, X₁₀ is V, and X₂₅ is Y. In an embodiment is a compound of Formula III, IIIa and IIIb, or a pharmaceutically acceptable salt thereof, wherein X₁₆ is E, X₁₃ is αMeL, X₂₅ is Y, and X₁₇ is K(2-[2-(2-Amino-ethoxy)-ethoxy]-acetyl)ₐ-(γ-Glu)_{b}-CO—(CH₂)_{q}—CO₂H, wherein a is 2; b is 1; and q is selected from the group consisting of 14 to 20. In an embodiment is a compound of Formula III, IIIa and IIIb, or a pharmaceutically acceptable salt thereof, wherein X₁₆ is E, X₁₃ is αMeL, X₁₀ is Y, and X₂₅ is Y and and X₁₇ is K(2-[2-(2-Amino-ethoxy)-ethoxy]-acetyl)ₐ-(γ-Glu)_{b}-CO—(CH₂)_{q}—CO₂H, wherein a is 2; b is 1; and q is selected from the group consisting of 16 to 20.

In an embodiment is a compound of Formula I selected from the group consisting of SEQ ID NO:10, SEQ ID NO:11, SEQ ID NO:12, SEQ ID NO:13, and SEQ ID NO:14, or a pharmaceutically acceptable salt thereof. In an embodiment is a compound of Formula I that is SEQ ID NO:10, or a pharmaceutically acceptable salt thereof. In an embodiment is a compound of Formula I that is SEQ ID NO:11, or a pharmaceutically acceptable salt thereof. In an embodiment is a compound of Formula I that is SEQ ID NO:12, or a pharmaceutically acceptable salt thereof. In an embodiment is a compound of Formula I that is SEQ ID NO:13, or a pharmaceutically acceptable salt thereof. In an embodiment is a compound of Formula I that is SEQ ID NO:14, or a pharmaceutically acceptable salt thereof.

In an embodiment is a compound of Formula I, or a pharmaceutically acceptable salt thereof, wherein X₁ is selected from the group consisting of Y, F, and D-Tyr; X₆ is F; and X₁₃ is selected from the group consisting of Aib, L, and αMeL.

In an embodiment, is a compound of Formula I, or a pharmaceutically acceptable salt thereof, wherein R₁ is absent; X₁ is selected from the group consisting of Y, F, and D-Tyr; X₆ is F; X₁₃ is selected from the group consisting of Aib, L, and αMeL; X₂ is Aib; X₃ is E; X₁₀ is Y; X₁₁ is S; X₁₂ is I; X₁₄ is L; X₁₆ is selected from the group consisting of K, E, Orn, Dab, Dap, S, T, H, Aib, αMeK, and R; X₁₇ is an amino acid conjugated to a C₁₆-C₂₂ fatty acid wherein said fatty acid is optionally conjugated to said amino acid via a linker; X₁₉ is Q; X₂₀ is selected from the group consisting of Aib, Q, H, and K; X₂₁ is selected from the group consisting of H, D, T, A, and E; X₂₂ is F; X₂₃ is I; X₂₄ is selected from the group consisting of D-Glu and E; X₂₆ is L; X₂₇ is I; X₂₈ is selected from the group consisting of E, A, S, and D-Glu; X₂₉ is selected from the group consisting of Aib, G, and A; X₃₀ is selected from the group consisting of C, G, and G-R₂; X₃₁ is absent or is selected from the group consisting of PX₃₂X₃₃X₃₄-R₂ (SEQ ID NO:4), PX₃₂X₃₃X₃₄X₃₅X₃₆X₃₇X₃₈X₃₉-R₂ (SEQ ID NO:5), and PX₃₂X₃₃X₃₄X₃₅X₃₆X₃₇X₃₈X₃₉X₄₀-R₂ (SEQ ID NO:6); wherein: X₃₂ is S; X₃₃ is S; X₃₄is selected from the group consisting of G and C; X₃₅ is A; X₃₆ is P; X₃₇is P; X₃₈ is P; X₃₉ is selected from the group consisting of C and S; and X₄₀ is C.

In an embodiment, is a compound of Formula I, or a pharmaceutically acceptable salt thereof, wherein X₁ is selected from the group consisting of Y, F, and D-Tyr; X₆ is F; and X₁₃ is selected from the group consisting of Aib, L, and αMeL; X₂₈ is A; X₂₉ G; X₃₀ is G; X₃₁ is PX₃₂X₃₃X₃₄X₃₅X₃₆X₃₇X₃₈X₃₉-R₂ (SEQ ID NO:5); X₃₄ is G; and X₃₉ is S.

In an embodiment, is a compound of Formula I, or a pharmaceutically acceptable salt thereof, wherein X₁ is selected from the group consisting of Y and D-Tyr; and X₁₃ αMeL.

In an embodiment is a compound of Formula I selected from the group consisting of SEQ ID NO:303, SEQ ID NO:304, SEQ ID NO:305, SEQ ID NO:306, SEQ ID NO:307, and SEQ ID NO:308, or a pharmaceutically acceptable salt thereof. In an embodiment is a compound of Formula I that is SEQ ID NO:303, or a pharmaceutically acceptable salt thereof. In an embodiment is a compound of Formula I that is SEQ ID NO:304, or a pharmaceutically acceptable salt thereof. In an embodiment is a compound of Formula I that is SEQ ID NO:305, or a pharmaceutically acceptable salt thereof. In an embodiment is a compound of Formula I that is SEQ ID NO:306, or a pharmaceutically acceptable salt thereof. In an embodiment is a compound of Formula I that is SEQ ID NO:307, or a pharmaceutically acceptable salt thereof. In an embodiment is a compound of Formula I that is SEQ ID NO:308, or a pharmaceutically acceptable salt thereof. In an embodiment is a compound of Formula I that is SEQ ID NO:386, or a pharmaceutically acceptable salt thereof.

In an embodiment is a compound of Formula I, or a pharmaceutically acceptable salt thereof, wherein:
X₁₀ is selected from the group consisting of A, L, H, 3Pal, 4Pal, V, Y, αMeF, αMeF(2F), I, αMeY, Q, D-His, E, cTA, and D-Tyr;
X₁₂ is selected from the group consisting of I, D-I1e, and S;
X₁₃ is selected from the group consisting of Nle, Aib, L, and αMeL;
X₁₄ is L;
X₁₆ is selected from the group consisting of K, E, Orn, Dab, Dap, S, T, H, Aib, αMeK, and R;
X₁₇ is selected from the group consisting of K, Q, and I;
X₁₉ is selected from the group consisting of Q and A;
X₂₀ is selected from the group consisting of Aib, Q, H, R, K, and αMeK;
X₂₁ is selected from the group consisting of H, Aad, D, Aib, T, A, E, and I;
X₂₃ is selected from the group consisting of I, L, A, G, F, H, E, and V;
X₂₄ is selected from the group consisting of S, Aad, D-Glu, E, Aib, H, V, A, Q, D, and P;
X₂₆ is selected from the group consisting of L and αMeL;
X₂₇ is selected from the group consisting of L and I;
X₂₈ is selected from the group consisting of E, A, S, and D-Glu; and
X₂₉ is selected from the group consisting of Aib, G, and A (hereafter a “Formula IV” compound).

In an embodiment of is a compound of Formula IV, or a pharmaceutically acceptable salt thereof wherein X₃₉ is C. In an embodiment is a compound of Formula IV, or a pharmaceutically acceptable salt thereof wherein X₄₀ is C.

In an embodiment is a compound of Formula IV, or a pharmaceutically acceptable salt thereof, wherein one, and only one, of X₃₀, X₃₄, X₃₉, and X₄₀ is C. In an embodiment is a compound of Formula IV, or a pharmaceutically acceptable salt thereof, wherein one, and only one, of X₃₀, X₃₄, X₃₉, and X₄₀ is C modified using time-extension technology. In an embodiment is a compound of Formula IV, or pharmaceutically acceptable salt thereof, wherein C is modified using time-extension technology wherein the time-extension technology is XTEN. In an embodiment is a compound of Formula IV, or pharmaceutically acceptable salt thereof, wherein C is modified using time-extension technology wherein the time-extension technology is a (Glu)ₘ biotin wherein m is 0, 1, 2, or 3. In an embodiment is a compound of Formula IV, or a pharmaceutically acceptable salt thereof, wherein:
X₁ is Y;
X₂ is Aib;
X₃ is E;
X₁₀ is selected from the group consisting of A, L, H, 3Pal, 4Pal, V, and Y;
X₁₁ is S;
X₁₂ is I;
X₁₆ is selected from the group consisting of K, E, Orn, Dab, and Dap;
X₁₉ is Q;
X₂₀ is selected from the group consisting of Aib and K;
X₂₁ is selected from the group consisting of H, Aad, D, Aib, T, A, and E;
X₂₂ is F;
X₂₃ is I;
X₂₄ is selected from the group consisting of S, Aad, D-Glu, and E;
X₂₆ is L; and
X₂₈ is selected from the group consisting of E and A; or a pharmaceutically acceptable salt thereof.

In an embodiment is a compound of Formula IV, or a pharmaceutically acceptable salt thereof, wherein
X₁ is Y;
X₂ is Aib;
X₃ is E;
X₁₀ is selected from the group consisting of A, L, H, 3Pal, 4Pal, V, and Y;
X₁₁ is S;
X₁₂ is I;
X₁₆ is selected from the group consisting of K, E, Orn, Dab, and Dap;
X₂₀ is Aib;
X₂₁ is selected from the group consisting of H, Aad, D, Aib, T, A, and E;
X₂₂ is F;
X₂₄ is selected from the group consisting of S, Aad, D-Glu, and E;
X₂₇ is I; and
X₂₈ is selected from the group consisting of E and A.

In an embodiment is a compound of Formula I, or a pharmaceutical salt thereof, wherein:
X₁4 is L;
X₁₇ is selected from the group consisting of K, Q, and I;
X₃₀ is selected from the group consisting of G-R₂ and G; and
q is selected from the group consisting of 16, 18, and 20;
wherein if X₃₀ is G, then X₃₁ is selected from the group consisting of:
PX₃₂X₃₃X₃₄-R₂ (SEQ ID NO:4), wherein:
   X₃₂ is S, X₃₃ is S, X₃₄ is G and R₂ is absent (SEQ ID NO:299) or
   X₃₂ is S, X₃₃ is S, X₃₄ is G and R₂ is NH₂ (SEQ ID NO:300);
and
PX₃₂X₃₃X₃₄X₃₅X₃₆X₃₇X₃₈X₃₉-R₂ (SEQ ID NO:5), wherein:
   X₃₂ is S, X₃₃ is S, X₃₄ is G, X₃₅ is A, X₃₆ is P, X₃₇ is P, X₃₈ is P, X₃₉ is S and
   R₂ is absent (SEQ ID NO:301) or
   X₃₂ is S, X₃₃ is S, X₃₄ is G, X₃₅ is A, X₃₆ is P, X₃₇ is P, X₃₈ is P, X₃₉ is S and
   R₂ is NH₂ (SEQ ID NO:302); and
wherein one of X₁₀, X₁₂, X₁₃, X₁₄, X₁₆, X₁₉, X₂₀, X₂₁, X₂₃, X₂₄, X₂₆, X₂₇, X₂₈, and X₂₉ is K(2-[2-(2-amino-ethoxy)-ethoxy]-acetyl)₂-γGlu-CO—(CH₂)qCO₂H (hereafter a “Formula V” compound).

In an embodiment is a compound of Formula V, or a pharmaceutically acceptable salt thereof, wherein:
X₁ is Y;
X₂ is Aib;
X₃ is E;
X₁₀ is selected from the group consisting of A, L, H, 3Pal, 4Pal, V, Y, E, cTA, and K(2-[2-(2-amino-ethoxy)-ethoxy]-acetyl)₂-(γ-Glu)-CO—(CH₂)qCO₂H;
X₁₁ is S;
X₁₂ is selected from the group consisting of I, D-I1e, and K(2-[2-(2-amino-ethoxy)-ethoxy]-acetyl)₂-(γ-Glu)-CO—(CH₂)qCO₂H;
X₁₆ is selected from the group consisting of K, E, Orn, Dab, Dap, and K(2-[2-(2-amino-ethoxy)-ethoxy]-acetyl)₂-(γ-Glu)-CO—(CH₂)qCO₂H;
X₁₇ is selected from the group consisting of K and I;
X₁₉ is selected from the group consisting of Q and K(2-[2-(2-amino-ethoxy)-ethoxy]-acetyl)₂-(γ-Glu)-CO—(CH₂)qCO₂H;
X₂₀ is selected from the group consisting of Aib and K(2-[2-(2-amino-ethoxy)-ethoxy]-acetyl)₂-(γ-Glu)-CO—(CH₂)qCO₂H;
X₂₁ is selected from the group consisting of H, Aad, D, Aib, T, A, E, and K(2-[2-(2-amino-ethoxy)-ethoxy]-acetyl)₂-(γ-Glu)-CO—(CH₂)qCO₂H;
X₂₂ is F;
X₂₄ is selected from the group consisting of S, Aad, D-Glu, E, and K(2-[2-(2-amino-ethoxy)-ethoxy]-acetyl)₂-(γ-Glu)-CO—(CH₂)qCO₂H;
X₂₆ is selected from the group consisting of L and K(2-[2-(2-amino-ethoxy)-ethoxy]-acetyl)₂-(γ-Glu)-CO—(CH₂)qCO₂H;
X₂₇ is selected from the group consisting of L and I; and
X₂₈ is selected from the group consisting of E, A, and K(2-[2-(2-amino-ethoxy)-ethoxy]-acetyl)₂-(γ-Glu)-CO—(CH₂)qCO₂H.

In an embodiment is a compound of Formula V, or a pharmaceutically acceptable salt thereof, wherein X₂₀ is K(2-[2-(2-amino-ethoxy)-ethoxy]-acetyl)₂-(γ-Glu)-CO—(CH₂)qCO₂H, wherein q is 16 or 18. In an embodiment is a compound of Formula V, or a pharmaceutically acceptable salt thereof, wherein X₃₁ is SEQ ID NO:301 or SEQ ID NO:302.

An embodiment provides a method of treating a condition selected from the group consisting of T2DM, obesity, nonalcoholic fatty liver disease (NAFLD), nonalcoholic steatohepatitis (NASH), dyslipidemia and metabolic syndrome, comprising administering to a subject in need thereof, an effective amount of a compound of Formula I or a pharmaceutically acceptable salt thereof. An embodiment provides a method for providing therapeutic weight loss comprising administering to a subject in need thereof, an effective amount of a compound of Formula I, or a pharmaceutically acceptable salt thereof. In one embodiment, the condition is NAFLD. In one embodiment, the condition is NASH.

An embodiment provides a compound of Formula I, or a pharmaceutically acceptable salt thereof, for use in therapy. An embodiment provides a compound of Formula I, or a pharmaceutically acceptable salt thereof, for use in therapy to treat a condition selected from the group consisting of T2DM, obesity, NAFLD, NASH, dyslipidemia and metabolic syndrome. In an embodiment, the condition is T2DM. In an embodiment, the condition is obesity. In an embodiment, the condition is NAFLD. In an embodiment, the condition is NASH. In an embodiment, the condition is metabolic syndrome.

The compounds of Formula I, or a pharmaceutically acceptable salt thereof, may be useful in the treatment of a variety of symptoms or disorders. For example, certain embodiments, provide a method for treatment of T2DM in a patient comprising administering to a subject in need of such treatment an effective amount of a compound of Formula L, or a pharmaceutically acceptable salt thereof. In an embodiment, is a method for treatment of obesity in a patient comprising administering to a subject in need of such treatment an effective amount of a compound of Formula I, or a pharmaceutically acceptable salt thereof. In an embodiment, the method is inducing non-therapeutic weight loss in a subject, comprising administering to a subject in need of such treatment an effective amount of a compound of Formula I, or a pharmaceutically acceptable salt thereof.

In certain embodiments, the present invention provides a method for treatment of metabolic syndrome in a patient comprising administering to a subject in need of such treatment an effective amount of a compound of Formula I, or a pharmaceutically acceptable salt thereof. In an embodiment, the method is treatment of NASH comprising administering to a subject in need of such treatment an effective amount of a compound of Formula I, or a pharmaceutically acceptable salt thereof.

Also provided herein is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, a growth differentiation factor 15 modulator (“GDF15”), a peptide tyrosine tyrosine modulator (“PYY”), a modified insulin, amylin, a dual amylin calcitonin receptor agonist, and oxyntomodulin agonist (“OXM”) in the treatment of a condition selected from the group consisting of T2DM, obesity, NAFLD, NASH, dyslipidemia and metabolic syndrome. In an embodiment, a compound of the present invention is provided in a fixed dose combination with one or more agents selected from metformin, a thiazolidinedione, a sulfonylurea, a dipeptidyl peptidase 4 inhibitor, a sodium glucose co-transporter, a SGLT-2 inhibitorGDF15, PYY, a modified insulin, amylin, a dual amylin calcitonin receptor agonist, and OXM. In an embodiment is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM in the treatment of a condition selected from the group consisting of T2DM and obesity. In an embodiment is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, and a SGLT-2 inhibitor in the treatment of a condition selected from the group consisting of T2DM and obesity.

In other embodiments, the compounds, or a pharmaceutically acceptable salt thereof, may be useful to improve bone strength in subjects in need thereof. The compounds of the present invention, or a pharmaceutically acceptable salt thereof, may be useful in the treatment of other disorders such as Parkinson's disease or Alzheimer's disease. Incretins and incretin analogs having activity at one or more of the GIP, GLP-1 and/or glucagon receptors have been described as having the potential to have therapeutic value in a number of other diseases or conditions, including for example obesity, NAFLD and NASH, dyslipidemia, metabolic syndrome, bone related disorders, Alzheimer's disease, and Parkinson's disease. See, e.g., Jall S., et. al, Monomeric GLP-1/GIP/glucagon triagonism corrects obesity, hepatosteatosis, and dyslipidemia in female mice, MOL. METAB. 6(5):440-446 (March 2017); Carbone L. J., et. al., Incretin-based therapies for the treatment of non-alcoholic fatty liver 5 disease: A systematic review and meta-analysis. J. GASTROENTEROL. HEPATOL., 31(1):23-31 (January 2016); B. Finan, et. al, Reappraisal of GIP Pharmacology for Metabolic Diseases. TRENDS MOL. MED., 22(5):359-76 (May 2016); Choi, I. Y., et al., Potent body weight loss and efficacy in a NASH animal model by a novel long-acting GLP-1/Glucagon/GIP triple-agonist (HM15211), ADA 2017 Poster 1139-P; Ding, K. H., Impact of glucose-dependent insulinotropic peptide on age-induced bone loss, J. BONE MINER. RES., 23(4):536-43 (2008); Tai, J. et. al, Neuroprotective effects of a triple GLP-1/GIP/glucagon receptor agonist in the APP/PS1transgenic mouse model of Alzheimer's disease, BRAIN RES. 1678, 64-74 (2018); T. D. Müller et al., The New Biology and Pharmacology of Glucagon, PHYSIOL. REV. 97: 721-766 (2017); Finan, B. et. al, Unimolecular Dual Incretins Maximize Metabolic Benefits in Rodents, Monkeys, and Humans, SCI. TRANSL. MED., 5:209 (October 2013); Hölscher C, Insulin, incretins and other growthfactors as potential novel treatments for Alzheimer's and Parkinson's diseases. BIOCHEM. SOC. TRANS. 42(2):593-0 (April 2014).

Another embodiment provides the use of a compound of the present invention, or a pharmaceutically acceptable salt thereof, in the manufacture of a medicament for the treatment of a condition selected from the group consisting of T2DM, obesity, NAFLD, NASH, dyslipidemia and metabolic syndrome. In an embodiment, the medicament is for the treatment of T2DM. In an embodiment, the medicament is for the treatment of obesity. In an embodiment, the medicament is for the treatment of NAFLD. In an embodiment, the medicament is for the treatment of NASH.

Another embodiment provides a pharmaceutical composition comprising a compound of Formula I, or a pharmaceutically acceptable salt thereof, and at least one selected from the group consisting of a carrier, diluent, and excipient.

In an embodiment is a pharmaceutical composition comprising a compound of Formula I, or a pharmaceutically acceptable salt thereof. at least one permeation enhancer and at least one protease inhibitor. In an embodiment, is a pharmaceutical composition comprising a compound of Formula I, or a pharmaceutically acceptable salt thereof, at least one permeation enhancer, and at least one selected from the group consisting of carrier, diluent, and excipient.

In an embodiment is a pharmaceutical composition comprising a compound of Formula I, or a pharmaceutically acceptable salt thereof, a permeation enhancer, a protease inhibitor, and at least one selected from the group consisting of carrier, diluent, and excipient. In an embodiment is a pharmaceutical composition comprising a compound of Formula I, or a pharmaceutically acceptable salt thereof, and a permeation enhancer. In an embodiment is a pharmaceutical composition comprising a compound of the present invention, or a pharmaceutically acceptable salt thereof, and a permeation enhancer. In an embodiment the permeation enhancer is selected from the group consisting of sodium decanoate (“C10”), sodium taurodeoxycholate (“NaTDC”), lauroyl carnitine (“LC”), dodecyl maltoside (“C12-maltoside”), dodecyl phosphatidylcholine (“DPC”), sodium N-[8-(2-hydroxybenzoyl)amino] caprylate (“SNAC”) and a Rhamnolipid. In an embodiment the permeation enhancer is selected from the group consisting of C10 and LC. In an embodiment a protease inhibitor is selected from the group consisting of soybean trypsin inhibitor (“SBTI”), soybean trypsin-chymotrypsin inhibitor (“SBTCI”), ecotin, sunflower trypsin inhibitor (“SFTI”), leupeptin, citric acid, ethylenediarminetetraacetic acid (“EDTA”), sodium glycocholate and 4-(2-aminoethyl)benzenesulfonyl fluoride hydrochloride (“AEBSF”). In an embodiment, a protease inhibitor is selected from the group consisting of SBTI, SBTCI, and SFTI. In an embodiment, a protease inhibitor is SBTI.

As used herein, the term “treating” or “to treat” includes restraining, slowing, stopping, or reversing the progression or severity of a symptom, condition, or disorder.

Certain compounds of the present invention are generally effective over a wide dosage range. For example, dosages for once weekly parenteral dosing may fall within the range of 0.05 mg to about 30 mg per person per week.

The compounds of the present invention include novel amino acid sequences having affinity for the respective GLP-1 and GIP receptors, with desired potency at each of these receptors. GLP-1 is a 36 amino acid peptide, the major biologically active fragment of which is produced as a 30-amino acid, C-terminal amidated peptide (GLP-1₇₋₃₆) (SEQ ID NO:2).

GIP is a 42 amino acid peptide (SEQ ID NO:1), which, like GLP-1, is also known as an incretin, and plays a physiological role in glucose homeostasis by stimulating insulin secretion from pancreatic beta cells in the presence of glucose.

The compounds provide desired potency at each of the GIP and GLP-1 receptors. In an embodiment, compounds are suitable for oral administration. In an embodiment, compounds have desirable GIP and GLP receptor extended time action. In an embodiment, compounds have desirable GIP and GLP receptor activity wherein the GIP agonist potency is from 2.5 to 5 times the GLP1 receptor potency as measured by the casein cAMP assay described herein below, wherein the potency is normalized against native GIP and GLP on the day the assay is run. In an embodiment, compounds have desirable GIP and GLP receptor activity wherein the GIP agonist potency is from 2.5 to 10 times the GLP1 receptor potency as measured by the casein cAMP assay, wherein the potency is normalized against native GIP and GLP on the day the assay is run.

As used herein the term “amino acid” means both naturally occurring amino acids and unnatural amino acids. The amino acids are typically depicted using standard one letter codes (e.g., L=leucine), as well as alpha-methyl substituted residues of natural amino acids (e.g., α-methyl leucine, or αMeL and α-methyl lysine, or αMeK) and certain other unnatural amino acids, such as alpha amino isobutyric acid, or “Aib,” “4Pal,” “Orn,” and the like. The structures of these amino acids appear below:

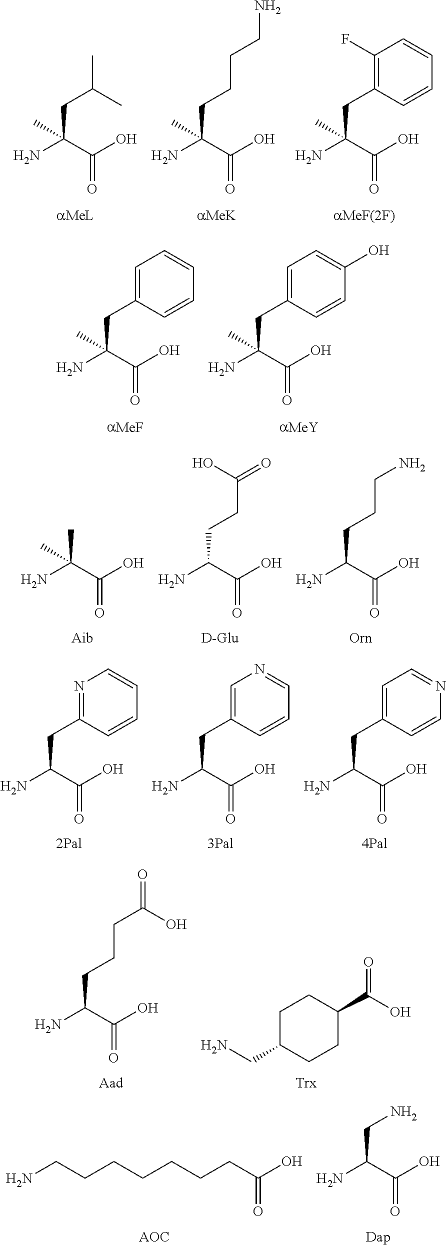

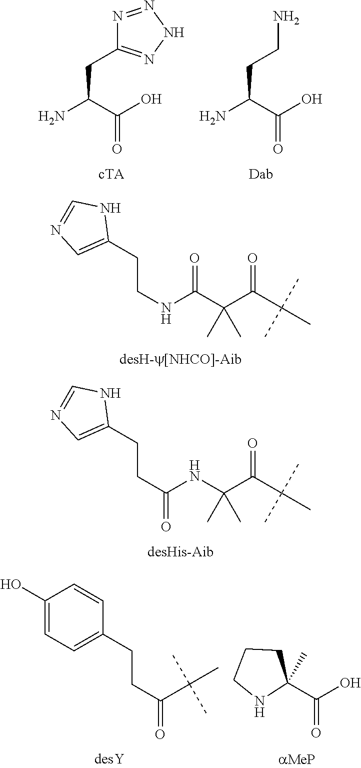

As used herein “Orn” means ornithine. As used herein “4Pal” means 3-(4-Pyridyl)-L-alanine. As used herein “αMeF(2F)” means alpha-methyl 2-F-phenylalanine. As used herein “αMeY,” “αMeK,” and “αMeL” mean alpha methyl tyrosine, alpha methyl lysine, and alpha methyl leucine, respectively. As used herein, “e” and “D-Glu” mean D-glutamic acid. As used herein “D-His” and “h” each mean D-histidine. As used herein “D-Tyr” and “y” each means D-tyrosine. As used herein “D-Ser” and “s” means means D-serine. As used herein “D-Ala” and “a” each means D-alanine. As used herein, “αMeF(2F)” means alpha-methyl-F(2F) and alpha-methyl-Phe(2F). As used herein, “αMeF”, means alpha-methyl-F and alpha-methyl-Phe. As used herein, “αMeY”, means alpha-methyl-Tyr. As used herein “αMeK”, means alpha-methyl-Lys. As used herein, “αMeL”, means alpha-methyl-Leu. As used herein, “αMeS”, means alpha-methyl-serine and alpha-methyl-Ser. As used herein “αMeP”, means alpha-methyl-proline and alpha-methyl-Pro. As used herein, “desH”, means desHis. As used herein, “desY”, means desTyr.

When X₁ is DesH and X₂is Aib, and the DesH and Aib can combine to form a group as illustrated above, DesH-ψ[NHCO]-Aib.

When used herein, the term “amino acid conjugated to a C₁₆-C₂₂ fatty acid” refers to any natural or unnatural amino acid with a functional group that has been chemically modified to conjugate to a fatty acid by way of a covalent bond to the fatty acid or, preferably, by way of a linker. Examples of such functional groups include amino, carboxyl, chloro, bromo, iodo, azido, alkynyl, alkenyl, and thiol groups. Examples of natural amino acids which include such functional groups include K (amino), C (thiol), E (carboxyl) and D (carboxyl). In an embodiment the conjugated amino acid is K.

As noted above, in an embodiment of a compound of Formula I, II, III, IV, and V are compounds of the present invention wherein a fatty acid moiety is conjugated via a linker or a direct bond. In an embodiment, compounds of the present invention include a fatty acid moiety conjugated, preferably via a linker, to a K at position 14 or 17. In an embodiment, the conjugation is an acylation. In an embodiment, the conjugation is to the epsilon-amino group of the K side-chain. In an embodiment of the compounds of the present invention include a fatty acid moiety conjugated, via a linker, to a K at position 17.

In an embodiment, compounds of the present invention include a fatty acid moiety conjugated directly, without a linker, to a natural or unnatural amino acid with a functional group available for conjugation. In certain preferred embodiments the conjugated amino acid is selected from the group consisting of K, C, E and D. In particularly preferred embodiments the conjugated amino acid is K. In such embodiments, the conjugation is to the epsilon-amino group of the K side-chain.

In an embodiment, the linker comprises one to four amino acids, an amino polyethylene glycol carboxylate, or mixtures thereof. In an embodiment, the amino polyethylene glycol carboxylate has the following formula:

H—{NH—CH₂—CH₂—[O—CH₂—CH₂]ₚ—O—(CH₂)_{z}—CO}ᵣ—OH, wherein p is any integer from 1 to 12, z is any integer from 1 to 20, and r is 1 or 2.

In an embodiment is a compound of Formula I which comprises an amino acid conjugated to a fatty acid via a linker, wherein the linker is one to two amino acids selected from the group consisting of Glu and γ-Glu. In an embodiment the linker is one to two (2-[2-(2-Amino-ethoxy)-ethoxy]-acetyl) moieties. The compounds of the present invention utilize a C₁₆-C₂₂ fatty acid chemically conjugated to the functional group of an amino acid either by a direct bond or by a linker. In an embodiment, the fatty acid moiety is conjugated to a lysine at position 17 via a linker between the lysine and the fatty acid. In an embodiment, the fatty acid moiety is conjugated to a lysine at position 20 via a linker between the lysine and fatty acid. In an embodiment, the fatty acid chain is any single chain C₁₆-C₂₂ fatty acid.

In an embodiment is a compound of Formula I, or a pharmaceutically acceptable salt thereof, wherein the fatty acid is conjugated with a linker, and the linker comprises one or more (2-[2-(2-Amino-ethoxy)-ethoxy]-acetyl) moieties, in combination with zero or one to four amino acids. In an embodiment, the linker may comprise one to four Glu or γ-Glu amino acid residues. In an embodiment, the linker may comprise 1 or 2 Glu or γ-Glu amino acid residues. In an embodiment is a compound of Formula I, or a pharmaceutically acceptable salt thereof, comprises a fatty acid conjugated via a linker wherein, the linker comprises either 1 or 2 γ-Glu amino acid residues. In an embodiment is a compound of Formula I, or a pharmaceutically acceptable salt thereof, comprises a fatty acid conjugated via a linker wherein the linker may comprise one to four amino acid residues (such as, for example Glu and [symbol] γ-Glu amino acids) used in combination with up to 36 (2-[2-(2-Amino-ethoxy)-ethoxy]-acetyl) moieties. Specifically, in an embodiment is a Formula I compound, or a pharmaceutically acceptable salt thereof, which comprises a fatty acid conjugated via a linker wherein, the linker constitutes combinations of one to four Glu and [symbol] -Glu amino acids and one to four (2-[2-(2-Amino-ethoxy)-ethoxy]-acetyl) moieties. In an embodiment is a Formula I compound, or a pharmaceutically acceptable salt thereof, which comprises a fatty acid conjugated via a linker wherein the linker is comprised of combinations of one or two γ-Glu amino acids and one or two (2-[2-(2-Amino-ethoxy)-ethoxy]-acetyl) moieties. In an embodiment is a Formula I compound, or a pharmaceutically acceptable salt thereof, which comprises a fatty acid conjugated via a linker wherein the linker and fatty acid components have the following formula:

(2-[2-(2-Amino-ethoxy)-ethoxy]-acetyl)ₐ-(γ-Glu)_{b}-CO—(CH₂)_{q}—CO₂H, wherein a is 1 or 2, b is 1 or 2 and q is 16 or 18. In an embodiment, a is 2, b is 1 and q is 18; and the structure is:

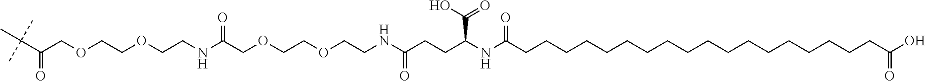

In an embodiment a is 1, b is 1, and q is 18; and the structure is:

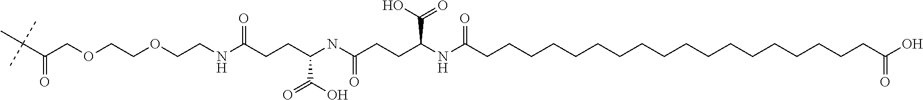

In an embodiment a is 1, b is 1, and q is 18; and the structure is:

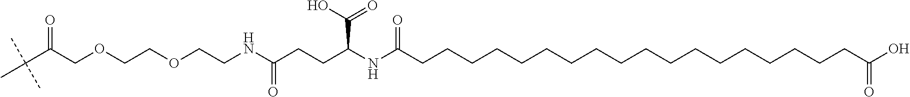

The term “C₁₆-C₂₂ fatty acid” as used herein means a carboxylic acid with between 16 and 22 carbon atoms. In an embodiment, the C₁₆-C₂₂ fatty acid suitable for use herein can be a saturated diacid. In an embodiment, the fatty acid is C₂₀-C₂₂. In an embodiment q is selected from the group consisting of 14, 16, 18, and 20. In an embodiment q is selected from 18 and 20. In an embodiment q is 18. In an embodiment q is 20.

In an embodiment, specific saturated C₁₆-C₂₂ fatty acids that are suitable for the compounds and uses thereof disclosed herein include, but are not limited to, hexadecanedioic acid (C₁₆ diacid), heptadecanedioic acid (C₁₇ diacid), octadecanedioic acid (C₁₈ diacid), nonadecanedioic acid (C₁₉ diacid), eicosanedioic acid (C₂₀ diacid), heneicosanedioic acid (C₂₁ diacid), docosanedioic acid (C₂₂ diacid), including branched and substituted derivatives thereof.

In an embodiment, the C₁₆-C₂₂ fatty acid is selected from the group consisting of a saturated C₁₈ diacid, a saturated C₁₉ diacid, a saturated C₂₀ diacid, and branched and substituted derivatives thereof. In an embodiment, the C₁₆-C₂₂ fatty acid is selected from the group consisting of stearic acid, arachadic acid and eicosanedioic acid. In an embodiment, the C₁₆-C₂₂ fatty acid is arachadic acid.

As shown in the chemical structures of Examples 1-5 below, in an embodiment the linker-fatty acid moieties described above link to the epsilon-amino group of the lysine side-chain.

In an embodiment, is a compound of Formula I, or a pharmaceutically acceptable salt thereof, wherein none of X₃₀, X₃₁, X₃₂, X₃₃, X₃₄, X₃₅, X₃₆, X₃₇, X₃₈, X₃₉, and X₄₀ is C or is a substituent that contains a fatty acid. In an embodiment, is a compound of Formula I, or a pharmaceutically acceptable salt thereof, wherein none of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ is a substituent that contains a fatty acid; and none of X₃₀, X₃₄, X₃₉, and X₄₀ is C. In an embodiment is a compound of Formula I, or a pharmaceutically acceptable salt thereof, wherein none of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃0,
X₃₁, X₃₂, X₃₃, X₃₄, X₃₅, X₃₆, X₃₇, X₃₈, X₃₉, and X₄₀ is a substituent that contains a fatty acid.

As used herein “time-extension technology” means a peptide time-extension technology for example, recombinant human serum albumin (“rHSA”), peptide conjugation to a pharmaceutically acceptable polymer, such as polymeric sequence of amino acids (“XTEN”), unsulfated heparin-like carbohydrate polymer (“HEP”), hydroxyl ethyl starch (“HES”), llama heavy-chain antibody fragments (“VHH”), pegylation, Fc conjugation, bovine serum albumin (“BSA”) (Sleep, D. Epert Opin Drug Del (2015) 12, 793-812; Podust V N et. al. J Control. Release, 2015; ePUB; Hey, T. et. al. in: R. Kontermann (Ed.), Therapeutic Proteins: Strategies to Modulate their Plasma Half-Lives, Wiley-VCH Verlag Gmbh & Co. KGaA, Weinheim, Germany, 2012, pp 117-140; DeAngelis, P L, Drug Dev Delivery (2013) January, Dec. 12, 2012. In an embodiment time-extension technology is applied using a linker group. In an embodiment, the time-extension technology is applied using 0, 1, 2, or 3 amino acids as linker.

In an embodiment is a compound of Formula I, or a pharmaceutically acceptable salt thereof, without a fatty acid (i.e., a compound where none of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ is a substituent that contains a fatty acid) or time-extension technology may be administered to a patient in need thereof via transdermal or infusion methods of administration. Further, a compound of Formula I, or a pharmaceutically acceptable salt thereof, without a fatty acid may be further modified using a peptide time-extension technology for example, recombinant human serum albumin (“rHSA”), peptide conjugation to a pharmaceutically acceptable polymer, such as polymeric sequence of amino acids (“XTEN”), unsulfated heparin-like carbohydrate polymer (“HEP”), and hydroxyl ethyl starch (“HES”). In an embodiment, a time-extension technology is applied using a cysteine amino acid in a Formula I compound, or a pharmaceutically acceptable salt thereof, without a fatty acid, using procedures known to the skilled artisan. In an embodiment, a time-extension technology is applied to one amino acid in a Formula I compound, or a pharmaceutically acceptable salt thereof, without a fatty acid. In an embodiment, wherein a time-extension technology is applied to a Formula I compound, or a pharmaceutically acceptable salt thereof, without a fatty acid, X₁₇ is selected from the group consisting of I, K and Q. In an embodiment wherein a time-extension technology is applied to a Formula I compound, or a pharmaceutically acceptable salt thereof, without a fatty acid, X₃₀ is C. In an embodiment wherein a time-extension technology is applied to a Formula I compound, or a pharmaceutically acceptable salt thereof, without a fatty acid, X₃₄ is C. In an embodiment wherein a time-extension technology is applied to a Formula I compound, or a pharmaceutically acceptable salt thereof, without a fatty acid, X₃₉ is C. In an embodiment wherein a time-extension technology is applied to a Formula I compound, or a pharmaceutically acceptable salt thereof, without a fatty acid, X₄₀ is C.

When used herein in reference to one or more of the GIP or GLP-1 receptors, the terms “activity,” “activate[s]” “activat[ing]” and the like refers to the capacity of a compound, or a pharmaceutically acceptable salt thereof, to bind to and induce a response at the receptor(s), as measured using assays known in the art, such as the in vitro assays described below.

The affinity of compounds, or a pharmaceutically acceptable salt thereof, of the present invention for each of the GIP and GLP-1 receptors may be measured using techniques known for measuring receptor binding levels in the art, including, for example those described in the examples below, and is commonly expressed as a Ki value. The activity of the compounds of the present invention at each of the receptors may also be measured using techniques known in the art, including for example the in vitro activity assays described below, and is commonly expressed as an EC₅₀ value, which is the concentration of compound causing half-maximal simulation in a dose response curve.

In an embodiment, a pharmaceutical composition of a compound of Formula I is suitable for administration by a parenteral route (e.g., subcutaneous, intravenous, intraperitoneal, intramuscular, or transdermal). In an embodiment, a pharmaceutical composition of a compound of Formula I is suitable for oral administration (e.g., tablet, capsule), Some pharmaceutical compositions and processes for preparing same are well known in the art. (See, e.g., Remington: The Science and Practice of Pharmacy (D. B. Troy, Editor, 21st Edition, Lippincott, Williams & Wilkins, 2006). Physiochemical properties of a peptide in addition to anatomical and physiological features of the gastrointestinal tract may provide challenges to efficient oral delivery of a peptide. In an embodiment a pharmaceutical composition for oral administration comprises of a compound of this invention, and a permeation enhancer. In an embodiment, a pharmaceutical composition for oral administration comprises a compound of Formula I or a pharmaceutically acceptable salt thereof, a permeation enharncer, and a protease inhibitor. In an embodiment, a pharmaceutical composition for oral administration comprises a compound of Formula I, or a pharmaceutically acceptable salt thereof, and a permeation enharncer,

Monolithic and multi-particulate dosage forms for compounds of the present invention are contemplated. In an embodiment, a compound of Formula I is provided as a monolithic composition. A monolithic composition is intended for release of all components in a single location. A multi-particuate composition is intended to achieve fast transit from the stomach to the intestine and allow for distribution of composition components over large surface of small intestine. Concurrent release of a compound and functional excipients is desired for monolithic and multi-particulate dosage compositions. In an embodiment a monolithic composition of a compound of Formula I, or a pharmaceutically acceptable salt thereof, is formulated as an enteric capsule, enteric coated capsule or an enteric coated tablet. Such multi-particulate composition may be formulated as an enteric coated minitablets, or enteric coated granules where the coating is generally intact in the stomach at low pH and dissolves at the higher pH of the intestine. Two types of coated minitablets or coated granules may be formulated for either delivery to proximal small intestine by dissolution above pH 5.5 or to distal small intestine by dissolution above pH 7-7.2. A coating system for distal small intestinal release can also be applied to monolithic capsules or tablets if distal small intestinal delivery is desired. Minitablets may be filled into a standard uncoated capsule.

As used herein the term “permeation enhancer” means permeation enhancer that enhances oral absorption of a compound of this invention. As used herein, permeation enhancer means permeation enhancers, such as sodium decanoate, sodium taurodeoxycholate, lauroyl carnitine, dodecyl maltoside, dodecyl phosphatidylcholine, SNAC, a Rhamnolipid, and permeation enhancers reported in the literature, such as for example, Permeant inhibitor of phosphatase, PIP-250 and PIP-640. See, Pharmaceutics. 2019 January; 11(1): 41, (See Biomaterials. 2012; 33: 3464-3474), ZOT (zonula occludens toxin), AG (fragment of ZOT) (See Int. J. Pharm. 2009; 365, 121-130). In an embodiment, a permeation enhancer is selected from the group consisting of sodium decanoate, sodium taurodeoxycholate, and lauroyl carnitine. In an embodiment, a permeation enhancer is selected from the group consisting of C10, LC, and NaTDC. In an embodiment a permeation enhancer is C10.

As used herein the term “protease inhibitor” means a protease inhibitor that may be selected from the group consisting of protein based, peptide based, and small molecule based. Protease inhibitors are well known and may include, for example, soybean trypsin inhibitor (“SBTI”), soybean trypsin-chymotrypsin inhibitor (“SBTCI”), ecotin, sunflower trypsin inhibitor (“SFTI”), leupeptin, citric acid, ethylenediaminetetraacetic acid (“EDTA”), sodium glycocholate and 4-(2-aminoethyl)benzenesulfonyl fluoride hydrochloride (“AEBSF”). In an embodiment a protease inhibitor is selected from the group consisting of SBTI, SBTCI and SFTI. In an embodiment, a protease inhibitor is SBTI.

In an embodiment is a compound of Formula I, or a pharmaceutically acceptable salt thereof, wherein the compound is a potent GIPR/GLP-1R dual agonist that is a partial agonist on the GLP-1R as demonstrated by a Cell Membrane Guanosine 5′-(gamma-thio)Triphosphate-[³⁵S](GTPγS) Binding Assay, and a partial agonist on the GLP-1R as demonstrated by a β-arrestin-2 recruitment assay. In an embodiment is a compound of Formula I, or pharmaceutically acceptable salt thereof, wherein the compound stimulates GLP-1R induced activation of Gas in the GLP-1R HEK293 Cell Membrane Guanosine 5′-(gamma-thio)Triphosphate-[³⁵S](GTPγS) Binding Assay. In an embodiment, is a compound showing partial agonism of 75% or less in the GLP-1R HEK293 Cell Membrane Guanosine 5′-(gamma-thio)Triphosphate-[³⁵S](GTPγS) Binding Assay, and 35% or less in the GLP-CHO Cell β-Arrestin.Recruitment Assay.

In an embodiment is a method for treating diabetes comprising administering an effective amount of a compound showing partial agonism of 75% or less in the GLP-1R HEK293 Cell Membrane Guanosine 5′-(gamma-thio) Triphosphate-[³⁵S](GTPγS) Binding Assay, and an effective amount of a compound that is a GIP agonist. In an embodiment, the compound showing partial agonism in the GLP-1R HEK293 Cell Membrane Guanosine 5′-(gamma-thio) Triphosphate-[³⁵S](GTPγS) Binding Assay is co-administered with a compound having GIP agonist activity. In an embodiment, the compound showing partial agonism in the GLP-1R HEK293 Cell Membrane Guanosine 5′-(gamma-thio) Triphosphate-[³⁵S](GTPγS) Binding Assay is administered as an active agent within one week before or after a compound having GIP agonist activity. In an embodiment, a method for treating diabetes comprises administering an effective amount of a compound showing 35% or less in the GLP-CHO Cell β-Arrestin.Recruitment Assay and administering an effective amount of a compound showing partial agonism of 75% or less in the GLP-1R HEK293 Cell Membrane Guanosine 5′-(gamma-thio) Triphosphate-[³⁵S](GTPγS) Binding Assay.

Compounds of the present invention may react with any of a number of inorganic and organic acids/bases to form pharmaceutically acceptable acid/base addition salts. Pharmaceutically acceptable salts and common methodology for preparing them are well known in the art. (See, e.g., P. Stahl, et al. Handbook of Pharmaceutical Salts: Properties, Selection and Use, 2nd Revised Edition (Wiley-VCH, 2011)). Pharmaceutically acceptable salts of the present invention include, but are not limited to, sodium, trifluoroacetate, hydrochloride, ammonium, and acetate salts. In an embodiment, a pharmaceutically acceptable salt of is selected from the group consisting of sodium, hydrochloride, and acetate salts.

The present invention also encompasses novel intermediates and processes useful for the synthesis of compounds of the present invention, or a pharmaceutically acceptable salt thereof. The intermediates and compounds of the present invention may be prepared by a variety of procedures known in the art. In particular, the Examples below describe a process using chemical synthesis. The specific synthetic steps for each of the routes described may be combined in different ways to prepare compounds of the present invention. The reagents and starting materials are readily available to one of ordinary skill in the art.

When used herein, the term “effective amount” refers to the amount or dose of a compound of the present invention, or a pharmaceutically acceptable salt thereof, which, upon single or multiple dose administration to the patient, provides the desired effect in the patient under diagnosis or treatment. An effective amount can be determined by a person of skill in the art using known techniques and by observing results obtained under analogous circumstances. In determining the effective amount for a subject, a number of factors are considered, including, but not limited to: the species of mammal; its size, age, and general health; the specific disease or disorder involved; the degree of or involvement or the severity of the disease or disorder; the response of the individual patient; the particular compound administered: the mode of administration; the bioavailability characteristics of the preparation administered; the dose regimen selected; the use of concomitant medication; and other relevant circumstances.

When used herein, the term “subject in need thereof” refers to a mammal, preferably a human, with a disease or condition requiring treatment or therapy, including for example those listed in the preceding paragraphs. As used herein “EDTA” means ethylenediaminetetraacetic acid. As used herein “DMSO” means dimethyl sulfoxide. As used herein “CPM” means counts per minute. As used herein “IBMX” means 3-isobutyl-1-methylxanthine. As used herein “LC/MS” means liquid chromatography/mass spectrometry. As used herein “HTRF” means homogeneous time-resolved fluorescence. As used herein “BSA” mean bovine serum albumin.

The invention is further illustrated by the following examples, which are not to be construed as limiting.

### PEPTIDE SYNTHESIS

### Example 1

### Y-Aib-EGT-αMeF(2F)-TSDYSI-αMeL-LDEK((2-[2-(2-Amino-ethoxy)-ethoxy]-acetyl)₂-(γ-Glu)-CO—(CH₂)₁₈—CO₂H)AQ-Aib-EFI-(D-Glu)-YLIEGGPSSGAPPPS-NH₂ (SEQ ID NO:10)

The structure of SEQ ID NO:10 is depicted below using the standard single letter amino acid codes with the exception of residues Aib2, αMeF(2F)6, αMeL13, K17, Aib20, D-Glu24, and Ser39 where the structures of these amino acid residues have been expanded:

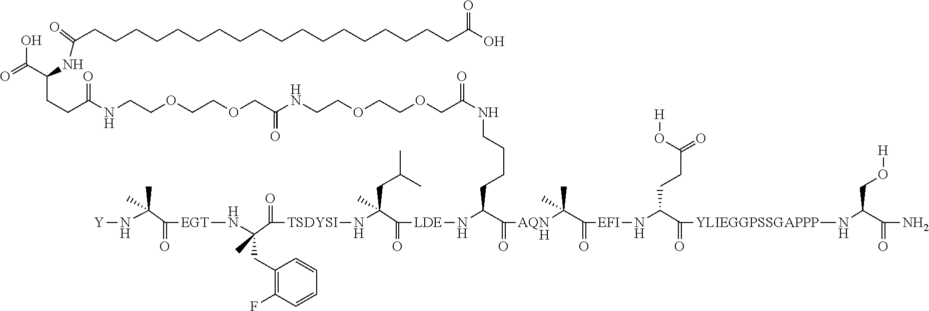

The peptide backbone of Example 1 is synthesized using Fluorenylmethyloxycarbonyl (Fmoc)/tert-Butyl (t-Bu) chemistry on a Symphony X peptide synthesizer (Gyros Protein Technologies. Tucson, AZ).

The resin consists of 1% DVB cross-linked polystyrene (Fmoc-Rink-MBHA Low Loading resin. 100-200 mesh, EMD Millipore) at a substitution of 0.3-0.4 meq/g. Standard side-chain protecting groups were used. Fmoc-Lys(Mtt)-OH is used for the lysine at position 17 and Boc-Tyr(tBu)-OH) was used for the tyrosine at position 1. Fmoc groups are removed prior to each coupling step (2×7 minutes) using 20% piperidine in DMF. All standard amino acid couplings are performed for 1 hour to a primary amine and 3 hour to a secondary amine, using an equal molar ratio of Fmoc amino acid (0.3 mM), diisopropylcarbodiimide (0.9 mM) and Oxyma (0.9 mM), at a 9-fold molar excess over the theoretical peptide loading. Exceptions are couplings to Ca-methylated amino acids, which are coupled for 3 hours. After completion of the synthesis of the peptide backbone, the resin is thoroughly washed with DCM for 6 times to remove residual DMF. The Mtt protecting group on the lysine at position 17 is selectively removed from the peptide resin using two treatments of 30% hexafluoroisopropanol (Oakwood Chemicals) in DCM (2×40-minute treatment).

Subsequent attachment of the fatty acid-linker moiety is accomplished by coupling of 2-[2-(2-Fmoc-amino-ethoxy)-ethoxy]-acetic acid (Fmoc-AEEA-OH, ChemPep, Inc.), Fmoc-glutamic acid α-t-butyl ester (Fmoc-Glu-OtBu, Ark Pharm, Inc.), mono-OtBu-eicosanedioic acid (WuXi AppTec, Shanghai, China). 3-Fold excess of reagents (AA:PyAOP:DIPEA=1:1:1 mol/mol) are used for each coupling that is 1-hour long.

After the synthesis is complete, the peptide resin is washed with DCM, and then thoroughly air-dried. The dry resin is treated with 10 mL of cleavage cocktail (trifluoroacetic acid:water:triisopropylsilane, 95:2.5:2.5 v/v) for 2 hours at room temperature. The resin is filtered off, washed twice each with 2 mL of neat TFA, and the combined filtrates are treated with 5-fold excess volume of cold diethyl ether (−20° C.) to precipitate the crude peptide. The peptide/ether suspension is then centrifuged at 3500 rpm for 2 min to form a solid pellet, the supernatant is decanted, and the solid pellet is triturated with ether two additional times and dried in vacuo. The crude peptide is solubilized in 20% acetonitrile/20% Acetic acid/60% water and purified by RP-HPLC on a Luna 5 μm Phenyl-Hexyl preparative column (21×250 mm, Phenomenex) with linear gradients of 100% acetonitrile and 0.1% TFA/water buffer system (30-50% acetonitrile in 60 min). The purity of peptide is assessed using analytical RP-HPLC and pooling criteria is >95%. The main pool purity of compound 1 is found to be 98.0%. Subsequent lyophilization of the final main product pool yielded the lyophilized peptide TFA salt. The molecular weight is determined by LC-MS (obsd: M+3=1657.2; Calc M+3=1657.0).

### Example 2

### Y-Aib-EGT-αMeF(2F)-TSDYSI-αMeL-LD-Orn-K((2-[2-(2-Amino-ethoxy)-ethoxy]-acetyl)₂-(γ-Glu)-CO—(CH₂)₁₆—CO₂H)AQ-Aib-EFI-(D-Glu)-YLIEGGPSSGAPPPS-NH₂ (SEQ ID NO:11)

The structure of SEQ ID NO:11 is depicted below using the standard single letter amino acid codes with the exception of residues Aib2, αMeF(2F)6, αMeL13, Orn16, K17, Aib20 D-Glu24, and Ser39 where the structures of these amino acid residues have been expanded:

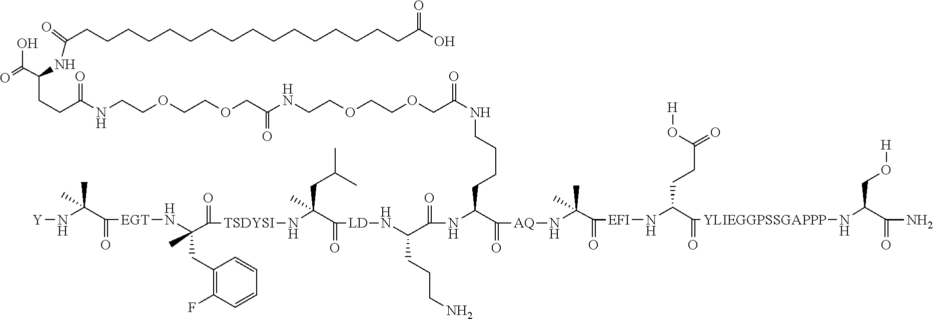

The compound according to SEQ ID NO:11 is prepared substantially as described by the procedures of Example 1. The molecular weight is determined by LC-MS (obsd: M+3=1642.6; Calc M+3=1642.8).

### Example 3

Example 3 is a compound represented by the following description:

### Y-Aib-EGT-αMeF(2F)-TSDYSI-αMeL-LD-Orn-K((2-[2-(2-Amino-ethoxy)-ethoxy]-acetyl)₂-(γ-Glu)-CO—(CH₂)₁₈—CO₂H)AQ-Aib-EFI-(D-Glu)-YLIEGGPSSGAPPPS-NH₂ (SEQ ID NO:12)

The structure of SEQ ID NO:12 is depicted below using the standard single letter amino acid codes with the exception of residues Aib2, αMeF(2F)6, αMeL13, Orn16, K17, Aib20, D-Glu24, and Ser39, where the structures of these amino acid residues have been expanded:

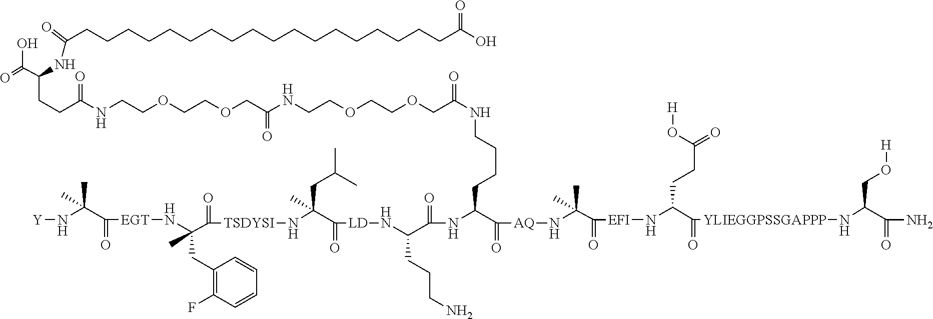

The compound according to SEQ ID NO:12 is prepared substantially as described by the procedures of Example 1. The molecular weight is determined by LC-MS (obsd: M+3=1651 0.8; Calc M+3=1652.2).

### Example 4

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO—(CH₂)₁₆—CO₂H)AQ-Aib-EFI-(D-Glu)-αMeY-LIEGGPSSGAPPPS-NH₂ (SEQ ID NO:13)

The structure of SEQ ID NO:13 is depicted below using the standard single letter amino acid codes with the exception of residues Aib2, αMeF(2F)6, 4Pal10, αMeL13, Orn16, K17, Aib20, D-Glu24 αMeY25, and Ser39, where the structures of these amino acid residues have been expanded:

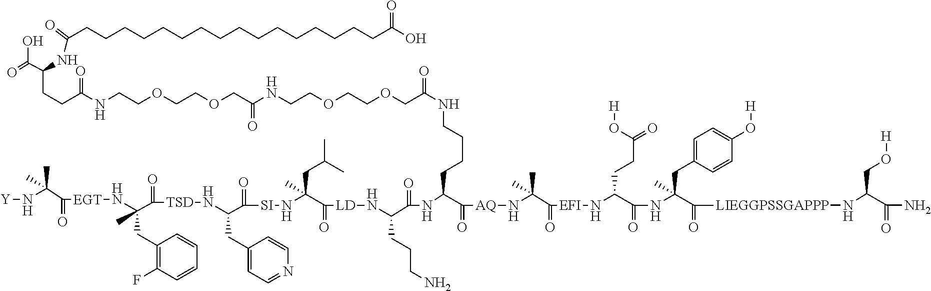

The compound according to SEQ ID NO:13 is prepared substantially as described by the procedures of Example 1. The molecular weight is determined by LC-MS (obsd: M+3=1642.5; Calc M+3=1642.1).

### Example 5

### Y-Aib-EGT-αMeF(2F)-TSDVSI-αMeL-LD-Orn-K((2-[2-(2-Amino-ethoxy)-ethoxy]-acetyl)₂-(γ-Glu)-CO—(CH₂)₁₆—CO₂H)AQ-Aib-EFI-(D-Glu)-αMeY-LIEGGPSSGAPPPS-NH₂ (SEQ ID NO:14)

The structure of SEQ ID NO:14 is depicted below using the standard single letter amino acid codes with the exception of residues Aib2, αMeF(2F)6, αMeL13, Orn16, K17, Aib20, D-Glu24,αMeY25, and Ser39, where the structures of these amino acid residues have been expanded:

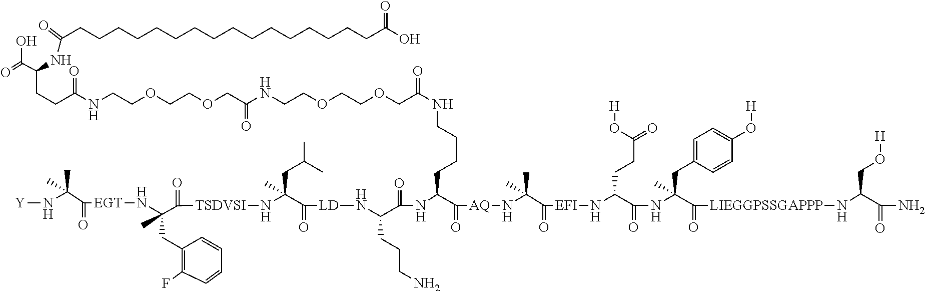

The compound according to SEQ ID NO:14 is prepared substantially as described by the procedures of Example 1. The molecular weight is determined by LC-MS (obsd: M+3=1626.1; Calc M+3=1626.1).

### Example 6 Through Example 287

The compounds according to Examples 6 (SEQ ID NO: 15) through Example 287 (SEQ ID NO:296) are prepared substantially as described by the procedures of Example 1.

| | | | Calculated | Found |
|---|---|---|---|---|
| | | SEQ | MW | MW |
| Example | Compound Name | ID NO | (average) | (average) |
| 6 | Y-Aib-EGTFTSDYSILLDKK((2-[2-(2- | 15 | 4863.5 | 4862.1 |
| | Amino-ethoxy)-ethoxy]-acetyl)₂-(γ-Glu)-CO—(CH₂)₁₈—CO₂H) | | | |
| | AQ-Aib- | | | |
| | AFIEYLLEGGPSSGAPPPS-NH₂ | | | |
| 7 | Y-Aib-EGTFTSDYSILLDSK((2-[2-(2- | 16 | 4822.4 | 4821.3 |
| | Amino-ethoxy)-ethoxy]-acetyl)₂-(γ-Glu)-CO—(CH₂)₁₈—CO₂H) | | | |
| | AQ-Aib- | | | |
| | AFIEYLLEGGPSSGAPPPS-NH₂ | | | |
| 8 | Y-Aib-EGTFTSDYSILLDKK((2-[2-(2- | 17 | 4863.5 | 4863.2 |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 9 | Y-Aib-EGTFTSDYSILLDSK((2-[2-(2- | 18 | 4822.4 | 4820.7 |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 10 | Y-Aib-EGTFTSDYSILLDSIAQ-Aib- | 19 | 4776.5 | 4775.4 |
| | AFIK((2-[2-(2-Amino-ethoxy)-ethoxy]- | | | |
| | acetyl)₂-(γ-Glu)-CO—(CH₂)₁₈—CO₂H)YLLA- | | | |
| | Aib-GPSSGAPPPS-NH₂ | | | |
| 11 | Y-Aib-EGTFTSDYSILLDSIAQ-Aib- | 20 | 4834.5 | 4834.8 |
| | AFIEYLLK((2-[2-(2-Amino-ethoxy)- | | | |
| | ethoxy]-acetyl)₂-(γ-Glu)-CO—(CH₂)₁₈—CO₂H)- | | | |
| | Aib-GPSSGAPPPS-NH₂ | | | |
| 12 | Y-Aib-EGTFTSDYSILLDKIAQK((2-[2-(2- | 21 | 4891.6 | 4890.0 |
| | Amino-ethoxy)-ethoxy]-acetyl)₂-(γ-Glu)-CO—(CH₂)₁₈—CO₂H)- | | | |
| | AFIEYLIEGGPSSGAPPPS- | | | |
| | NH₂ | | | |
| 13 | Y-Aib-EGTFTSDYSILLD-Aib-IAQK((2-[2- | 22 | 4848.5 | 4846.8 |
| | (2-Amino-ethoxy)-ethoxy]-acetyl)₂-(γ-Glu)- | | | |
| | CO—(CH₂)₁₈—CO₂H)- | | | |
| | AFIEYLIEGGPSSGAPPPS-NH₂ | | | |
| 14 | Y-Aib-EGTFTSDYSILLDKIAQK((2-[2-(2- | 23 | 4976.7 | 4975.5 |
| | Amino-ethoxy)-ethoxy]-acetyl)₂-(γ-Glu)-CO—(CH₂)₁₈—CO₂H) | | | |
| | EFIQYLLE-Aib- | | | |
| | GPSSGAPPPS-NH₂ | | | |
| 15 | H-Aib-EGTFTSDYSILLDKK((2-[2-(2- | 24 | 4865.5 | 4863.9 |
| | Amino-ethoxy)-ethoxy]-acetyl)₂-(γ-Glu)-CO—(CH₂)₁₈—CO₂H) | | | |
| | AQ-Aib-AFIEYLLE-Aib- | | | |
| | GPSSGAPPPS-NH₂ | | | |
| 16 | H-Aib-EGTFTSDYSILLDKK((2-[2-(2- | 25 | 4865.5 | 4863.9 |
| | Amino-ethoxy)-ethoxy]-acetyl)₂-(γ-Glu)-CO—(CH₂)₁₈—CO₂H) | | | |
| | AQ-Aib-AFIEYLIE-Aib- | | | |
| | GPSSGAPPPS-NH₂ | | | |
| 17 | H-Aib-EGTFTSDYSILLDKIAQK((2-[2-(2- | 26 | 4444.1 | 4442.7 |
| | Amino-ethoxy)-ethoxy]-acetyl)₂-(γ-Glu)-CO—(CH₂)₁₈—CO₂H) | | | |
| | AFIEYLLE-Aib-GPSSG-NH₂ | | | |
| 18 | H-Aib-EGTFTSDYSI-αMeL-LDKK(Dab-(2- | 27 | 4979.8 | 4978.8 |
| | [2-(2-Amino-ethoxy)-ethoxy]-acetyl)-Dab-(2- | | | |
| | [2-(2-Amino-ethoxy)-ethoxy]-acetyl)-CO—(CH₂)₁₈—CO₂H) | | | |
| | AQ-αMeK-AFIQYLLA-Aib- | | | |
| | GPSSGAPKPS-NH₂ | | | |
| 19 | H-Aib-EGTFTSDYSI-αMeL-LDKK(Dab-(2- | 28 | 4948.8 | 4947.2 |
| | [2-(2-Amino-ethoxy)-ethoxy]-acetyl)-Dab-(2- | | | |
| | [2-(2-Amino-ethoxy)-ethoxy]-acetyl)-CO—(CH₂)₁₈—CO₂H) | | | |
| | AQ-αMeK-AFIQYLLA-Aib- | | | |
| | GPSSGAPPPS-NH₂ | | | |
| 20 | Y-Aib-EGTFTSDYSI-αMeL-LDKK((2-[2- | 29 | 4877.5 | 4875.9 |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 21 | Y-Aib-EGTFTSDYSI-αMeL-LDEK((2-[2- | 30 | 4935.6 | 4934.1 |
| | (2-Amino-ethoxy)-ethoxy]-acetyl)₂-(γ-Glu)- | | | |
| | CO—(CH₂)₁₈—CO₂H)AQ-αMeK- | | | |
| | AFIEYLLEGGPSSGAPPPS-NH₂ | | | |
| 22 | Y-Aib-EGTFTSDYSI-αMeL-LDEK((2-[2- | 31 | 4963.6 | 4962.0 |
| | (2-Amino-ethoxy)-ethoxy]-acetyl)₂-(γ-Glu)- | | | |
| | CO—(CH₂)₁₈—CO₂H)AQ-αMeK-AFIEYLLE- | | | |
| | Aib-GPSSGAPPPS-NH₂ | | | |
| 23 | Y-Aib-EGTFTSDK((2-[2-(2-Amino-ethoxy)- | 32 | 4813.5 | 4812.9 |
| | ethoxy]-acetyl)₂-(γ-Glu)-CO—(CH₂)₁₈—CO₂H) | | | |
| | SILLDKIAQ-Aib- | | | |
| | AFIEYLIEGGPSSGAPPPS-NH₂ | | | |
| 24 | Y-Aib-EGTFTSDYK((2-[2-(2-Amino- | 33 | 4889.6 | 4888.6 |
| | ethoxy)-ethoxy]-acetyl)₂-(γ-Glu)-CO—(CH₂)₁₈—CO₂H) | | | |
| | ILLDKIAQ-Aib- | | | |
| | AFIEYLIEGGPSSGAPPPS-NH₂ | | | |
| 25 | Y-Aib-EGTFTSDYSK((2-[2-(2-Amino- | 34 | 4863.5 | 4862.5 |
| | ethoxy)-ethoxy]-acetyl)₂-(γ-Glu)-CO—(CH₂)₁₈—CO₂H) | | | |
| | LLDKIAQ-Aib- | | | |
| | AFIEYLIEGGPSSGAPPPS-NH₂ | | | |
| 26 | Y-Aib-EGTFTSDYSIK((2-[2-(2-Amino- | 35 | 4863.5 | N/I |
| | ethoxy)-ethoxy]-acetyl)₂-(γ-Glu)-CO—(CH₂)₁₈—CO₂H) | | | |
| | LDKIAQ-Aib- | | | |
| | AFIEYLIEGGPSSGAPPPS-NH₂ | | | |
| 27 | Y-Aib-EGTFTSDYSILK((2-[2-(2-Amino- | 36 | 4863.5 | N/I |
| | ethoxy)-ethoxy]-acetyl)₂-(γ-Glu)-CO—(CH₂)₁₈—CO₂H) | | | |
| | DKIAQ-Aib- | | | |
| | AFIEYLIEGGPSSGAPPPS-NH₂ | | | |
| 28 | Y-Aib-EGTFTSDYSILLK((2-[2-(2-Amino- | 37 | 4861.6 | N/I |
| | ethoxy)-ethoxy]-acetyl)₂-(γ-Glu)-CO—(CH₂)₁₈—CO₂H) | | | |
| | KIAQ-Aib- | | | |
| | AFIEYLIEGGPSSGAPPPS-NH₂ | | | |
| 29 | Y-Aib-EGTFTSDYSILLDK((2-[2-(2-Amino- | 38 | 4848.5 | N/I |
| | ethoxy)-ethoxy]-acetyl)₂-(γ-Glu)-CO—(CH₂)₁₈—CO₂H) | | | |
| | IAQ-Aib-AFIEYLIEGGPSSGAPPPS- | | | |
| | NH₂ | | | |
| 30 | Y-Aib-EGTFTSDYSILLDKIK((2-[2-(2- | 39 | 4905.6 | N/I |
| | Amino-ethoxy)-ethoxy]-acetyl)₂-(γ-Glu)-CO—(CH₂)₁₈—CO₂H) | | | |
| | Q-Aib- | | | |
| | AFIEYLIEGGPSSGAPPPS-NH₂ | | | |
| 31 | Y-Aib-EGTFTSDYSILLDKIAK((2-[2-(2- | 40 | 4848.5 | N/I |
| | Amino-ethoxy)-ethoxy]-acetyl)₂-(γ-Glu)-CO—(CH₂)₁₈—CO₂H)- | | | |
| | Aib- | | | |
| | AFIEYLIEGGPSSGAPPPS-NH₂ | | | |
| 32 | Y-Aib-EGTFTSDYSILLDKIAQ-Aib-K((2- | 41 | 4905.6 | N/I |
| | [2-(2-Amino-ethoxy)-ethoxy]-acetyl)₂-(γ- | | | |
| | Glu)-CO—CH₂)₁₈—CO₂H) | | | |
| | FIEYLIEGGPSSGAPPPS-NH₂ | | | |
| 33 | Y-Aib-EGTFTSDYSILLDKIAQ-Aib- | 42 | 4863.5 | N/I |
| | AFK((2-[2-(2-Amino-ethoxy)-ethoxy]- | | | |
| | acetyl)₂-(γ-Glu)-CO—(CH₂)₁₈—CO₂H) | | | |
| | EYLIEGGPSSGAPPPS-NH₂ | | | |
| 34 | Y-Aib-EGTFTSDYSILLDKIAQ-Aib- | 43 | 4847.6 | N/I |
| | AFIK((2-[2-(2-Amino-ethoxy)-ethoxy]- | | | |
| | acetyl)₂-(γ-Glu)-CO—(CH₂)₁₈—CO₂H) | | | |
| | YLIEGGPSSGAPPPS-NH₂ | | | |
| 35 | Y-Aib-EGTFTSDYSILLDKIAQ-Aib- | 44 | 4863.5 | N/I |
| | AFIEYK((2-[2-(2-Amino-ethoxy)-ethoxy]- | | | |
| | acetyl)₂-(γ-Glu)-CO—(CH₂)₁₈—CO₂H) | | | |
| | IEGGPSSGAPPPS-NH₂ | | | |
| 36 | Y-Aib-EGTFTSDYSILLDKIAQ-Aib- | 45 | 4863.5 | N/I |
| | AFIEYLK((2-[2-(2-Amino-ethoxy)-ethoxy]- | | | |
| | acetyl)₂-(γ-Glu)-CO—(CH₂)₁₈—CO₂H) | | | |
| | EGGPSSGAPPPS-NH₂ | | | |
| 37 | Y-Aib-EGTFTSDYSILLDKIAQ-Aib- | 46 | 4847.6 | N/I |
| | AFIEYLIK((2-[2-(2-Amino-ethoxy)-ethoxy]- | | | |
| | acetyl)₂-(γ-Glu)-CO—(CH₂)₁₈—CO₂H) | | | |
| | GGPSSGAPPPS-NH₂ | | | |
| 38 | Y-Aib-EGTFTSDYSILLDKIAQ-Aib- | 47 | 4919.6 | N/I |
| | AFIEYLIEK((2-[2-(2-Amino-ethoxy)- | | | |
| | ethoxy]-acetyl)₂-(γ-Glu)-CO—(CH₂)₁₈—CO₂H) | | | |
| | GPSSGAPPPS-NH₂ | | | |
| 39 | Y-Aib-EGTFTSDYSILLDKIAQ-Aib- | 48 | 4919.6 | N/I |
| | AFIEYLIEGK((2-[2-(2-Amino-ethoxy)- | | | |
| | ethoxy]-acetyl)₂-(γ-Glu)-CO—(CH₂)₁₈—CO₂H) | | | |
| | PSSGAPPPS-NH₂ | | | |
| 40 | Y-Aib-EGTFTSDYSILLDKIAQ-Aib- | 49 | 4879.5 | N/I |
| | AFIEYLIEGGK((2-[2-(2-Amino-ethoxy)- | | | |
| | ethoxy]-acetyl)₂-(γ-Glu)-CO—(CH₂)₁₈—CO₂H) | | | |
| | SSGAPPPS-NH₂ | | | |
| 41 | Y-Aib-EGTFTSDYSILLDKIAQ-Aib- | 50 | 4889.6 | N/I |
| | AFIEYLIEGGPK((2-[2-(2-Amino-ethoxy)- | | | |
| | ethoxy]-acetyl)₂-(γ-Glu)-CO—(CH₂)₁₈—CO₂H) | | | |
| | SGAPPPS-NH₂ | | | |
| 42 | Y-Aib-EGTFTSDYSILLDKIAQ-Aib- | 51 | 4889.6 | N/I |
| | AFIEYLIEGGPSK((2-[2-(2-Amino-ethoxy)- | | | |
| | ethoxy]-acetyl)₂-(γ-Glu)-CO—(CH₂)₁₈—CO₂H) | | | |
| | GAPPPS-NH₂ | | | |
| 43 | Y-Aib-EGTFTSDYSILLDKIAQ-Aib- | 52 | 4919.6 | N/I |
| | AFIEYLIEGGPSSK((2-[2-(2-Amino- | | | |
| | ethoxy)-ethoxy]-acetyl)₂-(γ-Glu)-CO—(CH₂)₁₈—CO₂H) | | | |
| | APPPS-NH₂ | | | |
| 44 | Y-Aib-EGTFTSDYSILLDKIAQ-Aib- | 53 | 4905.6 | N/I |
| | AFIEYLIEGGPSSGK((2-[2-(2-Amino- | | | |
| | ethoxy)-ethoxy]-acetyl)₂-(γ-Glu)-CO—(CH₂)₁₈—CO₂H) | | | |
| | PPPS-NH₂ | | | |
| 45 | Y-Aib-EGTFTSDYSILLDKIAQ-Aib- | 54 | 4879.5 | N/I |
| | AFIEYLIEGGPSSGAK((2-[2-(2-Amino- | | | |
| | ethoxy)-ethoxy]-acetyl)₂-(γ-Glu)-CO—(CH₂)₁₈—CO₂H) | | | |
| | PPS-NH₂ | | | |
| 46 | Y-Aib-EGTFTSDYSILLDKIAQ-Aib- | 55 | 4879.5 | N/I |
| | AFIEYLIEGGPSSGAPK((2-[2-(2-Amino- | | | |
| | ethoxy)-ethoxy]-acetyl)₂-(γ-Glu)-CO—(CH₂)₁₈—CO₂H) | | | |
| | PS-NH₂ | | | |
| 47 | Y-Aib-EGTFTSDYSILLDKIAQ-Aib- | 56 | 4879.5 | N/I |
| | AFIEYLIEGGPSSGAPPK((2-[2-(2-Amino- | | | |
| | ethoxy)-ethoxy]-acetyl)₂-(γ-Glu)-CO—(CH₂)₁₈—CO₂H)S—NH₂ | | | |
| 48 | Y-Aib-EGTFTSDYSILLDKIAQ-Aib- | 57 | 4889.6 | N/I |
| | AFIEYLIEGGPSSGAPPPK((2-[2-(2-Amino- | | | |
| | ethoxy)-ethoxy]-acetyl)₂-(γ-Glu)-CO—(CH₂)₁₈—CO₂H)—NH₂ | | | |
| 49 | Y-Aib-EGTFTSDYSILLDKIAQ-Aib- | 58 | 4976.7 | N/I |
| | AFIEYLIEGGPSSGAPPPSK((2-[2-(2- | | | |
| | Amino-ethoxy)-ethoxy]-acetyl)₂-(γ-Glu)-CO—(CH₂)₁₈—CO₂H)—NH₂ | | | |
| 50 | Y-Aib-EGTFTSDYSILLDKK((2-[2-(2- | 59 | 4414.0 | N/I |
| | Amino-ethoxy)-ethoxy]-acetyl)₂-(γ-Glu)-CO—(CH₂)₁₈—CO₂H) | | | |
| | AQ-Aib-AFIEYLIEGGPSSG- | | | |
| | NH₂ | | | |
| 51 | Y-Aib-EGTFTSDYSILLDKK((2-[2-(2- | 60 | 4085.7 | N/I |
| | Amino-ethoxy)-ethoxy]-acetyl)₂-(γ-Glu)-CO—(CH₂)₁₈—CO₂H) | | | |
| | AQ-Aib-AFIEYLIEGG-NH₂ | | | |
| 52 | Y-Aib-EGTFTSDYSI-αMeL-LDSK((2-[2-(2- | 61 | 4836.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 53 | H-Aib-EGTFTSDYSI-αMeL-LDKK((2-[2- | 62 | 4851.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 54 | H-Aib-EGTFTSDYSI-αMeL-LDKK((2-[2- | 63 | 4903.5 | N/I |
| | (2-Amino-ethoxy)-ethoxy]-acetyl)₂-(γ-Glu)- | | | |
| | CO—(CH₂)₁₈—CO₂H) | | | |
| | AQHAFIEYLIEGGPSSGAPPPS-NH₂ | | | |
| 55 | H-Aib-EGTFTSDYSI-αMeL-LDEK((2-[2- | 64 | 4904.5 | N/I |
| | (2-Amino-ethoxy)-ethoxy]-acetyl)₂-(γ-Glu)- | | | |
| | CO—(CH₂)₁₈—CO₂H) | | | |
| | AQHAFIEYLIEGGPSSGAPPPS-NH₂ | | | |
| 56 | Y-Aib-EGTFTSDYSI-αMeL-LDEK((2-[2- | 65 | 4930.5 | N/I |
| | (2-Amino-ethoxy)-ethoxy]-acetyl)₂-(γ-Glu)- | | | |
| | CO—(CH₂)₁₈—CO₂H) | | | |
| | AQHAFIEYLIEGGPSSGAPPPS-NH₂ | | | |
| 57 | Y-Aib-EGT-αMeF-TSDYSILLDKK((2-[2- | 66 | 4877.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 58 | Y-Aib-EGTFTSDYSSLLDKK((2-[2-(2- | 67 | 4837.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 59 | Y-Aib-EGTFTSDYSI-αMeL-LDEK((2-[2- | 68 | 4878.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 60 | Y-Aib-EGTFTSDYSI-αMeL-LD-Aib-K((2- | 69 | 4834.5 | N/I |
| | [2-(2-Amino-ethoxy)-ethoxy]-acetyl)₂-(γ- | | | |
| | Glu)-CO—(CH₂)₁₈—CO₂H)AQ-Aib- | | | |
| | AFIEYLIEGGPSSGAPPPS-NH₂ | | | |
| 61 | Y-Aib-EGTFTSDYSI-αMeL-LDSK((2-[2-(2- | 70 | 4836.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLLEGGPSSGAPPPS-NH₂ | | | |
| 62 | Y-Aib-EGTFTSDYSI-αMeL-LDKK((2-[2- | 71 | 4099.7 | N/I |
| | (2-Amino-ethoxy)-ethoxy]-acetyl)₂-(γ-Glu)- | | | |
| | CO—(CH₂)₁₈—CO₂H)AQ-Aib-AFIEYLIEGG- | | | |
| | NH₂ | | | |
| 63 | Y-Aib-EGTFTSDYSI-αMeL-LDEK((2-[2- | 72 | 4100.6 | N/I |
| | (2-Amino-ethoxy)-ethoxy]-acetyl)₂-(γ-Glu)- | | | |
| | CO—(CH₂)₁₈—CO₂H)AQ-Aib-AFIEYLIEGG- | | | |
| | NH₂ | | | |
| 64 | Y-Aib-EGTFTSDYSI-αMeL-LDSK((2-[2-(2- | 73 | 4058.6 | N/I |
| | Amino-ethoxy)-ethoxy]-acetyl)₂-(γ-Glu)-CO—(CH₂)₁₈—CO₂H) | | | |
| | AQ-Aib-AFIEYLIEGG-NH₂ | | | |
| 65 | Y-Aib-EGTFTSDYSI-αMeL-LDTK((2-[2- | 74 | 4072.6 | N/I |
| | (2-Amino-ethoxy)-ethoxy]-acetyl)₂-(γ-Glu)- | | | |
| | CO—(CH₂)₁₈—CO₂H)AQ-Aib-AFIEYLIEGG- | | | |
| | NH₂ | | | |
| 66 | Y-Aib-EGTFTSDYSI-αMeL-LDEK((2-[2- | 75 | 4878.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLLEGGPSSGAPPPS-NH₂ | | | |
| 67 | Y-Aib-EGTFTSDYSILLDKK((2-[2-(2- | 76 | 4877.5 | N/I |
| | Amino-ethoxy)-ethoxy]-acetyl)₂-(γ-Glu)-CO—(CH₂)₁₈—CO₂H) | | | |
| | AQ-Aib-A-αMeF- | | | |
| | IEYLIEGGPSSGAPPPS-NH₂ | | | |
| 68 | Y-Aib-EGTFTSDY-αMeS-ILLDKK((2-[2- | 77 | 4877.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 69 | Y-Aib-EGT-αMeF-TSDYSI-αMeL- | 78 | 4891.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 70 | Y-Aib-EGTFTSDK((2-[2-(2-Amino-ethoxy)- | 79 | 4035.7 | N/I |
| | ethoxy]-acetyl)₂-(γ-Glu)-CO—(CH₂)₁₈—CO₂H) | | | |
| | SILLDKIAQ-Aib-AFIEYLIEGG-NH₂ | | | |
| 71 | Y-Aib-EGTFTSDYSILK((2-[2-(2-Amino- | 80 | 4085.7 | N/I |
| | ethoxy)-ethoxy]-acetyl)₂-(γ-Glu)-CO—(CH₂)₁₈—CO₂H) | | | |
| | DKIAQ-Aib-AFIEYLIEGG-NH₂ | | | |
| 72 | Y-Aib-EGTFTSDYSILLDKIAQ-Aib-K((2- | 81 | 4127.8 | N/I |
| | [2-(2-Amino-ethoxy)-ethoxy]-acetyl)₂-(γ- | | | |
| | Glu)-CO—(CH₂)₁₈—CO₂H)FIEYLIEGG-NH₂ | | | |
| 73 | Y-Aib-EGTFTSDYSILLDKIAQ-Aib- | 82 | 4069.7 | N/I |
| | AFIEYLIK((2-[2-(2-Amino-ethoxy)-ethoxy]- | | | |
| | acetyl)₂-(γ-Glu)-CO—(CH₂)₁₈—CO₂H)GG-NH₂ | | | |
| 74 | Y-Aib-EGTFTSDYSI-αMeL-LDKK((2-[2- | 83 | 4891.6 | N/I |
| | (2-Amino-ethoxy)-ethoxy]-acetyl)₂-(γ-Glu)- | | | |
| | CO—(CH₂)₁₈—CO₂H)AQ-Aib-A-αMeF- | | | |
| | IEYLIEGGPSSGAPPPS-NH₂ | | | |
| 75 | Y-Aib-EGTFTSDYSI-αMeL-LDKK((2-[2- | 84 | 4891.6 | N/I |
| | (2-Amino-ethoxy)-ethoxy]-acetyl)₂-(γ-Glu)- | | | |
| | CO—(CH₂)₁₈—CO₂H)AQ-Aib-AFIEY-αMeL- | | | |
| | IEGGPSSGAPPPS-NH₂ | | | |
| 76 | Y-Aib-EGT-aMeF-TSDYSI-αMeL- | 85 | 4905.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | A-αMeF-IEYLIEGGPSSGAPPPS-NH₂ | | | |
| 77 | Y-Aib-EGTFTSDYSILLKIAQ-Aib- | 86 | 4764.5 | N/I |
| | AFIEYLIEGGPSSGAPPK((2-[2-(2-Amino- | | | |
| | ethoxy)-ethoxy]-acetyl)₂-(γ-Glu)-CO—(CH₂)₁₈—CO₂H)S—NH₂ | | | |
| 78 | (D-Tyr)-Aib-EGTFTSDYSILLDKK((2-[2-(2- | 87 | 4863.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 79 | Ac-(D-Tyr)-AEGTFTSDYSILLDKK((2-[2- | 88 | 4891.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 80 | Y-(D-Ala)-EGTFTSDYSILLDKK((2-[2-(2- | 89 | 4849.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 81 | Y-Aib-EGTFTSDY-(D-Ser)-ILLDKK((2-[2- | 90 | 4863.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 82 | Y-Aib-EGTFTSDYS-(D-Ile)-LLDKK((2-[2- | 91 | 4863.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 83 | Y-Aib-EGTFTSDYSILLDKK((2-[2-(2- | 92 | 4863.5 | N/I |
| | Amino-ethoxy)-ethoxy]-acetyl)₂-(γ-Glu)-CO—(CH₂)₁₈—CO₂H) | | | |
| | AQ-Aib-AFIEYLI-(D-Glu)- | | | |
| | GGPSSGAPPPS-NH₂ | | | |
| 84 | Y-Aib-EGTFTSDYSILLDKK((2-[2-(2- | 93 | 4863.5 | N/I |
| | Amino-ethoxy)-ethoxy]-acetyl)₂-(γ-Glu)-CO—(CH₂)₁₈—CO₂H) | | | |
| | AQ-Aib-AFI-(D-Glu)- | | | |
| | YLIEGGPSSGAPPPS-NH₂ | | | |
| 85 | Y-Aib-EGTFTSDASILLDKK((2-[2-(2- | 94 | 4771.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 86 | Y-Aib-EGTFTSDYSILLDKK((2-[2-(2- | 95 | 4877.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEAGPSSGAPPPS-NH₂ | | | |
| 87 | Y-αMePro-EGTFTSDYSILLDKK((2-[2-(2- | 96 | 4889.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 88 | Y-Pro-EGTFTSDYSILLDKK((2-[2-(2- | 97 | 4875.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 89 | Y-Aib-Aad-GTFTSDYSILLDKK((2-[2-(2- | 98 | 4877.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 90 | Y-Aib-NGTFTSDYSILLDKK((2-[2-(2- | 99 | 4848.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 91 | Y-Aib-(γ-Glu)-GTFTSDYSILLDKK((2-[2- | 100 | 4863.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 92 | Y-Aib-EGT-αMeF-TSDK((2-[2-(2-Amino- | 101 | 4049.7 | N/I |
| | ethoxy)-ethoxy]-acetyl)₂-(γ-Glu)-CO—(CH₂)₁₈—CO₂H) | | | |
| | SILLDKIAQ-Aib-AFIEYLIEGG-NH₂ | | | |
| 93 | Y-Aib-EGT-αMeF-TSDYSILK((2-[2-(2- | 102 | 4099.7 | N/I |
| | Amino-ethoxy)-ethoxy]-acetyl)₂-(γ-Glu)-CO—(CH₂)₁₈—CO₂H) | | | |
| | DKIAQ-Aib-AFIEYLIEGG- | | | |
| | NH₂ | | | |
| 94 | Y-Aib-EGT-αMeF-TSDYSILLDKIAQ-Aib- | 103 | 4141.8 | N/I |
| | K((2-[2-(2-Amino-ethoxy)-ethoxy]-acetyl)₂- | | | |
| | (γ-Glu)-CO—(CH₂)₁₈—CO₂H)FIEYLIEGG-NH₂ | | | |
| 95 | Y-Aib-EGT-αMeF-TSDYSILLDKIAQ-Aib- | 104 | 4083.7 | N/I |
| | AFIEYLIK((2-[2-(2-Amino-ethoxy)-ethoxy]- | | | |
| | acetyl)₂-(γ-Glu)-CO—(CH₂)₁₈—CO₂H)GG-NH₂ | | | |
| 96 | Y-Aib-EGTFTSDK((2-[2-(2-Amino-ethoxy)- | 105 | 4049.7 | N/I |
| | ethoxy]-acetyl)₂-(γ-Glu)-CO—(CH₂)₁₈—CO₂H) | | | |
| | SI-αMeL-LDKIAQ-Aib- | | | |
| | AFIEYLIEGG-NH₂ | | | |
| 97 | Y-Aib-EGTFTSDYSI-αMeL-LDKIAQ-Aib- | 106 | 4083.7 | N/I |
| | AFIEYLIK((2-[2-(2-Amino-ethoxy)-ethoxy]- | | | |
| | acetyl)₂-(γ-Glu)-CO—(CH₂)₁₈—CO₂H)GG-NH₂ | | | |
| 98 | Y-Aib-EGT-αMeF-TSDYSILLDKK((2-[2- | 107 | 4099.7 | N/I |
| | (2-Amino-ethoxy)-ethoxy]-acetyl)₂-(γ-Glu)- | | | |
| | CO—(CH₂)₁₈—CO₂H)AQ-Aib-AFIEYLIEGG- | | | |
| | NH₂ | | | |
| 99 | Y-Aib-EGT-αMeF-TSDYSI-αMeL- | 108 | 4113.7 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGG-NH₂ | | | |
| 100 | Y-Aib-EGT-αMeF-TSDYSI-αMeL- | 109 | 4114.7 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGG-NH₂ | | | |
| 101 | Y-Aib-EGT-αMeF(2F)-TSDYSI-Aib- | 110 | 4090.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGG-NH₂ | | | |
| 102 | Y-Aib-EGT-αMeF-TSDYSI-Aib-LDEK((2- | 111 | 4072.6 | N/I |
| | [2-(2-Amino-ethoxy)-ethoxy]-acetyl)₂-(γ- | | | |
| | Glu)-CO—(CH₂)₁₈—CO₂H)AQ-Aib- | | | |
| | AFIEYLIEGG-NH₂ | | | |
| 103 | Y-Aib-EGT-αMeF(2F)-TSDYSI-αMeL- | 112 | 4190.7 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGG-NH₂ | | | |
| 104 | Y-Aib-EGT-αMeF(2F)-TSDYSI-αMeL- | 113 | 4162.6 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFIEYLIEGG-NH₂ | | | |
| 105 | DesHis-ψ[NHCO]-Aib- | 114 | 4822.5 | N/I |
| | EGTFTSDYSILLDKK((2-[2-(2-Amino- | | | |
| | ethoxy)-ethoxy]-acetyl)₂-(γ-Glu)-CO—(CH₂)₁₈—CO₂H) | | | |
| | AQ-Aib-AFIEYLIEGGPSSGAPPPS- | | | |
| | NH₂ | | | |
| 106 | DesHis-Aib-EGTFTSDYSILLDKK((2-[2-(2- | 115 | 4822.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 107 | DesTyr-Aib-EGTFTSDYSILLDKK((2-[2-(2- | 116 | 4848.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 108 | Y-Aib-EGTFTSDYSILLDKK((2-[2-(2- | 117 | 4859.6 | N/I |
| | Amino-ethoxy)-ethoxy]-acetyl)-AOC-(γ- | | | |
| | Glu)-CO—(CH₂)₁₈—CO₂H)AQ-Aib- | | | |
| | AFIEYLIEGGPSSGAPPPS-NH₂ | | | |
| 109 | Y-Aib-EGTFTSDYSILLDKK(AOC-(2-[2-(2- | 118 | 4859.6 | N/I |
| | Amino-ethoxy)-ethoxy]-acetyl)-(γ-Glu)-CO—(CH₂)₁₈—CO₂H) | | | |
| | AQ-Aib- | | | |
| | AFIEYLIEGGPSSGAPPPS-NH2 | | | |
| 110 | Y-Aib-EGTFTSDYSILLDKK((2-[2-(2- | 119 | | N/I |
| | Amino-ethoxy)-ethoxy]-acetyl)-(γ-Glu)- | | | |
| | (Trx)-CO—(CH₂)₁₈—CO₂H)AQ-Aib- | | | |
| | AFIEYLIEGGPSSGAPPPS-NH₂ | | | |
| 111 | Y-Aib-EGTFTSDYSILLDKK((2-[2-(2- | 120 | | N/I |
| | Amino-ethoxy)-ethoxy]-acetyl)-(Trx)-(γ- | | | |
| | Glu)-CO—(CH₂)₁₈—CO₂H)AQ-Aib- | | | |
| | AFIEYLIEGGPSSGAPPPS-NH₂ | | | |
| 112 | Y-Aib-EGTFTSDYSILLDKK((2-[2-(2- | 121 | 4846.5 | N/I |
| | Amino-ethoxy)-ethoxy]-acetyl)-(εK)-(γ-Glu)- | | | |
| | CO—(CH₂)₁₈—CO₂H)AQ-Aib- | | | |
| | AFIEYLIEGGPSSGAPPPS-NH₂ | | | |
| 113 | Y-Aib-EGTFTSDYSILLDKK((2-[2-(2- | 122 | 4862.6 | N/I |
| | Amino-ethoxy)-ethoxy]-acetyl)₂-(εK)-CO—(CH₂)₁₈—CO₂H) | | | |
| | AQ-Aib- | | | |
| | AFIEYLIEGGPSSGAPPPS-NH₂ | | | |
| 114 | Y-Aib-EGTFTSDYSILLDKK((2-[2-(2- | 123 | 4845.6 | N/I |
| | Amino-ethoxy)-ethoxy]-acetyl)-(εK)-(εK)- | | | |
| | CO—(CH₂)₁₈—CO₂H)AQ-Aib- | | | |
| | AFIEYLIEGGPSSGAPPPS-NH₂ | | | |
| 115 | Y-Aib-EGT-αMeF-TSDYSI-αMeL- | 124 | 4892.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 116 | Y-Aib-EGT-αMeF-TSDYSI-αMeL- | 125 | 4950.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 117 | Y-Aib-EGT-αMeF-TSDYSI-Aib-LDEK((2- | 126 | 4850.4 | N/I |
| | [2-(2-Amino-ethoxy)-ethoxy]-acetyl)₂-(γ- | | | |
| | Glu)-CO—(CH₂)₁₈—CO₂H)AQ-Aib- | | | |
| | AFIEYLIEGGPSSGAPPPS-NH₂ | | | |
| 118 | Y-Aib-EGT-αMeF(2F)-TSDYSI-αMeL- | 127 | 4968.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 119 | F-Aib-EGT-αMeF-TSDYSI-αMeL- | 128 | 4876.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 120 | Y-Aib-cTA-GT-αMeF-TSDYSI-αMeL- | 129 | 4902.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 121 | Y-Aib-EGT-αMeF-TSDYSI-αMeL- | 130 | 4935.5 | N/I |
| | LDEK((2-[2-(2-Amino-ethoxy)-ethoxy]- | | | |
| | acetyl)₂-(γ-Glu)-CO—(CH₂)₁₈—CO₂H) | | | |
| | AQQAFIEYLIEGGPSSGAPPPS-NH₂ | | | |
| 122 | Y-Aib-EGT-αMeF-TSDYSI-αMeL- | 131 | 4963.6 | N/I |
| | LDEK((2-[2-(2-Amino-ethoxy)-ethoxy]- | | | |
| | acetyl)₂-(γ-Glu)-CO—(CH₂)₁₈—CO₂H) | | | |
| | AQQAFIEYLIE-Aib-GPSSGAPPPS- | | | |
| | NH₂ | | | |
| 123 | Y-Aib-EGT-αMeF-TSDYSI-αMeL- | 132 | 4500.1 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSG-NH₂ | | | |
| 124 | Y-Aib-EGT-αMeF-TSDYSI-αMeL- | 133 | 4501.0 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSG-NH₂ | | | |
| 125 | Y-Aib-EGT-αMeF-TSDYSI-αMeL- | 134 | 5020.7 | N/I |
| | LDKK((2-[2-(2-Amino-ethoxy)-ethoxy]- | | | |
| | acetyl)₂-(γ-Glu)₂-CO—(CH₂)₁₈—CO₂H)AQ-Aib- | | | |
| | AFIEYLIEGGPSSGAPPPS-NH₂ | | | |
| 126 | Y-Aib-EGT-αMeF-TSDYSI-αMeL- | 135 | 4905.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEAGPSSGAPPPS-NH₂ | | | |
| 127 | Y-Aib-EGT-αMeF-TSDISILLDKK((2-[2-(2- | 136 | 4827.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 128 | Y-Aib-EGT-αMeF-TSDHSILLDKK((2-[2- | 137 | 4851.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 129 | Y-Aib-EGT-αMeF-TSDLSILLDKK((2-[2- | 138 | 4827.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 130 | Y-Aib-EGT-αMeF-TSDESILLDKK((2-[2- | 139 | 4843.5 | N/I |
| | (2-Amino-ethoxy)-ethoxy]-acetyl)₂-(γ-Glu)- | | | |
| | CO—(CH₂)₁₈—CO₂H)AQ-Aib- | | | |
| | AFIEYLIEGGPSSGAPPPS-NH₂ | | | |
| 131 | Y-Aib-EGT-αMeF-TSD-αMeF- | 140 | 4875.6 | N/I |
| | SILLDKK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 132 | Y-Aib-EGT-αMeF-TSD-3Pal-SILLDKK((2- | 141 | 4862.5 | N/I |
| | [2-(2-Amino-ethoxy)-ethoxy]-acetyl)₂-(γ- | | | |
| | Glu)-CO—(CH₂)₁₈—CO₂H)AQ-Aib- | | | |
| | AFIEYLIEGGPSSGAPPPS-NH₂ | | | |
| 133 | DesTyr-Aib-EGT-αMeF-TSDYSI-Aib- | 142 | 4835.4 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 134 | DesTyr-Aib-EGT-αMeF(2F)-TSDYSI- | 143 | 4953.5 | N/I |
| | αMeL-LDEK((2-[2-(2-Amino-ethoxy)- | | | |
| | ethoxy]-acetyl)₂-(γ-Glu)-CO—(CH₂)₁₈—CO₂H) | | | |
| | AQ-Aib-EFIEYLIEGGPSSGAPPPS- | | | |
| | NH₂ | | | |
| 135 | H-Aib-NGTFTSDYSILLDKK((2-[2-(2- | 144 | 4822.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 136 | Y-Aib-EGTFTSDASILLDKK((2-[2-(2- | 145 | 4785.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEAGPSSGAPPPS-NH₂ | | | |
| 137 | Y-Aib-EGT-αMeF-TSDYSI-αMeL- | 146 | 4963.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | Aad-FIEYLIEGGPSSGAPPPS-NH₂ | | | |
| 138 | Y-Aib-EGT-αMeF-TSDYSI-αMeL- | 147 | 4907.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | SFIEYLIEGGPSSGAPPPS-NH₂ | | | |
| 139 | Y-Aib-EGT-αMeF-TSDYSI-αMeL- | 148 | 4921.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | TFIEYLIEGGPSSGAPPPS-NH₂ | | | |
| 140 | Y-Aib-EGT-αMeF-TSDYSI-αMeL- | 149 | 4935.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | DFIEYLIEGGPSSGAPPPS-NH₂ | | | |
| 141 | Y-Aib-EGT-αMeF-TSDYSI-αMeL- | 150 | 4933.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | IFIEYLIEGGPSSGAPPPS-NH₂ | | | |
| 142 | Y-Aib-EGT-αMeF-TSDYSI-αMeL- | 151 | 4957.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | HFIEYLIEGGPSSGAPPPS-NH₂ | | | |
| 143 | Y-Aib-EGT-αMeF-TSDYSI-αMeL- | 152 | 4905.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | Aib-FIEYLIEGGPSSGAPPPS-NH₂ | | | |
| 144 | Y-Aib-EGT-αMeF-TSDYSI-αMeL- | 153 | 4957.6 | N/I |
| | LDKK((2-[2-(2-Amino-ethoxy)-ethoxy]- | | | |
| | acetyl)₂-(γ-Glu)-CO—(CH₂)₁₈—CO₂H)AQH- | | | |
| | Aib-FIEYLIEGGPSSGAPPPS-NH₂ | | | |
| 145 | Y-Aib-EGT-αMeF-TSDASI-αMeL- | 154 | 4799.5 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | AFIEYLIEGGPSSGAPPPS-NH₂ | | | |
| 146 | Y-Aib-EGT-αMeF(2F)-TSDYSI-αMeL- | 155 | 4967.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIQYLIEGGPSSGAPPPS-NH₂ | | | |
| 147 | Y-Aib-EGT-αMeF(2F)-TSDYSI-αMeL- | 156 | 4982.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-Aad-YLIEGGPSSGAPPPS-NH₂ | | | |
| 148 | Y-Aib-EGT-αMeF(2F)-TSDYSI-αMeL- | 157 | 4910.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIAYLIEGGPSSGAPPPS-NH₂ | | | |
| 149 | Y-Aib-EGT-αMeF(2F)-TSDYSI-αMeL- | 158 | 4938.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIVYLIEGGPSSGAPPPS-NH₂ | | | |
| 150 | Y-Aib-EGT-αMeF(2F)-TSDYSI-αMeL- | 159 | 4926.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFISYLIEGGPSSGAPPPS-NH₂ | | | |
| 151 | Y-Aib-EGT-αMeF(2F)-TSDYSI-αMeL- | 160 | 4936.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIPYLIEGGPSSGAPPPS-NH₂ | | | |
| 152 | Y-Aib-EGT-αMeF(2F)-TSDYSI-αMeL- | 161 | 4924.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-Aib-YLIEGGPSSGAPPPS-NH₂ | | | |
| 153 | Y-Aib-EGT-αMeF(2F)-TSDYSI-αMeL- | 162 | 4976.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIHYLIEGGPSSGAPPPS-NH₂ | | | |
| 154 | Y-Aib-EGT-αMeF(2F)-TSDHSI-αMeL- | 163 | 4942.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 155 | Y-Aib-EGT-αMeF(2F)-TSD-cTA-SI-αMeL- | 164 | 4944.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 156 | Y-Aib-EGT-αMeF(2F)-TSD-2Pal-SI-αMeL- | 165 | 4953.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 157 | Y-Aib-EGT-αMeF(2F)-TSD-3Pal-SI-αMeL- | 166 | 4953.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 158 | Y-Aib-EGT-αMeF(2F)-TSD-4Pal-SI-αMeL- | 167 | 4953.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 159 | Y-Aib-EGT-αMeF(2F)-TSD-αMeF-SI- | 168 | 4938.5 | N/I |
| | αMeL-LDEK((2-[2-(2-Amino-ethoxy)- | | | |
| | ethoxy]-acetyl)₂-(γ-Glu)-CO—(CH₂)₁₈—CO₂H) | | | |
| | AQ-Aib-EFIEYLIEGGPSSGAPPPS- | | | |
| | NH₂ | | | |
| 160 | Y-Aib-EGT-αMeF(2F)-TSD-Aib-SI-αMeL- | 169 | 4862.4 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSGAPPPS-NH₂ | | | |
| 161 | Y-Aib-EGT-αMeF(2F)-TSDHSI-αMeL- | 170 | 4594.1 | N/I |
| | LDEK((2-[2-(2-Amino-ethoxy)-ethoxy]- | | | |
| | acetyl)₂-(γ-Glu)₂-CO—(CH₂)₁₈—CO₂H)AQ-Aib- | | | |
| | TFI-(D-Glu)-YLIEGGPSSG-NH₂ | | | |
| 162 | Y-Aib-EGT-αMeF(2F)-TSDHSI-αMeL- | 171 | 4568.1 | N/I |
| | LDEK((2-[2-(2-Amino-ethoxy)-ethoxy]- | | | |
| | acetyl)₂-(γ-Glu)₂-CO—(CH₂)₁₈—CO₂H)AQ-Aib- | | | |
| | TFI-(D-Glu)-HLIEGGPSSG-NH₂ | | | |
| 163 | H-Aib-EGT-αMeF(2F)-TSDYSI-αMeL- | 172 | 4942.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIEYLIEGGPSSGAPPPS-NH₂ | | | |
| 164 | Y-Aib-EGT-αMeF(2F)-TSDHSI-αMeL- | 173 | 4914.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLI-(D-Glu)-GGPSSGAPPPS- | | | |
| | NH₂ | | | |
| 165 | Y-Aib-EGT-αMeF(2F)-TSDHSI-αMeL- | 174 | 4912.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-αMeF-LI-(D-Glu)- | | | |
| | GGPSSGAPPPS-NH₂ | | | |
| 166 | Y-Aib-EGT-αMeF(2F)-TSDHSI-αMeL- | 175 | 4136.7 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGG-NH₂ | | | |
| 167 | Y-Aib-EGT-αMeF(2F)-TSDHSI-αMeL- | 176 | 4465.0 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSG-NH₂ | | | |
| 168 | Y-Aib-EGT-αMeF(2F)-TSDHSI-αMeL- | 177 | 4914.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSGAPPPS-NH₂ | | | |
| 169 | Y-Aib-EGT-αMeF(2F)-TSDHSI-αMeL- | 178 | 4886.4 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | TFI-(D-Glu)-YLIEGGPSSGAPPPS-NH₂ | | | |
| 170 | Y-Aib-EGT-αMeF(2F)-TSDHSI-αMeL- | 179 | 4858.4 | N/I |
| | LDEK((2-[2-(2-Amino-ethoxy)-ethoxy]- | | | |
| | acetyl)₂-(γ-Glu)-CO—(CH₂)₁₄—CO₂H)AQ-Aib- | | | |
| | TFI-(D-Glu)-YLIEGGPSSGAPPPS-NH₂ | | | |
| 171 | Y-Aib-EGT-αMeF(2F)-TSDHSI-αMeL-LD- | 180 | 4899.5 | N/I |
| | Orn-K((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSGAPPPS-NH₂ | | | |
| 172 | Y-Aib-EGT-αMeF(2F)-TSDHSI-αMeL-LD- | 181 | 4885.5 | N/I |
| | Dab-K((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSGAPPPS-NH₂ | | | |
| 173 | Y-Aib-EGT-αMeF(2F)-TSDHSI-αMeL-LD- | 182 | 4871.5 | N/I |
| | Dap-K((2-[2-(2-Amino-ethoxy)-ethoxy]- | | | |
| | acetyl)₂-(γ-Glu)-CO—(CH₂)₁₈—CO₂H)AQ-Aib- | | | |
| | TFI-(D-Glu)-YLIEGGPSSGAPPPS-NH₂ | | | |
| 174 | Y-Aib-EGT-αMeF(2F)-TSDHSI-αMeL- | 183 | 4785.4 | N/I |
| | LDEK((2-[2-(2-Amino-ethoxy)-ethoxy]- | | | |
| | acetyl)₂-CO—(CH₂)₁₈—CO₂H)AQ-Aib-TFI-(D- | | | |
| | Glu)-YLIEGGPSSGAPPPS-NH₂ | | | |
| 175 | Y-Aib-EGT-αMeF(2F)-TSDHSI-αMeL- | 184 | 4913.5 | N/I |
| | LDEK((2-[2-(2-Amino-ethoxy)-ethoxy]- | | | |
| | acetyl)₂-(εK)-CO—(CH₂)₁₈—CO₂H)AQ-Aib- | | | |
| | TFI-(D-Glu)-YLIEGGPSSGAPPPS-NH₂ | | | |
| 176 | Y-Aib-EGT-αMeF(2F)-TSDHSI-αMeL- | 185 | 4885.5 | N/I |
| | LDEK((2-[2-(2-Amino-ethoxy)-ethoxy]- | | | |
| | acetyl)₂-(εK)-CO—(CH₂)₁₆—CO₂H)AQ-Aib- | | | |
| | TFI-(D-Glu)-YLIEGGPSSGAPPPS-NH₂ | | | |
| 177 | Y-Aib-EGT-αMeF(2F)-TSDHSI-αMeL- | 186 | 4922.5 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | HFI-(D-Glu)-YLIEGGPSSGAPPPS-NH₂ | | | |
| 178 | Y-Aib-EGT-αMeF(2F)-TSDHSI-αMeL- | 187 | 4894.4 | N/I |
| | LDEK((2-[2-(2-Amino-ethoxy)-ethoxy]- | | | |
| | acetyl)₂-(γ-Glu)-CO—(CH₂)₁₄—CO₂H)AQ-Aib- | | | |
| | HFI-(D-Glu)-YLIEGGPSSGAPPPS-NH₂ | | | |
| 179 | Y-Aib-EGT-αMeF(2F)-TSDHSI-αMeL- | 188 | 4473.0 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | HFI-(D-Glu)-YLIEGGPSSG-NH₂ | | | |
| 180 | Y-Aib-EGT-αMeF(2F)-TSDHSI-αMeL- | 189 | 4144.6 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | HFI-(D-Glu)-YLIEGG-NH₂ | | | |
| 181 | Y-Aib-EGT-αMeF(2F)-TSD-3Pal-SI-αMeL- | 190 | 4953.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 182 | Y-Aib-EGT-αMeF(2F)-TSD-4Pal-SI-αMeL- | 191 | 4953.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 183 | Y-Aib-EGT-αMeF(2F)-TSDLSI-αMeL- | 192 | 4918.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 184 | Y-Aib-EGT-αMeF(2F)-TSD-(D-Tyr)-SI-αMeL- | 193 | 4968.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 185 | Y-Aib-EGT-αMeF(2F)-TSD-(D-His)-SI-αMeL- | 194 | 4942.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 186 | Y-Aib-EGT-αMeF(2F)-TSD-αMeY-SI-αMeL- | 195 | 4982.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 187 | Y-Aib-EGT-αMeF(2F)-TSDQSI-αMeL- | 196 | 4933.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 188 | Y-Aib-EGT-αMeF(2F)-TSD-3Pal-SI-αMeL- | 197 | 4909.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-Aib-YLIEGGPSSGAPPPS-NH₂ | | | |
| 189 | Y-Aib-EGT-αMeF(2F)-TSD-4Pal-SI-αMeL- | 198 | 4909.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-Aib-YLIEGGPSSGAPPPS-NH₂ | | | |
| 190 | H-Aib-EGT-αMeF(2F)-TSD-3Pal-SI-αMeL- | 199 | 4927.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 191 | Y-Aib-EGT-αMeF(2F)-TSDVSI-αMeL- | 200 | 4904.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 192 | Y-Aib-EGT-αMeF(2F)-TSDASI-αMeL- | 201 | 4876.4 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 193 | Y-αMePro-EGTFTSDYSILLDEK((2-[2-(2- | 202 | 4933.5 | N/I |
| | Amino-ethoxy)-ethoxy]-acetyl)₂-(γ-Glu)-CO—(CH₂)₁₈—CO₂H) | | | |
| | AQQAFIEYLIEGGPSSGAPPPS-NH₂ | | | |
| 194 | Y-αMePro-EGTFTSDYSILLDEK((2-[2-(2- | 203 | 4942.5 | N/I |
| | Amino-ethoxy)-ethoxy]-acetyl)₂-(γ-Glu)-CO—(CH₂)₁₈—CO₂H) | | | |
| | AQHAFIEYLIEGGPSSGAPPPS-NH₂ | | | |
| 195 | Y-αMePro-EGTFTSDYSILLDRK((2-[2-(2- | 204 | 4960.6 | N/I |
| | Amino-ethoxy)-ethoxy]-acetyl)₂-(γ-Glu)-CO—(CH₂)₁₈—CO₂H) | | | |
| | AQQAFIEYLIEGGPSSGAPPPS-NH₂ | | | |
| 196 | Y-αMePro-EGTFTSDYSILLDRK((2-[2-(2- | 205 | 4969.6 | N/I |
| | Amino-ethoxy)-ethoxy]-acetyl)₂-(γ-Glu)-CO—(CH₂)₁₈—CO₂H) | | | |
| | AQHAFIEYLIEGGPSSGAPPPS-NH₂ | | | |
| 197 | Y-αMePro-EGTFTSDYSILLDEK((2-[2-(2- | 206 | 4456.0 | N/I |
| | Amino-ethoxy)-ethoxy]-acetyl)₂-(γ-Glu)-CO—(CH₂)₁₆—CO₂H) | | | |
| | AQQAFIEYLIEGGPSSG- | | | |
| | NH₂ | | | |
| 198 | (D-Tyr)-αMePro-EGTFTSDYSILLDEK((2- | 207 | 4456.0 | N/I |
| | [2-(2-Amino-ethoxy)-ethoxy]-acetyl)₂-(γ- | | | |
| | Glu)-CO—(CH₂)₁₆—CO₂H) | | | |
| | AQQAFIEYLIEGGPSSG-NH₂ | | | |
| 199 | DesTyr-Aib-EGTFTSDYSILLDEK((2-[2-(2- | 208 | 4892.5 | N/I |
| | Amino-ethoxy)-ethoxy]-acetyl)₂-(γ-Glu)-CO—(CH₂)₁₈—CO₂H) | | | |
| | AQQAFIEYLIEGGPSSGAPPPS-NH₂ | | | |
| 200 | DesTyr-AEGTFTSDYSILLDEK((2-[2-(2- | 209 | 4878.4 | N/I |
| | Amino-ethoxy)-ethoxy]-acetyl)₂-(γ-Glu)-CO—(CH₂)₁₈—CO₂H) | | | |
| | AQQAFIEYLIEGGPSSGAPPPS-NH₂ | | | |
| 201 | DesHis-αMePro-EGTFTSDYSILLDEK((2- | 210 | 4892.5 | N/I |
| | [2-(2-Amino-ethoxy)-ethoxy]-acetyl)₂-(γ- | | | |
| | Glu)-CO—(CH₂)₁₈—CO₂H) | | | |
| | AQQAFIEYLIEGGPSSGAPPPS-NH₂ | | | |
| 202 | Y-Aib-EGT-αMeF(2F)-TSD-4Pal-SI-αMeL- | 211 | 4938.6 | N/I |
| | LDKK((2-[2-(2-Amino-ethoxy)-ethoxy]- | | | |
| | acetyl)-(γ-Glu)-(2-[2-(2-Amino-ethoxy)- | | | |
| | ethoxy]-acetyl)-CO—(CH₂)₁₈—CO₂H)AQ-Aib- | | | |
| | EFI-(D-Glu)-YLIEGGPSSGAPPPS-NH₂ | | | |
| 203 | Y-Aib-EGT-αMeF(2F)-TSD-4Pal-SI-αMeL- | 212 | 4952.6 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 204 | Y-Aib-EGT-αMeF(2F)-TSD-4Pal-SI-αMeL- | 213 | 4924.5 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 205 | Y-Aib-EGT-αMeF(2F)-TSD-4Pal-SI-αMeL- | 214 | 4795.4 | N/I |
| | LDEK((2-[2-(2-Amino-ethoxy)-ethoxy]- | | | |
| | acetyl)₂-CO—(CH₂)₁₆—CO₂H)AQ-Aib-EFI-(D- | | | |
| | Glu)-YLIEGGPSSGAPPPS-NH₂ | | | |
| 206 | Y-Aib-EGT-αMeF(2F)-TSD-4Pal-SI-αMeL- | 215 | 4823.5 | N/I |
| | LDEK((2-[2-(2-Amino-ethoxy)-ethoxy]- | | | |
| | acetyl)₂-CO—(CH₂)₁₈—CO₂H)AQ-Aib-EFI-(D- | | | |
| | Glu)-YLIEGGPSSGAPPPS-NH₂ | | | |
| 207 | Y-Aib-EGT-αMeF(2F)-TSD-4Pal-SI-αMeL- | 216 | 4923.6 | N/I |
| | LDEK((2-[2-(2-Amino-ethoxy)-ethoxy]- | | | |
| | acetyl)₂-(εK)-CO—(CH₂)₁₈—CO₂H)AQ-Aib- | | | |
| | EFI-(D-Glu)-YLIEGGPSSGAPPPS-NH₂ | | | |
| 208 | Y-Aib-EGT-αMeF(2F)-TSDYSI-αMeL- | 217 | 4912.4 | N/I |
| | LDEK((2-[2-(2-Amino-ethoxy)-ethoxy]- | | | |
| | acetyl)₂-(γ-Glu)-CO—(CH₂)₁₄—CO₂H)AQ-Aib- | | | |
| | EFI-(D-Glu)-YLIEGGPSSGAPPPS-NH₂ | | | |
| 209 | Y-Aib-EGT-αMeF(2F)-TSDYSI-αMeL-LD- | 218 | 4911.4 | N/I |
| | Dab-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 210 | Y-Aib-EGT-αMeF(2F)-TSDYSI-αMeL-LD- | 219 | 4897.5 | N/I |
| | Dap-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 211 | Y-Aib-EGT-αMeF(2F)-TSDYSI-αMeL- | 220 | 4953.6 | N/I |
| | LDKK((2-[2-(2-Amino-ethoxy)-ethoxy]- | | | |
| | acetyl)-(γ-Glu)-(2-[2-(2-Amino-ethoxy)- | | | |
| | ethoxy]-acetyl)-CO—(CH₂)₁₈—CO₂H)AQ-Aib- | | | |
| | EFI-(D-Glu)-YLIEGGPSSGAPPPS-NH₂ | | | |
| 212 | Y-Aib-EGT-αMeF(2F)-TSDYSI-αMeL- | 221 | 4967.5 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 213 | Y-Aib-EGT-αMeF-TSDYSI-αMeL- | 222 | 4922.4 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 214 | Y-Aib-EGT-αMeF(2F)-TSDYSI-αMeL- | 223 | 4811.4 | N/I |
| | LDEK((2-[2-(2-Amino-ethoxy)-ethoxy]- | | | |
| | acetyl)₂-CO—(CH₂)₁₆—CO₂H)AQ-Aib-EFI-(D- | | | |
| | Glu)-YLIEGGPSSGAPPPS-NH₂ | | | |
| 215 | Y-Aib-EGT-αMeF(2F)-TSDYSI-αMeL- | 224 | 4839.4 | N/I |
| | LDEK((2-[2-(2-Amino-ethoxy)-ethoxy]- | | | |
| | acetyl)₂-CO—(CH₂)₁₈—CO₂H)AQ-Aib-EFI-(D- | | | |
| | Glu)-YLIEGGPSSGAPPPS-NH₂ | | | |
| 216 | Y-Aib-EGT-αMeF(2F)-TSDYSI-αMeL- | 225 | 4967.5 | N/I |
| | LDEK((2-[2-(2-Amino-ethoxy)-ethoxy]- | | | |
| | acetyl)₂-(εK)-CO—(CH₂)₁₈—CO₂H)AQ-Aib- | | | |
| | EFI-(D-Glu)-YLIEGGPSSGAPPPS-NH₂ | | | |
| 217 | Y-Aib-EGT-αMeF(2F)-TSDYSI-αMeL-LD- | 226 | 4939.5 | N/I |
| | Dab-K((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 218 | Y-Aib-EGT-αMeF(2F)-TSDYSI-αMeL-LD- | 227 | 4925.5 | N/I |
| | Dap-K((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 219 | Y-Aib-EGT-αMeF(2F)-TSDYSI-αMeL- | 228 | 4491.0 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSG-NH₂ | | | |
| 220 | Y-Aib-EGT-αMeF(2F)-TSDYSI-αMeL- | 229 | 4162.6 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGG-NH₂ | | | |
| 221 | Y-Aib-EGT-αMeF(2F)-TSDYSI-αMeL- | 230 | 4940.5 | N/I |
| | LDEK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 222 | Y-Aib-EGT-αMeF(2F)-TSDYSI-αMeL- | 231 | 4982.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-αMeY-LIEGGPSSGAPPPS- | | | |
| | NH₂ | | | |
| 223 | Y-Aib-EGT-αMeF(2F)-TSDYSI-αMeL- | 232 | 4982.6 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFIE-αMeY-LIEGGPSSGAPPPS-NH₂ | | | |
| 224 | Y-Aib-EGT-αMeF(2F)-TSDYSI-Aib- | 233 | 4926.4 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 225 | Y-Aib-EGT-αMeF-TSDYSI-Aib-LDEK((2- | 234 | 4908.5 | N/I |
| | [2-(2-Amino-ethoxy)-ethoxy]-acetyl)₂-(γ- | | | |
| | Glu)-CO—(CH₂)₁₈—CO₂H)AQ-Aib-EFI-(D- | | | |
| | Glu)-YLIEGGPSSGAPPPS-NH₂ | | | |
| 226 | Y-Aib-EGT-αMeF(2F)-TSDYSILLDEK((2- | 235 | 4954.5 | N/I |
| | [2-(2-Amino-ethoxy)-ethoxy]-acetyl)₂-(γ- | | | |
| | Glu)-CO—(CH₂)₁₈—CO₂H)AQ-Aib-EFI-(D- | | | |
| | Glu)-YLIEGGPSSGAPPPS-NH₂ | | | |
| 227 | Y-Aib-EGT-αMeF-TSDYSI-αMeL- | 236 | 4950.5 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 228 | Y-Aib-EGT-αMeF-TSDYSI-αMeL- | 237 | 4500.1 | N/I |
| | LDKK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSG-NH₂ | | | |
| 229 | Y-Aib-EGT-αMeF(2F)-TSDYS-αMeL- | 238 | 4855.4 | N/I |
| | LDEK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 230 | Y-Aib-EGT-αMeF(2F)-TSDYSI-αMeL- | 239 | 4976.6 | N/I |
| | LDHK((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 231 | Y-Aib-EGT-αMeF(2F)-TSDYSI-αMeL- | 240 | 4939.5 | N/I |
| | LDKK((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 232 | Y-Aib-EGT-αMeF(2F)-TSD-4Pal-SI-αMeL- | 241 | 4910.5 | N/I |
| | LD-Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 233 | Y-Aib-EGT-αMeF(2F)-TSD-4Pal-SI-αMeL- | 242 | 4896.5 | N/I |
| | LD-Dab-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 234 | Y-Aib-EGT-αMeF(2F)-TSD-4Pal-SILLD- | 243 | 4882.4 | N/I |
| | Dab-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 235 | Y-Aib-EGT-αMeF-TSD-4Pal-SI-αMeL-LD- | 244 | 4878.5 | N/I |
| | Dab-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 236 | Y-Aib-EGT-αMeF(2F)-TSDYSI-αMeL-LD- | 245 | 4939.5 | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-αMeY-LIEGGPSSGAPPPS- | | | |
| | NH₂ | | | |
| 237 | Y-Aib-EGT-αMeF(2F)-TSDASI-αMeL-LD- | 246 | 4847.4 | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-αMeY-LIEGGPSSGAPPPS- | | | |
| | NH₂ | | | |
| 238 | Y-Aib-EGT-αMeF(2F)-TSDLSI-αMeL-LD- | 247 | 4889.5 | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-αMeY-LIEGGPSSGAPPPS- | | | |
| | NH₂ | | | |
| 239 | Y-Aib-EGT-αMeF(2F)-TSD-4Pal-SI-αMeL- | 248 | 4896.5 | N/I |
| | LD-Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | TFI-(D-Glu)-αMeY-LIEGGPSSGAPPPS- | | | |
| | NH₂ | | | |
| 240 | Y-Aib-EGT-αMeF(2F)-TSD-4Pal-SI-αMeL- | 249 | 4866.5 | N/I |
| | LD-Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | AFI-(D-Glu)-αMeY-LIEGGPSSGAPPPS- | | | |
| | NH₂ | | | |
| 241 | Y-Aib-EGT-αMeF(2F)-TSDVSI-αMeL-LD- | 250 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | TFI-(D-Glu)-αMeY-LIEGGPSSGAPPPS- | | | |
| | NH₂ | | | |
| 242 | Y-Aib-EGT-αMeF(2F)-TSDVSI-αMeL-LD- | 251 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | AFI-(D-Glu)-αMeY-LIEGGPSSGAPPPS- | | | |
| | NH₂ | | | |
| 243 | Y-Aib-EGT-αMeF(2F)-TSDLSI-αMeL-LD- | 252 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | TFI-(D-Glu)-αMeY-LIEGGPSSGAPPPS- | | | |
| | NH₂ | | | |
| 244 | Y-Aib-EGT-αMeF(2F)-TSDLSI-αMeL-LD- | 253 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | AFI-(D-Glu)-αMeY-LIEGGPSSGAPPPS- | | | |
| | NH₂ | | | |
| 245 | Y-Aib-EGT-αMeF(2F)-TSDASI-αMeL-LD- | 254 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | TFI-(D-Glu)-αMeY-LIEGGPSSGAPPPS- | | | |
| | NH₂ | | | |
| 246 | Y-Aib-EGT-αMeF(2F)-TSDASI-αMeL-LD- | 255 | N/I | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | AFI-(D-Glu)-αMeY-LIEGGPSSGAPPPS- | | | |
| | NH₂ | | | |
| 247 | Y-Aib-EGT-αMeF(2F)-TSDYSI-Aib-LD- | 256 | 4883.4 | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 248 | Y-Aib-EGT-αMeF(2F)-TSDYSILLD-Orn- | 257 | 4911.5 | N/I |
| | K((2-[2-(2-Amino-ethoxy)-ethoxy]-acetyl)₂- | | | |
| | (γ-Glu)-CO—(CH₂)₁₆—CO₂H)AQ-Aib-EFI-(D- | | | |
| | Glu)-YLIEGGPSSGAPPPS-NH₂ | | | |
| 249 | Y-Aib-EGT-αMeF(2F)-TSDYSI-Nle-LD- | 258 | 4911.5 | N/I |
| | Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-YLIEGGPSSGAPPPS-NH₂ | | | |
| 250 | Y-Aib-EGT-αMeF(2F)-TSDYSI-Aib-LD- | 259 | 4911.5 | N/I |
| | Orn-K((2-[2-(2-Amino-ethoxy)-ethoxy]- | | | |
| | acetyl)₂-(γ-Glu)-CO—(CH₂)₁₈—CO₂H)AQ-Aib- | | | |
| | EFI-(D-Glu)-YLIEGGPSSGAPPPS-NH₂ | | | |
| 251 | Y-Aib-EGT-αMeF(2F)-TSD-4Pal-SI-αMeL- | 260 | 4893.6 | N/I |
| | LD-Orn-K((2-[2-(2-Amino-ethoxy)-ethoxy]- | | | |
| | acetyl)₂-(εK)-CO—(CH₂)₁₈—CO₂H)AQ-Aib- | | | |
| | AFI-(D-Glu)-αMeY-LIEGGPSSGAPPPS- | | | |
| | NH₂ | | | |
| 252 | Y-Aib-EGT-αMeF(2F)-TSD-4Pal-SI-αMeL- | 261 | 4835.6 | N/I |
| | LD-Orn-K((2-[2-(2-Amino-ethoxy)-ethoxy]- | | | |
| | acetyl)₂-(εK)-CO—(CH₂)₁₈—CO₂H)AQ-Aib- | | | |
| | AFI-(D-Glu)-αMeY-LIAGGPSSGAPPPS- | | | |
| | NH₂ | | | |
| 253 | Y-Aib-EGTFTSDYSILLDKK((2-[2-(2- | 262 | 4849.5 | N/I |
| | Amino-ethoxy)-ethoxy]-acetyl)₂-(γ-Glu)-CO—(CH₂)₁₈—CO₂H) | | | |
| | AQAAFIEYLIEGGPSSGAPPPS-NH₂ | | | |
| 254 | Y-Aib-EGTFTSDYSILLDKK((2-[2-(2- | 263 | 4906.5 | N/I |
| | Amino-ethoxy)-ethoxy]-acetyl)₂-(γ-Glu)-CO—(CH₂)₁₈—CO₂H) | | | |
| | AQQAFIEYLIEGGPSSGAPPPS-NH₂ | | | |
| 255 | Y-Aib-EGTFTSDYSILLDKK((2-[2-(2- | 264 | 4915.5 | N/I |
| | Amino-ethoxy)-ethoxy]-acetyl)₂-(γ-Glu)-CO—(CH₂)₁₈—CO₂H) | | | |
| | AQHAFIEYLIEGGPSSGAPPPS-NH₂ | | | |
| 256 | Y-Aib-EGTFTSDYSILLDKK((2-[2-(2- | 265 | 4906.6 | N/I |
| | Amino-ethoxy)-ethoxy]-acetyl)₂-(γ-Glu)-CO—(CH₂)₁₈—CO₂H) | | | |
| | AQKAFIEYLIEGGPSSGAPPPS-NH₂ | | | |
| 257 | Y-Aib-EGTFTSDYSILLDKK((2-[2-(2- | 266 | 4934.6 | N/I |
| | Amino-ethoxy)-ethoxy]-acetyl)₂-(γ-Glu)-CO—(CH₂)₁₈—CO₂H) | | | |
| | AQRAFIEYLIEGGPSSGAPPPS-NH₂ | | | |
| 258 | Y-Aib-EGTFTSDYSILLDEK((2-[2-(2- | 267 | 4907.5 | N/I |
| | Amino-ethoxy)-ethoxy]-acetyl)₂-(γ-Glu)-CO—(CH₂)₁₈—CO₂H) | | | |
| | AQKAFIEYLIEGGPSSGAPPPS-NH₂ | | | |
| 259 | Y-Aib-EGTFTSDYSILLDEK((2-[2-(2- | 268 | 4864.4 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 260 | Y-Aib-EGTFTSDHSILLDKK((2-[2-(2- | 269 | 4837.5 | N/I |
| | Amino-ethoxy)-ethoxy]-acetyl)₂-(γ-Glu)-CO—(CH₂)₁₈—CO₂H) | | | |
| | AQ-Aib- | | | |
| | AFIEYLIEGGPSSGAPPPS-NH₂ | | | |
| 261 | Y-Aib-EGTFTSDYSILLDKK((2-[2-(2- | 270 | 4907.5 | N/I |
| | Amino-ethoxy)-ethoxy]-acetyl)₂-(γ-Glu)-CO—(CH₂)₁₈—CO₂H) | | | |
| | AQEAFIEYLIEGGPSSGAPPPS-NH₂ | | | |
| 262 | Y-Aib-EGTFTSDYSILLDKK((2-[2-(2- | 271 | 4879.5 | N/I |
| | Amino-ethoxy)-ethoxy]-acetyl)₂-(γ-Glu)-CO—(CH₂)₁₈—CO₂H) | | | |
| | AQTAFIEYLIEGGPSSGAPPPS-NH₂ | | | |
| 263 | Y-Aib-EGTFTSDYSILLDKK((2-[2-(2- | 272 | 4865.5 | N/I |
| | Amino-ethoxy)-ethoxy]-acetyl)₂-(γ-Glu)-CO—(CH₂)₁₈—CO₂H) | | | |
| | AQSAFIEYLIEGGPSSGAPPPS-NH₂ | | | |
| 264 | Y-Aib-EGT-αMeF(2F)-TSD-4Pal-SI-αMeL- | 273 | 4475.0 | N/I |
| | LD-Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-αMeY-LIEGGPSSG-NH₂ | | | |
| 265 | Y-Aib-EGT-αMeF(2F)-TSD-4Pal-SI-αMeL- | 274 | 4146.7 | N/I |
| | LD-Orn-K((2-[2-(2-Amino-ethoxy)-ethoxy]- | | | |
| | acetyl)₂-(γ-Glu)-CO—(CH₂)₁₆—CO₂H)AQ-Aib- | | | |
| | EFI-(D-Glu)-αMeY-LIEGG-NH₂ | | | |
| 266 | Y-Aib-EGTFTSDYSILLDKK((2-[2-(2- | 275 | 4385.94 | 4386.6 |
| | Amino-ethoxy)-ethoxy]-acetyl)₂-(γ-Glu)-CO—(CH₂)₁₆—CO₂H) | | | |
| | AQ-Aib-AFIEYLIEGGPSSG- | | | |
| | NH₂ | | | |
| 267 | Y-Aib-EGTFTSDYSILLDKK((2-[2-(2- | 276 | 4057.62 | N/I |
| | Amino-ethoxy)-ethoxy]-acetyl)₂-(γ-Glu)-CO—(CH₂)₁₆—CO₂H) | | | |
| | AQ-Aib-AFIEYLIEGG-NH₂ | | | |
| 268 | Y-Aib-EGTFTSDYSILLDEK((2-[2-(2- | 277 | 4386.88 | N/I |
| | Amino-ethoxy)-ethoxy]-acetyl)₂-(γ-Glu)-CO—(CH₂)₁₆—CO₂H) | | | |
| | AQ-Aib-AFIEYLIEGGPSSG- | | | |
| | NH₂ | | | |
| 269 | Y-Aib-EGTFTSDYSILLDEK((2-[2-(2- | 278 | 4058.56 | N/I |
| | Amino-ethoxy)-ethoxy]-acetyl)₂-(γ-Glu)-CO—(CH₂)₁₆—CO₂H) | | | |
| | AQ-Aib-AFIEYLIEGG-NH₂ | | | |
| 270 | Y-Aib-EGTFTSDYSILLDKK((2-[2-(2- | 279 | 4443.98 | N/I |
| | Amino-ethoxy)-ethoxy]-acetyl)₂-(γ-Glu)-CO—(CH₂)₁₆—CO₂H) | | | |
| | AQ-Aib-EFIEYLIEGGPSSG- | | | |
| | NH₂ | | | |
| 271 | Y-Aib-EGTFTSDYSILLDKK((2-[2-(2- | 280 | 4115.66 | N/I |
| | Amino-ethoxy)-ethoxy]-acetyl)₂-(γ-Glu)-CO—(CH₂)₁₆—CO₂H) | | | |
| | AQ-Aib-EFIEYLIEGG-NH₂ | | | |
| 272 | Y-Aib-EGTFTSDYSILLDKK((2-[2-(2- | 281 | 4327.91 | 4327.8 |
| | Amino-ethoxy)-ethoxy]-acetyl)₂-(γ-Glu)-CO—(CH₂)₁₆—CO₂H) | | | |
| | AQ-Aib-AFIEYLIAGGPSSG- | | | |
| | NH₂ | | | |
| 273 | Y-Aib-EGTFTSDYSILLDKK((2-[2-(2- | 282 | 3999.58 | N/I |
| | Amino-ethoxy)-ethoxy]-acetyl)₂-(γ-Glu)-CO—(CH₂)₁₆—CO₂H) | | | |
| | AQ-Aib-AFIEYLIAGG-NH₂ | | | |
| 274 | Y-Aib-EGTFTSDYSILLDKK((2-[2-(2- | 283 | 4397.95 | N/I |
| | Amino-ethoxy)-ethoxy]-acetyl)₂-(γ-Glu)-CO—(CH₂)₁₆—CO₂H) | | | |
| | AQPAFIEYLIEGGPSSG- | | | |
| | NH₂ | | | |
| 275 | Y-Aib-EGTFTSDYSILLDKK((2-[2-(2- | 284 | 4069.63 | N/I |
| | Amino-ethoxy)-ethoxy]-acetyl)₂-(γ-Glu)-CO—(CH₂)₁₆—CO₂H) | | | |
| | AQPAFIEYLIEGG-NH₂ | | | |
| 276 | Y-Aib-EGT-αMeF(2F)-TSDYSI-αMeL- | 285 | 4224.59 | N/I |
| | LDEKAQ-Aib-EFI-(D-Glu)- | | | |
| | YLIEGGPSSGAPPPS-NH₂ | | | |
| 277 | Y-Aib-EGT-αMeF(2F)-TSDYSI-αMeL- | 286 | 4224.55 | N/I |
| | LDEQAQ-Aib-EFI-(D-Glu)- | | | |
| | YLIEGGPSSGAPPPS-NH₂ | | | |
| 278 | Y-Aib-EGT-αMeF(2F)-TSDYSI-αMeL- | 287 | 4327.74 | N/I |
| | LDEKAQ-Aib-EFI-(D-Glu)- | | | |
| | YLIEGGPSSGAPPPSC-NH₂ | | | |
| 279 | Y-Aib-EGT-αMeF(2F)-TSDYSI-αMeL- | 288 | 4327.69 | N/I |
| | LDEQAQ-Aib-EFI-(D-Glu)- | | | |
| | YLIEGGPSSGAPPPSC-NH₂ | | | |
| 280 | Y-Aib-EGT-αMeF(2F)-TSDYSI-αMeL-LD- | 289 | 4210.61 | N/I |
| | Orn-KAQ-Aib-EFI-(D-Glu)- | | | |
| | YLIEGGPSSGAPPPS-NH₂ | | | |
| 281 | Y-Aib-EGT-αMeF(2F)-TSDYSI-αMeL-LD- | 290 | 4209.58 | N/I |
| | Orn-QAQ-Aib-EFI-(D-Glu)- | | | |
| | YLIEGGPSSGAPPPS-NH₂ | | | |
| 282 | Y-Aib-EGT-αMeF(2F)-TSDYSI-αMeL-LD- | 291 | 4312.77 | N/I |
| | Orn-KAQ-Aib-EFI-(D-Glu)- | | | |
| | YLIEGGPSSGAPPPSC-NH₂ | | | |
| 283 | Y-Aib-EGT-αMeF(2F)-TSDYSI-αMeL-LD- | 292 | 4312.73 | N/I |
| | Orn-QAQ-Aib-EFI-(D-Glu)- | | | |
| | YLIEGGPSSGAPPPSC-NH₂ | | | |
| 284 | Y-Aib-EGT-αMeF(2F)-TSD-4Pal-SI-αMeL- | 293 | 4208.64 | N/I |
| | LD-Orn-KAQ-Aib-EFI-(D-Glu)-αMeY- | | | |
| | LIEGGPSSGAPPPS-NH₂ | | | |
| 285 | Y-Aib-EGT-αMeF(2F)-TSD-4Pal-SI-αMeL- | 294 | 4208.6 | N/I |
| | LD-Orn-QAQ-Aib-EFI-(D-Glu)-αMeY- | | | |
| | LIEGGPSSGAPPPS-NH₂ | | | |
| 286 | Y-Aib-EGT-αMeF(2F)-TSD-4Pal-SI-αMeL- | 295 | 4311.78 | N/I |
| | LD-Orn-KAQ-Aib-EFI-(D-Glu)-αMeY- | | | |
| | LIEGGPSSGAPPPSC-NH₂ | | | |
| 287 | Y-Aib-EGT-αMeF(2F)-TSD-4Pal-SI-αMeL- | 296 | 4311.74 | N/I |
| | LD-Orn-QAQ-Aib-EFI-(D-Glu)-αMeY- | | | |
| | LIEGGPSSGAPPPSC-NH₂ | | | |

N/I means Not Included

### Example 288

### Y-Aib-EGTFTSDYSILLDKK((2-[2-(2-Amino-ethoxy)-ethoxy]-acetyl)₂-(γ-Glu)-CO—(CH₂)₁₈—CO₂H)AQ-Aib-AFIEYLIAGGPSSGAPPPS-NH₂ (SEQ ID NO:303)

The structure of SEQ ID NO:303 is depicted below using the standard single letter amino acid codes with the exception of residues Aib2, K17, Aib20, and Ser39, where the structures of these amino acid residues have been expanded:

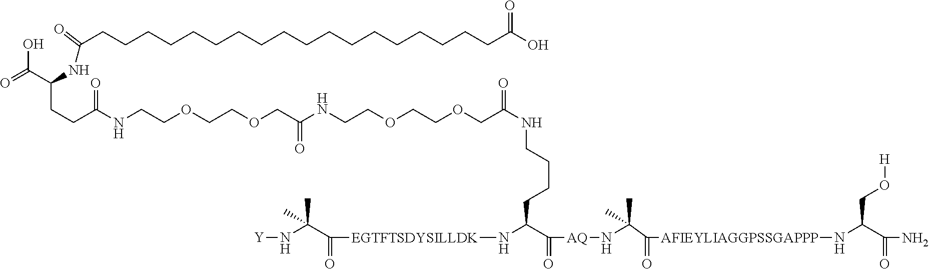

The compound according to SEQ ID NO:303 is prepared substantially as described by the procedures of Example 1. The molecular weight is determined by LC-MS (obsd: M+3=1602.5; Calc M+3=1602.8).

### Example 289

### Y-Aib-EGTFTSDYSI-αMeL-LDKK((2-[2-(2-Amino-ethoxy)-ethoxy]-acetyl)₂-(γ-Glu)-CO—(CH₂)₁₈—CO₂H)AQ-Aib-EFIEYLIAGGPSSGAPPPS-NH₂ (SEQ ID NO:304)

The structure of SEQ ID NO:304 is depicted below using the standard single letter amino acid codes with the exception of residues Aib2, αMeL13, K17, Aib20, and Ser39, where the structures of these amino acid residues have been expanded:

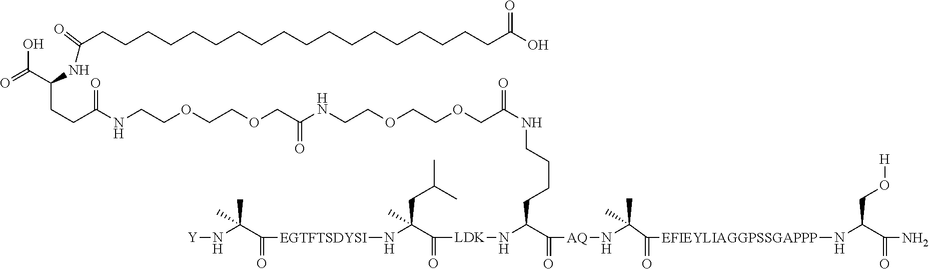

The compound according to SEQ ID NO:304 is prepared substantially as described by the procedures of Example 1. The molecular weight is determined by LC-MS (obsd: M+3-1626.8; Calc M+3=1626.8).

### Example 290

### (D-Tyr)-Aib-EGTFTSDYSI-αMeL-LDKK((2-[2-(2-Amino-ethoxy)-ethoxy]-acetyl)₂-(γ-Glu)-CO—(CH₂)₁₈—CO₂H)AQ-Aib-EFIEYLIAGGPSSGAPPPS-NH₂ (SEQ ID NO:305)

The structure of SEQ ID NO:305 is depicted below using the standard single letter amino acid codes with the exception of residues D-Tyr1, Aib2, αMeL13, K17, Aib20, and Ser39, where the structures of these amino acid residues have been expanded:

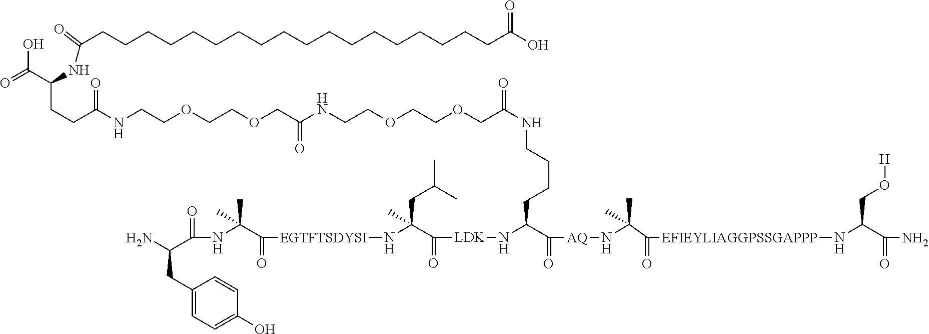

The compound according to SEQ ID NO:305 is prepared substantially as described by the procedures of Example 1. The molecular weight is determined by LC-MS (obsd: M+3=1626.6; Calc M+3=1626.8).

### Example 291

### (D-Tyr)-Aib-EGTFTSDYSI-αMeL-LD-Orn-K((2-[2-(2-Amino-ethoxy)-ethoxy]-acetyl)₂-(γ-Glu)-CO—(CH₂)₁₈—CO₂H)AQ-Aib-AFI-(D-Glu)-YLIAGGPSSGAPPPS-NH₂ (SEQ ID NO:306)

The structure of SEQ ID NO:306 is depicted below using the standard single letter amino acid codes with the exception of residues D-Tyr1, Aib2, αMeL13, Orn16, K17, Aib20, D-Glu24, and Ser39, where the structures of these amino acid residues have been expanded:

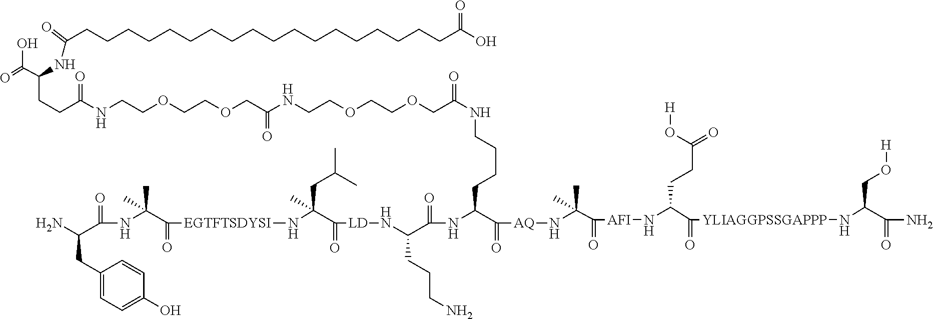

The compound according to SEQ ID NO:306 is prepared substantially as described by the procedures of Example 1. The molecular weight is determined by LC-MS (obsd: M-3=1602.4: Calc M+3=1602.8).

### Example 292

### (D-Tyr)-Aib-EGTFTSDYSI-αMeL-LDKK((2-[2-(2-Amino-ethoxy)-ethoxy]-acetyl)₂-(γ-Glu)-CO—(CH₂)₁₈—CO₂H)AQ-Aib-EFIE-αMeY-LIAGGPSSGAPPPS-NH₂ (SEQ ID NO:307)

The structure of SEQ ID NO:307 is depicted below using the standard single letter amino acid codes with the exception of residues D-Tyr1, Aib2, αMeL13, K17, Aib20, αMeY25, and Ser39, where the structures of these amino acid residues have been expanded:

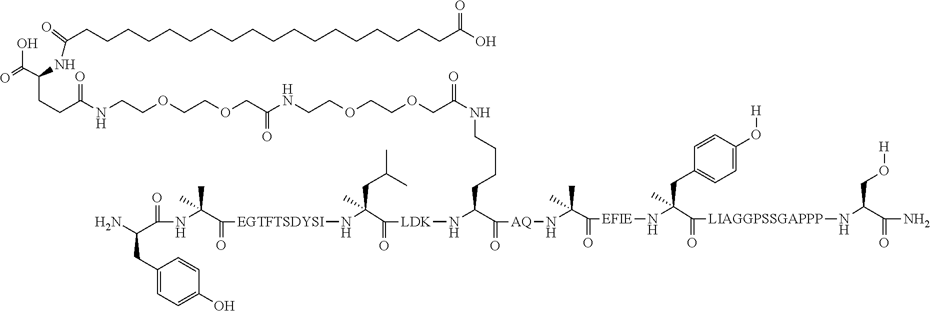

The compound according to SEQ ID NO:307 is prepared substantially as described by the procedures of Example 1. The molecular weight is determined by LC-MS (obsd: M+3=1631.3; Calc M+3=1631.5).

### Example 293

### (D-Tyr)-Aib-EGTFTSDYSI-αMeL-LD-Orn-K((2-[2-(2-Amino-ethoxy)-ethoxy]-acetyl)₂-(γ-Glu)-CO—(CH₂)₁₈—CO₂H)AQ-Aib-EFIE-αMeY-LIAGGPSSGAPPPS-NH₂ (SEQ ID NO:308)

The structure of SEQ ID NO:308 is depicted below using the standard single letter amino acid codes with the exception of residues D-Tyr1, Aib2, αMeL13, Orn16, K17, Aib20, αMeY25, and Ser39, where the structures of these amino acid residues have been expanded:

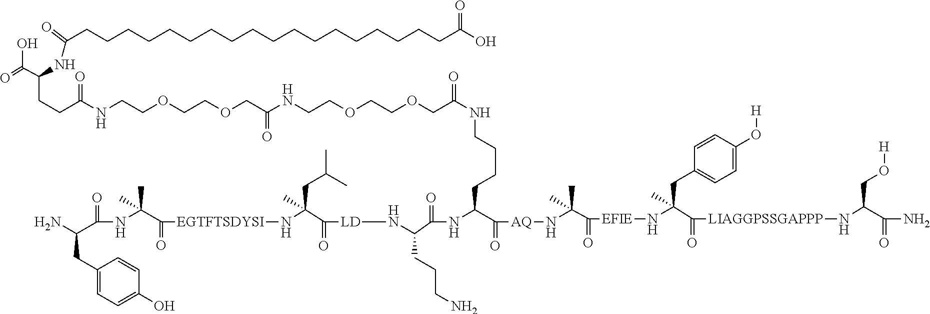

The compound according to SEQ ID NO:308 is prepared substantially as described by the procedures of Example 1. The molecular weight is determined by LC-MS (obsd: M+3==162.5; Calc M+3=1626.8).

### Example 294 Through Example 381

The compounds according to Examples 294 (SEQ ID NO:309) through Example 381 (SEQ ID NO:396) are prepared substantially as described by the procedures of Example 1.

| | | | Calculated | Found |
|---|---|---|---|---|
| | | SEQ | MW | MW |
| Example | Compound Name | ID NO | (average) | (average) |
| 294 | Y-Aib-EGTFTSDYSILLDKK((2-[2-(2- | 309 | 4100.6 | N/I |
| | Amino-ethoxy)-ethoxy]- | | | |
| | acetyl)₂-(γ-Glu)-CO- | | | |
| | (CH₂)₁₆-CO₂H)AQQAFIEYLIEGG-NH₂ | | | |
| 295 | Y-Aib-EGTFTSDYSILLDKK((2-[2-(2- | 310 | 4109.7 | N/I |
| | Amino-ethoxy)-ethoxy]- | | | |
| | acetyl)₂-(γ-Glu)-CO- | | | |
| | (CH₂)₁₆-CO₂H)AQHAFIEYLIEGG-NH₂ | | | |
| 296 | Y-Aib-EGTFTSDYSILLDKK((2-[2-(2- | 311 | 4429.0 | N/I |
| | Amino-ethoxy)-ethoxy]- | | | |
| | acetyl)₂-(γ-Glu)-CO- | | | |
| | (CH₂)₁₆-CO₂H)AQKAFIEYLIEGGPSSG- | | | |
| | NH₂ | | | |
| 297 | Y-Aib-EGTFTSDYSILLDKK((2-[2-(2- | 312 | 4057.6 | N/I |
| | Amino-ethoxy)-ethoxy]- | | | |
| | acetyl)₂-(γ-Glu)-CO- | | | |
| | (CH₂)₁₆-CO₂H)AQ-Aib-EFIEYLIAGG-NH₂ | | | |
| 298 | Y-Aib-EGTFTSDYSILLDKK((2-[2-(2- | 313 | 4313.9 | N/I |
| | Amino-ethoxy)-ethoxy]- | | | |
| | acetyl)₂-(γ-Glu)-CO- | | | |
| | (CH₂)₁₆-CO₂H)AQ-Aib- | | | |
| | AFVEYLIEGGPSSG-NH₂ | | | |
| 299 | Y-Aib-EGTFTSDYSILLDKK((2-[2-(2- | 314 | 4385.9 | N/I |
| | Amino-ethoxy)-ethoxy]- | | | |
| | acetyl)₂-(γ-Glu)-CO- | | | |
| | (CH₂)₁₆-CO₂H)AQ-Aib- | | | |
| | AFLEYLIEGGPSSG-NH₂ | | | |
| 300 | Y-Aib-EGTFTSDYSI-αMeL-LDKK((2-[2- | 315 | 4400.0 | N/I |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSG-NH₂ | | | |
| 301 | Y-Aib-EGT-αMeF-TSDYSILLDKK((2-[2- | 316 | 4400.0 | 4399.2 |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSG-NH₂ | | | |
| 302 | Y-Aib-EGT-αMeF(2F)- | 317 | 4418.0 | N/I |
| | TSDYSILLDKK((2- | | | |
| | [2-(2-Amino-ethoxy)-ethoxy]-acetyl)₂- | | | |
| | (γ-Glu)-CO-(CH₂)₁₆-CO₂H)AQ-Aib- | | | |
| | AFIEYLIEGGPSSG-NH₂ | | | |
| 303 | Y-Aib-EGTFTSDYSI-αMeL-LDEK((2-[2- | 318 | 4400.9 | 4400.7 |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSG-NH₂ | | | |
| 304 | Y-Aib-EGTFTSDYSI-αMeL-LDKK((2-[2- | 319 | 4458.0 | N/I |
| | (2-Amino-ethoxy)-ethoxy]-acetyl)₂-(γ-Glu)- | | | |
| | CO-(CH₂)₁₆-CO₂H)AQ-Aib- | | | |
| | EFIEYLIEGGPSSG-NH₂ | | | |
| 305 | Y-Aib-EGTFTSDYSI-αMeL-LDKK((2-[2- | 320 | 4341.9 | N/I |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIAGGPSSG-NH₂ | | | |
| 306 | Y-Aib-EGTFTSDYSI-αMeL-LDKK((2-[2- | 321 | 4400.0 | N/I |
| | (2-Amino-ethoxy)-ethoxy]-acetyl)₂-(γ-Glu)- | | | |
| | CO-(CH₂)₁₆-CO₂H)AQ-Aib- | | | |
| | EFIEYLIAGGPSSG-NH₂ | | | |
| 307 | Y-Aib-EGT-αMeF-TSDYSILLDEK((2-[2- | 322 | 4400.9 | N/I |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSG-NH₂ | | | |
| 308 | Y-Aib-EGT-αMeF-TSDYSILLDKK((2-[2- | 323 | 4458.0 | N/I |
| | (2-Amino-ethoxy)-ethoxy]-acetyl)₂-(γ-Glu)- | | | |
| | CO-(CH₂)₁₆-CO₂H)AQ-Aib- | | | |
| | EFIEYLIEGGPSSG-NH₂ | | | |
| 309 | Y-Aib-EGT-αMeF-TSDYSILLDKK((2-[2- | 324 | 4341.9 | N/I |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIAGGPSSG-NH₂ | | | |
| 310 | Y-Aib-EGT-αMeF- | 325 | 4400.0 | N/I |
| | TSDYSILLDKK((2-[2-(2-Amino- | | | |
| | ethoxy)-ethoxy]-acetyl)₂-(γ-Glu)- | | | |
| | CO-(CH₂)₁₆-CO₂H)AQ-Aib- | | | |
| | EFIEYLIAGGPSSG-NH₂ | | | |
| 311 | Y-Aib-EGT-αMeF(2F)- | 326 | 4418.9 | N/I |
| | TSDYSILLDEK((2- | | | |
| | [2-(2-Amino-ethoxy)-ethoxy]-acetyl)₂-(γ- | | | |
| | Glu)-CO-(CH₂)₁₆-CO₂H)AQ-Aib- | | | |
| | AFIEYLIEGGPSSG-NH₂ | | | |
| 312 | Y-Aib-EGT-αMeF(2F)- | 327 | 4476.0 | N/I |
| | TSDYSILLDKK((2- | | | |
| | [2-(2-Amino-ethoxy)-ethoxy]-acetyl)₂-(γ- | | | |
| | Glu)-CO-(CH₂)₁₆-CO₂H)AQ-Aib- | | | |
| | EFIEYLIEGGPSSG-NH₂ | | | |
| 313 | Y-Aib-EGT-αMeF(2F)- | 328 | 4359.9 | N/I |
| | TSDYSILLDKK((2- | | | |
| | [2-(2-Amino-ethoxy)-ethoxy]-acetyl)₂-(γ- | | | |
| | Glu)-CO-(CH₂)₁₆-CO₂H)AQ-Aib- | | | |
| | AFIEYLIAGGPSSG-NH₂ | | | |
| 314 | Y-Aib-EGT-αMeF(2F)- | 329 | 4418.0 | N/I |
| | TSDYSILLDKK((2- | | | |
| | [2-(2-Amino-ethoxy)-ethoxy]-acetyl)₂-(γ- | | | |
| | Glu)-CO-(CH₂)₁₆-CO₂H)AQ-Aib- | | | |
| | EFIEYLIAGGPSSG-NH₂ | | | |
| 315 | Y-Aib-EGTFTSDYSILLDKK((2-[2-(2- | 330 | 4835.5 | N/I |
| | Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSGAPPPS-NH₂ | | | |
| 316 | Y-Aib-EGTFTSDYSILLDKK((2-[2-(2- | 331 | 4777.4 | 4777.2 |
| | Amino-ethoxy)-ethoxy]-acetyl)₂-(γ-Glu)- | | | |
| | CO-(CH₂)₁₆-CO₂H)AQ-Aib- | | | |
| | AFIEYLIAGGPSSGAPPPS-NH₂ | | | |
| 317 | Y-Aib-EGTFTSDYSILLDKK((2-[2-(2- | 332 | 4356.0 | N/I |
| | Amino-ethoxy)-ethoxy]-acetyl)₂-(γ-Glu)- | | | |
| | CO-(CH₂)₁₈-CO₂H)AQ-Aib- | | | |
| | AFIEYLIAGGPSSG-NH₂ | | | |
| 318 | Y-Aib-EGTFTSDYSILLDKK((2-[2-(2- | 333 | 4414.0 | N/I |
| | Amino-ethoxy)-ethoxy]-acetyl)₂-(γ-Glu)- | | | |
| | CO-(CH₂)₁₈-CO₂H)AQ-Aib- | | | |
| | EFIEYLIAGGPSSG-NH₂ | | | |
| 319 | Y-Aib-EGTFTSDYSILLDKK((2-[2-(2- | 334 | 4863.5 | N/I |
| | Amino-ethoxy)-ethoxy]-acetyl)₂-(γ-Glu)- | | | |
| | CO-(CH₂)₁₈-CO₂H)AQ-Aib- | | | |
| | EFIEYLIAGGPSSGAPPPS-NH₂ | | | |
| 320 | Y-Aib-EGTFTSDYSILLDKK((2-[2-(2- | 335 | 4430.0 | N/I |
| | Amino-ethoxy)-ethoxy]-acetyl)₂-(γ-Glu)- | | | |
| | CO-(CH₂)₁₆-CO₂H)AQ-Aib- | | | |
| | DFIEYLIEGGPSSG-NH₂ | | | |
| 321 | Y-Aib-EGTFTSDYSILLDKK((2-[2-(2- | 336 | 4416.0 | N/I |
| | Amino-ethoxy)-ethoxy]-acetyl)₂-(γ-Glu)- | | | |
| | CO-(CH₂)₁₆-CO₂H)AQ-Aib- | | | |
| | TFIEYLIEGGPSSG-NH₂ | | | |
| 322 | Y-Aib-EGTFTSDYSILLDKK((2-[2-(2- | 337 | 4452.0 | N/I |
| | Amino-ethoxy)-ethoxy]-acetyl)₂-(γ-Glu)- | | | |
| | CO-(CH₂)₁₆-CO₂H)AQ-Aib- | | | |
| | HFIEYLIEGGPSSG-NH₂ | | | |
| 323 | Y-Aib-EGTFTSDYSI-αMeL-LDEK((2-[2- | 338 | 4850.4 | N/I |
| | (2-Amino-ethoxy)-ethoxy]-acetyl)₂-(γ-Glu)- | | | |
| | CO-(CH₂)₁₆-CO₂H)AQ-Aib- | | | |
| | AFIEYLIEGGPSSGAPPPS-NH₂ | | | |
| 324 | Y-Aib-EGTFTSDYSI-αMeL-LDKK((2-[2- | 339 | 4819.5 | N/I |
| | (2-Amino-ethoxy)-ethoxy]-acetyl)₂-(γ-Glu)- | | | |
| | CO-(CH₂)₁₈-CO₂H)AQ-Aib- | | | |
| | AFIEYLIAGGPSSGAPPPS-NH₂ | | | |
| 325 | Y-Aib-EGT-αMeF(2F)- | 340 | 4895.5 | N/I |
| | TSDYSILLDKK((2- | | | |
| | [2-(2-Amino-ethoxy)-ethoxy]-acetyl)₂-(γ- | | | |
| | Glu)-CO-(CH₂)₁₈-CO₂H)AQ-Aib- | | | |
| | AFIEYLIEGGPSSGAPPPS-NH₂ | | | |
| 326 | F-Aib-EGTFTSDYSILLDKK((2-[2-(2- | 341 | 4847.5 | N/I |
| | Amino-ethoxy)-ethoxy]-acetyl)₂-(γ-Glu)- | | | |
| | CO-(CH₂)₁₈-CO₂H)AQ-Aib- | | | |
| | AFIEYLIEGGPSSGAPPPS-NH₂ | | | |
| 327 | F-Aib-EGTFTSDYSI- | 342 | 4861.5 | N/I |
| | αMeL-LDKK((2-[2-(2- | | | |
| | Amino-ethoxy)-ethoxy]-acetyl)₂-(γ-Glu)- | | | |
| | CO-(CH₂)₁₈-CO₂H)AQ-Aib- | | | |
| | AFIEYLIEGGPSSGAPPPS-NH₂ | | | |
| 328 | F-Aib-EGTFTSDYSI- | 343 | 4847.5 | N/I |
| | αMeL-LDKK((2-[2-(2- | | | |
| | Amino-ethoxy)-ethoxy]-acetyl)₂-(γ-Glu)- | | | |
| | CO-(CH₂)₁₆-CO₂H)AQ-Aib- | | | |
| | AFIEYLIEAGPSSGAPPPS-NH₂ | | | |
| 329 | (D-Tyr)-Aib-EGT-αMeF- | 344 | 4877.5 | N/I |
| | TSDYSILLDKK((2-[2-(2-Amino-ethoxy)- | | | |
| | ethoxy]-acetyl)₂-(γ-Glu)-CO-(CH₂)₁₈- | | | |
| | CO₂H)AQ-Aib- | | | |
| | AFIEYLIEGGPSSGAPPPS-NH₂ | | | |
| 330 | Y-Aib-EGTFTSDYSILLDEK((2-[2-(2- | 345 | 4806.4 | 4805.4 |
| | Amino-ethoxy)-ethoxy]-acetyl)₂-(γ-Glu)- | | | |
| | CO-(CH₂)₁₈-CO₂H)AQ-Aib- | | | |
| | AFIEYLIAGGPSSGAPPPS-NH₂ | | | |
| 331 | Y-Aib-EGTFTSDYSI-αMeL-LDEK((2-[2- | 346 | 4878.5 | N/I |
| | (2-Amino-ethoxy)-ethoxy]-acetyl)₂-(γ-Glu)- | | | |
| | CO-(CH₂)₁₈-CO₂H)AQ-Aib- | | | |
| | EFIEYLIAGGPSSGAPPPS-NH₂ | | | |
| 332 | Y-Aib-EGTFTSDYSI-αMeL-LDEK((2-[2- | 347 | 4820.4 | N/I |
| | (2-Amino-ethoxy)-ethoxy]-acetyl)₂-(γ-Glu)- | | | |
| | CO-(CH₂)₁₈-CO₂H)AQ-Aib- | | | |
| | AFIEYLIAGGPSSGAPPPS-NH₂ | | | |
| 333 | Y-Aib-EGTFTSDYSI-αMeL-LDKK((2-[2- | 348 | 4935.6 | N/I |
| | (2-Amino-ethoxy)-ethoxy]-acetyl)₂-(γ-Glu)- | | | |
| | CO-(CH₂)₁₈-CO₂H)AQ-Aib- | | | |
| | EFIEYLIEGGPSSGAPPPS-NH₂ | | | |
| 334 | (D-Tyr)-Aib-EGTFTSDYSI-αMeL- | 349 | 4877.5 | N/I |
| | LDKK((2-[2-(2-Amino-ethoxy)-ethoxy]- | | | |
| | acetyl)₂-(γ-Glu)-CO- | | | |
| | (CH₂)₁₈-CO₂H)AQ-Aib- | | | |
| | AFIEYLIEGGPSSGAPPPS-NH₂ | | | |
| 335 | (D-Tyr)-Aib-EGT-αMeF-TSDYSI-αMeL- | 350 | 4891.6 | N/I |
| | LDKK((2-[2-(2-Amino-ethoxy)-ethoxy]- | | | |
| | acetyl)₂-(γ-Glu)-CO- | | | |
| | (CH₂)₁₈-CO₂H)AQ-Aib- | | | |
| | AFIEYLIEGGPSSGAPPPS-NH₂ | | | |
| 336 | (D-Tyr)-Aib-EGT-αMeF(2F)-TSDYSI- | 351 | 4909.5 | N/I |
| | αMeL-LDKK((2-[2-(2-Amino-ethoxy)- | | | |
| | ethoxy]-acetyl)₂-(γ-Glu)-CO-(CH₂)₁₈- | | | |
| | CO₂H)AQ-Aib- | | | |
| | AFIEYLIEGGPSSGAPPPS-NH₂ | | | |
| 337 | F-Aib-EGTFTSDYSI- | 352 | 4803.5 | N/I |
| | αMeL-LDKK((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFIEYLIAGGPSSGAPPPS-NH₂ | | | |
| 338 | F-Aib-EGTFTSDYSI- | 353 | 4861.5 | N/I |
| | αMeL-LDKK((2-[2-(2- | | | |
| | Amino-ethoxy)-ethoxy]-acetyl)₂-(γ-Glu)- | | | |
| | CO-(CH₂)₁₈-CO₂H)AQ-Aib- | | | |
| | EFIEYLIAGGPSSGAPPPS-NH₂ | | | |
| 339 | Y-Aib-EGTFTSDYSI-αMeL-LDKK((2-[2- | 354 | 4732.4 | 4732.2 |
| | (2-Amino-ethoxy)-ethoxy]-acetyl)-(γ-Glu)- | | | |
| | CO-(CH₂)₁₈-CO₂H)AQ-Aib- | | | |
| | EFIEYLIAGGPSSGAPPPS-NH₂ | | | |
| 340 | (D-Tyr)-Aib-EGTFTSDYSI-αMeL- | 355 | 4819.5 | 4818.8 |
| | LDKK((2-[2-(2-Amino-ethoxy)-ethoxy]- | | | |
| | acetyl)₂-(γ-Glu)-CO-(CH₂)₁₈-CO₂H)AQ- | | | |
| | Aib-AFIEYLIAGGPSSGAPPPS-NH₂ | | | |
| 341 | (D-Tyr)-Aib-EGTFTSDYSI-αMeL- | 356 | 4820.4 | N/I |
| | LDEK((2-[2-(2-Amino-ethoxy)-ethoxy]- | | | |
| | acetyl)₂-(γ-Glu)-CO-(CH₂)₁₈-CO₂H)AQ- | | | |
| | Aib-AFIEYLIAGGPSSGAPPPS-NH₂ | | | |
| 342 | (D-Tyr)-Aib-EGTFTSDYSI-αMeL- | 357 | 4878.5 | N/I |
| | LDEK((2-[2-(2-Amino-ethoxy)-ethoxy]- | | | |
| | acetyl)₂-(γ-Glu)-CO-(CH₂)₁₈-CO₂H)AQ- | | | |
| | Aib-EFIEYLIAGGPSSGAPPPS-NH₂ | | | |
| 343 | Y-Aib-EGTFTSDYSI-αMeL-LDEK((2-[2- | 358 | 4820.4 | N/I |
| | (2-Amino-ethoxy)-ethoxy]-acetyl)₂-(γ-Glu)- | | | |
| | CO-(CH₂)₁₈-CO₂H)AQ-Aib-AFI-(D-Glu)- | | | |
| | YLIAGGPSSGAPPPS-NH₂ | | | |
| 344 | Y-Aib-EGTFTSDYSI-αMeL-LDEK((2-[2- | 359 | 4864.4 | N/I |
| | (2-Amino-ethoxy)-ethoxy]-acetyl)₂-(γ-Glu)- | | | |
| | CO-(CH₂)₁₈-CO₂H)AQ-Aib- | | | |
| | DFIEYLIAGGPSSGAPPPS-NH₂ | | | |
| 345 | (D-Tyr)-Aib-EGT-αMeF-TSDYSI-αMeL- | 360 | 4891.6 | N/I |
| | LDKK((2-[2-(2-Amino-ethoxy)-ethoxy]- | | | |
| | acetyl)₂-(γ-Glu)-CO-(CH₂)₁₈-CO₂H)AQ- | | | |
| | Aib-EFIEYLIAGGPSSGAPPPS-NH₂ | | | |
| 346 | (D-Tyr)-Aib-EGTFTSDYSI- | 361 | 4805.5 | 4804.8 |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFIEYLIAGGPSSGAPPPS-NH₂ | | | |
| 347 | (D-Tyr)-Aib-EGTFTSDYSI- | 362 | 4791.4 | N/I |
| | αMeL-LD-Dab-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFIEYLIAGGPSSGAPPPS-NH₂ | | | |
| 348 | (D-Tyr)-Aib-EGTFTSDYSI- | 363 | 4807.5 | N/I |
| | αMeL-LD-Dap-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFIEYLIAGGPSSGAPPPS-NH₂ | | | |
| 349 | (D-Tyr)-Aib-EGTFTSDYSI- | 364 | 4863.5 | 4862.7 |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | EFIEYLIAGGPSSGAPPPS-NH₂ | | | |
| 350 | (D-Tyr)-Aib-EGTFTSDYSI- | 365 | 4849.5 | N/I |
| | αMeL-LD-Dab-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | EFIEYLIAGGPSSGAPPPS-NH₂ | | | |
| 351 | (D-Tyr)-Aib-EGTFTSDYSI- | 366 | 4835.5 | N/I |
| | αMeL-LD-Dap-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | EFIEYLIAGGPSSGAPPPS-NH₂ | | | |
| 352 | (D-Tyr)-Aib-EGTFTSDYSI-αMeL- | 367 | 4819.5 | N/I |
| | LDKK((2-[2-(2-Amino-ethoxy)-ethoxy]- | | | |
| | acetyl)₂-(γ-Glu)-CO-(CH₂)₁₈- | | | |
| | CO₂H)AQ-Aib-AFI-(D-Glu)- | | | |
| | YLIAGGPSSGAPPPS-NH₂ | | | |
| 353 | (D-Tyr)-Aib-EGTFTSDYSI-αMeL- | 368 | 4935.6 | N/I |
| | LDKK((2-[2-(2-Amino-ethoxy)-ethoxy]- | | | |
| | acetyl)₂-(γ-Glu)-CO-(CH₂)₁₈- | | | |
| | CO₂H)AQ-Aib-EFI-(D-Glu)- | | | |
| | YLIEGGPSSGAPPPS-NH₂ | | | |
| 354 | (D-Tyr)-Aib-EGTFTSDYSI- | 369 | 4863.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈- | | | |
| | CO₂H)AQ-Aib-EFI-(D- | | | |
| | Glu)-YLIAGGPSSGAPPPS-NH₂ | | | |
| 355 | (D-Tyr)-Aib-EGTFTSDYSI- | 370 | 4921.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈- | | | |
| | CO₂H)AQ-Aib-EFI-(D- | | | |
| | Glu)-YLIEGGPSSGAPPPS-NH₂ | | | |
| 356 | (D-Tyr)-Aib-EGTFTSDYSI-αMeL- | 371 | 4877.5 | N/I |
| | LDKK((2-[2-(2-Amino-ethoxy)-ethoxy]- | | | |
| | acetyl)₂-(γ-Glu)-CO-(CH₂)₁₈- | | | |
| | CO₂H)AQ-Aib- | | | |
| | AFI-(D-Glu)-YLIEGGPSSGAPPPS-NH₂ | | | |
| 357 | (D-Tyr)-Aib-EGTFTSDYSI- | 372 | 4863.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈- | | | |
| | CO₂H)AQ-Aib-AFI-(D- | | | |
| | Glu)-YLIEGGPSSGAPPPS-NH₂ | | | |
| 358 | (D-Tyr)-Aib-EGTFTSDYSI- | 373 | 4791.4 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFVEYLIAGGPSSGAPPPS-NH₂ | | | |
| 359 | (D-Tyr)-Aib-EGTFTSDYSI- | 374 | 4849.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | EFVEYLIAGGPSSGAPPPS-NH₂ | | | |
| 360 | (D-Tyr)-Aib-EGTFTSDYSI- | 375 | 4849.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | AFVEYLIEGGPSSGAPPPS-NH₂ | | | |
| 361 | (D-Tyr)-Aib-EGTFTSDYSI-αMeL- | 376 | 4805.5 | N/I |
| | LDKK((2-[2-(2-Amino-ethoxy)-ethoxy]- | | | |
| | acetyl)₂-(γ-Glu)-CO-(CH₂)₁₈- | | | |
| | CO₂H)AQ-Aib- | | | |
| | AFV-(D-Glu)-YLIAGGPSSGAPPPS-NH₂ | | | |
| 362 | (D-Tyr)-Aib-EGTFTSDYSI- | 377 | 4791.4 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib-AFV- | | | |
| | (D-Glu)-YLIAGGPSSGAPPPS-NH₂ | | | |
| 363 | (D-Tyr)-Aib-EGTFTSDYSI- | 378 | 4777.4 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₆-CO₂H)AQ-Aib- | | | |
| | AFI-(D-Glu)-YLIAGGPSSGAPPPS-NH₂ | | | |
| 364 | (D-Tyr)-Aib-EGTFTSDYSI- | 379 | 4763.4 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₆-CO₂H)AQ-Aib-AFV- | | | |
| | (D-Glu)-YLIAGGPSSGAPPPS-NH₂ | | | |
| 365 | (D-Tyr)-Aib-EGTFTSDYSI-αMeL- | 380 | 4833.5 | 4832.4 |
| | LDKK((2-[2-(2-Amino-ethoxy)-ethoxy]- | | | |
| | acetyl)₂-(γ-Glu)-CO- | | | |
| | (CH₂)₁₈-CO₂H)AQ-Aib- | | | |
| | AFIE-αMeY-LIAGGPSSGAPPPS-NH₂ | | | |
| 366 | (D-Tyr)-Aib-EGTFTSDYSI- | 381 | 4819.5 | 4818.3 |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib-AFIE- | | | |
| | αMeY-LIAGGPSSGAPPPS-NH₂ | | | |
| 367 | Y-Aib-EGTFTSDYSI-αMeL-LDKK((2[2- | 382 | 4891.6 | N/I |
| | (2-Amino-ethoxy)-ethoxy]-acetyl)₂-(γ-Glu)- | | | |
| | CO-(CH₂)₁₈-CO₂H)AQ-Aib-EFIE-αMeY- | | | |
| | LIAGGPSSGAPPPS-NH₂ | | | |
| 368 | (D-Tyr)-Aib-EGTFTSDYSI- | 383 | 4835.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₆-CO₂H)AQ-Aib- | | | |
| | EFIEYLIAGGPSSGAPPPS-NH₂ | | | |
| 369 | (D-Tyr)-Aib-EGTFTSDYSI- | 384 | 4849.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | DFIEYLIAGGPSSGAPPPS-NH₂ | | | |
| 370 | (D-Tyr)-Aib-EGTFTSDYSI- | 385 | 4414.0 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib- | | | |
| | EFIEYLIAGGPSSG-NH₂ | | | |
| 371 | (D-Tyr)-Aib-EGTFTSDYSI-αMeL- | 386 | 4718.3 | N/I |
| | LD-Orn-K((2-[2-(2-Amino-ethoxy)- | | | |
| | ethoxy]-acetyl)-(γ- | | | |
| | Glu)-CO-(CH₂)₁₈-CO₂H)AQ-Aib- | | | |
| | EFIEYLIAGGPSSGAPPPS-NH₂ | | | |
| 372 | (D-Tyr)-Aib-EGTFTSDYSI-αMeL- | 387 | 4746.4 | N/I |
| | LDKK((2-[2-(2-Amino-ethoxy)-ethoxy]- | | | |
| | acetyl)-(γ-Glu)-CO-(CH₂)₁₈-CO₂H)AQ-Aib- | | | |
| | EFIE-αMeY-LIAGGPSSGAPPPS-NH₂ | | | |
| 373 | (D-Tyr)-Aib-EGTFTSDYSI-αMeL- | 388 | 4688.3 | N/I |
| | LDKK((2-[2-(2-Amino-ethoxy)-ethoxy]- | | | |
| | acetyl)-(γ-Glu)-CO-(CH₂)₁₈-CO₂H)AQ-Aib- | | | |
| | AFIE-αMeY-LIAGGPSSGAPPPS-NH₂ | | | |
| 374 | (D-Tyr)-Aib-EGTFTSDYSI-αMeL- | 389 | 4863.5 | N/I |
| | LDKK((2-[2-(2-Amino-ethoxy)-ethoxy]- | | | |
| | acetyl)₂-(γ-Glu)-CO- | | | |
| | (CH₂)₁₆-CO₂H)AQ-Aib- | | | |
| | EFIE-αMeY-LIAGGPSSGAPPPS-NH₂ | | | |
| 375 | (D-Tyr)-Aib-EGTFTSDYSI- | 390 | 4849.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₆-CO₂H)AQ-Aib-EFIE- | | | |
| | αMeY-LIAGGPSSGAPPPS-NH₂ | | | |
| 376 | (D-Tyr)-Aib-EGTFTSDYSI-αMeL- | 391 | 4805.5 | N/I |
| | LDKK((2-[2-(2-Amino-ethoxy)-ethoxy]- | | | |
| | acetyl)₂-(γ-Glu)-CO- | | | |
| | (CH₂)₁₆-CO₂H)AQ-Aib- | | | |
| | AFIE-αMeY-LIAGGPSSGAPPPS-NH₂ | | | |
| 377 | (D-Tyr)-Aib-EGTFTSDYSI- | 392 | 4791.4 | 4790.7 |
| | αMeL-LD-Orn-K((2-[2-(2- | | | |
| | Amino-ethoxy)-ethoxy]-acetyl)₂- | | | |
| | (γ-Glu)-CO-(CH₂)₁₆-CO₂H)AQ-Aib-AFIE- | | | |
| | αMeY-LIAGGPSSGAPPPS-NH₂ | | | |
| 378 | (D-Tyr)-Aib-EGTFTSDYSI-αMeL- | 393 | 4732.4 | N/I |
| | LDKK((2-[2-(2-Amino-ethoxy)-ethoxy]- | | | |
| | acetyl)-(γ-Glu)-CO-(CH₂)₁₈-CO₂H)AQ-Aib- | | | |
| | EFIEYLIAGGPSSGAPPPS-NH₂ | | | |
| 379 | (D-Tyr)-Aib-EGTFTSDYSI-αMeL- | 394 | 4949.5 | N/I |
| | LDKK((2-[2-(2-Amino-ethoxy)-ethoxy]- | | | |
| | acetyl)₂-(γ-Glu)-CO- | | | |
| | (CH₂)₁₈-CO₂H)AQ-Aib- | | | |
| | EFIE-αMeY-LIEGGPSSGAPPPS-NH₂ | | | |
| 380 | (D-Tyr)-Aib-EGTFTSDYSI- | 395 | 4935.5 | N/I |
| | αMeL-LD-Orn-K((2-[2-(2- | | | |
| | Amino-ethoxy)-ethoxy]-acetyl)₂- | | | |
| | (γ-Glu)-CO-(CH₂)₁₈-CO₂H)AQ-Aib-EFIE- | | | |
| | αMeY-LIEGGPSSGAPPPS-NH₂ | | | |
| 381 | (D-Tyr)-Aib-EGTFTSDYSI-αMeL- | 396 | 4934.4 | N/I |
| | LDEK((2-[2-(2-Amino-ethoxy)-ethoxy]- | | | |
| | acetyl)₂-(γ-Glu)-CO- | | | |
| | (CH₂)₁₈-CO₂H)AQ-Aib- | | | |
| | AFIE-αMeY-LIAGGPSSGAPPPS-NH₂ | | | |

N/I means Not Included

### Binding Assays

Glucagon (referred to as Gcg) is a Reference Standard prepared at Eli Lilly and Company. GLP-1, 7-36-NH₂ (referred to as GLP-1) is obtained from CPC Scientific (Sunnyvale, CA, 97.200 purity, 100 μM aliquots in 100% DMSO). GIP 1-42 (referred to as GIP) is prepared at Lilly Research Laboratories using peptide synthesis and HPLC chromatography as described above (>80% purity, 100 μM aliquots in 100% DMSO). [¹²⁵I]-radiolabeled Gcg, GLP-1, or GIP is prepared using [¹²⁵I]-lactoperoxidase and obtained from Perkin Elmer (Boston, MA).

Stably transfected cell lines are prepared by subcloning receptor cDNA into a pcDNA3 expression plasmid and transfected into human embryonic kidney (HEK) 293 (hGcgR and hGLP-1R) or Chinese Hamster Ovary (CHO) (hGIPR) cells followed by selection with Geneticin (hGLP-1R and hGIPR) or hygromycin B (hGcgR).

Two methods are used for the preparation of crude cell membranes.

Method 1: Frozen cell pellets are lysed on ice in hypotonic buffer containing 50 mM Tris HCl, pH 7.5, and Roche Complete™ Protease Inhibitors with EDTA. The cell suspension is disrupted using a glass Potter-Elvehjem homogenizer fitted with a Teflon® pestle for 25 strokes. The homogenate is centrifuged at 4° C. at 1100×g for 10 minutes. The supernatant is collected and stored on ice while the pellets are resuspended in homogenization buffer and rehomogenized as described above. The homogenate is centrifuged at 1100×g for 10 minutes. The second supernatant is combined with the first supernatant and centrifuged at 35000×g for 1 hour at 4° C. The resulting membrane pellet is resuspended in homogenization buffer containing protease inhibitors at approximately 1 to 3 mg/mL, quick frozen in liquid nitrogen and stored as aliquots in a −80° C. freezer until use.

Method 2: Frozen cell pellets are lysed on ice in hypotonic buffer containing 50 mM Tris HCl, pH 7.5, 1 mM MgCl₂, Roche Complete™ EDTA-free Protease Inhibitors and 25 units/ml DNAse I (Invitrogen). The cell suspension is disrupted using a glass Potter-Elvehjem homogenizer fitted with a Teflon® pestle for 20 to 25 strokes. The homogenate is centrifuged at 4° C. at 1800×g for 15 minutes. The supernatant is collected and stored on ice while the pellets are resuspended in homogenization buffer (without DNAse I) and rehomogenized as described above. The homogenate is centrifuged at 1800×g for 15 minutes. The second supernatant is combined with the first supernatant and centrifuged an additional time at 1800×g for 15 minutes. The overall supernatant is then centrifuged at 25000×g for 30 minutes at 4° C. The resulting membrane pellet is resuspended in homogenization buffer (without DNAse I) containing protease inhibitors at approximately 1 to 3 mg/mL and stored as aliquots in a −80° C. freezer until use.

### Binding Determination Methods

The equilibrium binding dissociation constants (K_{d}) for the various receptor/radioligand interactions are determined from homologous competition binding analysis instead of saturation binding due to high propanol content in the [¹²⁵I] stock material. The K_{d} values determined for the receptor preparations were as follows: hGcgR (3.9 nM), hGLP-1R (1.2 nM) and hGIPR (0.14 nM).

### [¹²⁵I]-Glucagon Binding

The human Gcg receptor binding assays are performed using a Scintillation Proximity Assay (SPA) format with wheat germ agglutinin (WGA) beads (Perkin Elmer). The binding buffer contains 25 mM 4-(2-hydroxyethyl)-1-piperazineethanesulfonic acid (HEPES), pH 7.4, 2.5 mM CaCl₂), 1 mM MgCl₂, 0.1% (w/v) bacitracin (Research Products), 0.003% (w/v) Polyoxyethylenesorbitan monolaurate (TWEEN®-20), and Roche Complete™ Protease Inhibitors without EDTA. Peptides and Gcg are thawed and 3-fold serially diluted in 100% DMSO (10 point concentration response curves). Next, 5 μL serially diluted compound or DMSO is transferred into Corning® 3632 clear bottom assay plates containing 45 μL assay binding buffer or unlabeled Gcg control (non-specific binding or NSB, at 1 μM final). Then, 50 μL [¹²⁵I]-Gcg (0.15 nM final), 50 μL human GcgR membranes (1.5 μg/well) and 50 μL of WGA SPA beads (80 to 150 μg/well) are added with a Biotek Multiflo dispenser. Plates are sealed and mixed on a plate shaker (setting 6) for 1 minute and read with a PerkinElmer Trilux MicroBeta® scintillation counter after 12 hours of incubation/settling time at room temperature. Final assay concentration ranges for peptides tested in response curves is typically 1150 nM to 0.058 nM and for the control Gcg from 1000 nM to 0.05 nM.

### [¹²⁵I]-GLP-1 Binding

The human GLP-1 receptor binding assay is performed using an SPA format with WGA beads. The binding buffer contains 25 mM HEPES, pH 7.4, 2.5 mM CaCl₂), 1 mM MgCl₂, 0.1% (w/v) bacitracin, 0.003% (w/v) TWEEN®-20, and Roche Complete™ Protease Inhibitors without EDTA. Peptides and GLP-1 are thawed and 3-fold serially diluted in 100% DMSO (10 point concentration response curves). Next, 5 μL serially diluted compound or DMSO is transferred into Corning® 3632 clear bottom assay plates containing 45 μL assay binding buffer or unlabeled GLP-1 control (non-specific binding or NSB, at 0.25 μM final). Then, 50 μL [¹²⁵I]-GLP-1 (0.15 nM final), 50 μL human GLP-1R membranes (0.5 μg/well and 50 μL of WGA SPA beads (100 to 150 μg/well) are added with a Biotek Multiflo dispenser. Plates are sealed and mixed on a plate shaker (setting 6) for 1 minute and read with a PerkinElmer Trilux MicroBeta® scintillation counter after 5 to 12 hours of incubation/settling time at room temperature. Final assay concentration ranges for peptides tested in response curves are typically 1150 nM to 0.058 nM and for the control GLP-1, 250 nM to 0.013 nM.

### [125I]-GIP Binding

The human GIP receptor binding assay is performed using an SPA format with WGA beads. The binding buffer contains 25 mM HEPES, pH 7.4, 2.5 mM CaCl₂), 1 mM MgCl₂, 0.1% (w/v) bacitracin, 0.003% (w/v) TWEEN®-20, and Roche Complete™ Protease Inhibitors without EDTA. Peptides and GIP are thawed and 3 fold serially diluted in 100% DMSO (10 point concentration response curves). Next, 5 μL serially diluted compound or DMSO is transferred into Corning® 3632 clear bottom assay plates containing 45 μL assay binding buffer or unlabeled GIP control (non-specific binding or NSB, at 0.25 μM final). Then, 50 μL [¹²⁵I]-GIP (0.075-0.15 nM final), 50 μL human GIPR membranes (3 μg/well) and 50 μL of WGA SPA beads (100 to 150 μg/well) are added with a Biotek Multiflo dispenser. Plates are sealed and mixed on a plate shaker (setting 6) for 1 minute and read with a PerkinElmer Trilux MicroBeta® scintillation counter after 2.5 to 12 hours of incubation/settling time at room temperature. Final assay concentration ranges for peptides tested in response curves is typically 1150 to 0.058 nM or 115 nM to 0.0058 nM and for the control GIP, 250 nM to 0.013 nM.

### Binding Assay Data Analysis

Raw CPM data for concentration curves of peptides, Gcg, GLP-1, or GIP are converted to percent inhibition by subtracting nonspecific binding (binding in the presence of excess unlabeled Gcg, GLP-1, or GIP, respectively) from the individual CPM values and dividing by the total binding signal, also corrected by subtracting nonspecific binding. Data are analyzed using four-parameter (curve maximum, curve minimum, IC₅₀, Hill slope) nonlinear regression routines (Genedata Screener, version 12.0.4, Genedata AG, Basal, Switzerland). The affinity constant (Kᵢ) is calculated from the absolute IC₅₀ value based upon the equation Kᵢ=IC₅₀/(1+D/K_{d}) where D is the concentration of radioligand used in the experiment, IC₅₀ is the concentration causing 50% inhibition of binding and K_{d} is the equilibrium binding dissociation constant of the radioligand (described above). Values for Kᵢ are reported as the geometric mean, with error expressed as the standard error of the mean (SEM) and n is equal to the number of independent replicates (determined in assays performed on different days). Geometric Means are calculated as follows:

Geometric Mean=10^{(Arithmetic Mean of Log Ki Values))}

The Ki Ratio (Ki for native control peptide/Ki for test compound) at each receptor and each species is calculated. The Ki Ratio is a rapid indication of the apparent affinity of a peptide compared to the native control peptide. A Ki Ratio <1 indicates that the test peptide has a lower affinity (higher Ki value) for the receptor than the native peptide, whereas a Ki Ratio >1 indicates that the test peptide has a higher affinity (lower Ki value) for the receptor than the native peptide.

n=1/x means that only one value out of the total number of replicates (x) is used to express the mean. SEM is only calculated when n=2 or greater non-qualified results exist. Means are expressed as GeoMetric means with the standard error of the mean (SEM) and the number of replicates (n) indicated in parenthesis.

TABLE 1
In vitro Binding Affinity (Kᵢ) of indicated Examples
and comparator molecules for human GLP-1R, GogR and GIPR.

| | hGLcgR | hGIPR | hGLP1R |
|---|---|---|---|
| Example or | Ki, nM | Ki, nM | Ki, (nM) |
| comparator | (SEM, n) | (SEM, n) | (SEM, n) |
| hGcg | 3.65 | | |
| | (0.26, n = 10) | | |
| hGIP amide | | 0.0922 | |
| | | (0.0085, n = 11) | |
| hGLP-1 amide | | | 0.614 |
| | | | (0.066, n = 12) |
| 1 | 207 | 0.0546 | 6.67 |
| | (13.8, n = 5) | (0.0120, n = 5) | (1.25, n = 6) |
| 2 | 361 | 0.0600 | 2.35 |
| | (55.1, n = 5) | (0.0150, n = 5) | (0.220, n = 5) |
| 3 | 242 | 0.0458 | 2.23 |
| | (56.2, n = 6) | (0.00357, n = 6) | (0.366, n = 6) |
| 4 | 686 | 0.0528 | 1.63 |
| | (n = ⅕) | (0.00647, n = 5) | (0.260, n = 5) |
| 5 | 519 | 0.0611 | 0.902 |
| | (109, n = 4) | (0.00592, n = 4) | (0.114, n = 4) |
| 6 | 55.8 | 0.0835 | 6.71 |
| | (10.2, n = 2) | (0.00437, n = 2) | (1.25, n = 2) |
| 7 | 198 | 0.252 | 43.3 |
| 8 | 206 | 0.0772 | 2.84 |
| | (25.7, n = 2) | (0.0155, n = 2) | (0.753, n = 2) |
| 9 | 375 | 0.127 | 14.9 |
| | (87.5, n = 2) | (0.0118, n = 2) | (2.15, n = 2) |
| 10 | 226 | 0.109 | 9.33 |
| | (67.4, n = 2) | (0.0927, n = 2) | (1.49, n = 2) |
| 11 | 174 | 0.226 | 15.7 |
| | (25.3, n = 2) | (0.0728, n = 2) | (4.37, n = 2) |
| 12 | 684 | 0.167 | 12.9 |
| | (141, n = 2) | (0.0853, n = 2) | (2.71, n = 2) |
| 13 | >1060 | 0.296 | 31.1 |
| | (n = ½) | (0.0291, n = 2) | (11.9, n = 2) |
| 14 | 160 | 0.0494 | 29.6 |
| 15 | 130 | 0.284 | 2.19 |
| 16 | 371 | 0.0841 | 2.78 |
| 17 | 261 | 0.606 | 7.63 |
| | (115, n = 2) | (0.363, n = 2) | (2.47, n = 2) |
| 18 | 50.1 | 0.0798 | 0.319 |
| 19 | 60.5 | 0.0518 | 0.24 |
| 20 | 228 | 0.0849 | 3.30 |
| | (65.3, n = 2) | (0.0168, n = 2) | (1.01, n = 2) |
| 21 | 149 | 0.529 | 14.5 |
| 22 | 53.4 | 0.624 | 23.1 |
| 23 | >1010 | 0.258 | 6.32 |
| 24 | 49.8 | 0.232 | 5.04 |
| 25 | 81.1 | 0.179 | 4.8 |
| 26 | >960 | 0.176 | 4.22 |
| 27 | 315 | 0.103 | 3.68 |
| 28 | >902 | 0.24 | 21.1 |
| 29 | 132 | 0.377 | 8 |
| 30 | 123 | 0.151 | 6.2 |
| 31 | 290 | 0.0275 | 6.58 |
| 32 | 44.7 | 0.0205 | 3.96 |
| 33 | >979 | 6.4 | 361 |
| 34 | 134 | 0.0467 | 3.41 |
| 35 | >964 | 0.0358 | 54.6 |
| 36 | 413 | 0.141 | 16.4 |
| 37 | 255 | 0.0523 | 3.84 |
| 38 | >974 | 0.104 | 31.3 |
| 39 | 161 | 0.0499 | 16.8 |
| 40 | 150 | 0.0345 | 7.56 |
| 41 | 165 | 0.0551 | 13.4 |
| 42 | 160 | 0.0514 | 13.2 |
| 43 | 134 | 0.101 | 11.8 |
| 44 | 121 | 0.0516 | 10.6 |
| 45 | 11.1 | 0.0463 | 5.65 |
| 46 | 133 | 0.0852 | 13.4 |
| 48 | 111 | 0.074 | 15.7 |
| 49 | 236 | 0.087 | 12.3 |
| 50 | 220 | 0.0568 | 4.71 |
| | (61.2, n = 2) | (0.00744, n = 2) | (1.22, n = 2) |
| 51 | 195 | 0.0620 | 5.62 |
| | (65.9, n = 2) | (0.0131, n = 2) | (0.658, n = 2) |
| 52 | >1100 | 0.0342 | 5.81 |
| 53 | 216 | 0.188 | 1.23 |
| 54 | 333 | 0.965 | 1.66 |
| 55 | >1100 | 6.24 | 7.29 |
| 56 | >1060 | 0.148 | 10.3 |
| 57 | 26.1 | 0.0583 | 3.00 |
| | (4.31, n = 2) | (0.0131, n = 2) | (0.293, n = 2) |
| 58 | 339 | 0.105 | 2.77 |
| 59 | 292 | 0.136 | 8.20 |
| | (11.8, n = 2) | (0.00422, n = 2) | (4.13, n = 2) |
| 60 | 237 | 0.0655 | 9.55 |
| 61 | 110 | 0.102 | 11.1 |
| 62 | 168 | 0.0545 | 2.03 |
| 63 | 273 | 0.141 | 7.79 |
| 64 | 260 | 0.0866 | 4.86 |
| 65 | 194 | 0.0643 | 4.53 |
| 66 | 93.7 | 0.106 | 7.53 |
| 67 | 270 | 0.061 | 10.2 |
| 68 | 99.2 | 0.0243 | 1.58 |
| 69 | 22.1 | 0.0300 | 1.22 |
| | (4.18, n = 3) | (0.00657, n = 3) | (0.353, n = 3) |
| 74 | 69.8 | 0.0279 | 5.99 |
| 75 | 283 | 0.103 | 24.4 |
| 76 | 14.4 | 0.0659 | 2.64 |
| 78 | 215 | 0.163 | 3.94 |
| | (66.2, n = 3) | (0.0356, n = 2) | (1.21, n = 2) |
| 79 | 429 (n = ½) | 0.0313 | 2.69 |
| 80 | 347 (n = ½) | 0.0931 | 2.16 |
| 81 | 344 | 0.198 | 2.88 |
| 82 | >1060 | 14.9 | 6.82 |
| 83 | 320 | 0.142 | 7.1 |
| 84 | >1100 | 0.143 | 10.2 |
| 85 | >894 (n = ½) | 0.621 | 1.87 |
| 86 | >1060 | 0.0401 | 3.74 |
| 87 | 278 | 0.0340 | 1.79 |
| | (n = ½) | (0.00150, n = 2) | (0.417, n = 2) |
| 88 | 545 | 0.0717 | 4.24 |
| | (57.8, n = 2) | | |
| 89 | 324 | 0.045 | 2.64 |
| | (22.9, n = 2) | | |
| 90 | 245 | 0.0472 | 4.76 |
| | (7.55, n = 2) | | |
| 91 | 540 | 1.8 | 5.23 |
| 93 | 15.7 | 0.0859 | 1.89 |
| 99 | 23.6 | 0.027 | 1.15 |
| 100 | 44 | 0.115 | 4.13 |
| 101 | 117 | 0.0953 | 8.1 |
| 103 | 40.3 | 0.0645 | 6.68 |
| 104 | 123 | 0.0565 | 3.91 |
| | (17.1, n = 5) | (0.0153, n = 5) | (0.955, n = 5) |
| 105 | 20.4 | 0.119 | 0.871 |
| 106 | 515 | 0.179 | 1.2 |
| 107 | 303 | 0.0425 | 0.867 |
| 108 | 171 | 0.0732 | 3 |
| 109 | 43.1 | 0.0279 | 1.34 |
| 110 | 73.9 | 0.0395 | 4.38 |
| 115 | 9.89 | 0.0302 | 3.43 |
| 116 | 137 | 0.0597 | 6.80 |
| | (13.9, n = 2) | (0.0486, n = 2) | (1.85, n = 2) |
| 117 | 192 | 0.0497 | 6.96 |
| | (14.6, n = 3) | (0.0111, n = 3) | (1.95, n = 3) |
| 118 | 53.0 | 0.0859 | 6.10 |
| | (7.07, n = 3) | (0.00402, n = 3) | (0.870, n = 3) |
| 119 | 30.6 | 0.0925 | 9.87 |
| 120 | 93.6 | 0.11 | 11.7 |
| 121 | 51.9 | 0.177 | 3.16 |
| 122 | 43.3 | 0.190 | 3.36 |
| | (8.07, n = 2) | (0.0189, n = 2) | (0.799, n = 2) |
| 123 | 80.1 | 0.0469 | 1.31 |
| | (11.7, n = 6) | (0.00804, n = 6) | (0.197, n = 6) |
| 124 | 41.5 | 0.0424 | 4.87 |
| | (9.39, n = 2) | (0.00200, n = 2) | (0.277, n = 2) |
| 125 | 54.4 | 0.0624 | 3.19 |
| | (0.365, n = 2) | (0.0117, n = 2) | (0.123, n = 2) |
| 126 | 101 | 0.0644 | 1.46 |
| | (11.5, n = 2) | (0.0267, n = 2) | (0.299, n = 2) |
| 127 | 43.6 | 0.126 | 1.86 |
| 128 | 433 | 0.0625 | 1.88 |
| | (203, n = 2) | (0.0355, n = 2) | (0.296, n = 2) |
| 129 | 14.9 | 0.0278 | 1 |
| 130 | >1060 | 0.177 | 3.66 |
| 133 | 216 | 0.157 | 11.4 |
| | | | (2.31, n = 2) |
| 134 | 60.5 | 0.14 | 12.7 |
| | | | (0.947, n = 2) |
| 135 | 454 | 0.161 | 3.01 |
| 137 | 98.1 | 0.0373 | 1.24 |
| | (14.8, n = 3) | (0.00200, n = 3) | (0.341, n = 3) |
| 138 | 61.2 | 0.0295 | 0.926 |
| | (4.65, n = 2) | (0.00145, n = 2) | (0.201, n = 2) |
| 139 | 105 | 0.0360 | 1.25 |
| | (6.68, n = 2) | (0.00446, n = 2) | (0.0904, n = 2) |
| 140 | 175 | 0.0474 | 1.46 |
| | (40.1, n = 3) | (0.00461, n = 3) | (0.0630, n = 3) |
| 142 | 53.1 | 0.0275 | 1.06 |
| | (1.60, n = 2) | (0.00210, n = 2) | (0.300, n = 2) |
| 143 | 65.5 | 0.0304 | 1.15 |
| 144 | 77 | 0.0341 | 1.78 |
| 145 | 158 | 0.0652 | 2.22 |
| 147 | 64.9 | 0.0981 | 4.47 |
| | (19.9, n = 2) | (0.0285, n = 2) | (0.742, n = 2) |
| 149 | 127 | 0.0708 | 26.1 |
| 150 | 63.2 | 0.0649 | 30.5 |
| 152 | 93.4 | 0.117 | 48 |
| 153 | 43.8 | 0.0578 | 22.2 |
| 154 | 762 | 0.0610 | 5.64 |
| | (51.7, n = 3) | (0.00457, n = 3) | (2.52, n = 3) |
| 157 | 179 | 0.0937 | 8.97 |
| | (82.9, n = 3) | (0.0160, n = 3) | (2.28, n = 3) |
| 158 | 285 | 0.114 | 11.8 |
| | (17.4, n = 2) | (0.0193, n = 2) | (5.32, n = 2) |
| 160 | >1060 | 5.98 | 14.4 |
| | (n = ½) | (1.46, n = 2) | (3.72, n = 2) |
| 163 | 117 | 0.116 | 10.8 |
| 181 | 413 | 0.145 | 7.28 |
| | (132, n = 2) | (0.0856, n = 2) | (0.798, n = 2) |
| 182 | 565 | 0.0669 | 4.64 |
| | (335, n = 2) | (0.0311, n = 2) | (0.655, n = 2) |
| 183 | 304 | 0.0869 | 4.11 |
| | (128, n = 2) | (0.0118, n = 2) | (0.369, n = 2) |
| 189 | 146 | 0.128 | 8.81 |
| | (7.81, n = 2) | (0.0817, n = 2) | (0.434, n = 2) |
| 191 | 348 | 0.144 | 4.52 |
| | (54.7, n = 2) | (0.0676, n = 2) | (1.95, n = 2) |
| 192 | >1110 | 0.118 | 2.89 |
| | (n = ½) | (0.108, n = 2) | (0.516, n = 2) |
| 202 | 394 | 0.0579 | 5.38 |
| 203 | 845 | 0.0337 | 3.90 |
| | (n = ½) | (0.00260, n = 2) | (1.10, n = 2) |
| 204 | >1150 | 0.0704 | 1.9 |
| 205 | 438 | 0.0367 | 3.05 |
| 206 | 176 | 0.0814 | 5.27 |
| | (126, n = 2) | (0.00608, n = 2) | (0.359, n = 2) |
| 207 | 74.2 | 0.0786 | 1.37 |
| 208 | >1060 | 0.0537 | 2.13 |
| 209 | >1060 | 0.0664 | 1.43 |
| | (n = ½) | (0.0267, n = 2) | (0.466, n = 2) |
| 210 | >1010 | 0.0399 | 1.58 |
| 211 | 131 | 0.0243 | 2.64 |
| 212 | 205 | 0.0978 | 2.76 |
| | (1.77, n = 2) | (0.0730, n = 2) | (0.561, n = 2) |
| 213 | 544 | 0.365 | 2.75 |
| 214 | 126 | 0.0304 | 1.99 |
| 215 | 75.2 | 0.0666 | 6.85 |
| 216 | 45.2 | 0.0559 | 2.34 |
| 217 | 516 | 0.0376 | 2.02 |
| 218 | 270 | 0.0593 | 2.54 |
| 219 | 373 | 0.0689 | 2.01 |
| 220 | 377 | 0.0919 | 2.71 |
| 221 | 154 | 0.0414 | 1.77 |
| | (n = ½) | (0.00291, n = 2) | (0.900, n = 2) |
| 222 | 71.3 | 0.0495 | 3.59 |
| | (11.9, n = 2) | (0.0210, n = 2) | (0.660, n = 2) |
| 223 | 46.5 | 0.0921 | 5.62 |
| 224 | 627 | 0.0482 | 6.86 |
| | (267, n = 2) | (0.0174, n = 2) | (1.85, n = 2) |
| 225 | 714 | 0.0622 | 8.79 |
| | (n = ½) | (0.0208, n = 2) | (4.24, n = 2) |
| 226 | 200 | 0.0254 | 4.1 |
| 227 | 113 | 0.0146 | 2.01 |
| 228 | 182 | 0.028 | 2.43 |
| 229 | >1100 | 2.47 | 36.2 |
| 230 | 494 | 0.042 | 4.68 |
| 231 | 440 | 0.0394 | 3.03 |
| 232 | >1150 | 0.0544 | 5.62 |
| 233 | >1150 | 0.0445 | 5.99 |
| 234 | >1100 | 0.0563 | 10.9 |
| 235 | >1200 | 0.0581 | 7.65 |
| 236 | 200 | 0.0425 | 1.05 |
| | (15.1, n = 2) | (0.00194, n = 2) | (0.173, n = 2) |
| 237 | >1060 | 0.131 | 1.04 |
| 238 | 230 | 0.0403 | 0.548 |
| 239 | 596 | 0.101 | 2.71 |
| | (215, n = 3) | (0.0172, n = 3) | (0.0420, n = 3) |
| 240 | 204 | 0.0284 | 0.552 |
| 241 | 167 | 0.0420 | 0.799 |
| | (45.6, n = 2) | (0.0118, n = 2) | (0.401, n = 2) |
| 242 | 95.9 | 0.0604 | 0.853 |
| | (14.1, n = 2) | (0.00642, n = 2) | (0.0475, n = 2) |
| 243 | 145 | 0.0325 | 0.670 |
| | (5.05, n = 2) | (0.00840, n = 2) | (0.0478, n = 2) |
| 244 | 87.8 | 0.0308 | 0.820 |
| | (2.39, n = 2) | (0.0150, n = 2) | (0.141, n = 2) |
| 246 | >1010 | 0.0509 | 0.812 |
| | (n = ⅓) | (0.0147, n = 3) | (0.0900, n = 3) |
| 247 | >1100 | >55.1 | 4.39 |
| 248 | >1050 | 0.0397 | 2.4 |
| 249 | >1000 | 0.0394 | 2.35 |
| 250 | 198 | 0.0171 | 1.72 |
| 251 | 21.2 | 0.0249 | 1.09 |
| 252 | 26 | 0.00971 | 0.383 |
| 253 | >912 | 0.138 | 2.57 |
| 254 | 148 | 0.108 | 2.58 |
| 255 | 257 | 0.0772 | 2.58 |
| 264 | 388 | 0.015 | 0.412 |
| 265 | 567 | 0.0224 | 0.537 |
| 266 | 193 | 0.0666 | 2.01 |
| | (28.2, n = 2) | (0.0189, n = 2) | (0.256, n = 2) |
| 267 | 349 | 0.0628 | 1.57 |
| | (178, n = 2) | (0.00765, n = 2) | (0.109, n = 2) |
| 268 | >1190 | 0.0814 | 3.98 |
| 269 | >1100 | 0.152 | 7.1 |
| 270 | >1190 | 0.117 | 8.27 |
| 271 | >1150 | 0.107 | 5.09 |
| 272 | 550 | 0.0353 | 1.22 |
| | (243, n = 2) | (0.00276, n = 2) | (0.291, n = 2) |
| 273 | 724 | 0.0698 | 1.13 |
| 288 | 345 | 0.0580 | 1.60 |
| | (35.7, n = 3) | (0.0105, n = 3) | (0.866, n = 3) |
| 289 | >1050 | 0.0457 | 2.63 |
| | (n = ⅓) | (0.0220, n = 3) | (1.74, n = 3) |
| 290 | 308 | 0.0617 | 2.44 |
| | (n = ⅓) | (0.0115, n = 3) | (0.162, n = 3) |
| 291 | >872 | 0.129 | 3.16 |
| | (n = ½) | (0.0346, n = 3) | (0.270, n = 3) |
| 292 | 595 | 0.0547 | 1.19 |
| 293 | 668 | 0.0775 | 1.64 |
| 294 | 629 | 0.205 | 2.92 |
| 295 | >1000 | 0.181 | 4.12 |
| 296 | >1000 | 0.444 | 3.33 |
| 297 | >1240 | 0.0958 | 2.98 |
| 298 | >1370 | 0.0578 | 3.03 |
| 299 | >1040 | 0.734 | 54.3 |
| 300 | 251 | 0.0504 | 2.13 |
| 301 | 44.4 | 0.0273 | 0.875 |
| | (6.17, n = 3) | (0.00127, n = 3) | (0.0889, n = 3) |
| 302 | 18.5 | 0.0289 | 0.617 |
| 303 | 502 | 0.0580 | 3.69 |
| | (189, n = 3) | (0.0151, n = 3) | (1.86, n = 3) |
| 304 | >855 | 0.0499 | 4.44 |
| 305 | 352 | 0.0250 | 0.830 |
| | (30.8, n = 2) | (0.00586, n = 2) | (0.481, n = 2) |
| 306 | >1040 | 0.0349 | 2.86 |
| 307 | 117 | 0.0773 | 5.1 |
| 308 | 94.2 | 0.0288 | 1.01 |
| 309 | 64.1 | 0.0264 | 1.04 |
| 310 | 174 | 0.0315 | 1.70 |
| | (8.97, n = 2) | (0.00162, n = 2) | (0.144, n = 2) |
| 311 | 115 | 0.0497 | 11.5 |
| 312 | 106 | 0.0348 | 1.63 |
| 313 | 27.6 | 0.0261 | 0.815 |
| 314 | 116 | 0.027 | 0.717 |
| 315 | 539 | 0.0677 | 2.28 |
| 316 | 654 | 0.0418 | 0.957 |
| | (76.5, n = 3) | (0.00224, n = 3) | (0.180, n = 3) |
| 317 | 253 | 0.0215 | 2.63 |
| 318 | 730 (n = ½) | 0.0452 | 7.52 |
| | | (0.00883, n = 2) | (0.256, n = 2) |
| 319 | >984 | 0.0349 | 3.61 |
| 320 | >1040 | 0.136 | 5.17 |
| 321 | 770 | 0.064 | 4.2 |
| 322 | 1030 | 0.175 | 2.31 |
| 323 | 300 | 0.0516 | 1.65 |
| 324 | 449 | 0.0278 | 0.609 |
| 325 | 13 | 0.0209 | 0.475 |
| 326 | 207 | 0.251 | 2.92 |
| 327 | 114 | 0.0667 | 2.10 |
| | (8.53, n = 2) | (0.0211, n = 2) | (0.287, n = 2) |
| 328 | >1450 | 0.136 | 3.98 |
| | (n = ½) | (0.0602, n = 2) | (0.339, n = 2) |
| 329 | 17.0 | 0.0439 | 3.28 |
| | (2.51, n = 2) | (0.0105, n = 2) | (0.327, n = 2) |
| 330 | >1050 | 0.114 | 12.7 |
| 331 | >969 | 0.0851 | 11.4 |
| | (n = ½) | (0.00508, n = 2) | (0.160, n = 2) |
| 332 | 397 | 0.0497 | 7.87 |
| | (272, n = 2) | (0.00681, n = 2) | (0.333, n = 2) |
| 333 | 578 | 0.0634 | 4.25 |
| | (68.8, n = 2) | (0.00255, n = 2) | (0.180, n = 2) |
| 334 | 192 | 0.0646 | 2.17 |
| 335 | 27.1 | 0.0444 | 2.54 |
| 336 | 17.1 | 0.0277 | 2.44 |
| 337 | 335 | 0.0363 | 1.61 |
| 338 | >1060 | 0.0831 | 3.23 |
| 339 | 873 | 0.0388 | 2.69 |
| | (19.4, n = 2) | (0.0198, n = 2) | (0.218, n = 2) |
| 340 | 250 | 0.0507 | 2.08 |
| | (60.8, n = 2) | (0.0177, n = 2) | (0.0742, n = 2) |
| 341 | 39.7 | 0.0559 | 6.49 |
| 342 | >1000 | 0.129 | 15.4 |
| 343 | >1070 | 0.0374 | 13.3 |
| 344 | >1080 | 0.0507 | 14.8 |
| 346 | 310 | 0.0559 | 1.67 |
| | (26.0, n = 3) | (0.0248, n = 2) | (0.911, n = 2) |
| 349 | >1060 | 0.0800 | 1.72 |
| | (n = ⅓) | (0.0215, n = 3) | (0.0730, n = 3) |
| 352 | >1030 | 0.0726 | 3.03 |
| | (n = ½) | (0.00687, n = 3) | (0.673, n = 3) |
| 354 | >953 | 0.175 | 7.85 |
| | | (0.0209, n = 2) | (0.190, n = 2) |
| 356 | >1010 | 0.350 | 10.3 |
| | | (0.0397, n = 2) | (1.53, n = 2) |
| 357 | >977 | 0.316 | 7.02 |
| | | (0.0233, n = 2) | (1.19, n = 2) |
| 358 | 915 | 0.0636 | 1.37 |
| | (n = ½) | (0.00684, n = 3) | (0.189, n = 3) |
| 359 | >982 | 0.0874 | 2.59 |
| | (n = ½) | (0.0159, n = 3) | (0.833, n = 3) |
| 360 | 485 | 0.128 | 1.74 |
| | | (0.00895, n = 2) | (0.0269, n = 2) |
| 362 | >1050 | 0.337 | 6.95 |
| | | (0.00484, n = 2) | (0.446, n = 2) |
| 363 | >1020 | 0.170 | 3.89 |
| | (n = ½) | (0.0113, n = 3) | (0.864, n = 3) |
| 364 | >1150 | 0.672 | 17.2 |
| | (n = ½) | (0.0431, n = 3) | (2.26, n = 3) |
| 367 | 777 | 0.0282 | 0.809 |

Functional Activity (with BSA)

Functional activity is determined in hGLP-1R, hGcgR and hGIP-R expressing HEK-293 clonal cell lines. Each receptor over-expressing cell line is treated with peptide (20 point CRC, 2.75-fold Labcyte Echo direct dilution) in DMEM (Gibco Cat #31053) supplemented with 1λ GlutaMAX™ supplement (L-alanyl-L-glutamine dipeptide Gibco®, 0.25% FBS (Fetal Bovine Serum), 0.05% fraction V BSA (Bovine Serum Albumin), 250 μM 3-isobutyl-1-methylxanthine (IBMX) and 20 mM 4-(2-hydroxyethyl)-1-piperazineethanesulfonic acid (HEPES) in a 20 μl assay volume.

After 60-minute incubation at room temperature, the resulting increase in intracellular cAMP is quantitatively determined using the CisBio cAMP Dynamic 2 homogeneous time-resolved fluorescence (HTRF) Assay Kit. The cAMP levels within the cell are detected by adding the cAMP-d2 conjugate in cell lysis buffer followed by the antibody anti-cAMP-Eu³⁺-Cryptate, also in cell lysis buffer. The resulting competitive assay is incubated for at least 60 minutes at room temperature and then detected using an instrument with excitation at 320 nm and emission at 665 nm and 620 nm. Envision units (emission at 665 nm/620 nm*10,000) are inversely proportional to the amount of cAMP present and are converted to nM cAMP per well using a cAMP standard curve.

The amount of cAMP generated (nM) in each well is converted to a percent of the maximal response observed with either human GLP-1(7-36)NH₂, human Gcg, or human GIP(1-42)NH₂. A relative EC₅₀ value is derived by non-linear regression analysis using the percent maximal response vs. the concentration of peptide added, fitted to a four-parameter logistic equation.

EC₅₀ determination of human GLP-1(7-36)NH₂ at human GLP-1R, human Gcg at human GcgR, and human GIP(1-42)NH₂ at human GIP-R: the peptide concentration ranges were 448 pM to 99.5 nM. EC₅₀ determination of Examples at human GLP-1R, human GcgR, and human GIP-R: the peptide concentration ranges are 51.5 fM to 11.4 μM.

TABLE 2
Functional cAMP Potency (EC₅₀) for Example and comparator peptides
(hGcg, hGIP amide, and hGLP-1 amide) in the presence of FBS.

| Example or | cAMP EC₅₀, nM (SEM, n) | | | | | |
|---|---|---|---|---|---|---|
| comparator | GcgR | | GIPR | | GLP-1R | |
| hGcg | 0.0125 + 0.0011 | (n = 12) | | | | |
| hGIP amide | | | 0.0979 | (0.0088, n = 12) | | |
| hGLP-1 | | | | | 0.0424 | (0.0043, n = 12) |
| amide | | | | | | |
| Example 1 | >11400 | (n = 1/9) | 38.5 | (20.2, n = 12) | 53.3 | (21.4, n = 12) |
| Example 2 | >10900 | (n = 1/12) | 2.64 | (0.696, n = 12) | 6.52 | (1.76, n = 11) |
| Example 3 | >10900 | (n = 1/7) | 9.24 | (2.23, n = 7) | 19.4 | (7.02, n = 7) |
| Example 4 | >10900 | (n = 1/5) | 1.03 | (0.181, n = 5) | 2.02 | (0.596, n = 4) |

| | | | |
|---|---|---|---|
| Example 5 | >10900 | 1.98 | 1.61 |

cAMP Pharmacological Functional Assay in Presence of Casein

An additional set of cAMP assays are conducted in HEK293 cells expressing the human GLP-1 receptor (GLP-1R), gastric inhibitory peptide receptor (GIPR), Glucagon receptor (GcgR). Pharmacological activity of the hGLP1R/GIPR peptides are determined in HEK293 cells stably expressing the human GLP-1 receptor (GLP-1R), gastric inhibitory peptide receptor (GIPR), or GLP-2 receptor (GLP-2R). Each receptor over-expressing cell line (20 μl) is treated with the test peptide in DMEM (Gibco Cat #31053) supplemented with 0.1% Casein (Sigma Cat #C4765), 250 μM IBMX, 1× GlutaMAX™ (Gibco Cat #35050), and 20 mM HEPES (HyClone Cat #SH30237.01) in a 20 μl assay volume. After 60 minute incubation at room temperature, the resulting increase in intracellular cAMP is quantitatively determined using the CisBio cAMP Dynamic 2 HTRF Assay Kit (62AM4PEJ). The Lysis buffer containing cAMP-d2 conjugate (20 μl) and the antibody anti-cAMP-Eu3+-Cryptate (20 μl) are then added to determine the cAMP level. After 1 h-incubation at room temperature, HTRF signal is detected with an Envision 2104 plate reader (PerkinElmer). Fluorescent emission at 620 nm and at 665 nm is measured and the ratio between 620 nm and at 665 nm is calculated and then are converted to nM cAMP per well using a cAMP standard curve. Dose response curves of compounds are plotted as the percentage of stimulation normalized to minimum (buffer only) and maximum (maximum concentration of each control ligand) values and analyzed using a four parameter non-liner regression fit with a variable slope (Genedata Screener 13). EC50 is the concentration of compound causing half-maximal simulation in a dose response curve. A relative EC₅₀ value is derived by non-linear regression analysis using the percent maximal response vs. the concentration of peptide added, fitted to a four-parameter logistic equation.

Using Homogeneous Time Resolved Fluorescence methods, assays are conducted to determine the intrinsic potency of Example and comparator molecules performed in the presence of casein (instead of serum albumin) as a nonspecific blocker, which does not interact with the fatty acid moieties of the analyzed molecules.

Intracellular cAMP levels are determined by extrapolation using a standard curve. Dose response curves of compounds are plotted as the percentage of stimulation normalized to minimum (buffer only) and maximum (maximum concentration of each control ligand) values and analyzed using a four parameter non-linear regression fit with a variable slope (Genedata Screener 13). EC₅₀ is the concentration of compound causing half-maximal simulation in a dose response curve. Each relative EC50 value for the Geometric mean calculation is determined from a curve fitting.

Concentration response curves of compounds are plotted as the percentage of stimulation normalized to minimum (buffer only) and maximum (maximum concentration of each control ligand) values and analyzed using a four parameter non-liner regression fit with a variable slope (Genedata Screener 13). EC50 is the concentration of compound causing half-maximal simulation in a dose response curve.

The EC₅₀ summary statistics are computed as follows:

### Geometric Mean:

GM=10\hat{ }(arithmetic mean of log₁₀ transformed EC₅₀ values).

The standard error of the mean is reported:

SEM=geometric mean ×(standard deviation of log₁₀ transformed EC₅₀ values/square root of the # of runs)×logₑ of 10.

The log transform accounts for the EC₅₀ values falling on a multiplicative, rather than an arithmetic scale.

Each day, the assay is run, the test peptides are run plus the native ligands GIP and GLP-1, buffer only as baseline (minimum) and the highest concentration of the respective GIP and GLP-1 standard is used as maximum for calculations. For illustration, as shown by Example 1, the test peptide is tested in 8 runs of the assay. For avoidance of doubt, hGIP amide and hGLP-1 amide EC50 in Table 3 are illustrative of geometric mean values from a series of 18 assay values, and values will vary each day compared to the zero buffer. Accordingly, each Example will use the geometric mean of those values to normalize the Example assay runs.

TABLE 3
Functional activation of hGLP-1R, hGIPR, hGcgR in the presence of 0.1% Casein.

| | | | | hGLP1R |
|---|---|---|---|---|
| Example | hGIPR cAMP | hGIPR cAMP | hGLP1R cAMP | cAMP |
| or | Rel EC₅₀ nM | EC₅₀ ratio | Rel EC₅₀ nM | EC₅₀ ratio |
| comparator | (SEM, n) | (SEM, n) | (SEM, n) | (SEM, n) |
| hGIP amide | 0.170 | | | |
| | (0.012, n = 18) | | | |
| hGLP-1 | | | 0.0396 | |
| amide | | | (0.0030, n = 16) | |
| 1 | 0.0356 | 4.65 | 0.0410 | 1.12 |
| | (0.00576, n = 8) | (0.514, n = 8) | (0.00720, n = 7) | (0.0949, n = 7) |
| 2 | 0.0339 | 5.89 | 0.0441 | 0.888 |
| | (0.00650, n = 5) | (1.10, n = 5) | (0.00670, n = 5) | (0.0993, n = 5) |
| 3 | 0.0411 | 4.51 | 0.0338 | 1.25 |
| | (0.00541, n = 5) | (0.355, n = 5) | (0.00156, n = 4) | (0.0916, n = 4) |
| 4 | 0.0272 | 5.95 | 0.0297 | 1.41 |
| | (0.00358, n = 6) | (0.466, n = 6) | (0.00319, n = 6) | (0.241, n = 6) |
| 5 | 0.0309 | 6.27 | 0.0164 | 2.69 |
| | (0.00402, n = 5) | (0.808, n = 5) | (0.00219, n = 5) | (0.547, n = 5) |
| 6 | 0.0899 | 2.09 | 0.374 | 0.185 |
| | (0.0196, n = 2) | (0.301, n = 2) | (0.0100, n = 2) | (0.00340, n = 2) |
| 7 | 0.461 | 0.317 | 0.470 | 0.130 |
| | | | (0.0988, n = 2) | (0.0215, n = 2) |
| 8 | 0.0848 | 1.97 | 0.148 | 0.419 |
| | (0.00744, n = 6) | (0.170, n = 6) | (0.00926, n = 6) | (0.0352, n = 6) |
| 9 | 0.210 | 0.768 | 0.194 | 0.314 |
| | (0.0335, n = 6) | (0.122, n = 6) | (0.0284, n = 6) | (0.0384, n = 6) |
| 10 | 1.28 | 0.151 | 7.64 | 0.00912 |
| | (0.270, n = 2) | (0.0416, n = 2) | (0.786, n = 2) | (0.000859, |
| | | | | n = 2) |
| 11 | 0.486 | 0.399 | 6.89 | 0.0111 |
| | (0.108, n = 2) | (0.114, n = 2) | (2.68, n = 3) | (0.00387, n = 3) |
| 12 | 0.300 | 0.659 | 1.15 | 0.0603 |
| | (0.0827, n = 2) | (0.221, n = 2) | (0.00296, n = 2) | (0.000359, |
| | | | | n = 2) |
| 13 | 1.05 | 0.180 | 5.39 | 0.0133 |
| | (0.236, n = 2) | (0.0272, n = 2) | (1.35, n = 2) | (0.00338, n = 2) |
| 14 | 0.284 | 0.545 | 1.87 | 0.0306 |
| | (0.0828, n = 2) | (0.161, n = 2) | (0.534, n = 2) | (0.00519, n = 2) |
| 15 | 0.613 | 0.273 | 0.0336 | 1.65 |
| | (0.141, n = 2) | (0.00678, n = 2) | (0.0000222, | (0.0634, n = 2) |
| | | | n = 2) | |
| 16 | 0.975 | 0.157 | 0.0437 | 1.43 |
| | (0.241, n = 2) | (0.0401, n = 2) | (0.00494, n = 4) | (0.260, n = 3) |
| 17 | 5.81 | 0.0257 | 0.152 | 0.398 |
| | (0.758, n = 2) | (0.00368, n = 2) | (0.0166, n = 2) | (0.0260, n = 2) |
| 18 | 0.610 | 0.275 | 0.0945 | 0.589 |
| | (0.200, n = 2) | (0.0204, n = 2) | (0.00823, n = 2) | (0.0732, n = 2) |
| 19 | 0.386 | 0.436 | 0.104 | 0.532 |
| | (0.0583, n = 2) | (0.0449, n = 2) | (0.00342, n = 2) | (0.0375, n = 2) |
| 20 | 0.0556 | 2.81 | 0.117 | 0.577 |
| | (0.00518, n = 5) | (0.293, n = 5) | (0.0121, n = 8) | (0.0706, n = 8) |
| 21 | 0.0748 | 1.95 | 0.160 | 0.402 |
| | (0.00682, n = 8) | (0.145, n = 8) | (0.00495, n = 7) | (0.0178, n = 7) |
| 22 | 0.0842 | 1.86 | 0.206 | 0.290 |
| | (0.0103, n = 4) | (0.109, n = 4) | (0.0172, n = 5) | (0.0233, n = 5) |
| 23 | 0.204 | 0.754 | 0.190 | 0.342 |
| | (0.0160, n = 5) | (0.0348, n = 5) | (0.00766, n = 7) | (0.0255, n = 7) |
| 24 | 0.762 | 0.228 | 13.0 | 0.00416 |
| | (0.206, n = 2) | (0.0443, n = 2) | (n = ½) | (n = ½) |
| 25 | 0.230 | 0.636 | 0.356 | 0.197 |
| | (0.0191, n = 6) | (0.0564, n = 6) | (0.0415, n = 6) | (0.0287, n = 6) |
| 26 | 0.251 | 0.585 | 0.293 | 0.238 |
| | (0.0264, n = 6) | (0.0531, n = 6) | (0.0442, n = 5) | (0.0309, n = 5) |
| 27 | 0.0789 | 1.90 | 0.557 | 0.123 |
| | (0.00792, n = 6) | (0.295, n = 6) | (0.0717, n = 5) | (0.0102, n = 5) |
| 28 | 1.66 | 0.106 | 32.7 | 0.00166 |
| | (0.257, n = 2) | (0.0238, n = 2) | (n = 1/2) | (n = 1/2) |
| 29 | 0.320 | 0.536 | 0.999 | 0.0671 |
| | (0.0548, n = 2) | (0.0522, n = 2) | (0.336, n = 2) | (0.0283, n = 2) |
| 30 | 0.114 | 1.50 | 1.84 | 0.0331 |
| | (0.00650, n = 2) | (0.0246, n = 2) | (0.280, n = 2) | (0.00127, n = 2) |
| 31 | 0.388 | 0.456 | 0.891 | 0.0708 |
| | (0.0773, n = 2) | (0.121, n = 2) | (0.147, n = 2) | (0.0192, n = 2) |
| 32 | 0.179 | 0.840 | 0.498 | 0.136 |
| | (0.0200, n = 5) | (0.0750, n = 5) | (0.0124, n = 5) | (0.00728, n = 5) |
| 33 | 63.3 | 0.00222 | 251 | 0.000256 |
| | (6.65, n = 2) | (0.000125, | (n = ½) | (n = ½) |
| | | n = 2) | | |
| 34 | 0.360 | 0.393 | 1.56 | 0.0392 |
| | (0.0200, n = 2) | (0.00105, n = 2) | (0.132, n = 2) | (0.00119, n = 2) |
| 35 | 0.0905 | 1.58 | 8.38 | 0.00728 |
| | (0.00717, n = 2) | (0.215, n = 2) | (0.707, n = 2) | (0.000221, |
| | | | | n = 2) |
| 36 | 0.309 | 0.458 | 2.29 | 0.0268 |
| | (0.0126, n = 2) | (0.00789, n = 2) | (0.390, n = 2) | (0.00309, n = 2) |
| 37 | 0.269 | 0.532 | 1.15 | 0.0533 |
| | (0.0546, n = 2) | (0.0765, n = 2) | (0.216, n = 2) | (0.00707, n = 2) |
| 38 | 0.147 | 0.967 | 2.82 | 0.0217 |
| | (0.0217, n = 2) | (0.0861, n = 2) | (0.0832, n = 2) | (0.00181, n = 2) |
| 39 | 0.133 | 1.07 | 1.92 | 0.0323 |
| | (0.0242, n = 2) | (0.132, n = 2) | (0.447, n = 2) | (0.00571, n = 2) |
| 40 | 0.136 | 1.06 | 0.746 | 0.0801 |
| | (0.0164, n = 2) | (0.188, n = 2) | (0.118, n = 2) | (0.0105, n = 2) |
| 41 | 0.229 | 0.637 | 0.960 | 0.0637 |
| | (0.0451, n = 2) | (0.159, n = 2) | (0.0203, n = 2) | (0.00478, n = 2) |
| 42 | 0.161 | 0.897 | 1.45 | 0.0471 |
| | (0.00967, n = 2) | (0.149, n = 2) | (0.328, n = 2) | (0.0107, n = 2) |
| 43 | 0.112 | 1.28 | 0.862 | 0.0777 |
| | (0.0129, n = 2) | (0.0105, n = 2) | (0.0963, n = 2) | (0.00826, n = 2) |
| 44 | 0.128 | 1.11 | 0.752 | 0.0918 |
| | (0.0118, n = 2) | (0.0174, n = 2) | (0.201, n = 2) | (0.0245, n = 2) |
| 45 | 0.106 | 1.75 | 1.03 | 0.0569 |
| | (0.0158, n = 2) | (0.164, n = 2) | (0.194, n = 2) | (0.00591, n = 2) |
| 46 | 0.172 | 0.828 | 0.687 | 0.0969 |
| | (0.0231, n = 2) | (0.0222, n = 2) | (0.0147, n = 2) | (0.00159, n = 2) |
| 47 | 0.287 | 0.654 | 0.702 | 0.0890 |
| | (0.0529, n = 2) | (0.0819, n = 2) | (0.197, n = 2) | (0.0310, n = 2) |
| 48 | 0.168 | 0.863 | 0.691 | 0.0966 |
| | (0.0126, n = 2) | (0.156, n = 2) | (0.0491, n = 2) | (0.00733, n = 2) |
| 49 | 0.0868 | 1.69 | 0.597 | 0.112 |
| | (0.0288, n = 2) | (0.372, n = 2) | (0.0314, n = 2) | (0.00642, n = 2) |
| 50 | 0.0794 | 1.96 | 0.0961 | 0.675 |
| | (0.0104, n = 5) | (0.179, n = 5) | (0.00519, n = 7) | (0.0492, n = 7) |
| 51 | 0.0960 | 1.69 | 0.153 | 0.426 |
| | (0.0106, n = 7) | (0.201, n = 7) | (0.0110, n = 7) | (0.0376, n = 7) |
| 52 | 0.0997 | 1.40 | 0.132 | 0.514 |
| | (0.0119, n = 6) | (0.154, n = 6) | (0.0143, n = 5) | (0.0413, n = 5) |
| 53 | 0.628 | 0.287 | 0.0339 | 1.80 |
| | (0.161, n = 2) | (0.0912, n = 2) | (0.000266, n = 2) | (0.190, n = 2) |
| 54 | 1.57 | 0.110 | 0.0242 | 2.55 |
| | (0.144, n = 2) | (0.0181, n = 2) | (0.00711, n = 2) | (0.456, n = 2) |
| 55 | 5.45 | <0.00159 | 0.0286 | 2.33 |
| | (n = ½) | | (0.000346, n = 2) | (0.0282, n = 2) |
| 56 | 0.167 | 1.02 | 0.136 | 0.491 |
| | (0.0132, n = 2) | (0.00567, n = 2) | (0.00964, n = 2) | (0.0347, n = 2) |
| 57 | 0.0849 | 1.74 | 0.0668 | 0.996 |
| | (0.0107, n = 6) | (0.148, n = 6) | (0.00654, n = 7) | (0.0730, n = 7) |
| 58 | 0.266 | 0.522 | 0.180 | 0.378 |
| | (0.0307, n = 5) | (0.0439, n = 5) | (0.0151, n = 5) | (0.0347, n = 5) |
| 59 | 0.0922 | 1.54 | 0.0840 | 0.812 |
| | (0.0134, n = 5) | (0.186, n = 5) | (0.00742, n = 5) | (0.0816, n = 5) |
| 60 | 0.135 | 1.06 | 0.287 | 0.237 |
| | (0.0107, n = 2) | (0.0296, n = 2) | (0.0377, n = 3) | (0.0270, n = 3) |
| 61 | 0.0739 | 1.94 | 0.371 | 0.187 |
| | (0.0140, n = 2) | (0.158, n = 2) | (0.0664, n = 3) | (0.0341, n = 3) |
| 62 | 0.0601 | 2.33 | 0.159 | 0.463 |
| | (0.00469, n = 5) | (0.234, n = 5) | (0.0151, n = 5) | (0.0644, n = 5) |
| 63 | 0.0925 | 1.58 | 0.190 | 0.386 |
| | (0.0106, n = 7) | (0.156, n = 7) | (0.0195, n = 5) | (0.0466, n = 5) |
| 64 | 0.0916 | 1.57 | 0.172 | 0.429 |
| | (0.0104, n = 6) | (0.192, n = 6) | (0.0184, n = 5) | (0.0598, n = 5) |
| 65 | 0.143 | 1.04 | 0.289 | 0.233 |
| | (0.0264, n = 2) | (0.295, n = 2) | (0.0215, n = 3) | (0.0144, n = 3) |
| 66 | 0.0743 | 1.89 | 0.285 | 0.255 |
| | (0.00740, n = 5) | (0.209, n = 5) | (0.0291, n = 5) | (0.0245, n = 5) |
| 67 | 0.0913 | 1.61 | 0.447 | 0.123 |
| | (0.00577, n = 2) | (0.0648, n = 2) | (0.0958, n = 2) | (0.0314, n = 2) |
| 68 | 0.0881 | 1.67 | 0.153 | 0.354 |
| | (0.00725, n = 2) | (0.0990, n = 2) | (0.0169, n = 2) | (0.0550, n = 2) |
| 69 | 0.0712 | 2.73 | 0.0844 | 0.850 |
| | (0.0156, n = 4) | (0.431, n = 3) | (0.00548, n = 4) | (0.0870, n = 4) |
| 70 | 0.480 | 0.351 | 0.283 | 0.207 |
| | (0.0706, n = 2) | (0.0135, n = 2) | (0.0281, n = 3) | (0.0330, n = 3) |
| 71 | 0.166 | 1.02 | 1.02 | 0.0604 |
| | (0.0417, n = 2) | (0.0668, n = 2) | (0.227, n = 3) | (0.0170, n = 3) |
| 72 | 0.252 | 0.715 | 0.906 | 0.0645 |
| | (0.0456, n = 2) | (0.251, n = 2) | (0.127, n = 3) | (0.0103, n = 3) |
| 73 | 0.979 | 0.174 | 2.55 | 0.0225 |
| | (0.317, n = 2) | (0.0239, n = 2) | (0.337, n = 3) | (0.00178, n = 3) |
| 74 | 0.0866 | 1.70 | 0.467 | 0.115 |
| | (0.00653, n = 2) | (0.166, n = 2) | (0.00217, n = 2) | (0.00580, n = 2) |
| 75 | 0.214 | 0.690 | 1.49 | 0.0359 |
| | (0.0234, n = 2) | (0.0905, n = 2) | (0.00142, n = 2) | (0.00168, n = 2) |
| 76 | 0.124 | 1.24 | 0.125 | 0.630 |
| | (0.000853, | (0.0255, n = 2) | (0.00942, n = 2) | (0.0992, n = 2) |
| | n = 2) | | | |
| 77 | 135 | 0.00102 | >2000 | <0.0000325 |
| | (22.8, n = 2) | (0.000331, | (n = ½) | (n = ½) |
| | | n = 2) | | |
| 78 | 0.532 | 0.293 | 0.544 | 0.143 |
| | (0.120, n = 2) | (0.0571, n = 2) | (0.0263, n = 2) | (0.00495, n = 2) |
| 79 | 0.0732 | 2.11 | 2.02 | 0.0410 |
| | (0.0127, n = 2) | (0.305, n = 2) | (0.562, n = 2) | (0.0142, n = 2) |
| 80 | 0.140 | 1.09 | 0.351 | 0.227 |
| | (0.0130, n = 2) | (0.0709, n = 2) | (0.0522, n = 2) | (0.0516, n = 2) |
| 81 | 0.428 | 0.373 | 1.29 | 0.0630 |
| | (0.0445, n = 2) | (0.0131, n = 2) | (0.00950, n = 2) | (0.00192, n = 2) |
| 82 | 24.3 | 0.00679 | 26.1 | 0.00325 |
| | (8.06, n = 2) | (0.00175, n = 2) | (6.81, n = 2) | (0.000941, |
| | | | | n = 2) |
| 83 | 0.182 | 0.804 | 0.387 | 0.166 |
| | (0.0176, n = 2) | (0.0637, n = 2) | (0.0648, n = 2) | (0.0265, n = 2) |
| 84 | 0.0915 | 1.59 | 0.374 | 0.173 |
| | (0.00451, n = 2) | (0.0514, n = 2) | (0.0762, n = 2) | (0.0337, n = 2) |
| 85 | 1.67 | 0.111 | 0.0518 | 1.13 |
| | (0.143, n = 2) | (0.00296, n = 2) | (0.00318, n = 2) | (0.0246, n = 2) |
| 86 | 0.0452 | 3.29 | 0.271 | 0.243 |
| | (0.00765, n = 2) | (0.605, n = 2) | (0.0756, n = 2) | (0.0647, n = 2) |
| 87 | 0.0945 | 1.1 | 0.0801 | 0.786 |
| | (0.0277, n = 2) | | | |
| 88 | 0.475 | 0.392 | 0.640 | 0.0918 |
| | (0.00187, n = 2) | (0.0215, n = 2) | (0.00544, n = 2) | (0.00840, n = 2) |
| 89 | 0.119 | 1.57 | 0.335 | 0.175 |
| | (0.0150, n = 2) | (0.107, n = 2) | (0.00771, n = 2) | (0.0105, n = 2) |
| 90 | 0.0361 | 5.17 | 0.532 | 0.110 |
| | (0.000915, | (0.434, n = 2) | (0.0564, n = 2) | (0.00250, n = 2) |
| | n = 2) | | | |
| 91 | 19.2 | 0.0103 | >5000 | <0.0000108 |
| | (n = ½) | (n = ½) | (n = ½) | (n = ½) |
| 92 | 0.338 | 0.546 | 0.257 | 0.258 |
| 93 | 0.185 | 0.956 | 0.115 | 0.498 |
| | (0.0241, n = 2) | (0.292, n = 2) | (0.0143, n = 3) | (0.0199, n = 3) |
| 94 | 0.425 | 0.434 | 0.338 | 0.196 |
| 95 | 1.17 | 0.145 | 1.01 | 0.0576 |
| | (0.0326, n = 2) | (0.0227, n = 2) | (0.229, n = 3) | (0.00824, n = 3) |
| 96 | 0.712 | 0.238 | 0.590 | 0.0977 |
| | (0.0736, n = 2) | (0.0195, n = 2) | (0.0509, n = 3) | (0.0101, n = 3) |
| 97 | 1.85 | 0.0953 | 2.18 | 0.0268 |
| | (0.214, n = 2) | (0.0278, n = 2) | (0.347, n = 3) | (0.00442, n = 3) |
| 98 | 0.0718 | 2.43 | 0.157 | 0.384 |
| 99 | 0.0643 | 2.27 | 0.0986 | 0.629 |
| | (0.0120, n = 5) | (0.208, n = 5) | (0.0104, n = 4) | (0.0938, n = 4) |
| 100 | 0.120 | 1.48 | 0.0790 | 0.724 |
| | (0.0169, n = 2) | (0.191, n = 2) | (0.00439, n = 2) | (0.0809, n = 2) |
| 101 | 0.0704 | 2.07 | 0.0579 | 1.06 |
| | (0.00913, n = 5) | (0.138, n = 5) | (0.00589, n = 4) | (0.131, n = 4) |
| 102 | 0.178 | 0.983 | 0.0628 | 0.913 |
| | | | (0.00458, n = 2) | (0.118, n = 2) |
| 103 | 0.0693 | 2.01 | 0.101 | 0.655 |
| | (0.0165, n = 4) | (0.183, n = 4) | (0.0132, n = 3) | (0.108, n = 3) |
| 104 | 0.0323 | 4.93 | 0.0248 | 1.85 |
| | (0.00474, n = 8) | (0.595, n = 8) | (0.00679, n = 6) | (0.242, n = 6) |
| 105 | 1.85 | 0.101 | 0.0401 | 1.51 |
| | (0.133, n = 2) | (0.00134, n = 2) | (0.00740, n = 2) | (0.396, n = 2) |
| 106 | 6.54 | 0.0302 | 0.0413 | 1.42 |
| | (n = ½) | (n = ½) | (0.000823, n = 2) | (0.0898, n = 2) |
| 107 | 0.241 | 0.747 | 0.0539 | 1.10 |
| | (0.0209, n = 5) | (0.0859, n = 5) | (0.00678, n = 5) | (0.128, n = 5) |
| 108 | 0.0885 | 2.19 | 0.294 | 0.218 |
| | (0.00413, n = 2) | (0.0798, n = 2) | (0.0441, n = 2) | (0.0253, n = 2) |
| 109 | 0.109 | 1.83 | 0.350 | 0.182 |
| | (0.0252, n = 2) | (0.397, n = 2) | (0.0142, n = 2) | (0.0134, n = 2) |
| 110 | 0.218 | 0.929 | 0.179 | 0.358 |
| | (0.0657, n = 2) | (0.263, n = 2) | (0.0168, n = 2) | (0.0452, n = 2) |
| 111 | 0.141 | 1.1 | 0.308 | 0.209 |
| | (0.0253, n = 2) | | (0.0144, n = 2) | (0.0151, n = 2) |
| 112 | 0.124 | 1.53 | 0.160 | 0.402 |
| | (0.00217, n = 2) | | (0.0115, n = 2) | (0.0185, n = 2) |
| 113 | 0.166 | 1.39 | 0.233 | 0.275 |
| | (0.0364, n = 2) | | (0.0133, n = 2) | (0.0228, n = 2) |
| 114 | 0.133 | 1.65 | 0.267 | 0.234 |
| | (0.0220, n = 2) | | | |
| 115 | 0.102 | 1.85 | 0.0880 | 0.743 |
| | (0.0163, n = 5) | (0.319, n = 5) | (0.00660, n = 4) | (0.0516, n = 4) |
| 116 | 0.0867 | 2.13 | 0.0703 | 0.956 (0.147, |
| | (0.0141, n = 5) | (0.255, n = 5) | (0.0111, n = 4) | n = 4) |
| 117 | 0.0648 | 2.44 | 0.0615 | 0.998 |
| | (0.00602, n = 7) | (0.263, n = 6) | (0.00275, n = 7) | (0.0681, n = 7) |
| 118 | 0.0538 | 3.42 | 0.0588 | 1.13 |
| | (0.00395, n = 7) | (0.247, n = 6) | (0.00577, n = 6) | (0.115, n = 6) |
| 119 | 0.216 | 0.901 | 0.0913 | 0.702 |
| | (0.0107, n = 2) | (0.0541, n = 2) | (0.00729, n = 2) | (0.0788, n = 2) |
| 120 | 0.122 | 1.67 | 0.201 | 0.319 |
| | (0.0397, n = 2) | (0.509, n = 2) | (0.0318, n = 2) | (0.0398, n = 2) |
| 121 | 0.0760 | 1.44 | 0.0849 | 0.760 |
| | (0.0189, n = 2) | | (0.0136, n = 2) | (0.128, n = 2) |
| 122 | 0.0923 | 1.73 | 0.0760 | 0.856 |
| | (0.0122, n = 2) | (0.399, n = 2) | (0.0221, n = 2) | (0.223, n = 2) |
| 123 | 0.0423 | 4.04 | 0.0344 | 1.58 |
| | (0.00604, n = 8) | (0.586, n = 8) | (0.00562, n = 5) | (0.180, n = 5) |
| 124 | 0.0762 | 2.18 | 0.0475 | 1.29 |
| | (0.00816, n = 4) | (0.283, n = 4) | (0.00318, n = 4) | (0.0473, n = 4) |
| 125 | 0.0381 | 3.79 | 0.0478 | 1.31 |
| | (0.00482, n = 5) | (0.152, n = 5) | (0.00569, n = 4) | (0.229, n = 4) |
| 126 | 0.0685 | 2.53 | 0.0715 | 0.869 |
| | (0.00653, n = 4) | (0.361, n = 3) | (0.00249, n = 4) | (0.0332, n = 4) |
| 127 | 0.158 | 0.917 | 0.145 | 0.443 |
| | (0.0401, n = 2) | | (0.00951, n = 2) | (0.0176, n = 2) |
| 128 | 0.0694 | 2.46 | 0.0924 | 0.700 |
| | (0.00856, n = 4) | (0.344, n = 3) | (0.0147, n = 4) | (0.119, n = 4) |
| 129 | 0.106 | 1.88 | 0.121 | 0.543 |
| | (0.00707, n = 2) | | (0.0220, n = 2) | (0.112, n = 2) |
| 130 | 1.55 | 0.116 | 0.368 | 0.182 |
| 131 | 0.183 | 1.03 | 0.156 | 0.425 |
| 132 | 0.0657 | 2.88 | 0.26 | 0.255 |
| 133 | 0.0879 | 1.89 | 0.0436 | 1.52 |
| | (0.0298, n = 2) | (1.13, n = 2) | | |
| 134 | 0.104 | 1.43 | 0.0654 | 0.942 |
| | (0.00639, n = 2) | (0.244, n = 2) | (0.00394, n = 2) | (0.00985, n = 2) |
| 135 | 0.373 | 0.482 | 0.131 | 0.509 |
| 136 | 3.39 | 0.0544 | 0.06 | 1.1 |
| 137 | 0.0333 | 4.47 | 0.0397 | 1.25 |
| | (0.00461, n = 8) | (0.381, n = 8) | (0.00540, n = 5) | (0.114, n = 5) |
| 138 | 0.0581 | 2.18 | 0.0471 | 1.12 |
| | (0.00498, n = 3) | (0.423, n = 3) | (0.00784, n = 3) | (0.105, n = 3) |
| 139 | 0.0370 | 3.61 | 0.0417 | 1.13 |
| | (0.00460, n = 5) | (0.444, n = 5) | (0.00760, n = 5) | (0.105, n = 5) |
| 140 | 0.0349 | 3.76 | 0.0424 | 1.16 |
| | (0.00428, n = 5) | (0.415, n = 5) | (0.00487, n = 5) | (0.0926, n = 5) |
| 141 | 0.2 | 0.538 | 0.0975 | 0.414 |
| 142 | 0.0489 | 2.84 | 0.0426 | 1.22 |
| | (0.00436, n = 2) | (0.637, n = 2) | (0.00626, n = 2) | (0.0942, n = 2) |
| 143 | 0.0515 | 2.78 | 0.0745 | 0.714 |
| | (0.0105, n = 2) | (0.920, n = 2) | (0.0109, n = 2) | (0.152, n = 2) |
| 144 | 0.0375 | 3.60 | 0.0608 | 0.851 |
| | (0.00249, n = 3) | (0.373, n = 3) | (0.00352, n = 3) | (0.0845, n = 3) |
| 145 | 0.0514 | 2.46 | 0.0427 | 1.22 |
| | (0.0124, n = 3) | (0.300, n = 3) | (0.00108, n = 2) | (0.116, n = 2) |
| 146 | 0.0583 | 1.98 | 0.0604 | 0.951 |
| 147 | 0.0304 | 4.14 | 0.0527 | 1.13 |
| | (0.00146, n = 3) | (0.241, n = 3) | (0.00623, n = 3) | (0.117, n = 3) |
| 148 | 0.0430 | 2.70 | 0.0626 | 0.859 |
| | (0.00713, n = 3) | (0.462, n = 3) | (0.00751, n = 3) | (0.167, n = 3) |
| 149 | 0.0711 | 1.82 | 0.0916 | 0.484 |
| | (0.0109, n = 2) | (0.0572, n = 2) | (0.0149, n = 2) | (0.0343, n = 2) |
| 150 | 0.0511 | 2.64 | 0.0575 | 0.790 |
| | (0.00569, n = 2) | (0.761, n = 2) | (0.00855, n = 2) | (0.186, n = 2) |
| 151 | 0.41 | 0.262 | 1.60 | 0.0278 |
| | | | (0.338, n = 2) | (0.00331, n = 2) |
| 152 | 0.0504 | 2.57 | 0.0915 | 0.484 |
| | (0.00566, n = 2) | (0.188, n = 2) | (0.0128, n = 2) | (0.0236, n = 2) |
| 153 | 0.0634 | 1.7 | 0.0904 | 0.501 |
| | | | (0.0113, n = 2) | (0.107, n = 2) |
| 154 | 0.0266 | 5.90 | 0.0393 | 1.23 |
| | (0.00348, n = 7) | (0.513, n = 7) | (0.00511, n = 5) | (0.117, n = 5) |
| 155 | 0.266 | 0.584 | 0.495 | 0.0978 |
| 156 | >30.0 | <0.00519 | 0.638 | 0.0759 |
| 157 | 0.0453 | 3.15 | 0.0431 | 1.25 |
| | (0.00495, n = 6) | (0.444, n = 6) | (0.00557, n = 4) | (0.135, n = 4) |
| 158 | 0.0454 | 3.22 | 0.0374 | 1.34 |
| | (0.0102, n = 5) | (0.527, n = 5) | (0.00374, n = 5) | (0.0947, n = 5) |
| 159 | 4.6 | 0.039 | 0.33 | 0.176 |
| 160 | 21.0 | 0.00712 | 0.0461 | 1.24 |
| | (0.568, n = 2) | (0.00165, n = 2) | (0.00206, n = 2) | (0.0824, n = 2) |
| 161 | 0.254 | 0.706 | 4.8 | 0.0121 |
| 162 | 4.56 | 0.0393 | 36 | 0.00161 |
| 163 | 0.121 | 1.29 | 0.0316 | 1.54 |
| 164 | 3.09 | 0.0581 | 15.6 | 0.00373 |
| 165 | 1.04 | 0.172 | 5.81 | 0.01 |
| 166 | 0.355 | 0.504 | 4.08 | 0.0143 |
| 167 | 0.617 | 0.291 | 1.2 | 0.0487 |
| 168 | 0.572 | 0.313 | 1.8 | 0.0323 |
| 169 | 0.86 | 0.122 | 3.37 | 0.0136 |
| 170 | 0.569 | 0.185 | 10.4 | 0.00438 |
| 171 | 0.223 | 0.471 | 0.75 | 0.0609 |
| 172 | 1.05 | 0.1 | 1.2 | 0.038 |
| 173 | 0.586 | 0.179 | 1.23 | 0.0373 |
| 174 | 0.217 | 0.483 | 0.472 | 0.0968 |
| 175 | 0.0881 | 1.19 | 1.11 | 0.0412 |
| 176 | 0.523 | 0.201 | 1.07 | 0.0427 |
| 177 | 1.4 | 0.0749 | 6.79 | 0.00673 |
| 178 | 3.08 | 0.0341 | 13.1 | 0.00349 |
| 179 | 1.83 | 0.0575 | 2.7 | 0.0169 |
| 180 | 0.79 | 0.133 | 2.16 | 0.0212 |
| 181 | 0.0459 | 4.10 | 0.0592 | 0.718 |
| | (0.0122, n = 5) | (0.698, n = 5) | (0.00966, n = 6) | (0.0586, n = 6) |
| 182 | 0.0442 | 4.37 | 0.0463 | 0.873 |
| | (0.0133, n = 5) | (0.940, n = 5) | (0.00578, n = 6) | (0.0433, n = 6) |
| 183 | 0.0615 | 3.10 | 0.0551 | 0.843 |
| | (0.0175, n = 5) | (0.600, n = 5) | (0.0125, n = 5) | (0.121, n = 5) |
| 184 | 0.0477 | 2.45 | 0.392 | 0.135 |
| 185 | 0.632 | 0.185 | 7.16 | 0.00739 |
| 186 | | | 0.0939 | 0.572 |
| 187 | 0.0371 | 4.61 | 0.0577 | 0.702 |
| | (0.00593, n = 6) | (0.839, n = 6) | (0.00695, n = 6) | (0.0431, n = 6) |
| 188 | 0.121 | 0.969 | 0.13 | 0.408 |
| 189 | 0.0775 | 2.41 | 0.0608 | 0.668 |
| | (0.0140, n = 5) | (0.314, n = 5) | (0.00976, n = 6) | (0.0450, n = 6) |
| 190 | 0.738 | 0.158 | 0.0241 | 2.2 |
| 191 | 0.0645 | 2.53 | 0.0356 | 1.60 |
| | (0.0139, n = 3) | (0.131, n = 3) | (0.00490, n = 3) | (0.182, n = 3) |
| 192 | 0.0615 | 2.83 | 0.0289 | 1.73 |
| | (0.0139, n = 5) | (0.340, n = 5) | (0.00261, n = 5) | (0.0510, n = 5) |
| 193 | 0.336 | 0.538 | 0.162 | 0.359 |
| 194 | 0.423 | 0.427 | 0.284 | 0.205 |
| 195 | 0.193 | 0.936 | 0.0966 | 0.602 |
| 196 | 0.277 | 0.653 | 0.175 | 0.332 |
| 197 | 0.211 | 0.855 | 0.248 | 0.189 |
| 198 | >30.0 | <0.00602 | 0.139 | 0.337 |
| 199 | >30.0 | <0.00602 | 0.0422 | 1.11 |
| 200 | 12.3 | 0.0146 | 0.0818 | 0.573 |
| 201 | >30.0 | <0.00602 | 0.0385 | 1.22 |
| 202 | 0.0392 | 4.79 | 0.0608 | 0.712 |
| | (0.00958, n = 3) | (0.700, n = 3) | (0.00408, n = 2) | (0.0866, n = 2) |
| 203 | 0.0387 | 4.95 | 0.0679 | 0.671 |
| | (0.00465, n = 4) | (0.383, n = 4) | (0.0116, n = 3) | (0.130, n = 3) |
| 204 | 0.0424 | 4.58 | 0.0654 | 0.689 |
| | (0.0132, n = 3) | (1.01, n = 3) | (0.0166, n = 2) | (0.207, n = 2) |
| 205 | 0.0281 | 5.71 | 0.0261 | 1.46 |
| | (0.000581, | (0.519, n = 2) | (0.00703, n = 3) | (0.102, n = 3) |
| | n = 2) | | | |
| 206 | 0.0409 | 4.75 | 0.0270 | 1.42 |
| | (0.00271, n = 2) | (0.0828, n = 2) | (0.00477, n = 3) | (0.139, n = 3) |
| 207 | 0.0395 | 4.76 | 0.0359 | 1.23 |
| | (0.0103, n = 3) | (0.715, n = 3) | (0.00622, n = 2) | (0.275, n = 2) |
| 208 | 0.0371 | 5.33 | 0.0753 | 0.587 |
| | (0.00797, n = 4) | (0.808, n = 4) | (0.00608, n = 3) | (0.0362, n = 3) |
| 209 | 0.0308 | 5.73 | 0.0374 | 1.12 |
| | (0.00636, n = 5) | (0.721, n = 5) | (0.00451, n = 4) | (0.108, n = 4) |
| 210 | 0.0383 | 5.40 | 0.0432 | 1.03 |
| | (0.0124, n = 4) | (1.22, n = 4) | (0.00554, n = 3) | (0.118, n = 3) |
| 211 | 0.0442 | 4.59 | 0.0337 | 1.28 |
| | (0.00939, n = 5) | (0.656, n = 5) | (0.00481, n = 4) | (0.203, n = 4) |
| 212 | 0.0501 | 4.17 | 0.0572 | 0.771 |
| | (0.0132, n = 4) | (0.999, n = 4) | (0.00467, n = 3) | (0.0330, n = 3) |
| 213 | 0.0523 | 3.87 | 0.0710 | 0.694 |
| | (0.0140, n = 4) | (0.721, n = 4) | (0.0146, n = 4) | (0.110, n = 4) |
| 214 | 0.0251 | 6.92 | 0.0221 | 1.74 |
| | (0.00459, n = 4) | (0.628, n = 4) | (0.00364, n = 5) | (0.122, n = 5) |
| 215 | 0.0525 | 3.53 | 0.0529 | 0.813 |
| | (0.00720, n = 3) | (0.379, n = 3) | (0.00177, n = 2) | (0.0175, n = 2) |
| 216 | 0.0401 | 4.91 | 0.0327 | 1.31 |
| | (0.0151, n = 3) | (1.22, n = 3) | (0.00291, n = 2) | (0.0447, n = 2) |
| 217 | 0.0563 | 3.41 | 0.0383 | 1.14 |
| | (0.0165, n = 3) | (0.665, n = 3) | (0.00448, n = 2) | (0.193, n = 2) |
| 218 | 0.0413 | 4.43 | 0.0423 | 1.02 |
| | (0.00450, n = 3) | (0.113, n = 3) | (0.00335, n = 2) | (0.137, n = 2) |
| 219 | 0.0341 | 5.86 | 0.0403 | 1.03 |
| | (0.00595, n = 5) | (0.825, n = 5) | (0.00221, n = 4) | (0.0601, n = 4) |
| 220 | 0.0315 | 6.30 | 0.0312 | 1.36 |
| | (0.00564, n = 5) | (0.786, n = 5) | (0.00267, n = 4) | (0.176, n = 4) |
| 221 | 0.0445 | 4.50 | 0.0570 | 0.795 |
| | (0.0102, n = 4) | (0.773, n = 4) | (0.0101, n = 3) | (0.138, n = 3) |
| 222 | 0.0306 | 5.84 | 0.0248 | 1.55 |
| | (0.00648, n = 4) | (0.924, n = 4) | (0.00375, n = 5) | (0.110, n = 5) |
| 223 | 0.0670 | 2.93 | 0.0363 | 0.962 |
| | (0.00561, n = 2) | (0.487, n = 2) | (0.00532, n = 3) | (0.0435, n = 3) |
| 224 | 0.0545 | 3.52 | 0.0349 | 1.06 |
| | (0.00995, n = 3) | (0.580, n = 3) | (0.00788, n = 4) | (0.145, n = 4) |
| 225 | 0.101 | 1.99 | 0.0670 | 0.523 |
| | (0.0194, n = 2) | (0.536, n = 2) | (0.0107, n = 3) | (0.0355, n = 3) |
| 226 | 0.0461 | 4.27 | 0.0284 | 1.25 |
| | (0.00446, n = 2) | (0.762, n = 2) | (0.00805, n = 3) | (0.167, n = 3) |
| 227 | 0.0414 | 4.73 | 0.0329 | 1.07 |
| | (0.00954, n = 2) | (0.688, n = 2) | (0.00627, n = 3) | (0.0870, n = 3) |
| 228 | 0.0503 | 3.86 | 0.0282 | 1.10 |
| | (0.00265, n = 2) | (0.119, n = 2) | (0.00274, n = 2) | (0.218, n = 2) |
| 229 | 4.79 | 0.0298 | 2.11 | 0.0139 |
| 230 | 0.0431 | 3.31 | 0.0491 | 0.599 |
| 231 | 0.0253 | 5.64 | 0.0611 | 0.481 |
| 232 | 0.027 | 5.28 | 0.0724 | 0.406 |
| 233 | 0.0288 | 4.95 | 0.0549 | 0.535 |
| 234 | 0.0372 | 3.83 | 0.0926 | 0.317 |
| 235 | 0.0372 | 3.83 | 0.136 | 0.216 |
| 236 | 0.0249 | 6.76 | 0.0231 | 1.65 |
| | (0.00475, n = 5) | (0.804, n = 5) | (0.00333, n = 5) | (0.260, n = 5) |
| 237 | 0.0883 | 2.16 | 0.0187 | 2.34 |
| 238 | 0.0296 | 7.33 | 0.0241 | 1.15 |
| 239 | 0.0353 | 5.27 | 0.0376 | 0.987 |
| | (0.00282, n = 4) | (0.434, n = 4) | (0.00836, n = 4) | (0.213, n = 4) |
| 240 | 0.0223 | 9.73 | 0.0393 | 0.706 |
| 241 | 0.0257 | 7.12 | 0.0175 | 1.89 |
| | (0.00164, n = 3) | (0.471, n = 3) | (0.00373, n = 3) | (0.340, n = 3) |
| 242 | 0.0333 | 5.58 | 0.0164 | 1.96 |
| | (0.00196, n = 3) | (0.823, n = 3) | (0.00209, n = 3) | (0.186, n = 3) |
| 243 | 0.0214 | 8.69 | 0.0265 | 1.22 |
| | (0.00212, n = 3) | (1.21, n = 3) | (0.00423, n = 3) | (0.158, n = 3) |
| 244 | 0.0225 | 8.27 | 0.0252 | 1.29 |
| | (0.00136, n = 3) | (1.30, n = 3) | (0.00415, n = 3) | (0.174, n = 3) |
| 245 | 0.0552 | 2.82 | 0.0222 | 1.55 |
| 246 | 0.0258 | 6.36 | 0.0144 | 2.56 |
| | (0.00180, n = 6) | (0.340, n = 6) | (0.00106, n = 5) | (0.216, n = 5) |
| 247 | 0.0622 | 2.58 | 0.0491 | 0.614 |
| 248 | 0.0328 | 5.08 | 0.0428 | 0.800 |
| | (0.000561, | (0.118, n = 2) | (0.00493, n = 2) | (0.00908, n = 2) |
| | n = 2) | | | |
| 249 | 0.0437 | 3.84 | 0.0446 | 0.778 |
| | (0.00337, n = 2) | (0.449, n = 2) | (0.0131, n = 2) | (0.129, n = 2) |
| 250 | 0.0376 | 4.27 | 0.0306 | 0.985 |
| 251 | 0.0292 | 5.12 | 0.081 | 1.37 |
| 252 | 0.093 | 1.61 | 0.0483 | 1.44 |
| 253 | 0.219 | 0.684 | 0.109 | 0.638 |
| 254 | 0.215 | 0.695 | 0.0553 | 1.26 |
| 255 | 0.102 | 1.47 | 0.0407 | 1.71 |
| 256 | 0.643 | 0.233 | 0.0506 | 1.38 |
| 257 | 0.474 | 0.316 | 0.0779 | 0.895 |
| 258 | 2.43 | 0.0616 | 0.174 | 0.401 |
| 259 | 0.257 | 0.582 | 0.145 | 0.482 |
| 260 | 0.617 | 0.242 | 0.408 | 0.171 |
| 261 | 0.16 | 0.936 | 0.0948 | 0.75 |
| 262 | 0.13 | 1.15 | 0.0943 | 0.754 |
| 263 | 0.317 | 0.473 | 0.0785 | 0.906 |
| 264 | 0.0196 | 8.40 | 0.0229 | 3.19 |
| | (0.00197, n = 2) | (1.51, n = 2) | (0.00180, n = 2) | (0.327, n = 2) |
| 265 | 0.0229 | 7.56 | 0.0223 | 3.27 |
| | (0.00918, n = 2) | (2.21, n = 2) | (0.00115, n = 2) | (0.0894, n = 2) |
| 266 | 0.0442 | 2.82 | 0.0883 | 0.627 |
| | (0.0109, n = 6) | (0.430, n = 6) | (0.0104, n = 6) | (0.103, n = 6) |
| 267 | 0.108 | 1.50 | 0.0540 | 0.974 |
| | (0.0203, n = 5) | (0.0726, n = 5) | (0.0118, n = 5) | (0.256, n = 5) |
| 268 | 0.239 | 0.851 | 0.0572 | 0.935 |
| | (0.00366, n = 3) | (0.0652, n = 3) | (0.0135, n = 5) | (0.247, n = 5) |
| 269 | 0.257 | 0.825 | 0.0595 | 0.828 |
| | (0.0546, n = 3) | (0.187, n = 3) | (0.0158, n = 3) | (0.202, n = 3) |
| 270 | 0.328 | 0.627 | 0.128 | 0.400 |
| | (0.0226, n = 3) | (0.0795, n = 3) | (0.0355, n = 4) | (0.132, n = 4) |
| 271 | 0.334 | 0.614 | 0.0352 | 1.35 |
| | (0.0609, n = 3) | (0.0761, n = 3) | (0.00104, n = 2) | (0.0587, n = 2) |
| 272 | 0.0464 | 2.97 | 0.0435 | 1.19 |
| | (0.0119, n = 6) | (0.286, n = 6) | (0.0117, n = 4) | (0.355, n = 4) |
| 273 | 0.0790 | 2.15 | 0.0352 | 1.85 |
| | (0.0165, n = 5) | (0.333, n = 5) | (0.0230, n = 3) | (0.845, n = 3) |
| 274 | >30.0 | <0.00741 | 14.5 | 0.00353 |
| | | | (5.14, n = 2) | (0.000807, |
| | | | | n = 2) |
| 275 | >30.0 | <0.00741 | 14.0 | 0.00357 |
| | | | (0.390, n = 2) | (0.000336, |
| | | | | n = 2) |
| 276 | 0.0757 | 1.43 | 0.108 | 0.491 |
| | | | (0.0236, n = 2) | (0.117, n = 2) |
| 277 | 0.0554 | 2.02 | 0.133 | 0.536 |
| 278 | 0.293 | 0.845 | 0.123 | 0.517 |
| | | | (0.0121, n = 2) | (0.0592, n = 2) |
| 279 | 0.564 | 0.439 | 0.11 | 0.461 |
| 280 | 0.204 | 1.53 | 0.0767 | 0.663 |
| 281 | 0.166 | 1.87 | 0.16 | 0.318 |
| 282 | 0.323 | 0.962 | 0.247 | 0.23 |
| 283 | 0.301 | 1.03 | 0.155 | 0.329 |
| 284 | 0.113 | 2.74 | 0.0462 | 1.1 |
| 285 | 0.0884 | 3.52 | 0.072 | 0.706 |
| 286 | 0.184 | 1.69 | 0.0602 | 0.845 |
| 287 | 0.15 | 2.08 | 0.112 | 0.455 |
| 288 | 0.0732 | 1.20 | 0.172 | 0.393 |
| | (0.0140, n = 7) | (0.268, n = 7) | (0.0288, n = 4) | (0.128, n = 4) |
| 289 | 0.0228 | 4.13 | 0.0544 | 1.33 |
| | (0.00219, n = 5) | (0.741, n = 5) | (0.00661, n = 6) | (0.325, n = 6) |
| 290 | 0.0629 | 1.09 | 0.179 | 0.279 |
| | (0.0118, n = 5) | (0.176, n = 5) | (0.0336, n = 6) | (0.0844, n = 6) |
| 291 | 0.118 | 0.746 | 0.150 | 0.188 |
| | (0.0226, n = 4) | (0.0858, n = 4) | (0.0253, n = 5) | (0.0294, n = 5) |
| 292 | 0.0682 | 1.10 | 0.183 | 0.143 |
| | (0.0192, n = 2) | (0.0631, n = 2) | (0.0328, n = 2) | (0.0191, n = 2) |
| 293 | 0.0562 | 1.36 | 0.132 | 0.197 |
| | (0.00736, n = 2) | (0.278, n = 2) | (0.000967, n = 2) | (0.00743, n = 2) |
| 294 | 0.183 | 0.75 | 0.185 | 0.249 |
| 295 | 0.281 | 0.481 | 0.116 | 0.398 |
| 296 | 0.198 | 0.564 | 0.0867 | 0.82 |
| 297 | 0.0451 | 3.00 | 0.111 | 0.445 |
| | (0.00597, n = 4) | (0.569, n = 4) | (0.0151, n = 5) | (0.0482, n = 5) |
| 298 | 0.0430 | 2.87 | 0.0957 | 0.507 |
| | (0.00578, n = 6) | (0.349, n = 6) | (0.0139, n = 7) | (0.0734, n = 7) |
| 299 | 0.932 | 0.146 | 2.54 | 0.0244 |
| | (0.507, n = 2) | (0.0780, n = 2) | (0.629, n = 4) | (0.00551, n = 4) |
| 300 | 0.0234 | 5.38 | 0.0867 | 0.636 |
| | (0.00482, n = 7) | (1.40, n = 7) | (0.0105, n = 9) | (0.0568, n = 9) |
| 301 | 0.0346 | 4.11 | 0.0441 | 1.61 |
| | (0.00764, | (0.921, n = 11) | (0.00684, n = 7) | (0.433, n = 7) |
| | n = 12) | | | |
| 302 | 0.0308 | 3.78 | 0.0275 | 1.96 |
| | (0.00225, n = 8) | (0.554, n = 7) | (0.00247, n = 6) | (0.175, n = 6) |
| 303 | 0.0254 | 4.20 | 0.104 | 0.602 |
| | (0.00381, n = 9) | (1.10, n = 9) | (0.0157, n = 6) | (0.0702, n = 6) |
| 304 | 0.0296 | 2.68 | 0.191 | 0.481 |
| | (0.00440, n = 7) | (0.425, n = 7) | (0.0386, n = 5) | (0.151, n = 5) |
| 305 | 0.0225 | 4.28 | 0.107 | 0.685 |
| | (0.00306, n = 7) | (0.953, n = 7) | (0.0149, n = 6) | (0.0980, n = 6) |
| 306 | 0.0191 | 3.97 | 0.0711 | 0.912 |
| | (0.00502, n = 4) | (0.582, n = 4) | (0.0202, n = 4) | (0.339, n = 4) |
| 307 | 0.0285 | 2.48 | 0.0437 | 1.29 |
| | (0.00345, n = 5) | (0.481, n = 5) | (0.00943, n = 4) | (0.238, n = 4) |
| 308 | 0.0262 | 4.06 | 0.0392 | 1.45 |
| | (0.00432, n = 8) | (1.01, n = 8) | (0.00818, n = 4) | (0.307, n = 4) |
| 309 | 0.0389 | 1.64 | 0.0330 | 1.27 |
| | (0.00473, n = 4) | (0.313, n = 4) | (0.00555, n = 3) | (0.312, n = 3) |
| 310 | 0.0176 | 5.56 | 0.0283 | 1.43 |
| | (0.00109, n = 4) | (1.23, n = 4) | (0.00985, n = 3) | (0.262, n = 3) |
| 311 | 0.0334 | 3.50 | 0.0393 | 1.04 |
| | (0.00431, n = 4) | (0.928, n = 4) | (0.0132, n = 3) | (0.220, n = 3) |
| 312 | 0.0207 | 4.76 | 0.0262 | 1.53 |
| | (0.00251, n = 5) | (1.32, n = 5) | (0.00799, n = 3) | (0.342, n = 3) |
| 313 | 0.0233 | 2.87 | 0.0388 | 0.867 |
| | (0.00223, n = 2) | (0.830, n = 2) | (0.00647, n = 3) | (0.197, n = 3) |
| 314 | 0.0290 | 2.81 | 0.0290 | 1.48 |
| | (0.0123, n = 2) | (0.669, n = 2) | (0.00867, n = 3) | (0.476, n = 3) |
| 315 | 0.0408 | 2.06 | 0.0651 | 1.00 |
| | (0.00771, n = 4) | (0.452, n = 4) | (0.0141, n = 4) | (0.245, n = 4) |
| 316 | 0.0240 | 3.75 | 0.122 | 0.644 ( |
| | (0.00478, n = 7) | (0.723, n = 7) | (0.00791, n = 6) | 0.146, n = 6) |
| 317 | 0.0948 | 1.01 | 0.172 | 0.333 |
| | (0.0240, n = 4) | (0.478, n = 4) | (0.00643, n = 3) | (0.104, n = 3) |
| 318 | 0.0547 | 1.69 | 0.124 | 0.482 |
| | (0.00365, n = 4) | (0.536, n = 4) | (0.0271, n = 4) | (0.188, n = 4) |
| 319 | 0.0540 | 2.77 | 0.113 | 0.522 |
| | (0.0220, n = 4) | (2.07, n = 4) | (0.00689, n = 3) | (0.171, n = 3) |
| 320 | 0.161 | 0.241 | 0.397 | 0.224 |
| 321 | 0.0752 | 0.517 | 0.204 | 0.437 |
| 322 | 0.146 | 0.266 | 0.711 | 0.125 |
| 323 | 0.0251 | 3.24 | 0.0597 | 1.32 |
| | (0.00551, n = 4) | (0.508, n = 4) | (0.00797, n = 4) | (0.527, n = 4) |
| 324 | 0.0374 | 2.13 | 0.0926 | 0.798 |
| | (0.00637, n = 4) | (0.185, n = 4) | (0.0147, n = 4) | (0.264, n = 4) |
| 325 | 0.0301 | 2.65 | 0.0586 | 1.16 |
| | (0.00366, n = 5) | (0.180, n = 5) | (0.0119, n = 5) | (0.224, n = 5) |
| 326 | 0.0754 | 1.5 | 0.0369 | 1.4 |
| 327 | 0.0548 | 1.47 | 0.27 | 0.191 |
| | (0.0274, n = ⅔) | (0.273, n = ⅔) | | |
| 328 | 0.0965 | 0.94 | 0.117 | 0.575 |
| 329 | 0.132 | 0.687 | 0.125 | 0.535 |
| 330 | 0.0919 | 0.562 | 0.199 | 0.255 |
| 331 | 0.0547 | 1.40 | 0.0929 | 0.808 |
| | (0.0162, n = 2) | (0.145, n = 2) | (0.0237, n = 2) | (0.277, n = 2) |
| 332 | 0.0745 | 0.949 | 0.184 | 0.345 |
| | (0.0143, n = 3) | (0.0533, n = 3) | (0.0348, n = 3) | (0.0711, n = 3) |
| 333 | 0.0492 | 2.31 | 0.131 | 0.487 |
| 334 | 0.0718 | 0.844 | 0.303 | 0.151 |
| 335 | 0.0477 | 1.27 | 0.122 | 0.374 |
| 336 | 0.0312 | 1.95 | 0.0874 | 0.523 |
| 337 | 0.0515 | 1.18 | 0.173 | 0.265 |
| 338 | 0.0472 | 1.29 | 0.174 | 0.262 |
| 339 | 0.0219 | 3.18 | 0.0986 | 0.574 |
| | (0.00722, n = 3) | (0.768, n = 3) | (0.0180, n = 4) | (0.231, n = 4) |
| 340 | 0.0823 | 0.852 | 0.252 | 0.183 |
| | (0.0288, n = 3) | (0.312, n = 3) | (0.00594, n = 4) | (0.0554, n = 4) |
| 341 | 0.238 | 0.213 | 0.373 | 0.0674 |
| 342 | 0.159 | 0.32 | 0.127 | 0.198 |
| 343 | 0.0422 | 1.84 | 0.124 | 0.662 |
| 344 | 0.0433 | 1.79 | 0.044 | 1.86 |
| 345 | 0.0649 | 2.16 | 0.035 | 0.937 |
| 346 | 0.144 | 0.604 | 0.128 | 0.210 |
| | (0.0284, n = 2) | (0.00648, n = 2) | (0.0378, n = 3) | (0.0493, n = 3) |
| 347 | 0.0827 | 0.872 | 0.102 | 0.245 |
| | | | (0.0247, n = 2) | (0.0142, n = 2) |
| 348 | 0.193 | 0.373 | 0.113 | 0.224 |
| | | | (0.00703, n = 2) | (0.0269, n = 2) |
| 349 | 0.117 | 0.756 | 0.121 | 0.214 |
| | (0.00129, n = 2) | (0.131, n = 2) | (0.0287, n = 3) | (0.0378, n = 3) |
| 350 | 0.189 | 0.741 | 0.107 | 0.307 |
| 351 | 0.298 | 0.47 | 0.149 | 0.22 |
| 352 | 0.127 | 0.815 | 0.142 | 0.201 |
| | (0.0116, n = 2) | (0.193, n = 2) | (0.0267, n = 3) | (0.0439, n = 3) |
| 353 | 0.497 | 0.145 | 1.09 | 0.0231 |
| | | | (0.0881, n = 2) | (0.00235, n = 2) |
| 354 | 0.233 | 0.441 | 0.540 | 0.0517 |
| | (0.0309, n = 2) | (0.0871, n = 2) | (0.126, n = 3) | (0.00657, n = 3) |
| 355 | 0.685 | 0.111 | 0.896 | 0.0285 |
| | (0.318, n = 2) | (0.0498, n = 2) | (0.00185, n = 2) | (0.00509, n = 2) |
| 356 | 0.386 | 0.364 | 0.506 | 0.0647 |
| 357 | 0.384 | 0.366 | 0.181 | 0.181 |
| 358 | 0.183 | 0.662 | 0.117 | 0.260 |
| | (0.0347, n = 2) | (0.0284, n = 2) | (0.0257, n = 2) | (0.0901, n = 2) |
| 359 | 0.172 | 0.706 | 0.131 | 0.227 |
| | (0.0235, n = 2) | (0.00679, n = 2) | (0.0201, n = 2) | (0.0647, n = 2) |
| 360 | 0.197 | 0.53 | 0.145 | 0.171 |
| 361 | 0.401 | 0.261 | 0.955 | 0.0259 |
| 362 | 0.302 | 0.347 | 0.365 | 0.0805 |
| | (n = 1/2) | (n = 1/2) | (0.0425, n = 2) | (0.0202, n = 2) |
| 363 | 0.145 | 0.835 | 0.208 | 0.146 |
| | (0.0185, n = 2) | (0.0159, n = 2) | (0.0485, n = 2) | (0.0522, n = 2) |
| 364 | 0.351 | 0.347 | 1.00 | 0.0303 |
| | (0.0809, n = 2) | (0.0290, n = 2) | (0.213, n = 2) | (0.0103, n = 2) |
| 365 | 0.0678 | 0.784 | 0.175 | 0.155 |
| 366 | 0.0889 | 0.598 | 0.366 | 0.074 |
| 367 | 0.0179 | 5.85 | 0.0641 | 0.386 |
| 368 | 0.0757 | 0.109 | 0.108 | 0.299 |
| 369 | 0.166 | 0.499 | 0.101 | 0.319 |
| 370 | 0.117 | 0.704 | 0.186 | 0.172 |
| 371 | 0.135 | 0.393 | 0.132 | 0.206 |
| 372 | 0.0781 | 0.68 | 0.365 | 0.0741 |
| 373 | 0.185 | 0.287 | 0.436 | 0.0621 |
| 374 | 0.0468 | 1.76 | 0.190 | 0.169 |
| 375 | 0.0471 | 1.13 | 0.152 | 0.178 |
| 376 | 0.0723 | 0.734 | 0.170 | 0.159 |
| 377 | 0.0544 | 0.976 | 0.136 | 0.199 |
| 378 | 0.067 | 0.793 | 0.191 | 0.142 |
| 379 | 0.079 | 0.672 | 0.238 | 0.114 |
| 380 | 0.142 | 0.374 | 0.236 | 0.115 |

As demonstrated by data in Table 3, Example compounds stimulate cAMP from human GLP-1R and GIPR in the presence of 0.1%0 casein.

### IN VIVO STUDIES

### Pharmacokinetics in Male CD-1 Mice

The pharmacokinetics of select Examples are evaluated following a single subcutaneous administration of 200 nMol/kg to male CD-1 mice. Blood samples are collected over 168 hours and resulting individual plasma concentrations are used to calculate pharmacokinetic parameters. Plasma (K3 EDTA) concentrations are determined using a qualified LC/MS method that measures the intact mass of the Examples. Each Example and an analog as an internal standard are extracted from 100% mouse plasma using immunoaffinity based precipitation with anti-GIP/GLP1 antibodies. Instruments are combined for LC/MS detection. Mean pharmacokinetic parameters are shown in Table 4.

TABLE 4
Mean Pharmacokinetic Parameters of peptides Following a Single Subcutaneous
Administration of 200 nMol/kg to Male CD-1 mice (N = 2/timepoint non-serial sampling).

| | T_{1/2} | Tₘₐₓ | Cₘₐₓ/D | AUCINF_D_obs | Cl/F |
|---|---|---|---|---|---|
| Example | (hr) | (hr) | (kg*nmol/L/nmol) | (hr*kg*nmol/L/nmol) | (mL/hr/Kg) |
| Example 1 | 17.54 | 12 | 4.84 | 135.61 | 7.37 |
| Example 2 | 7.55 | 6 | 5.4 | 77.23 | 12.95 |
| Example 3 | 15.04 | 6 | 4.42 | 158.49 | 6.31 |

Abbreviations: T_{1/2} = half-life, Tₘₐₓ = time to maximal concentration, Cₘₐₓ = maximal plasma concentration, AUCINF_D_obs = AUCinf divided by dose, CL/F = clearance/bioavailability.
Notes:
Data are the mean, where n = 2/timepoint/group.

Results from this study for Examples tested are consistent with an extended pharmacokinetic profile.
Pharmacokinetics in male Cynomolgus Monkeys

The pharmacokinetics of select Examples are evaluated following a single subcutaneous administration of 50 nMol/kg to male cynomolgus monkeys. Blood samples are collected over 336 hours and resulting individual plasma concentrations are used to calculate pharmacokinetic parameters. Peptide plasma (K₃ EDTA) concentrations are determined using a qualified LC/MS method that measured the intact mass of the compound. Each peptide and an analog as an internal standard are extracted from 100% cynomolgus monkey plasma using immunoaffinity based precipitation with anti-GIP/GLG1 antibodies. Instruments are combined for LC/MS detection. Mean pharmacokinetic parameters are shown in Table 5.

TABLE 5
Mean Pharmacokinetic Parameters of peptides
Following a Single Subcutaneous Administration
of 50 nMol/kg to Male Cynomolgus Monkeys.

| | | | | AUCINF_ | |
|---|---|---|---|---|---|
| | | | Cₘₐₓ/D | D_obs | |
| | T_{1/2} | Tₘₐₓ | (kg*nmol/ | (hr*kg*nmol/ | Cl/F |
| Example | (hr) | (hr) | L/nmol) | L/nmol) | (mL/hr/Kg) |
| Example 1 | 125.0 | 18 | 6.5 | 1458 | 0.69 |
| Example 2 | 102.1 | 24 | 11.7 | 2059 | 0.49 |
| Example 3 | 180.6 | 30 | 11.38 | 3420 | 0.29 |

Abbreviations: T_{1/2} = half-life, Tₘₐₓ = time to maximal concentration, Cₘₐₓ = maximal plasma concentration, AUCINF_D_obs = AUCinf divided by dose, CL/F = clearance/bioavailability.
Notes:
Data are the mean, where n = 2/group.
Notes:
Data are the mean, where n = 2/group. As seen in Table 5, results from this study for Example peptides tested are consistent with an extended pharmacokinetic profile.

### Pharmacokinetics in Male Sprague Dawley Rats Following Subcutaneous or Intrajejunal Administration

The pharmacokinetics of select Examples are evaluated following a single subcutaneous (SC) administration of 50 nMol/kg (dissolved in PBS, pH 7.4) or single 1 μmol/kg (mixed with 250 mM sodium decanoate (“C10”) and 12 mg/mL soybean trypsin inhibitor (SBTI)) intrajejunal (IJ) administration to male Sprague Dawley rats. Blood samples are collected over 168 hours following SC administration and 72 hours following IJ dosing. Pharmacokinetic parameters are calculated using individual plasma concentrations. A qualified LC/MS method that measures the intact mass of the Example is used to determine plasma (K₃ EDTA) concentrations. Each Example is tested with an analog peptide as an internal standard. Immunoaffinity based precipitation with anti-GIP/GLP1 antibodies is used to extract each test peptide and analog. Mean pharmacokinetic parameters for the Examples are shown in Table 6 and Table 7.

TABLE 6
Mean (+/− SD) Pharmacokinetic Parameters of peptides Following a Single
Subcutaneous Administration of 50 nMol/kg to Male Sprague Dawley rats.

| | T_{1/2} | Tₘₐₓ | Cₘₐₓ/D | AUCINF_D_obs | Cl/F |
|---|---|---|---|---|---|
| Example | (hr) | (hr) | (kg*nmol/L/nmol) | (hr*kg*nmol/L/nmol) | (mL/hr/Kg) |
| Example 1 | 44.7 (6.2) | 21.3 (4.6) | 3.34 (0.22) | 294.2 (30.0) | 3.42 (0.33) |
| Example 2 | 20.3 (0.9) | 14.7 (2.3) | 5.19 (0.20) | 231.7 (9.6) | 4.32 (0.17) |
| Example 3 | 32.1 (1.9) | 21.3 (4.6) | 4.71 (0.50) | 371.8 (21.8) | 2.70 (0.16) |

Abbreviations: T_{1/2} = half-life, Tₘₐₓ = time to maximal concentration, Cₘₐₓ = maximal plasma concentration, AUCINF_D_obs = AUCinf divided by dose, CL/F = clearance/bioavailability.
Notes:
Data are the mean, where n = 3/group (Table 6) As seen in table 6, results from this study using these Example peptides are consistent with an extended pharmacokinetic profile.

TABLE 7
Mean (+/− SD) Pharmacokinetic Parameters
of peptides Following a Single
Intrajejunal Administration of 1
μmol/kg to Male Sprague Dawley rats.

| | | Cₘₐₓ/D | AUCINF_D_obs |
|---|---|---|---|
| Example | Tₘₐₓ (hr) | (kg*nmol/L/nmol) | (hr*kg*nmol/L/nmol) |
| Example 1 | 1.33 (0.82) | 0.08 (0.05) | 1.31 (0.85) |
| Example 2 | 0.25 (0.13) | 0.56 (0.40) | 6.6 (4.4) |
| Example 3 | 0.33 (0) | 0.47 (0.16) | 8.45 (3.1) |

As illustrated by results in Table 7, these Examples are consistent with an exposure following intrajejunal administration. Intrajejunal exposure in this assay supports that the Examples may be suitable for oral formulation and administration.

### In Vivo Effect on Insulin Secretion in Male Wistar Rats

Male Wistar rats with femoral artery and femoral vein canulas (Envigo, Indianapolis, IN) (280-320 grams) are single-housed in polycarbonate cages with filter tops. Rats maintained on a 12:12 h light-dark cycle (lights on at 6:00 A.M.) at 21° C. and receive food and deionized water ad libitum. Rats are randomized by body weight and dosed 1.5 ml/kg s.c. at doses of 0.04, 0.1, 0.3, 1, 3, and 10 nmol/kg 16 hours prior to glucose administration then fasted. Animals are weighed and anesthetized with sodium pentobarbital dosed i.p. (65 mg/kg, 30 mg/ml). A time 0 blood sample is collected into EDTA tubes after which glucose is administered i.v. (0.5 mg/kg, 5 ml/kg). Blood samples are collected for glucose and insulin levels at time 2, 4, 6, 10, 20 and 30 min post intravenous administration of glucose. Plasma glucose levels are determined using a clinical chemistry analyzer. Plasma insulin is determined using an electrochemiluminescence assay (Meso Scale, Gaithersburg, MD). Glucose and insulin AUC are examined compared to the vehicle control with n=5 animals per group. Results are presented (SEM)(N).

TABLE 8
The effect of Example compounds on insulin secretion
during intravenous glucose tolerance test.
Dose (nmol/kg, s.c.)

| Ex- | | | | | | | |
|---|---|---|---|---|---|---|---|
| ample | 0.0 | 0.04 | 0.1 | 0.3 | 1.0 | 3 | 10 |
| 1 | 31.3 | 32.2 | 31.5 | 24.7 | 35.1 | 43.5 | 63.9 |
| | (2.8)(5) | (5.7)(5) | (4.5)(5) | (3.0)(5) | (4.0)(5) | (4.9)(5) | (6.5)(5) |
| 2 | 18.9 | 32.8 | 49.1 | 82.2 | 110.9 | 108.2 | 77.3 |
| | (4.3)(5) | (3.9)(5) | (4.8)(5) | (21.1)(5) | (23.1)(5) | (20.2)(5) | (8.8)(5) |
| 3 | 18.5 | 26.0 | 24.6 | 44.9 | 60.1 | 95.5 | 87.7 |
| | (1.0)(5) | (3.4)(5) | (3.9)(5) | (9.6)(5) | (4.0)(5) | (18.4)(5) | (7.9)(5) |
| 4 | 33.7 | 34.0 | 42.0 | 86.3 | 90.2 | 108.7 | 114.6 |
| | (5.3, 5) | (3.4, 5) | (3.8, 5) | (4.5, 5) | (9.2, 5) | (9.8, 5) | (16.1, 5) |
| 5 | 24.4 | 28.2 | 40.2 | 41.1 | 44.1 | 54.3 | 94.2 |
| | (3.0, 5) | (4.2, 5) | (6.0, 5) | (2.7, 5) | (4.5, 5) | (11.9, 5) | (10.1, 5) |

The data provided by Table 8 demonstrate a dose dependent increase in insulin secretion.

TABLE 9
ivGTT Insulin Secretion shown
by the following data:
Insulin secretion (ivGTT)

| Example | (ED₅₀, nmol/kg) (SEM, n) |
|---|---|
| 1 | >10 |
| 2 | 0.1 (0.05, 5) |
| 3 | 0.7 (0.3, 5) |
| 4 | 0.2 (0.05, 5) |
| 5 | 3 < ED₅₀ < 10 |

The data provided by Table 9 demonstrate dose dependent increase in insulin secretion.

### Studies in Diet-Induced Obese C57/B16 Mice

C57/B16 diet-induced obese (DIO) male mice (Taconic, Germantown, NY) weighing 41-50 g are used. Animals are individually housed in a temperature-controlled (24° C.) facility with a 12 hour light/dark photoperiod (lights off at 10:00 AM and lights on at 10:00 PM), with free access to food and water. After 2 week acclimatization to the facility, mice are randomized to treatment groups (n=6/group) based on body weight so each group has similar starting mean body weight.

Mice are treated with either vehicle (40 mM Tris-HCl at pH 8.0) or several peptides between the dose ranges of 0.03 nmol/kg to 10 nmol/kg. Treatments are subcutaneously administered to ad libitum fed DIO mice 30-90 minutes prior to the onset of the dark cycle daily (QD) for 14 days. During the course of the study, body weight and food intake are monitored daily.

All data are expressed as mean±SEM of 5-6 rats per group. Statistical analyses are assessed by one-way ANOVA followed by Dunnett's multiple comparison test to compare treatment groups to vehicle group or each other. Significant differences are identified at p<0.05.

$$Percent Body Weight = \frac{Body weight after 14 - day treatment}{Body weight before treatment started} \times 100$$

“0” dose group represents the vehicle-treated mice during each study. All data are expressed as mean±SEM of 5-6 mice per group. Statistical analyses are assessed by one-way ANOVA followed by Dunnett's multiple comparison test to compare treatment groups to ‘0’ dose (vehicle). *Significant differences are identified at p<0.05. Body weight change after treatment with Example compounds after 15 days. “Δ from vehicle” refers to difference between body weight at day 15 between test and vehicle groups. “% change” refers to percent decrease in body weight between days 1 and 15 in test groups. Percent decrease in body weight for animals receiving vehicle is recorded, and is less than about 100 in each study. The Δ from vehicle and % change data are statistically significantly different (p<0.05) than control for all Examples at all doses tested.

TABLE 10
The effect of GIP/GLP-1 receptor co-agonists on percent body
weight in diet-induced obese mice after 14-day of treatment.

| | Dose (nmol/kg, s.c., QD) | | | | | | |
|---|---|---|---|---|---|---|---|
| Peptide | 0 | 0.03 | 0.1 | 0.3 | 1 | 3 | 10 |
| Example 1 | 99.2 ± | 96.2 ± | 95.6 ± | 86.7 ± | 86.3 ± | 74.0 ± | 64.7 ± |
| | 0.8 | 1.2 | 0.9 | 1.3* | 1.9* | 3.8* | 2.6* |
| Example 2 | 100.5 ± | 101.5 ± | 95.0 ± | 86.5 ± | 76.4 ± | 76.4 ± | 68.1 ± |
| | 1.4 | 0.2 | 1.2 | 0.8* | 4.0* | 2.4* | 3.1* |
| Example 3 | 98.0 ± | 99.1 ± | 95.6 ± | 93.0 ± | 85.6 ± | 75.9 ± | 73.6 ± |
| | 0.7 | 1.3 | 1.3 | 1.1 | 0.8* | 4.3* | 1.7* |
| Example 4 | 98.3 ± | 96.6 ± | 94.7 ± | 88.5 ± | 76.9 ± | 66.6 ± | 64.5 ± |
| | 1.1 | 0.5 | 1.8 | 1.2* | 1.4* | 3.9* | 2.2* |
| Example 5 | 98.3 ± | 96.0 ± | 96.7 ± | 94.1 ± | 82.4 ± | 83.8 ± | 74.9 ± |
| | 1.1 | 1.3 | 1.1 | 1.8 | 1.6* | 1.6* | 2.3* |
| Example 104 | 99.2 ± | 94.0 ± | 94.1 ± | 89.0 ± | 82.7 ± | 70.8 ± | 71.3 ± |
| | 0.8 | 0.6 | 0.8 | 0.9* | 1.5* | 4.2* | 4.1* |
| Example 123 | 99.2 ± | 94.7 ± | 90.5 ± | 86.5 ± | 81.3 ± | 75.1 ± | 68.6 ± |
| | 0.8 | 0.9 | 1.6* | 1.1* | 2.0* | 1.8* | 1.9* |

As illustrated by data provided in Table 10 above, Example compounds tested in the assay dose-dependently reduce body weight in the studies described.

### Proteolytic Stability Assay

The proteolytic stability assay is a useful for assessing potential for oral delivery of peptides. The stability of peptides are compared in 1% rat small intestinal fluid (rSIF). The amount of intact peptide is measured for a sample peptide at 0, 3, 15, and 30 minutes to assess proteolytic stability. The amount of intact peptide for a sample peptide is measured in 90% pig small intestinal fluid (pSIF) at 0, 30, 45, and 60 minutes to assess the proteolytic stability.

Sample preparation when rat small intestinal fluid (rSIF) is used:

Peptides are prepared at 0.4 mg/mL in 50 mM Tris pH8.0. Rat small intestinal fluid is added at a ratio of 1% (v/v). The mixture is incubated at 37° C. at 150 rpm. Thirty μL of each sample are removed and placed into a new tube before the rSIF is added and at 3, 15, and 60 min. At each time point, the reaction was quenched by 1% TFA in 50% ACN at 1:1. The samples are diluted 100 times using dilution buffer (1:1 of 1% TFA in 50% ACN: 50 mM Tris pH8) and ready for analysis using mass spectrometry (MS).

Sample preparation when pig small intestinal fluid (pSIF) is used:

Peptides are diluted to a concentration of 0.4 mg/mL in 90% pig small intestinal fluid. After the mixing, 20 μL are immediately removed (time 0 for the time point of pre-incubation). The mixture is then incubated at 37° C. at 150 rpm. Twenty μL of each sample are removed and placed into a new tube at 30, 45, and 60 min. At each time point (0, 30, 45, 60), the reaction is quenched by 1% TFA in 50% ACN at 1:1. The sample is centrifuged at 20,000×g for 20 min at 4° C. The supernatant is diluted 100 times using dilution buffer (1:1 of 1% TFA in 50% ACN: 50 mM Tris pH 8) and ready for analysis using mass spectrometry (MS).

MS Conditions: The liquid chromatography separation is carried out on a Waters Acquity UPLC using mobile phase A (0.1% formic acid in water) and B (0.1% formic acid in acetonitrile and an ACQUITY UPLC Protein BEH C4 Column (300 Å, 1.7 μm, 1 mm×50 mm) at 40° C. The gradient is 5% of B during 0-1.5, 5-90% of B during 1.5-1.8, 90-95% of B during 1.8-3.0, 95-95% of B during 3.0-3.5, 95-5% of B during 3.5-4.0, and 5-5% of B during 4.0-5.0. The MS analysis is carried out on a Waters Xevo G2-XS QTOF. The data is acquired using MSe Continuum in the range of 50-2000 m/z in positive and sensitivity mode. The data analysis is performed using MassLynx.

TABLE 11
The percentage of each peptide not
cleaved at different time points using rSIF.

| | 0 min | 3 min | 15 min | 60 min |
|---|---|---|---|---|
| Example 1 | 100 | 82.4 | 41.4 | 1.6 |
| Example 2 | 100 | 75.5 | 18.3 | 0.3 |
| Example 3 | 100 | 68.8 | 25.8 | 0.3 |
| Example 4 | 100 | 97.9 | 99.3 | 89.4 |
| Example 69 | 100 | 2.2 | 0.0 | 0.0 |

The proteolytic peptide results provided in Table 11 suggest that the peptide of Example 4 may be suitable for oral formulation and delivery.

TABLE 12
The percentage of each peptide not
cleaved at different time points using pSIF.

| | 0 min | 30 min | 45 min | 60 min |
|---|---|---|---|---|
| Example 4 | 100 | 73.4 | 56.4 | 60.0 |
| Example 5 | 100 | 76.9 | 56.8 | 60.7 |

The proteolytic peptide results provided in Table 12 suggest that both the peptides of Examples 4 and 5 may be suitable for oral formulation and delivery.

### In Vivo Studies

The purpose of this study is to determine the relative potential for clinical immunogenicity of a compound.

### Methods:

CD8+ T cell depleted peripheral blood mononuclear cells are prepared and labeled with Carboxyfluorescein Diacetate Succinimidyl Ester (CFSE, Invitrogen) from a cohort of 10 healthy donors. Samples are tested in triplicate with 2.0 mL media control, keyhole limpet hemocyanin (“KLH”) (0.33 μM), anti-chemokine receptor type 4 (“CD4+”) (0.33 μM), and a compound of Examples 1, 2, and 3 (10 μM). Cultures are incubated for 7 days at 37° C. with 5% CO₂. On day 7, samples are analyzed by flow cytometry using High Throughput Sampler (HTS). Data is analyzed using FlowJo® Software (FlowJo, LLC, TreeStar).

### Results and Discussion

All donors produce a positive T cell response against KLH (100%). Analysis of the frequency and magnitude of the CD4+ T cell response for Example compounds is shown in Table 13.

TABLE 13
CD4+ T Cell Responses for Example
compounds and Positive Control (KLH).

| | % Donor | Median Response Strength |
|---|---|---|
| | Response | in positive donors (CDI) |
| KLH | 100% (n = 11) | 391 (n = 10) |
| Example 1 (GG-212) | 9% (n = 11) | 0.7 (n = 1) |
| Example 2 (GG-353) | 22% (n = 9) | 3.68 (n = 2) |
| Example 3 (GG-362) | 0% (n = 9) | NA (n = 0) |
| Example 4 (GG-427) | 0% (n = 9) | NA (n = 0) |
| Example 288 (GG-709) | 10% (n = 10) | 5.42 (n = 1) |
| Example 289 (GG-731) | 0% (n = 10) | NA (n = 0) |
| Example 301 (GG-650) | 0% (n = 10) | NA (n = 0) |
| Example 303 (GG-679) | 0% (n = 10) | NA (n = 0) |
| Example 316 (GG-698) | 0% (n = 10) | NA (n = 0) |

Cell Division Index (“CDI”): proportion of divided CD4+ T cells to the total number of CD4+ T cells in stimulated versus unstimulated samples.

These data show that the frequency of positive CD+ T cell response (CDI>2.5) was low for the compounds of Examples 1, 2, 3, 4, 288, 289, 301, 303 and 316, and the magnitude of the response in the few positive donors was low (CDI<6), indicating a low risk of immunogenicity using the CD4+ T cell assay.

### GLP-1R HEK293 Cell Membrane [³⁵S]GTPγS Binding Assay

The GLP-1 receptor is a G-protein coupled receptor that increases GTP-bound Gαₛ upon ligand induced receptor activation. The potency of peptides to stimulate—GLP-1R induced activation of Gαₛ is determined using preparations of purified membranes from HEK293 cells expressing the human GLP-1R. The assay is performed similarly to that as previously described (Bueno et al., J. Biol. Chem., (2016) 291, 10700 and Willard et al., Mol. Pharmacol. (2012) 82, 1066). The test peptides are solubilized in DMSO and diluted in reaction buffer containing 5 g of membrane in 20 mM HEPES pH 7.4, 50 mM NaCl, 5 mM MgCl₂, 40 g/ml saponin, 0.1% BSA, and 500 μM ³⁵S-labeled GTPγS for 30 minutes at room temperature. Reactions are terminated by addition of 0.2% Nonidet P-40 detergent containing rabbit anti-Gαₛ polyclonal antibody and 0.5 mg of anti-rabbit polyvinyltoluene beads. Mixtures are developed for 30 minutes, centrifuged at 80×g for 10 minutes, and counted for 1 minute/well using a MicroBeta TriLux instrument. Peptide concentration-response curves are fit to a four-parameter logistic model to calculate potency as an EC₅₀. Data normalization to % stimulation is performed using DMSO and GLP-1(7-36) as minimum and maximum controls for the receptor (Campbell et al, Assay Guidance Manual 2017). The potency of a sample peptide to stimulate GIPR induced activation of Gαₛ is reported in the Table 14. Assay results identify a petpide that is a partial agonist on the GLP-1R with respect to GLP-1R induced activation of Gαₛ.

### GLP-1R CHO Cell β-Arrestin Recruitment Assay

Activated G-protein coupled receptors can interact with the β-arrestin family of signalling proteins. The potency of peptides for GLP-1R induced arrestin recruitment is determined using the PathHunter Enzyme Fragment Complementation approach substantially as described (von Degenfeld et al., FASEB J., 2007 (14):3819-26 and Hamdouchi et al., J. Med Chem., 2016 59(24):10891-10916). CHO-K1 cells expressing Pro-Link-tagged Human GLP-1R and enzyme-acceptor-tagged β-arrestin-2 may be obtained from DiscoveRx and prepared as assay-ready frozen cells. Test peptides are solubilized in DMSO and serial dilutions are performed using the Echo acoustic dispenser (LabCyte). Assay media is the PathHunter Cell Assay Buffer (DiscoveRx) containing 0.1% w/v hydrolyzed Casein (Sigma). 100 nl of peptide is dispensed into 10 μl of assay media in a 384 well plate and then 10 μl of cells in assay media are added to give 5000 cells per well. Plates are incubated for 90 minutes in a 37′C/5% C02 incubator and 10 μl of PathHunter detection reagent is added (DiscoveRx) and plates are incubated at room temperature for 60 minutes. Luminescence signal is measured. Peptide concentration-response curves fit to a four-parameter logistic model to calculate potency as an EC₅₀. Data normalization to % stimulation is performed using DMSO and GLP-1(7-36) as minimum and maximum controls (Campbell et al, Assay Guidance Manual 2017). The potency of a sample peptide to stimulate GLP-1R induced β-arrestin recruitment is reported in Table 14. The assay results identify a peptide that is a partial agonist on the GLP-1R with respect to β-arrestin-2 recruitment.

TABLE 14

| | hGLP1R | hGLP1R | hGLP1R B- | |
|---|---|---|---|---|
| | GTPgS Rel | GTPgS % | Arrestin2 Rel | hGLP1R B- |
| | EC50 nM | Top (SEM, | EC50 uM | Arrestin2% |
| Example | (SEM, n) | n) | (SEM, n) | Top (SEM, n) |
| | 0.475 | 99.2 | 0.00274 | 104 |
| | (0.0322, | (0.659, | (0.000359, n = 42) | (3.45, n = 42) |
| | n = 115) | n = 115) | | |
| 1 | 0.235 | 91.1 | 0.005 | 105 |
| | (0.0201, n = 5) | (1.77, n = 5) | | |
| 2 | 0.642 | 95.9 | 0.00882 | 96.1 |
| | (0.0294, n = 2) | (0.553, n = 2) | (0.00269, n = 2) | (0.742, n = 2) |
| 3 | 0.421 | 95.4 | | |
| | (0.181, n = 2) | (2.20, n = 2) | | |
| 4 | 0.245 | 86.9 | 0.00480 | 92.4 |
| | (0.0638, n = 3) | (5.93, n = 3) | (0.000138, n = 2) | (14.0, n = 2) |
| 5 | 0.196 | 91.3 | | |
| | (0.0375, n = 3) | (6.90, n = 3) | | |
| 266 | 0.865 | 63.4 | 0.016 | 17.1 |
| | (0.328, n = 2) | (1.31, n = 2) | | |
| 267 | 0.867 | 62.3 | 0.00901 | 16.5 |
| 272 | 0.651 | 66.5 | >12.0 | ND |
| | (0.0427, n = 2) | (0.741, n = 2) | | |
| 298 | 1.03 | 57.3 | | |
| 300 | 0.405 | 85.6 | 0.0054 | 38.4 |
| 301 | 0.435 | 91.4 | 0.00267 | 93.7 |
| | (0.0848, n = 3) | (3.63, n = 3) | | |
| 302 | 0.268 | 98.6 | 0.00219 | 98.4 |
| 303 | 0.547 | 74.3 | 0.0179 | 47.7 |
| | (0.0998, n = 2) | (2.99, n = 2) | | |
| 304 | 0.561 | 77.1 | | |
| 305 | 0.389 | 76.3 | | |
| 306 | 0.378 | 76.1 | | |
| 315 | 0.601 | 44.2 | 0.0199 | 25.4 |
| 316 | 0.766 | 56.7 | 0.00608 | 26.1 |
| | (0.0469, n = 2) | (3.14, n = 2) | | |
| 317 | 0.536 | 53.7 | | |
| 318 | 0.415 | 58.4 | | |
| 288 | 0.666 | 66.7 | 0.00674 | 21.3 |
| | (0.104, n = 3) | (4.09, n = 3) | (0.00278, n = 3) | (1.94, n = 3) |
| 319 | 0.657 | 65.7 | | |
| 323 | 0.79 | 81.9 | | |
| 324 | 0.475 | 84.5 | | |
| 289 | 0.404 | 83.7 | 0.0124 | 51.3 |
| | (0.0247, n = 3) | (3.81, n = 3) | (0.00151, n = 3) | (6.05, n = 3) |
| 325 | 0.414 | 97.9 | | |
| 326 | 0.663 | 61.6 | | |
| 327 | 0.287 | 75.6 | 0.00379 | 41.6 |
| 328 | 0.481 | 66.3 | | |
| 329 | 0.343 | 83.6 | 0.00473 | 63.4 |
| 330 | 1.05 | 47.8 | >10.9 | ND |
| | (0.275, n = 2) | (1.16, n = 2) | | |
| 331 | 0.375 | 80.9 | 0.0128 | 44.3 |
| | (0.0274, n = 4) | (2.54, n = 4) | | |
| 332 | 0.453 | 81.5 | 0.0171 | 45.6 |
| | (0.0479, n = 4) | (4.65, n = 4) | | |
| 333 | 0.442 | 83.4 | 0.0548 | 58.2 |
| | (0.00535, n = 2) | (0.439, n = 2) | | |
| 334 | 0.432 | 70.2 | >10.3 | ND |
| 335 | 0.285 | 89.3 | 0.00531 | 73 |
| 336 | 0.377 | 90.2 | 0.00778 | 82.5 |
| 290 | 0.466 | 66.2 | 0.0238 | 20.0 |
| | (0.0664, n = 9) | (3.12, n = 9) | (0.00530, n = 4) | (1.15, n = 4) |
| 337 | 0.322 | 59.5 | 0.0174 | 34.1 |
| 338 | | | 0.0189 | 47 |
| 339 | 0.326 | 74.1 | 0.0107 | 44.0 |
| | (0.0357, n = 3) | (7.97, n = 3) | (0.00238, n = 2) | (2.40, n = 2) |
| 340 | 0.450 | 67.4 | 0.0107 | 20.1 |
| | (0.0182, n = 5) | (5.12, n = 5) | (0.00711, n = 4) | (2.31, n = 4) |
| 341 | 0.496 | 78.9 | 0.0188 | 21.9 |
| 342 | 0.414 | 77.2 | 0.035 | 20.7 |
| 343 | 0.522 | 74.9 | 0.0455 | 41.4 |
| 344 | 0.423 | 85.8 | 0.0343 | 46 |
| 345 | 0.684 | 62 | 0.00308 | 74.7 |
| | | | (0.000666, n = 2) | (2.86, n = 2) |
| 346 | 0.737 | 56.6 | 0.00325 | 19.3 |
| | (0.201, n = 3) | (3.94, n = 3) | (0.00105, n = 3) | (0.767, n = 3) |
| 347 | 0.759 | 46.9 | 0.00542 | 24.6 |
| | | | (0.00152, n = 2) | (2.81, n = 2) |
| 348 | 0.66 | 47.7 | 0.00346 | 19.8 |
| | | | (n = ½) | |
| 349 | 0.464 | 64.5 | 0.0151 | 18.4 |
| | (0.0290, n = 4) | (1.68, n = 4) | (0.00111, n = 2) | (0.337, n = 2) |
| 350 | 0.589 | 64.1 | 0.0108 | 21.8 |
| 351 | 0.563 | 66.5 | 0.0196 | 23.5 |
| 352 | 0.552 | 63.5 | 0.00421 | 17.4 |
| | (0.0267, n = 2) | (1.51, n = 2) | (n = ½) | |
| 353 | 1.96 | 63.1 | 0.559 | 23.0 |
| | | | (0.108, n = 2) | (0.408, n = 2) |
| 291 | 0.466 | 65.0 | >10.0 | ND |
| | (0.0476, n = 6) | (2.15, n = 6) | (n = ¼) | |
| 354 | 0.967 | 53.3 | 0.255 | 22.7 |
| | | | (n = ½) | |
| 355 | 1.76 | 50.6 | 0.363 | 18.3 |
| 356 | | | >10.5 | ND |
| 357 | | | 0.118 | 18.2 |
| 358 | 0.414 | 72.3 | 0.00938 | 21.4 |
| | (0.00356, n = 2) | (1.27, n = 2) | (0.00413, n = 3) | (2.47, n = 3) |
| 359 | 0.496 | 69.7 | 0.0841 | 28.4 |
| 360 | | | 0.0395 | 25.8 |
| 361 | | | 0.269 | 20.2 |
| | | | (n = ½) | |
| 362 | | | >11.0 | ND |
| 363 | 0.943 | 69.6 | 0.135 | 22.1 |
| 364 | | | >12.0 | ND |
| 292 | 0.429 | 71.5 | 0.00774 | 29.0 |
| | (0.0190, n = 4) | (3.33, n = 4) | (0.00199, n = 4) | (3.96, n = 4) |
| 293 | 0.368 | 70.6 | 0.00719 | 29.5 |
| | (0.0304, n = 4) | (0.715, n = 4) | (0.00168, n = 4) | (6.23, n = 4) |
| 365 | 0.464 | 66.9 | 0.00703 | 20.8 |
| | (0.0178, n = 3) | (1.35, n = 3) | (0.00233, n = 4) | (1.78, n = 4) |
| 366 | 0.409 | 67.6 | 0.00557 | 21.2 |
| | (0.0308, n = 3) | (3.55, n = 3) | (0.00363, n = 2) | (0.163, n = 2) |
| 367 | 0.289 | 89.7 | 0.00666 | 70.6 |
| | | | (0.00118, n = 2) | (8.61, n = 2) |
| 368 | 0.495 | 68.9 | 0.0479 | 21.3 |
| | | | (0.0205, n = 2) | (4.03, n = 2) |
| 369 | 0.381 | 58.8 | 0.0414 | 19.9 |
| | | | (n = ½) | |
| 370 | 0.428 | 63.7 | 0.00990 | 23.0 |
| | | | (0.00149, n = 2) | (0.470, n = 2) |
| 371 | 0.27 | 62.5 | 0.0142 | 21.7 |
| | | | (0.00333, n = 2) | (0.711, n = 2) |
| 372 | 0.379 | 69.1 | 0.00981 | 32.9 |
| | | | (0.00630, n = 2) | (1.65, n = 2) |
| 373 | 0.336 | 65.6 | 0.00954 | 22.9 |
| | | | (0.00348, n = 2) | (5.88, n = 2) |
| 374 | 0.345 | 67.1 | 0.0218 | 34.7 |
| 375 | 0.419 | 70.4 | 0.0114 | 24.2 |
| 376 | 0.326 | 72.6 | 0.0123 | 25.6 |
| 377 | 0.356 | 68.4 | 0.00532 | 16.7 |
| 378 | 0.359 | 68.6 | >10.2 | ND |
| 379 | 0.239 | 71 | 0.0181 | 31.9 |
| 380 | 0.188 | 66.8 | 0.0137 | 35 |
| 381 | 0.273 | 73.6 | 0.0155 | 25.8 |
| Comparator | 0.442 | 62.9 | >10.5 (n = ⅕) | ND |
| Tirzepatide | (se = 0.0311, | (se = 1.28, | | |
| | n = 9( | n = 9) | | |

| | hGLP1R B-Arrestin2 | hGLP1R B- |
|---|---|---|
| | Rel EC50 uM (SEM, | Arrestin2 % Top |
| Example | n) | (SEM, n) |
| | 0.00274 | 104 |
| | (0.000359, n = 42) | (3.45, n = 42) |
| 1 | 0.005 | 105 |
| 2 | 0.00882 | 96.1 |
| | (0.00269, n = 2) | (0.742, n = 2) |
| 3 | | |
| 4 | 0.00480 | 92.4 |
| | (0.000138, n = 2) | (14.0, n = 2) |
| 266 | 0.016 | 17.1 |
| 267 | 0.00901 | 16.5 |
| 272 | >12.0 | ND |
| 300 | 0.0054 | 38.4 |
| 301 | 0.00267 | 93.7 |
| 302 | 0.00219 | 98.4 |
| 303 | 0.0179 | 47.7 |
| 315 | 0.0199 | 25.4 |
| 316 | 0.00608 | 26.1 |
| 288 | 0.00674 | 21.3 |
| | (0.00278, n = 3) | (1.94, n = 3) |
| 289 | 0.0124 | 51.3 |
| | (0.00151, n = 3) | (6.05, n = 3) |
| 327 | 0.00379 | 41.6 |
| 329 | 0.00473 | 63.4 |
| 330 | >10.9 | ND |
| 331 | 0.0128 | 44.3 |
| 332 | 0.0171 | 45.6 |
| 333 | 0.0548 | 58.2 |
| 334 | >10.3 | ND |
| 335 | 0.00531 | 73 |
| 336 | 0.00778 | 82.5 |
| 290 | 0.0238 | 20.0 |
| | (0.00530, n = 4) | (1.15, n = 4) |
| 337 | 0.0174 | 34.1 |
| 338 | 0.0189 | 47 |
| 339 | 0.0107 | 44.0 |
| | (0.00238, n = 2) | (2.40, n = 2) |
| 340 | 0.0107 | 20.1 |
| | (0.00711, n = 4) | (2.31, n = 4) |
| 341 | 0.0188 | 21.9 |
| 342 | 0.035 | 20.7 |
| 343 | 0.0455 | 41.4 |
| 344 | 0.0343 | 46 |
| 345 | 0.00308 | 74.7 |
| | (0.000666, n = 2) | (2.86, n = 2) |
| 346 | 0.00325 | 19.3 |
| | (0.00105, n = 3) | (0.767, n = 3) |
| 347 | 0.00542 | 24.6 |
| | (0.00152, n = 2) | (2.81, n = 2) |
| 348 | 0.00346 | 19.8 |
| | (n = ½) | |
| 349 | 0.0151 | 18.4 |
| | (0.00111, n = 2) | (0.337, n = 2) |
| 350 | 0.0108 | 21.8 |
| 351 | 0.0196 | 23.5 |
| 352 | 0.00421 | 17.4 |
| | (n = ½) | |
| 353 | 0.559 | 23.0 |
| | (0.108, n = 2) | (0.408, n = 2) |
| 291 | >10.0 | ND |
| | (n = ¼) | |
| 354 | 0.255 | 22.7 |
| | (n = ½) | |
| 355 | 0.363 | 18.3 |
| 356 | >10.5 | ND |
| 357 | 0.118 | 18.2 |
| 358 | 0.00938 | 21.4 |
| | (0.00413, n = 3) | (2.47, n = 3) |
| 359 | 0.0841 | 28.4 |
| 360 | 0.0395 | 25.8 |
| 361 | 0.269 | 20.2 |
| | (n = ½) | |
| 362 | >11.0 | ND |
| 363 | 0.135 | 22.1 |
| 364 | >12.0 | ND |
| 292 | 0.00774 | 29.0 |
| | (0.00199, n = 4) | (3.96, n = 4) |
| 293 | 0.00719 | 29.5 |
| | (0.00168, n = 4) | (6.23, n = 4) |
| 365 | 0.00703 | 20.8 |
| | (0.00233, n = 4) | (1.78, n = 4) |
| 366 | 0.00557 | 21.2 |
| | (0.00363, n = 2) | (0.163, n = 2) |
| 367 | 0.00666 | 70.6 |
| | (0.00118, n = 2) | (8.61, n = 2) |
| 368 | 0.0479 | 21.3 |
| | (0.0205, n = 2) | (4.03, n = 2) |
| 369 | 0.0414 | 19.9 |
| | (n = ½) | |
| 370 | 0.00990 | 23.0 |
| | (0.00149, n = 2) | (0.470, n = 2) |
| 371 | 0.0142 | 21.7 |
| | (0.00333, n = 2) | (0.711, n = 2) |
| 372 | 0.00981 | 32.9 |
| | (0.00630, n = 2) | (1.65, n = 2) |
| 373 | 0.00954 | 22.9 |
| | (0.00348, n = 2) | (5.88, n = 2) |
| 374 | 0.0218 | 34.7 |
| 375 | 0.0114 | 24.2 |
| 376 | 0.0123 | 25.6 |
| 377 | 0.00532 | 16.7 |
| 378 | >10.2 | ND |
| 379 | 0.0181 | 31.9 |
| 380 | 0.0137 | 35 |
| 381 | 0.0155 | 25.8 |

### Composition for Oral Administration

A peptide is dissolved in Tris buffer (pH 8.0, 50 mM). A Permeation enhancer (“PE”) is prepared as follows: C10 is dissolved in Tris buffer (pH 8.0, 50 mM), LC, DPC, C12-maltoside and Rhamnolipid are each dissolved in phosphate buffered saline (“PBS”) (1×, pH 7.2). A solution of peptide, a PE, and a protease inhibitor is mixed to reach a final peptide concentration of 300 μM, PE at 100 mM (5% w/v for Rhamnolipid) and 1% (v/v) for the protease inhibitor. .

A peptide is incubated at 37° C. in 1% (v/v) rat small intestinal fluid or 50% (v/v) pig small intestinal fluid with and without a peptidase inhibitor. At different time points, samples are taken out, followed by quenching with 1% TFA in 50% ACN/water to stop the enzyme activity. The intact peptide at different time points is analyzed by high-performance liquid chromatography (HPLC) equipped with an ultraviolet (UV) detector or LC-MS/MS and normalized to the amount of peptide before mixing with the enzyme solution. A study using a petide of Example 2 and a peptide of Example 4 are reported in Table 15.

TABLE 15

| | % peptide intact | | | | |
|---|---|---|---|---|---|
| Recombinant protease inhibitor | Small | 0 | 15 | 30 | 60 |
| (concentration) | intestinal fluid | min | min | min | min |
| rSBTI (5 mg/mL) + Peptide (Example 4) | 50% v/v pig | 100.00 | 96.96 | 96.28 | 88.57 |
| Peptide Example 4 (no PI; control) | 50% v/v pig | 100.00 | 72.52 | 41.44 | 18.98 |
| rSBTI (5 mg/mL) + Peptide Example 2 | 50% v/v pig | 100.00 | 103.02 | 112.69 | 87.33 |
| Peptide Example 2 (no PI; control) | 50% v/v pig | 100.00 | 2.42 | 1.90 | 3.09 |
| rSBTCI (0.5 mg/mL) + Peptide Example 2 | 50% v/v pig | 100.00 | 131.71 | 126.53 | 123.70 |

Table 15 results support that an oral formulation composition for a peptide of Example 4 may be prepared using a PE and no PI.

### Oral Formulation Composition

Examples of formulation compositions for a peptide of this invention are provided by Table 16. The formulation compositions for peptides of this invention are in no way limited by the examples provided.

TABLE 16

| Formulation | Formulation composition | Concentration | |
|---|---|---|---|
| 1 | Peptide (Example 1; or | 2.4 | mg/mL |
| | Example 4 or Example 3) | | |
| | C10 | 250 | mM |
| | SBTI | 75 | mg/mL |
| 2 | Peptide (Example 1) | 2.4 | mg/mL |
| | LC | 500 | mM |
| | Citric acid | 500 | mM |
| 3 | Peptide (Example 1) | 2.4 | mg/mL |
| | NaTDC | 250 | mM |
| | SBTI | 75 | mg/mL |
| 4 | Peptide (Example 1, | 2.4 | mg/mL |
| | Example 2, or Example 4) | | |
| | C10 | 250 | mM |
| | SBTI | 12 | mg/mL |
| 5 | Peptide (Example 1 or | 2.4 | mg/mL |
| | Example 2) | | |
| | C10 | 125 | mM |
| | SBTI | 12 | mg/mL |
| | Peptide (Example 1) | 2.4 | mg/mL |
| 6 | C10 | 125 | mM |
| | SBTI | 24 | mg/mL |
| 7 | Peptide (Example 4) | 2.4 | mg/mL |
| | C10 | 250 | mM |
| | SFTI | 12 | mg/mL |

The effect of formulation composition on a peptide exposure is evaluated in rats via intrajejunal (IJ) administration using liquid formulations. To prepare liquid formulations for a rat IJ administration, a peptide, C10 or NaTDC and SBTI is dissolved in 50 mM Tris buffer pH 8.0 and mixed to achieve final desired concentration. For LC/citric acid formulation, LC and citric acid are dissolved in water and mixed with a peptide dissolved in Tris buffer. Formulation compositions provided in Table 16 may be administered as an oral composition.

### Enteric Capsules

An enteric capsule composition may be desired for certain peptides of this invention and may be prepared using methods for example, as set forth by Table 17. Enteric compositions may be prepared by blending ingredients together and filling the blend in enteric capsules.

An enteric composition of Table 17 is prepared adding half of the stated amount of sodium decanoate to a mortar. SBTI (for Examples 382-385) or SFTI (for Examples 386 and 387), and a peptide (peptides of Examples 1-4), as shown in Table 17. A remaining half of the sodium decanoate is added. A mixture is gently blended together using pestle, and spatula. If desired, additional mixing using pestle provides a homogenous blend. A capsule may be manually filled by individually weighing the required amount of blend, filling in capsules, and securely closing the capsule caps to the capsule bodies.

Dissolution testing of a single capsule is completed using known methods. A peptide of this invention may be formulated as an entric oral composition.

TABLE 17
Composition of Individual Enteric Capsule for Formulation

| | Enteric | Example | Enteric | Example | Enteric | Enteric |
|---|---|---|---|---|---|---|
| Peptide- | Example | Enteric | Example | Enteric | Example | Example |
| Component | 382 | 383 | 384 | 385 | 386 | 387 |
| Example 2 | 12.50 | | | | 12.50 | |
| Example 4 | | 12.50 | | | | 12.50 |
| Example 1 | | | 12.50 | | | |
| Example 3 | | | | 12.50 | | |
| Sodium | 250.00 | 250.00 | 250.00 | 250.00 | 250.00 | 250.00 |
| decanoate | | | | | | |
| (C10) | | | | | | |
| SBTI | 62.50 | 62.50 | 62.50 | 62.50 | | |
| SFTI | | | | | 62.50 | 62.50 |
| Total | 325.00 | 325.00 | 325.00 | 325.00 | 325.00 | 325.00 |
| Capsule Fill | | | | | | |
| Weight | | | | | | |
| Capsule Size | Size 00 | Size 00 | Size 00 | Size 00 | Size 00 | Size 00 |

Amino Acid Sequences
GIP (Human)
SEQ ID NO: 1
YAEGTFISDYSIAMDKIHQQDFVNWLLAQKGKKNDWKHNITQ
GLP-1 (7-36) (Human)
SEQ ID NO: 2
HAEGTFTSDVSSYLEGQAAKEFIAWLVKGR-NH₂
SEQ ID NO: 3
R₁X₁ X₂ X₃GT X₆TSD X₁₀ X₁₁ X₁₂ X₁₃ X₁₄D
X₁₆X₁₇AX₁₉ X₂₀ X₂₁ X₂₂X₂₃ X₂₄ X₂₅ X₂₆ X₂₇ X₂₈ X₂₉
X₃₀X₃₁
SEQ ID NO: 4
PX₃₂ X₃₃ X₃₄-R₂
SEQ ID NO: 5
PX₃₂X₃₃X₃₄X₃₅X₃₆X₃₇X₃₈X₃₉-R₂
SEQ ID NO: 6
PX₃₂ X₃₃ X₃₄ X₃₅X₃₆ X₃₇ X₃₈ X₃₉ X₄₀-R₂
SEQ ID NO: 7
K[(2-[2-(2-Amino-ethoxy)-ethoxy]-acetyl)₂-(γ-
Glu)-CO-(CH₂)_{q}-CO₂H] X₃₂ X₃₃ X₃₄-R₂
SEQ ID NO: 8
K[(2-[2-(2-Amino-ethoxy)-ethoxy]-acetyl)₂-(γ-
Glu)-CO-(CH₂)_{q}-CO₂H] X₃₂ X₃₃ X₃₄ X₃₅X₃₆ X₃₇ X₃₈
X₃₉-R₂
SEQ ID NO: 9
K[(2-[2-(2-Amino-ethoxy)-ethoxy]-acetyl)₂-(γ-
Glu)-CO-(CH₂)_{q}-CO₂H] X₃₂ X₃₃ X₃₄ X₃₅X₃₆ X₃₇ X₃₈
X₃₉ X₄₀-R₂
Example 1
SEQ ID NO: 10
Y-Aib-EGT-αMeF(2F)-TSDYSI-αMeL-LDEK((2-[2-(2-
Amino-ethoxy)-ethoxy]-acetyl)₂-(γ-Glu)-CO-
(CH₂)₁₈-CO₂H)AQ-Aib-EFI-(D-Glu)-YLIEGGPSSGAPPPS-
NH₂
Example 2
SEQ ID NO: 11
Y-Aib-EGT-αMeF(2F)-TSDYSI-αMeL-LD-Orn-K((2-[2-(2-
Amino-ethoxy)-ethoxy]-acetyl)₂-(γ-Glu)-CO-
(CH₂)₁₆-CO₂H)AQ-Aib-EFI-(D-Glu)-
YLIEGGPSSGAPPPS-NH₂
Example 3
SEQ ID NO: 12
Y-Aib-EGT-αMeF(2F)-TSDYSI-αMeL-LD-Orn-K((2-[2-(2-
Amino-ethoxy)-ethoxy]-acetyl)₂-(γ-Glu)-CO-
(CH₂)₁₈-CO₂H)AQ-Aib-EFI-(D-Glu)-YLIEGGPSSGAPPPS-
NH₂
Example 4
SEQ ID NO: 13
Y-Aib-EGT-αMeF(2F)-TSD-4Pal-SI-αMeL-LD-Orn-K((2-
[2-(2-Amino-ethoxy)-ethoxy]-acetyl)₂-(γ-Glu)-CO-
(CH₂)₁₆-CO₂H)AQ-Aib-EFI-(D-Glu)-αMeY-
LIEGGPSSGAPPPS-NH₂
Example 5
SEQ ID NO: 14
Y-Aib-EGT-αMeF(2F)-TSDVSI-αMeL-LD-Orn-K((2-[2-(2-
Amino-ethoxy)-ethoxy]-acetyl)₂-(γ-Glu)-CO-
(CH₂)₁₆-CO₂H)AQ-Aib-EFI-(D-Glu)-αMeY-
LIEGGPSSGAPPPS-NH₂
SEQ ID NO: 297
PSSG-R₂
SEQ ID NO: 298
PSSGAPPPS-R₂
SEQ ID NO: 299
PSSG
SEQ ID NO: 300
PSSG-NH₂
SEQ ID NO: 301
PSSGAPPPS
SEQ ID NO: 302
PSSGAPPPS-NH₂

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A compound, or a pharmaceutically acceptable salt thereof, of the formula:
R₁X₁X₂X₃GTX₆TSDX₁₀X₁₁X₁₂X₁₃X₁₄DX₁₆X₁₇AX₁₉X₂₀X₂₁X₂₂X₂₃X₂₄X₂₅X₂₆X₂₇X₂₈X₂₉X₃₀X₃₁ (SEQ ID NO:3) wherein:
R₁ is a modification of the N-terminal amino group wherein the modification is selected from the group consisting of Ac and absent;
X₁ is selected from the group consisting of Y, F, D-Tyr, and desY;
X₂ is selected from the group consisting of Aib, αMeP, A, P, and D-Ala;
or X₁ and X₂ combine to form desH-ψ[NHCO]-Aib;
X₃ is selected from the group consisting of E, N, Aad, and cTA;
X₆ is F;
X₁₀ is selected from the group consisting of A, L, H, 3Pal, 4Pal, V, Y, E, αMeF,
αMeF(2F), I, αMeY, Q, D-His, D-Tyr, cTA, and K(2-[2-(2-amino-ethoxy)-ethoxy]-acetyl)₂,-(γ-Glu)-CO—(CH₂)qCO₂H;
X₁₁ is selected from the group consisting of S, αMeS, and D-Ser;
X₁₂ is selected from the group consisting of I, S, D-I1e, and K(2-[2-(2-amino-ethoxy)-ethoxy]-acetyl)₂-(γ-Glu)-CO—(CH₂)qCO₂H;
X₁₃ is selected from the group consisting of Aib, L, and αMeL:
X₁₄ is selected from the group consisting of L and K, wherein K is conjugated to a C₁₆-C₂₂ fatty acid wherein said fatty acid is optionally conjugated to said K via a linker;
X₁₆ is selected from the group consisting of K, E, Om, Dab, Dap, S, T, H, Aib, αMeK, R, and K(2-[2-(2-amino-ethoxy)-ethoxy]-acetyl)₂-(γ-Glu)-CO—(CH₂)qCO₂H;
X₁₇ is selected from the group consisting of K, Q, I, and an amino acid conjugated to a C₁₆-C₂₂ fatty acid wherein said fatty acid is optionally conjugated to said amino acid via a linker;
X₁₉ is selected from the group consisting of Q, A, and K(2-[2-(2-amino-ethoxy)-ethoxy]-acetyl)₂-(γ-Glu)-CO—(CH₂)qCO₂H;
X₂₀ is selected from the group consisting of Aib, Q, H, R, K, αMeK, and K(2-[2-(2-amino-ethoxy)-ethoxy]-acetyl)₂-(γ-Glu)-CO—(CH₂)qCO₂H;
X₂₁ is selected from the group consisting of H, Aad, D, Aib, T, A, E, I, and K(2-[2-(2-amino-ethoxy)-ethoxy]-acetyl)₂-(γ-Glu)-CO—(CH₂)qCO₂H;
X₂₂ is selected from the group consisting of F and αMeF:
X₂₃ is selected from the group consisting of I, L, A, G, F, H, E, V, and K(2-[2-(2-amino-ethoxy)-ethoxy]-acetyl)₂-(γ-Glu)-CO—(CH₂)qCO₂H;
X₂₄ is selected from the group consisting of S, Aad, D-Glu, E, Aib, H, V, A, Q, D, P, and K(2-[2-(2-amino-ethoxy)-ethoxy]-acetyl)₂-(γ-Glu)-CO—(CH₂)qCO₂H;
X₂₅ is selected from the group consisting of Y and αMeY;
X₂₆ is selected from the group consisting of L, αMeL, and K(2-[2-(2-amino-ethoxy)-ethoxy]-acetyl)₂-(γ-Glu)-CO—(CH₂)qCO₂H;
X₂₇ is selected from the group consisting of L, I, and K(2-[2-(2-amino-ethoxy)-ethoxy]-acetyl)₂-(γ-Glu)-CO—(CH₂)qCO₂H;
X₂₈ is selected from the group consisting of E, A, S, D-Glu, and K(2-[2-(2-amino-ethoxy)-ethoxy]-acetyl)₂-(γ-Glu)-CO—(CH₂)qCO₂H;
X₂₉ is selected from the group consisting of Aib, G, A, and K(2-[2-(2-amino-ethoxy)-ethoxy]-acetyl)₂-(γ-Glu)-CO—(CH₂)qCO₂H;
X₃₀ is selected from the group consisting of C, G, G-R₂ and K(2-[2-(2-amino-ethoxy)-ethoxy]-acetyl)₂-(γ-Glu)-CO—(CH₂)_{q}—CO₂H;
X₃₁ is absent or is selected from the group consisting of PX₃₂X₃₃X₃₄—R₂ (SEQ ID NO:4), PX₃₂X₃₃X₃₄X₃₅X₃₆X₃₇X₃₈X₃₉—R₂ (SEQ ID NO:5), PX₃₂X₃₃X₃₄X₃₅X₃₆X₃₇X₃₈X₃₉X₄₀-R₂ (SEQ ID NO:6), K[(2-[2-(2-amino-ethoxy)-ethoxy]-acetyl)₂-(γ-Glu)-CO—(CH₂)_{q}—CO₂H]X₃₂X₃₃X₃₄-R₂ (SEQ ID NO:7), K[(2-[2-(2-amino-ethoxy)-ethoxy]-acetyl)₂-(γ-Glu)-CO—(CH₂)_{q}—CO₂H]X₃₂X₃₃X₃₄X₃₅X₃₆X₃₇X₃₈X₃₉-R₂ (SEQ ID NO:8), and K[(2-[2-(2-amino-ethoxy)-ethoxy]-acetyl)₂-(γ-Glu)-CO—(CH₂)_{q}—CO₂H]X₃₂X₃₃X₃₄X₃₅X₃₆X₃₇X₃₈X₃₉X₄₀—R₂ (SEQ ID NO:9); wherein:
X₃₂is S or K[(2-[2-(2-amino-ethoxy)-ethoxy]-acetyl)₂-(γ-Glu)-CO—(CH₂)_{q}—CO₂H];
X₃₃ is S or K[(2-[2-(2-amino-ethoxy)-ethoxy]-acetyl)₂-(γ-Glu)-CO—(CH₂)_{q}—CO₂H];
X₃₄ is selected from the group consisting of G, C, and K[(2-[2-(2-amino-ethoxy)-ethoxy]-acetyl)₂-(γ-Glu)-CO—(CH₂)_{q}CO₂H];
X₃₅ is A or K[(2-[2-(2-amino-ethoxy)-ethoxy]-acetyl)₂-(γ-Glu)-CO—(CH₂)_{q}—CO₂H];
X₃₆ is P or K[(2-[2-(2-amino-ethoxy)-ethoxy]-acetyl)₂-(γ-Glu)-CO—(CH₂)_{q}—CO₂H];
X₃₇ is P or K[(2-[2-(2-amino-ethoxy)-ethoxy]-acetyl)₂-(γ-Glu)-CO—(CH₂)_{q}—CO₂H];
X₃₈ is P or K[(2-[2-(2-amino-ethoxy)-ethoxy]-acetyl)₂-(γ-Glu)-CO—(CH₂)_{q}—CO₂H];
X₃₉ is selected from the group consisting of C, S, and K[(2-[2-(2-amino-ethoxy)-ethoxy]-acetyl)₂-(γ-Glu)-CO—(CH₂)_{q}—CO₂H];
X₄₀ is selected from the group consisting of C and K[(2-[2-(2-amino-ethoxy)-ethoxy]-acetyl)₂-(γ-Glu)-CO—(CH₂)_{q}—CO₂H];
q is selected from the group consisting of 14, 15, 16, 17, 18, 19, and 20; and
R₂ is a modification of the C-terminal group, wherein the modification is NH₂ or absent;
or a pharmaceutically acceptable salt thereof;
wherein if X₃₀ is G-R₂, then X₃₁ is absent;
wherein no more than one of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ may be a substituent that contains a fatty acid; and
wherein no more than one of X₃₀, X₃₄, X₃₉, and X₄₀ may be C; and
wherein if one of X₃₀, X₃₄, X₃₉, and X₄₀ is C, then none of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ is a substituent that contains a fatty acid.

56. A compound, or pharmaceutically acceptable salt thereof, as claimed by claim 55 wherein
R₁ is absent;
X₂ is Aib;
X₃ is E;
X₁₀ is Y;
X₁₁ is S;
X₁₂ is I;
X₁₄ is L;
X₁₆ is selected from the group consisting of K, E, Orn, Dab, Dap, S, T, H, Aib, αMeK, and R
X₁₇ is an amino acid conjugated to a C₁₆-C₂₂ fatty acid wherein said fatty acid is optionally conjugated to said amino acid via a linker;
X₁₉ is Q;
X₂₀ is selected from the group consisting of Aib, Q, H, and K;
X₂₁ is selected from the group consisting of H, D, T, A, and E;
X₂₂ is F;
X₂₃ is I;
X₂₄ is selected from the group consisting of D-Glu and E;
X₂₆ is L;
X₂₇ is I;
X₂₈ is selected from the group consisting of E, A, S, and D-Glu;
X₂₉ is selected from the group consisting of Aib, G, and A;
X₃₀ is selected from the group consisting of C, G, and G-R₂,
X₃₁ is absent or is selected from the group consisting of PX₃₂X₃₃X₃₄-R₂ (SEQ ID NO:4), PX₃₂X₃₃X₃₄X₃₅X₃₆X₃₇X₃₈X₃₉-R₂ (SEQ ID NO:5), and PX₃₂X₃₃X₃₄X₃₅X₃₆X₃₇X₃₈X₃₉X₄₀-R₂ (SEQ ID NO:6); wherein:
X₃₂is S;
X₃₃ is S;
X₃₄ is selected from the group consisting of G and C;
X₃₅ is A;
X₃₆ is P;
X₃₇ is P;
X₃₈ is P;
X₃₉ is selected from the group consisting of C and S; and
X₄₀ is C.

57. A compound, or a pharmaceutically acceptable salt thereof, as claimed by claim 56 wherein X₁₇ is K(2-[2-(2-amino-ethoxy)-ethoxy]-acetyl)₂-(γ-Glu)-CO—(CH₂)_{q}—CO₂H.

58. A compound, or a pharmaceutically acceptable salt thereof, as claimed by claim 57 wherein PX₃₂X₃₃X₃₄X₃₅X₃₆X₃₇X₃₈X₃₉-R₂ is selected from the group consisting of PSSGAPPPS (SEQ ID NO:301) and PSSGAPPPS-NH₂ (SEQ ID NO:302).

59. A compound, or pharmaceutically acceptable salt thereof, as claimed by claim 57 wherein
X₂₈ is A;
X₂₉ G;
X₃₀ is G;
(SEQ ID NO: 5)
X₃₁ is PX₃₂X₃₃X₃₄X₃₅X₃₆X₃₇X₃₈X₃₉-R₂
X₃₄is G; and
X₃₉ is S.

60. A compound, or pharmaceutically acceptable salt thereof, as claimed by claim 55 wherein
X₁ is selected from the group consisting of Y and D-Tyr; and X₁₃ is αMeL.

61. A compound, or pharmaceutically acceptable salt thereof, as claimed by claim 55 wherein q is 16.

62. A compound, or pharmaceutically acceptable salt thereof, as claimed by claim 55 wherein q is 18.

63. A compound, or pharmaceutically acceptable salt thereof, as claimed by claim 55 wherein the compound is selected from the group consisting of SEQ ID NO:303, SEQ ID NO:304, SEQ ID NO:305, SEQ ID NO:306, SEQ ID NO:307, SEQ ID NO.:308, and SEQ ID NO:392.

64. A compound, or pharmaceutically acceptable salt thereof, as claimed by claim 63 wherein the compound is SEQ ID NO:305.

65. A compound, or pharmaceutically acceptable salt thereof, as claimed by claim 63 wherein the compound is SEQ ID NO:307.

66. A compound, or pharmaceutically acceptable salt thereof, as claimed by claim 63 wherein the compound is SEQ ID NO:308.

67. A compound, or pharmaceutically acceptable salt thereof, as claimed by claim 63 wherein the compound is SEQ ID NO:392.

68. A compound, or pharmaceutically acceptable salt thereof, as claimed by claim 55 wherein the compound is a partial agonist on the GLP-IR.

69. A compound, or pharmaceutically acceptable salt thereof, as claimed by claim 68 wherein the compound stimulates GLP-1R induced activation of Gαₛ in the GIPR and GLP-1R HEK293 Cell Membrane Guanosine 5′-(gamma-thio) Triphosphate-[³⁵S](GTPγS) Binding Assay.

70. A compound, or pharmaceutically acceptable salt thereof, as claimed by claim 69 wherein the compound is a partial agonist on the GLP-1R with respect to the 3-arrestin-2 recruitment assay.

71. A method for treating a condition selected from the group consisting of type 2 diabetes mellitus, obesity, NAFLD, nonalcoholic steatohepatitis, dyslipidemia, and metabolic syndrome, comprising administering to a patient in need thereof, an effective amount of a compound, or a pharmaceutically acceptable salt thereof, as claimed by claim 55.

72. A method for treating obesity, comprising administering to a patient in need thereof, an effective amount of a compound, or a pharmaceutically acceptable salt thereof, as claimed by claim 55.

73. A method for providing therapeutic weight loss, comprising administering to a subject in need thereof, an effective amount of a compound, or a pharmaceutically acceptable salt thereof, as claimed by claim 55.

74. A method for treating type 2 diabetes mellitus comprising administering to a subject in need thereof, an effective amount of the compound, or a pharmaceutically acceptable salt thereof, as claimed by claim 55.

75. A pharmaceutical composition comprising the compound, or a pharmaceutically acceptable salt thereof, as claimed by claim 55 and at least one pharmaceutically acceptable carrier, diluent, or excipient.

76. A pharmaceutical composition as claimed by claim 75 wherein the composition is administered as a subcutaneous injection.

77. A pharmaceutical composition as claimed by claim 75 wherein the composition is administered orally.

78. A pharmaceutical composition as claimed by claim 77 wherein the composition comprises a permeation enhancer and at least one pharmaceutically acceptable carrier, diluent, or excipient.

79. A pharmaceutical composition as claimed by claim 78 wherein the permeation enhancer is selected from the group consisting of sodium decanoate (“C10”), sodium taurodeoxycholate (“NaTDC”), lauroyl carnitine (“LC”), dodecyl maltoside (“C12-maltoside”), dodecyl phosphatidylcholine (“DPC”), sodium taurodeoxycholate (“NaTDC”), and a Rhamnolipid.

80. A pharmaceutical composition as claimed by claim 79 wherein the permeation enhancer is selected from the group consisting of C10 and LC.

81. A pharmaceutical composition as claimed by claim 80 wherein the permeation enhancer is C10.

82. A pharmaceutical composition as claimed by claim 81 wherein the composition comprises a permeation enhancer and a protease inhibitor, and at least one pharmaceutically acceptable carrier, diluent, or excipient.

83. A pharmaceutical composition as claimed by claim 82 wherein the protease inhibitor is selected from the group consisting of soybean trypsin inhibitor (“SBTI”), soybean trypsin-chymotrypsin inhibitor (“SBTCI”), ecotin, sunflower trypsin inhibitor (“SFTI”), leupeptin, citric acid, ethylenediaminetetraacetic acid (“EDTA”), sodium glycocholate and 4-(2-aminoethyl)benzenesulfonyl fluoride hydrochloride (“AEBSF”).

84. A pharmaceutical composition a claimed by claim 83 wherein the protease inhibitor is selected from the group consisting of SBTI, SBTICI, and SFTI.

85. A pharmaceutical composition as claimed by claim 84 wherein the protease inhibitor is SBTI.

86. A pharmaceutical composition as claimed by claim 78 wherein the composition is a monolithic formulation.

87. A pharmaceutical composition as claimed by claim 78 wherein the composition is a multiparticulate formulation.

88. A pharmaceutical composition as claimed by claim 78 wherein the composition is a capsule or tablet.

89. A pharmaceutical composition as claimed by claim 86 wherein the composition is an enteric capsule or tablet.

90. (canceled)

91. (canceled)

92. (canceled)

93. (canceled)

94. (canceled)

95. (canceled)
